(19) United States
(12) Patent Application Publication
(10) Pub. No.: US 2023/0331804 A1
(43) Pub. Date: Oct. 19, 2023

(54) NANOPARTICLE SYSTEMS TO STIMULATE AND MAINTAIN IMMUNE SYSTEM RESPONSIVENESS AT TREATMENT SITES

(71) Applicants: Fred Hutchinson Cancer Center, Seattle, WA (US); Tidal Therapeutics, Inc., Cambridge, MA (US)

(72) Inventors: Matthias Stephan, Seattle, WA (US); Ulrik Nielsen, Quincy, MA (US)

(73) Assignees: Fred Hutchinson Cancer Center, Seattle, WA (US); Tidal Therapeutics, Inc., Cambridge, MA (US)

(21) Appl. No.: 17/758,296

(22) PCT Filed: Dec. 31, 2020
(86) PCT No.: PCT/US20/67729

§ 371 Date: Jun. 30, 2022

Related U.S. Application Data
(60) Provisional application No. 62/956,033, filed on Dec. 31, 2019.

(51) Int. Cl. C07K 14/725; A61P 35/00; A61K 47/68; A61K 47/69

(52) U.S. Cl. CPC C07K 14/7051; A61P 35/00; A61K 47/6849; A61K 47/6929

(57) ABSTRACT

Nanoparticle systems that genetically modify monocytes/macrophages in vivo to (1) recruit additional immune cells to a treatment site; (2) remain activated at the treatment site providing an on-going stimulatory signal to other immune cells; and (3) secrete bispecific immune-cell engaging antibodies that bind antigens on cells of interest at the treatment site and also bind and activate the recruited immune cells to destroy the bound cell. The systems can also inhibit the activity of transforming growth factor beta (TGFβ).

### CROSS-REFERENCE TO RELATED APPLICATION

This application is a U.S. National Phase Application based on International Patent Application No. PCT/US2020/067729, filed on Dec. 31, 2020, which claims priority to U.S. Provisional Patent Application No. 62/956,033, filed on Dec. 31, 2019, each of which is incorporated herein by reference in its entirety as if fully set forth herein.

### REFERENCE TO SEQUENCE LISTING

The Sequence Listing associated with this application is provided in text format in lieu of a paper copy, and is hereby incorporated by reference into the specification. The name of the text file containing the Sequence Listing is 2PT2710.TXT. The text is 204 KB, was created on Jun. 29, 2022, and is being submitted electronically via EFS-Web.

### FIELD OF THE DISCLOSURE

The disclosure provides nanoparticle systems that genetically modify monocytes/macrophages in vivo to (1) recruit additional immune cells to a treatment site; (2) remain activated at the treatment site providing an on-going stimulatory signal to other immune cells; and (3) secrete multi-specific immune-cell engaging molecules that bind antigens on targeted cells at the treatment site and also bind and activate the recruited immune cells to destroy the bound cell. The systems can also inhibit the activity of transforming growth factor beta (TGFβ).

### BACKGROUND OF THE DISCLOSURE

Macrophages are key immune effector cells that infiltrate cancerous tissue in high numbers. Within the tumor microenvironment, however, macrophages undergo a switch from an activated tumoricidal state to an immunosuppressive phenotype that actually facilitates tumor growth and metastasis. Pollard, Nat Rev Cancer 4, 71-78 (2004); Mantovani, et al., Nat Rev Clin Oncol (2017).

Understanding that immunosuppressed macrophages within the tumor microenvironment facilitate cancer growth and metastasis, much effort has been devoted to developing therapies that target immunosuppressive tumor-associated macrophages (TAMs). Many efforts to address TAMs have focused on killing the TAMs to alleviate immunosuppression in the tumor microenvironment. With this approach, however, the TAMs are simply replaced with newly-arriving macrophages at the tumor environment. Moreover, even when successful at killing some TAMs, most therapeutics developed to date have not been able to sufficiently penetrate into the tumor microenvironment. While some small molecule drugs and antibodies have shown some success, these approaches have suppressed all macrophages in the body, inducing dangerous side effects. Bowman & Joyce, Immunotherapy 6, 663-666 (2014). Thus, as is understood by everyone affected by cancer, more effective treatment strategies with fewer side effects are greatly needed.

Significant progress has been made in genetically engineering T cells of the immune system to target and kill cell types of interest, such as cancer cells. Many of these T cells have been genetically engineered to express a chimeric antigen receptor (CAR). CARs are proteins including several distinct subcomponents that allow the genetically modified T cells to recognize and kill cancer cells. The subcomponents include at least an extracellular component and an intracellular component. The extracellular component includes a binding domain that specifically binds a marker that is preferentially present on the surface of cells of interest. When the binding domain binds such markers, the intracellular component signals the T cell to destroy the bound cell. CARs additionally include a transmembrane domain that can link the extracellular component to the intracellular component, and other subcomponents that can increase the CAR's function. For example, the inclusion of one or more linker sequences, such as a spacer region, can allow the CAR to have additional conformational flexibility, often increasing the binding domain's ability to bind the targeted cell marker.

Clinical trials with CAR-expressing T cells (CAR-T) have shown positive responses in patients with refractory large B-cell lymphoma when conventional treatments had failed (Neelapu, et al 2017 N Engl J Med 377:2531-2544). However, while genetically engineered CAR-T cells result in cancer cell destruction, they have failed to provide prolonged anti-cancer activity in vivo for some indications. One reason for this failure could be based on the immunosuppressive effects of the tumor microenvironment.

Bispecific T-cell engaging antibodies bind both a cancer antigen on cancer cells and a T cell activating epitope, with the goal of bringing T cells to cancer cells to destroy the cancer cells. See, for example, US 2008/0145362. Most current bispecific T-cell engaging antibody therapeutics include paired monospecific, antibody-derived binding domains. Some have explored use of such antibodies in combinations that target two different T cell activating epitopes (e.g., CD3 and CD28). Many of these antibodies have short in vivo half-lives, however, so dosing remains a challenge.

Several groups have explored avenues to overcome some of the challenges associated with administration of bispecific T-cell engaging antibodies. For example, Stadler et al., (Nature. Medicine 23, 815-817) described injecting nanocarriers that deliver nucleic acids encoding bispecific T-cell engaging antibodies. By expressing these antibodies in vivo, this approach was able to achieve a sustained level of circulating bispecific T-cell engaging antibodies thereby avoiding infusion pumps for continuous delivery. Nonetheless, the circulating bispecific T-cell engaging antibodies do not penetrate solid tumors efficiently and the activity of T-cells recruited to and entering the tumor microenvironment is suppressed by myeloid suppressor cells.

Choi et al., (Nature Biotechnology, 37, 1049-1058, 2019) explored genetically engineering T cells to produce and secrete bispecific T-cell engaging antibodies. In fact, Choi et al., explored genetically engineering T cells to express CAR as well as to secrete bispecific T-cell engaging antibodies. These T cells, however, required ex vivo genetic engineering. Furthermore, CAR T-cells also do not efficiently infiltrate solid tumors and expand at the tumor site (often as a result of the myeloid suppressor cells). Thus, while there have been significant advances made in cancer treatment strategies, significant challenges nonetheless remain.

Furthermore, the transforming growth factor β (TGF-β) family of protein factors are found at high levels in solid tumors and contribute to immune dysfunction in the tumor microenvironment.

### SUMMARY OF THE DISCLOSURE

The current disclosure provides systems and methods to reverse the immunosuppressive, tumor supporting state of tumor-associated macrophages (TAMs) and turn these TAMs into highly activated, tumor cell-killing macrophages. Thus, the systems and methods disclosed herein do not simply aim to kill TAMs, but instead redirect their activity from tumor-promoting to tumor-destroying. In particular embodiments, the systems and methods are used as a therapeutic to induce the killing of cancer cells and/or to reduce or prevent the growth or development of new cancer cells. Data disclosed herein shows that these systems and methods are able to completely eradicate and suppress ovarian cancer, a notoriously difficult cancer type to control.

Use of the TAM-activating strategies disclosed herein has been shown to recruit immune cells to the tumor site. However, many of the recruited immune cells do not bind cancer antigens expressed by the tumor, and thus these recruited cells provide less benefit to the anti-cancer response than could be otherwise achieved. To address this issue, the current disclosure provides for genetically engineering the activated TAM to express multi-specific immune-cell engaging molecules. The activated TAM then provide three critical aspects to the success of the cancer therapies described herein. They (1) recruit immune cells to the tumor site; (2) remain activated at the tumor site providing an on-going stimulatory signal to other immune cells; and (3) secrete multi-specific immune-cell engaging molecules that bind cancer antigens at the tumor site and also bind and activate the recruited immune cells to destroy the bound cancer cell. The approach described to kill cancer cells can also be applied to other cell types of interest, such as diseased cells, autoreactive cells, infected cells, and microbial cells, to name a few.

Particular embodiments alter or maintain the activation states of macrophages in vivo by utilizing a nanoparticle system to deliver nucleotides encoding activation regulators, such as transcription factors. Particularly useful nanoparticles have a positive core and a neutral or negatively-charged surface and deliver nucleotides encoding (i) a transcription factor that creates and/or maintains the activation status of a macrophage; (ii) a kinase; and/or (iii) a multi-specific immune-cell engaging molecule. In preferred embodiments, the systems will include nanoparticles that deliver nucleotides encoding each of these components. A nanoparticle size of <130 nm ensures tumor infiltration. The nanoparticles can optionally include a TAM targeting ligand to direct more selective uptake of the nanoparticles by TAMs. As one example, TAMs express CD206, a cellular surface receptor that can be targeted by including mannose on the surface of the nanoparticles.

Particular embodiments include a nanoparticle that is <130 nm in diameter, has a positively-charged polymer core, and a neutral or negatively-charged coating. Nucleotides encoding interferon-regulatory factor 5 (IRF5); the kinase, IKKβ; a multi-specific antibody; and optionally a TGFβ inhibitor are encapsulated within the positively-charged polymer core. In this example, a bi-specific antibody binds a cancer antigen selected from EpCam or Tyrosinase related protein 1 (TYRP1/gp75) and an immune cell activating epitope selected from CD3, CD28, or 4-1BB.

Systems disclosed herein can additionally include a transforming growth factor beta (TGFβ) inhibitor.

### BRIEF DESCRIPTION OF THE SEVERAL VIEWS OF THE DRAWINGS

Some of the drawings submitted herein may be better understood in color. Applicants consider the color versions of the drawings as part of the original submission and reserve the right to present color images of the drawings in later proceedings.

FIGS. 1A-1D. Scheme to genetically transform tumor-associated macrophages (TAMs) into tumoricidal cells using targeted mRNA nanoparticles. (FIG. 1A) An injectable nanocarrier was developed to deliver in vitro transcribed mRNA encoding M1-polarizing transcription factors as a new method to rationally reprogram TAMs for therapeutic purposes without causing systemic toxicity. Illustrated is the first planned clinical application, designed to treat ovarian cancer patients with repeated intraperitoneal infusions of mRNA nanoparticles. (FIG. 1B) Scheme to genetically reprogram intracranial TAMs into tumoricidal macrophages using targeted mRNA nanoparticles. (FIG. 1C) Scheme to genetically transform tumor-associated macrophages (TAMs) into tumoricidal and bi-specific antibody-secreting cells using targeted mRNA nanoparticles. An injectable nanocarrier co-delivering in vitro transcribed mRNA encoding M1-polarizing transcription factors and antibodies that redirect T cells toward tumor antigen provides a new method to rationally reprogram TAMs and activate the host adaptive immune response for therapeutic purposes without causing systemic toxicity. (FIG. 1D) Exemplary formats of bi-specific binding molecules in Fc and non-Fc formats.

FIGS. 2A-2K. Nanoparticles carrying mRNA encoding IRF5 and IKKβ can imprint a pro-inflammatory M1-like phenotype. (FIG. 2A) Design of macrophage-targeted polymeric NPs formulated with mRNAs encoding key regulators of macrophage polarization. The nanoparticles consist of a PbAE-mRNA polyplex core coated with a layer of PGA-Di-mannose, which targets the nanoparticles to mannose receptors (CD206) expressed by M2-like macrophages. Also depicted is the synthetic mRNA encapsulated in the NP, which is engineered to encode the reprogramming transcription factors. (FIG. 2B) Transmission electron microscopy of a population of NPs (scale bar 200 nm) and a single NP (inset, scale bar 50 nm). (FIG. 2C) Size distributions of NPs, measured using a NanoSight NS300 instrument. (FIG. 2D) NPs demonstrated high transfection (46%) of bone marrow-derived macrophages (BMDMs) after 1 h exposure. (FIG. 2E) Gene-transfer efficiencies into bone marrow derived macrophages (BMDM) measured by flow cytometry 24 hours after nanoparticle transfection. (FIG. 2F) Relative viability of NP transfected and untransfected macrophages (assessed by staining with Annexin V and PI). N.s.; non-significant. (FIG. 2G) Expression kinetics of codon-optimized IRF5 mRNA (blue, left Y axis) and endogenous IRF5 mRNA (black, right Y axis) measured by qRT-PCR, n=3 for each time point. (FIG. 2H) Timelines depicting NP transfection protocols and culture conditions for the BMDMs used in FIGS. 2I-2K. (FIG. 2I) Gene expression profiles of IRF5/IKKβ. NP-transfected macrophages compared to signature M1 cells stimulated with the Toll-like Receptor 6 agonist MPLA. Results are depicted as a Volcano plot that shows the distribution of the fold changes in gene expression. M1 signature genes are indicated. P value of overlap between IRF5/IKKβ. NP-transfected macrophages and the M1 signature gene set was determined by GSEA. (FIG. 2J) Heat map of M1 signature gene expression in macrophages cultured in IL-4 versus cells cultured in IL-4 and transfected with IRF5/IKKβ. NPs. (FIG. 2K) Box plots showing mean counts for indicated genes and S.E.M.

FIG. 3. In vitro screening of the effect of different members of the interferon-regulatory factor (IRF) family (delivered in combination with or without their activating kinase) on the phenotype of mouse macrophages. BMDMs from C57BL/6 mice were incubated in M-CSF conditioning media and transfected with mRNA-PBAE NPs carrying synthetic mRNA encoding (1) control GFP, (2) murine IRF5, (3) murine IRF5 and the IKKβ kinase, which phosphorylates IRF5, (4) murine IRF8 and the IKKβ kinase, (5) murine IRF8 K310R, which is a mutant of IRF8, with a Lys-310 to Arg (K310R) conversion (White et al., J Biol Chem. 2016 Jun. 24), or (6) murine IRF7/3 (5D). This fusion protein includes the DNA binding domain (DBD) and constitutively active domain (CAD) of IRF-7 and the nuclear export signal (NES) and IRF association domain of IRF3 (Lin et al., Molecular and Cellular Biology. 18.5, 1998). Two days after NP transfection, cells were harvested for flow cytometric analysis for the TAM-associated macrophage marker Egr2 and the activated macrophage marker CD38. Based on this in vitro screen, NPs co-delivering mRNA encoding mIRF5 and IKKβ kinase were chosen for the remainder of in vitro and therapeutic in vivo experiments described herein.

FIGS. 4A-4J. Repeated intraperitoneal injections of mRNA nanocarriers delivering IRF5 and IKKβ genes into macrophages more than doubles mean survival of mice with disseminated ovarian cancer. (FIG. 4A) Time lines and dosing regimens. Arrows indicate time of I.P. injection. (FIG. 4B) Sequential bioluminescence imaging of tumor growth in control and treated mice. (FIG. 4C) Kaplan-Meier survival curves for treated versus control mice. Statistical analysis was performed using the log-rank test. (FIG. 4D) Flow cytometric quantitation of in vivo transfection rates in different immune cell subpopulations 48 hours after a single i.p. dose of D-mannose-coated NPs carrying GFP mRNA as a control: macrophages (CD45+, CD11b+, MHCII+, CD11c−, Ly6C−/low, Ly6G−), monocytes (CD45+, CD11b+, MHCII+, CD11c−, Ly6C+, Ly6G−), neutrophils (CD45+, CD11b+, MHCII+, CD11c−, Ly6G+), CD4+ T cells (CD45+, TCR−/3 chain+, CD4+, CD8−), CD8+ T cells (CD45+, TCR-β chain+, CD4−, CD8+), and natural killer cells (CD45+, TCR-β chain−, CD49b+) were measured. (FIG. 4E) Flow cytometric analysis of macrophage phenotypes in the peritoneum of mice with disseminated 1D8 ovarian cancer. Animals were either treated with 4 doses of IRF5/IKKβ. NPs or PBS. (FIG. 4F) Box plots summarizing relative percent (left panel) and absolute numbers (right panel) of Ly6C−, F4/80+, and CD206+(M2-like) macrophages. (FIG. 4G) Corresponding numbers for Ly6C−, F4/80+, and CD206− (M1-like) macrophages. (FIG. 4H) Representative hematoxylin and eosin-stained sections of ovarian tumor-infiltrated mesenteries isolated from PBS controls (top panel) or IRF5/IKKβ. NP-treated animals (bottom panel; scale bar 100 μm). 10-fold magnifications of representative malignant lesions are shown on the right (scale bar 50 μm). (FIG. 4I) Luminex assay measuring cytokines produced by isolated peritoneal macrophages from each treatment group. CD11b+, F4/80+ peritoneal macrophages were isolated by fluorescence activated cell sorting, and cultured ex vivo. After 24 hours, cell culture supernatants were collected. In parallel experiments, FACS-sorted CD11b+, F4/80+ peritoneal macrophages were directly analyzed by pRT-PCR to determine expression levels of four master regulators of the macrophage phenotypes (SerpinB2, Retnla, Ccl11, and Ccl5). Results are summarized as box plots in FIG. 4J.

FIGS. 5A-5F. Macrophage-programming mRNA nanocarriers are highly biocompatible and safe for repeated dosing. (FIG. 5A) In vivo biodistribution of macrophage-targeted IRF5/IKKβ. NPs following i.p. administration. NP-delivered (codon optimized) mRNA was detected by qPCR 24 hours after a single injection of nanoparticles containing 50 μg mRNA. (FIG. 5B) Schematic representation of the experimental timeline. *Twenty-four hours after the last dose, mice were euthanized by CO₂ inhalation. Blood was collected through retro-orbital bleeding into heparin coated tubes for serum chemistry and complete blood count. Necropsy was performed for histological analysis of liver, spleen, pancreas, mesentery and omentum, stomach, and urinary bladder. (FIG. 5C) Representative hematoxylin and eosin-stained sections of various organs isolated from controls or NP-treated animals. Scale bar, 100 μm. Lesions found in the NP-treated animals are shown and described here based on analysis by a Comparative Pathologist. The relevant findings for each numbered image is: [1] Discrete foci of cellular infiltrates largely composed of mononuclear cells admixed with a few granulocytes; Mild extramedullary hematopoiesis. [2] In a few locally extensive areas, hepatocytes are mild to moderately swollen. [3] Moderate myeloid (predominant), erythroid and megakaryocyte hyperplasia within the red pulp. [4] Mild hypocellularity of the white pulp. [5] Within the mesentery, there are moderate, multifocal infiltrates of macrophages, lymphocytes, plasma cells and granulocytes. [6] Mild to moderate infiltrates of macrophages admixed with lymphocytes, plasma cells and granulocytes; Mild dissociation of the acini and acinar loss; Mild diffuse loss of zymogen granules from the acinar cells. [7] Dense aggregates of lymphocytes admixed with macrophages around fat tissue. [8] Mild multifocal vacuolar degeneration of the chief and parietal cells within the gastric mucosa. (FIG. 5D) Serum chemistry and blood counts. (FIGS. 5E, 5F) Luminex assay measurements of serum IL-6 (FIG. 5E) and TNF-α (FIG. 5F) cytokines 4 or 8 days after a single i.p. injection of IRF5/IKKβ NPs.

FIGS. 6A-61. Intravenously infused IRF5/IKKβ nanoparticles can control tumor metastases in the lung. (FIG. 6A) In vivo biodistribution of macrophage-targeted IRF5/IKKβ. NPs following i.v. administration. Codon-optimized mRNA was measured by qPCR 24 hours after a single i.v. injection of nanoparticles containing 50 μg mRNA. (FIGS. 6B-6H) C57BL/6 albino mice were injected via tail vein with 1×10⁶ B16F10 firefly luciferase-expressing melanoma cells to establish lung metastases. After 7 days, animals were randomly assigned to either the IRF5/IKKβ. NP treatment group, the control GFP NP group, or the PBS control. (FIG. 6B) Time lines and dosing regimens. (FIG. 6C) Confocal microscopy of healthy lungs (left panel) and B16F10 tumor-infiltrated lungs (right panel). Infiltrating macrophage populations fluoresce in green. (FIG. 6D) Sequential bioluminescence tumor imaging. (FIG. 6E) Kaplan-Meier survival curves for each treatment group. ms indicates median survival. Statistical analysis was performed using the log-rank test, and P<0.05 was considered significant. (FIG. 6F) Representative photographs (top row) and micrographs of lungs containing B16F10 melanoma metastases representing each group following 2 weeks of treatment. (FIG. 6G) Counts of lung tumor foci. (FIG. 6H) Phenotypic characterization of monocyte/macrophage populations in bronchoalveolar lavage from each treatment group. (FIG. 6I) Summary of the relative percentages of suppressive and activated macrophages.

FIGS. 7A-7F. Macrophage reprogramming improves the outcome of radiotherapy in glioma. (FIG. 7A) T2 MRI scan, and histological staining following initiation of a PDGFβ-driven glioma in RCAS-PDGF-B/Nestin-Tv-a; Ink4a/Arf−/−; Pten−/−transgenic mice on post-induction day 21. (FIG. 7B) Confocal microscopy of CD68+ TAMs infiltrating the glioma margin. Scale bar 300 μm. (FIG. 7C) Flow cytometry analysis of macrophage (F4/80+, CD11b+) populations in healthy brain tissue versus glioma. (FIGS. 7D-7E) Kaplan-Meier survival curves of mice with established gliomas receiving IRF5/IKKβ treatments as a monotherapy (FIG. 7D) or combined with brain tumor radiotherapy (FIG. 7E). Time lines and dosing regimens are shown on top. Ms, median survival. Statistical analysis was performed using the log-rank test, and P<0.05 was considered statistically significant. (FIG. 7F) Sequential bioluminescence imaging of tumor progression.

FIGS. 8A-8E. IVT mRNA-carrying nanoparticles encoding human IRF5/IKKβ efficiently reprogram human macrophages. (FIG. 8A) Time line and culture conditions to differentiate the human THP-1 monocytic cell line into suppressive M2-like macrophages. (FIG. 8B) Bioluminescent imaging of M2-differentiated THP1-Lucia cells cultured in 24 wells and transfected with indicated concentrations of NPs carrying human IRF5/IKKβ mRNA versus control GFP mRNA. Levels of IRF-induced Lucia luciferase were determined 24 hours after transfection using Quanti-Luc. (FIG. 8C) Summary of bioluminescent counts. (FIGS. 8D-8E) Differences in IL-1β cytokine secretion (FIG. 8D) and surface expression (FIG. 8E) of the M1-macrophage marker CD80.

FIG. 9. List of antibodies used in myeloid and lymphoid immunophenotyping panels described in Example 1.

FIGS. 10A, 10B. T cells contribute to anti-tumor effects achieved with macrophage-programming nanoparticles. (10A) Nanoparticle-mediated macrophage programming increases T cell recruitment into tumor lesions. Shown are representative confocal images of peritoneal metastases of ID8 ovarian cancer cells in the mesentery. Tissues were collected after 6 bi-weekly i.p. injections of PBS or IRF5/IKKβ NPs (50 μg mRNA/dose) and were stained for the indicated lymphocyte- and myeloid-markers (a, c). Tu=Tumor, Mes=Mesentery. Scale bar: 100 μm. (10B) Box plots showing fluorescent signals for each phenotypic marker using Halo™ image analysis software. N=5. The boxes represent the mean values and the line in the box represents median. The bars across the boxes show the minimum and maximum values. Whiskers represent 95% confidence intervals. N=5 biologically independent samples.

FIG. 11. Exemplary sequences supporting the disclosure.

FIG. 12. Protein/encoding sequence pairings with associated notes.

### DETAILED DESCRIPTION

Macrophages are key immune effector cells that infiltrate cancerous tissue in high numbers. However, within an immunosuppressive tumor milieu, they undergo a switch from an activated tumoricidal state to an immunosuppressive phenotype, which facilitates tumor growth and metastasis. These tumor-associated immunosuppressed macrophages (TAMs) are associated with poor prognosis (Komohara Y et al. (2014) Cancer science 105(1): 1-8). They induce angiogenesis, lymphogenesis, and stroma remodeling. They also play a key role in promoting tumor invasion and metastasis through secretion of the enzymes plasmin, uPA, matrix metalloproteinases (MMPs) and cathepsin B (Komohara, Y et al. (2016) Advanced drug delivery reviews 99: 180-185; Gocheva V et al. (2010) Genes Dev 24: 241-255; Wang R et al. (2011) Lung Cancer 74: 188-196). Apart from mediating tumor growth and progression, TAMs can also interact with other immune cells and suppress innate and adaptive antitumor immune responses.

Several small molecule drugs focus on blocking the localization of TAM-precursor cells to tumors by targeting the pathways involved in cell recruitment or expansion (i.e. inhibitors of the CSF-1/CSF-1R pathway (Pyon; teck et al. Nat Med 19, 1264-1272 (2013); Tap et al. N Engl J Med 373, 428-437 (2015)) or the CCL2 pathway (Nywening, et al. Lancet Oncol 17, 651-662 (2016)). These approaches require repeated systemic exposure to large doses of the small molecule drugs. Furthermore, clinical trials of these drugs showed low responses unless they were combined with cytoreductive therapies. Nywening, et al. Lancet Oncol 17, 651-662 (2016); Butowski et al. Neuro Oncol 18, 557-564 (2016). Furthermore, these small molecule approaches do not actively promote macrophage anti-tumor activity.

Conventional nanocarriers such as liposomes have been formulated with bisphosphonates or other antiproliferative agents to systemically destroy macrophages within a tumor (i.e. liposomal-clodronate) (Fritz et al., Front Immunol 5, 587 (2014)). Oncolytic viruses have also been used to deliver siRNA to silence immune-evasion pathways within tumors and indirectly promote phagocytosis of TAMs. (Chao et al., Curr Opin Immunol 24, 225-232 (2012)). The macrophages that are destroyed using these approaches, however, are naturally replaced by newly-arriving macrophages that similarly become immunosuppressive.

Antibodies have been developed to induce functional activation of TAMs. These approaches utilize antibodies to target defined antigen types within the tumor. Mantovani, et al., Nat Rev Clin Oncol (2017) Success of these antibodies, however, is limited by their low tumor penetration and heterogeneous distribution. Thurber et al., Adv Drug Deliv Rev 60, 1421-1434 (2008). They also do not address tumor escape variants that lack the antigen targeted by the antibody.

None of the described approaches directly and effectively program or reprogram TAMs to remain or become activated tumoricidal macrophages, as disclosed herein. The systems and methods disclosed herein are significantly innovative because they allow the reprogramming of TAMs to become tumor-clearing macrophages while simultaneously reducing the tumor-promoting TAM burden. Currently, no other method exists that allow physicians to rationally reprogram TAMs for these therapeutic purposes. Mantovani, et al., Nat Rev Clin Oncol (2017); Gabrilovich & Nagaraj, Nat Rev Immunol 9, 162-174 (2009). This in and of itself can provide therapeutic benefit in the treatment of tumors. In fact, efficacy of the approaches disclosed herein has been demonstrated in models of ovarian cancer, melanoma, and glioblastoma. More particularly, infusions of nanoparticles formulated with nucleotides encoding interferon regulatory factor 5 (IRF5) in combination with its activating kinase IKKβ reversed the immunosuppressive, tumor-supporting state of TAMs and reprogrammed them to a phenotype that induced anti-tumor immunity and promoted tumor regression.

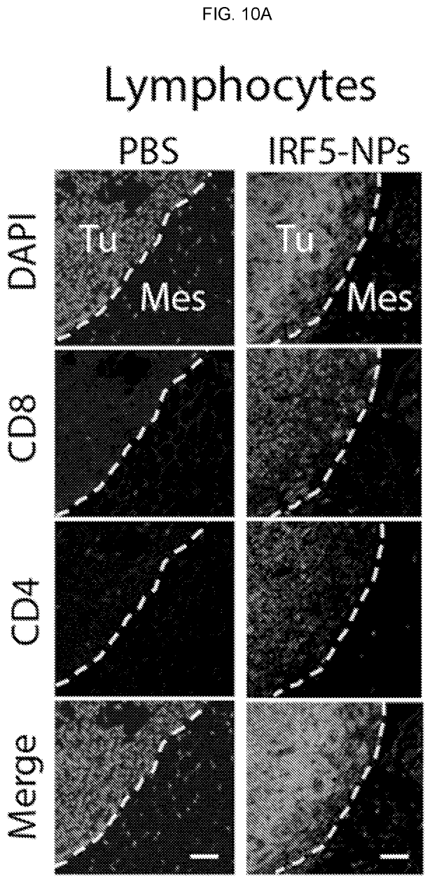

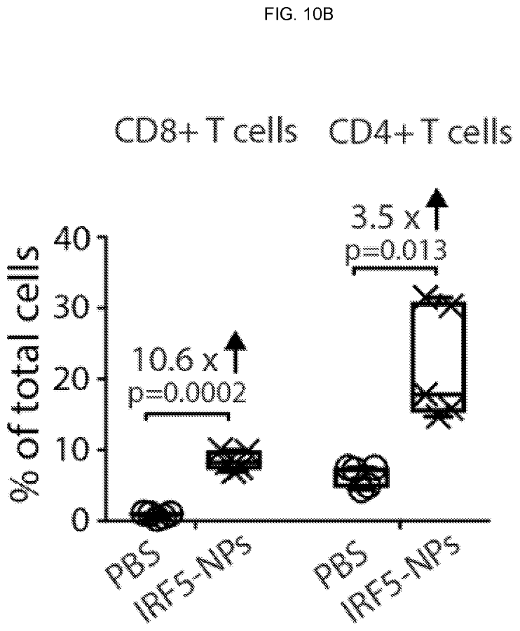

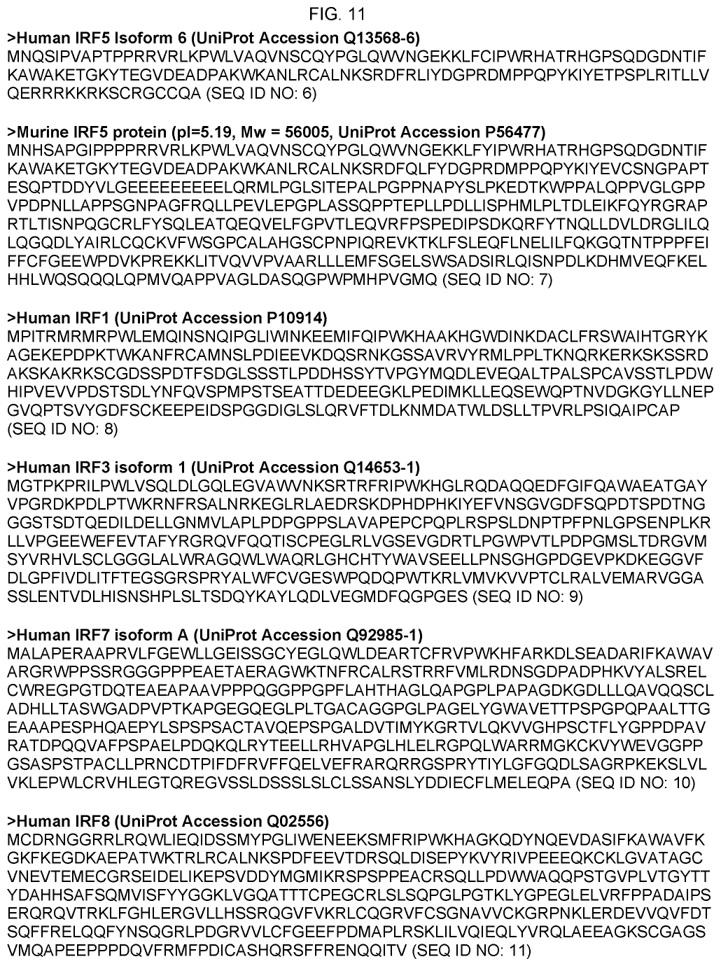

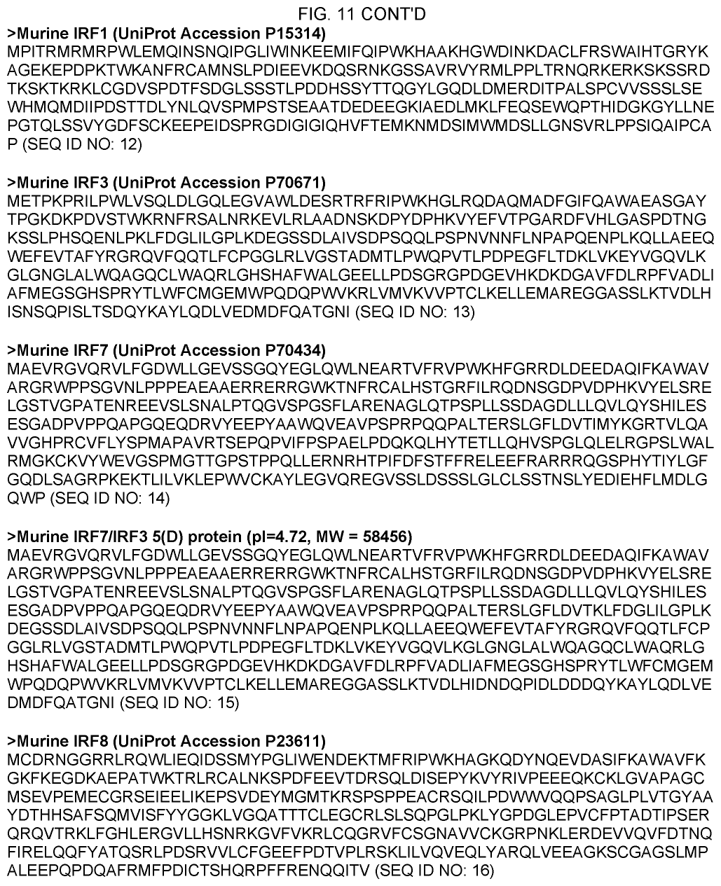

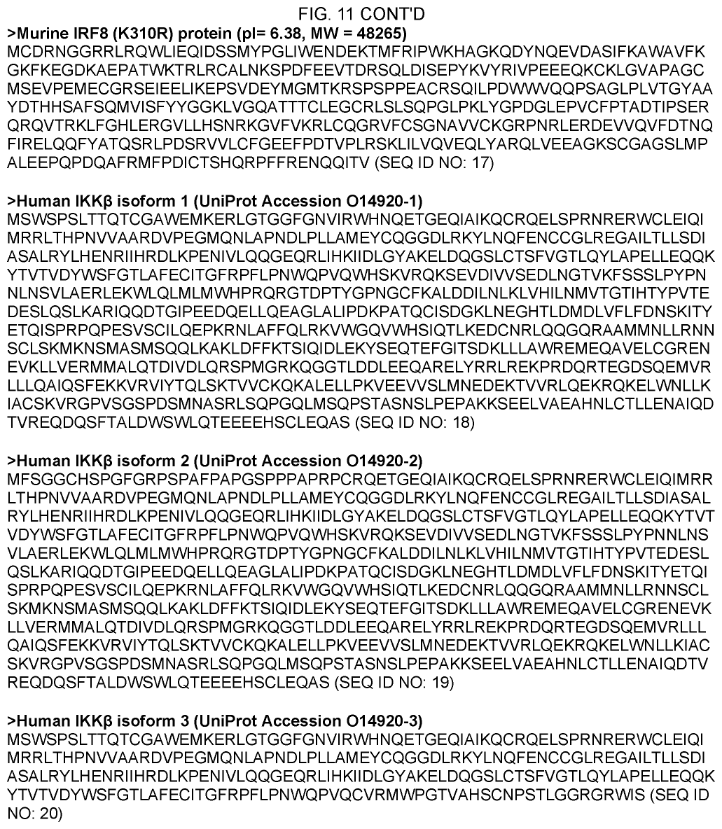

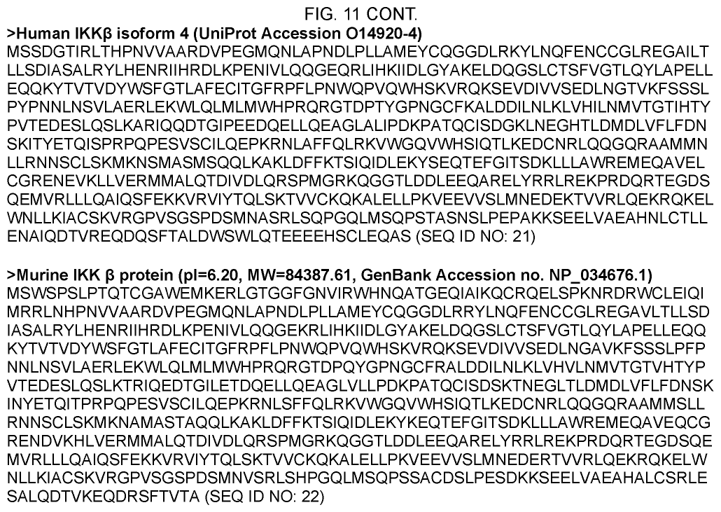

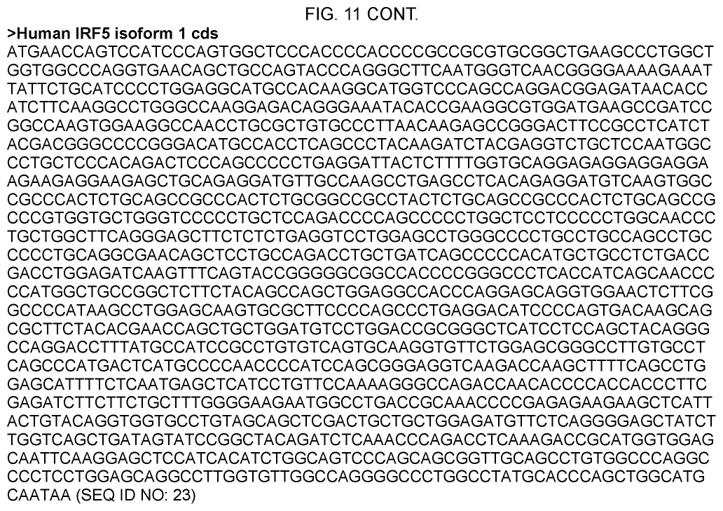

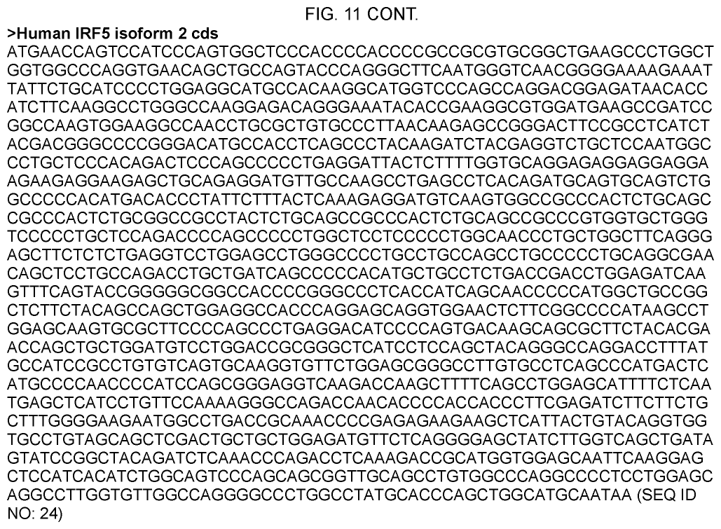

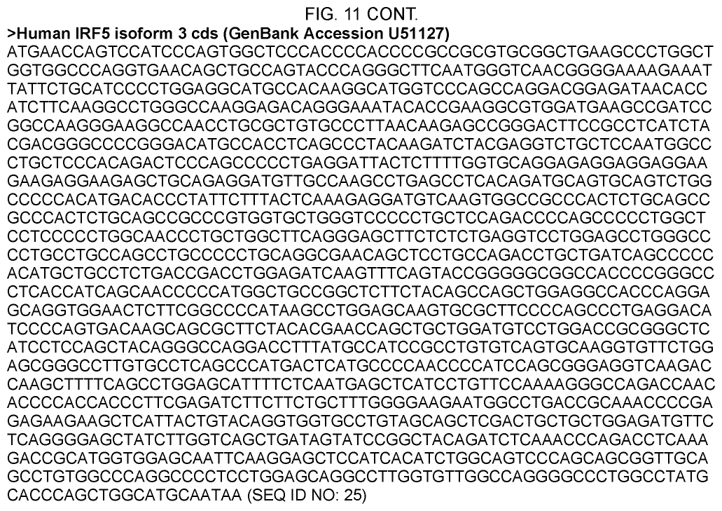

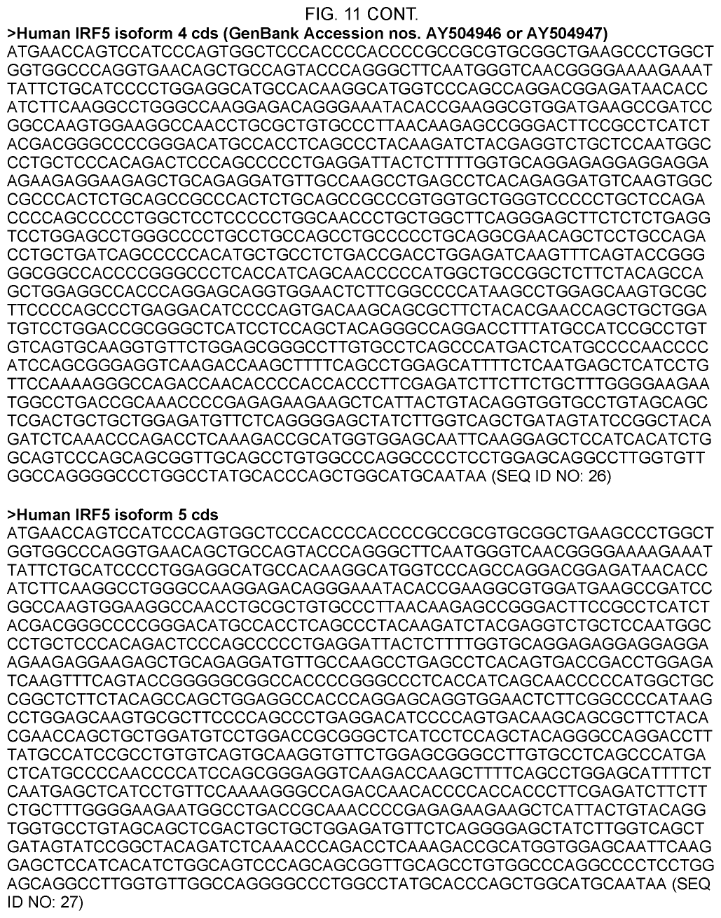

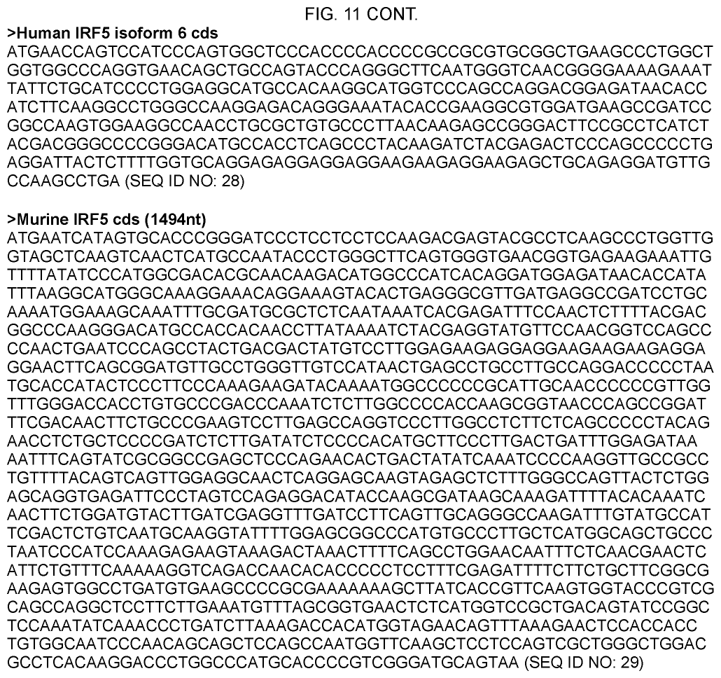

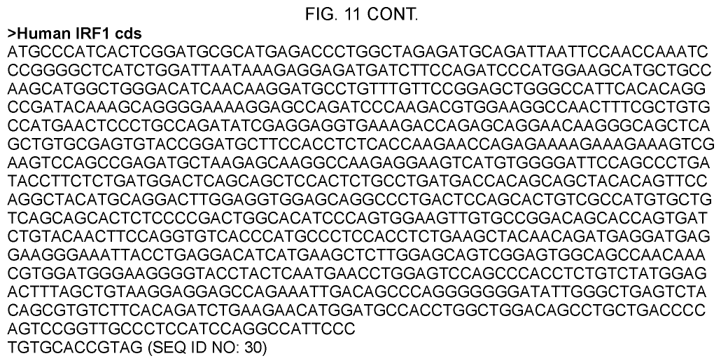

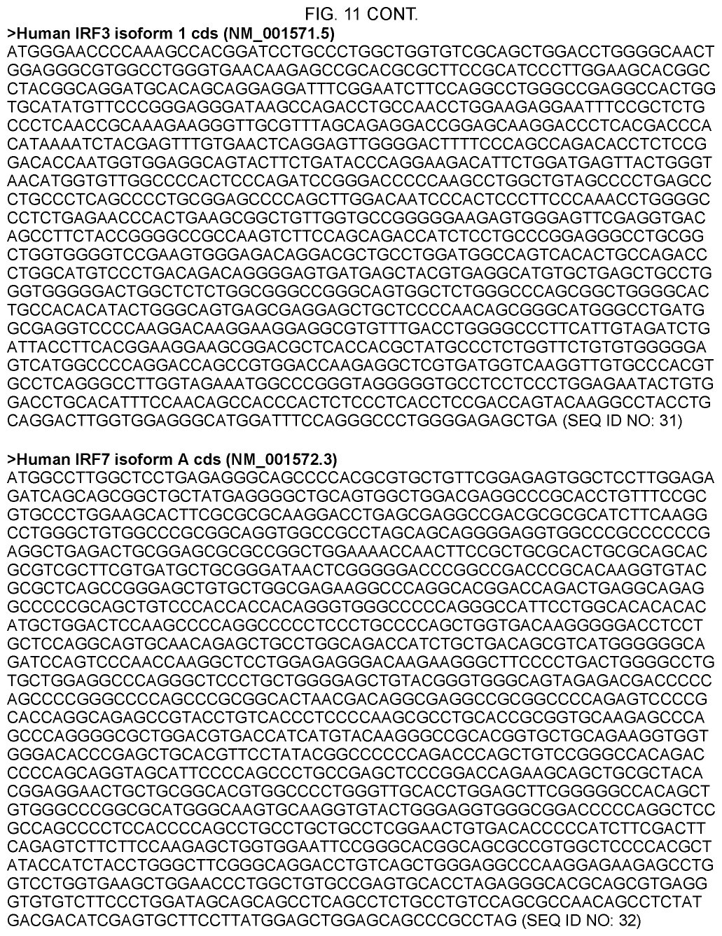

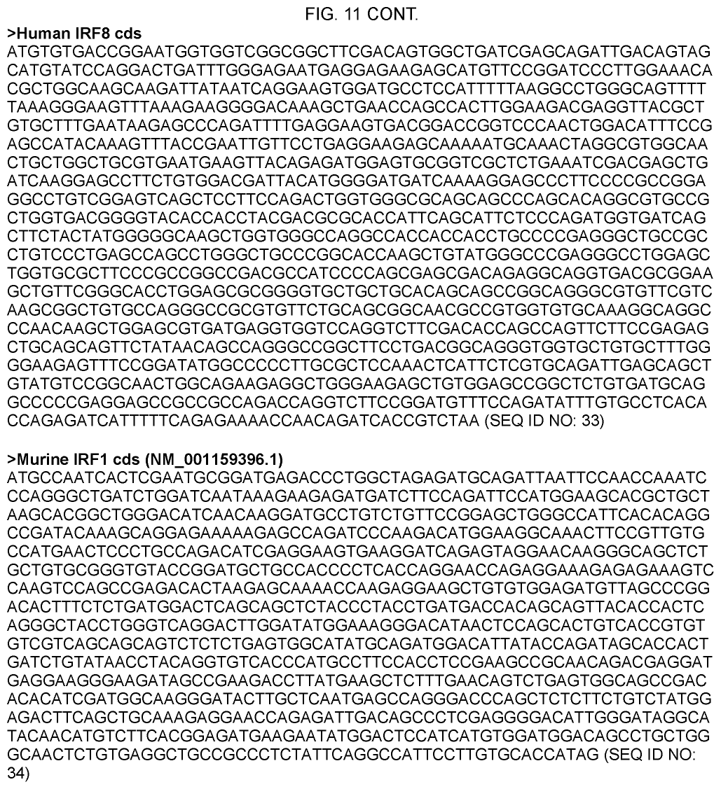

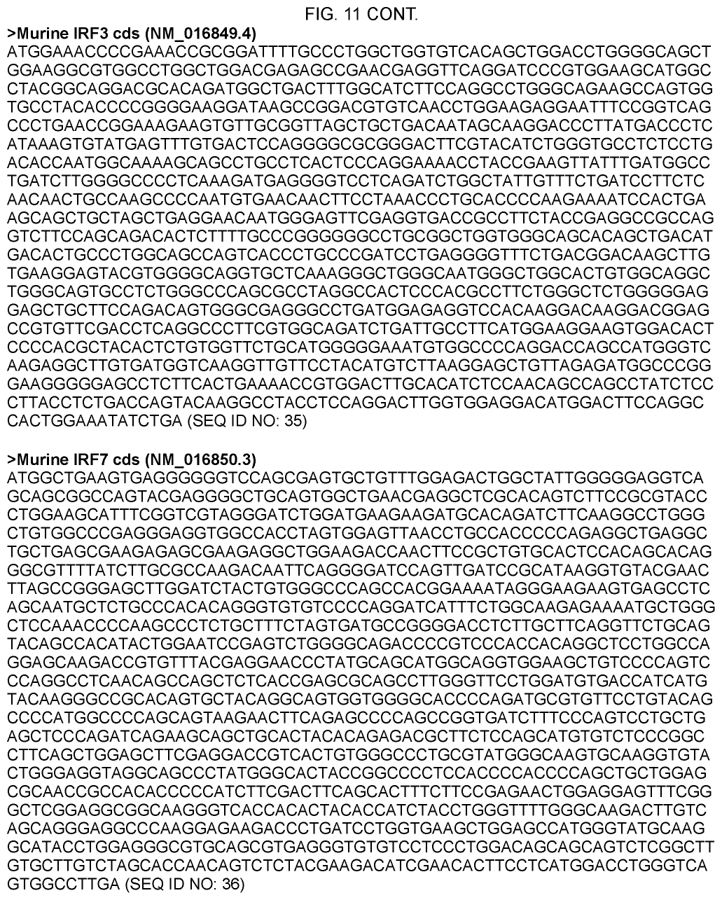

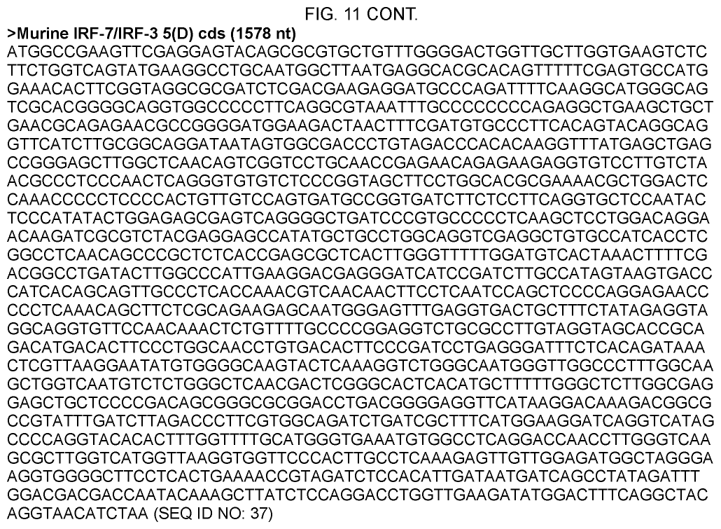

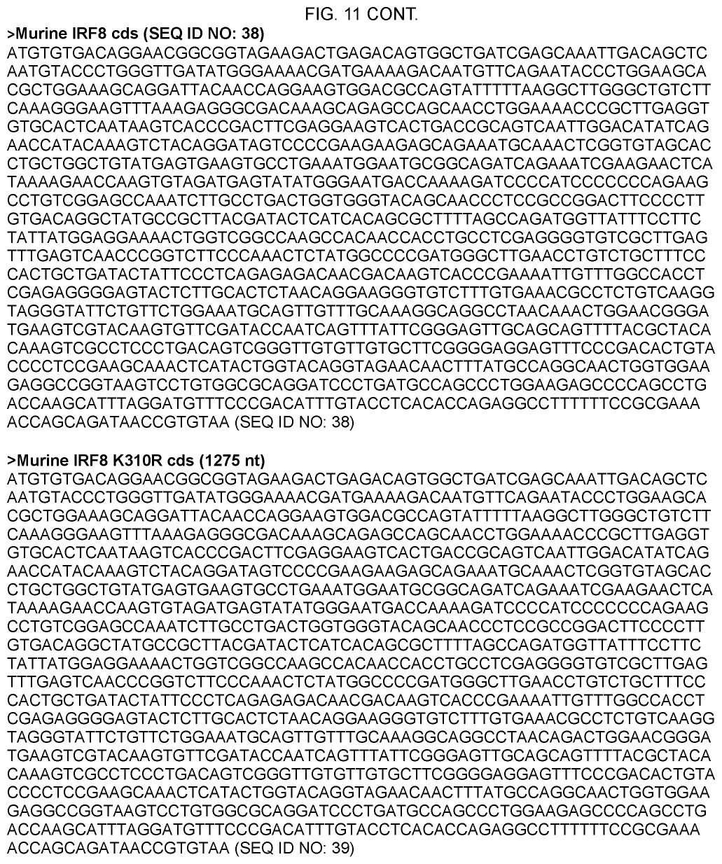

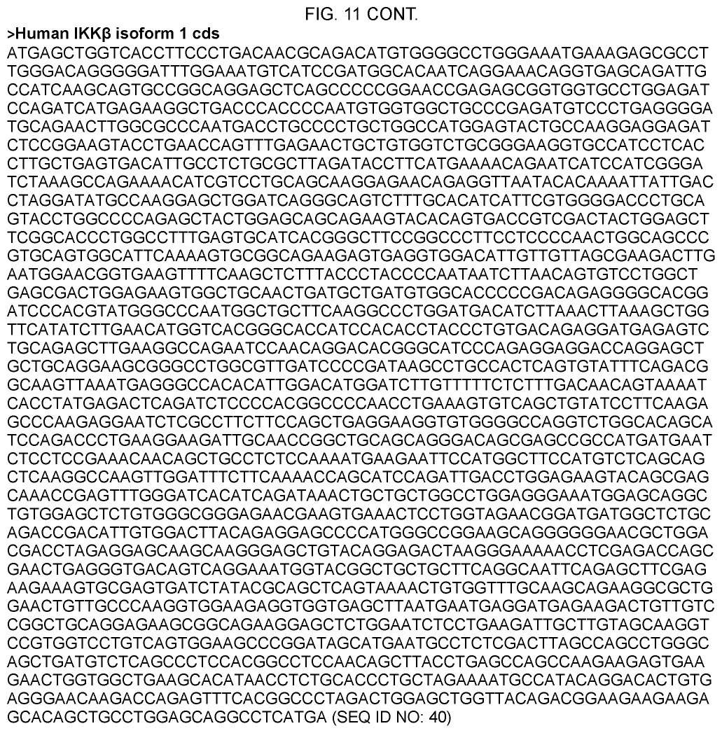

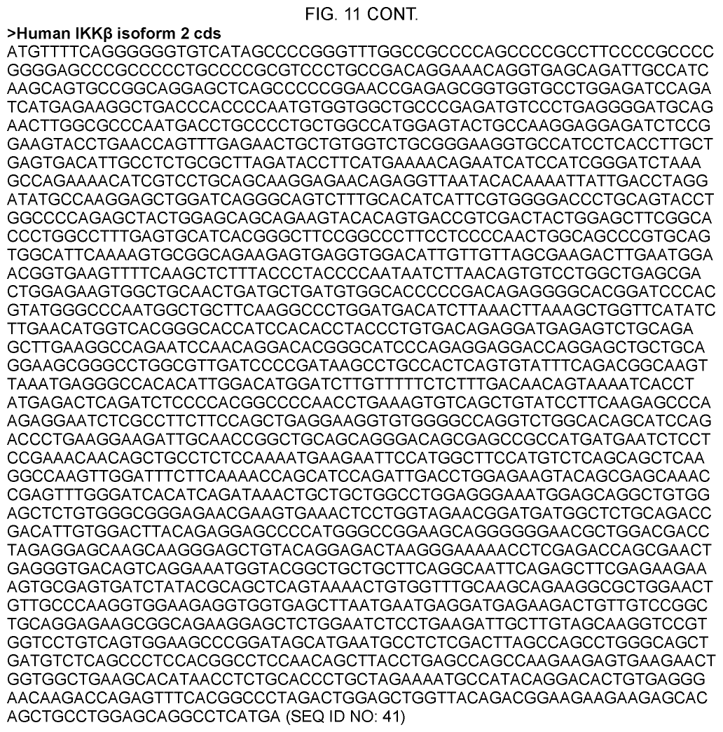

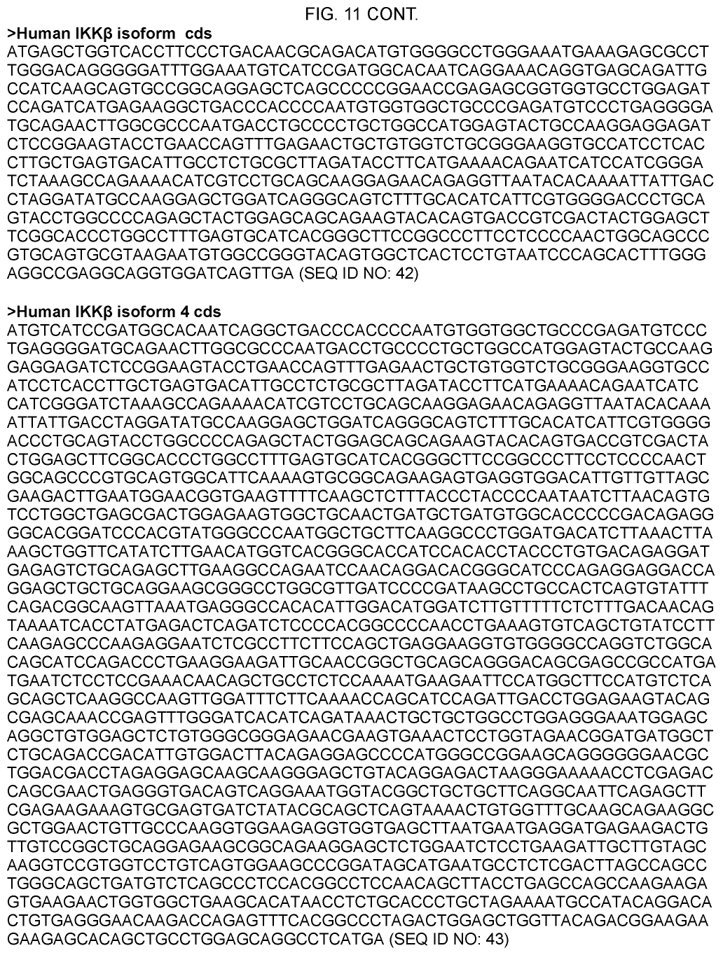

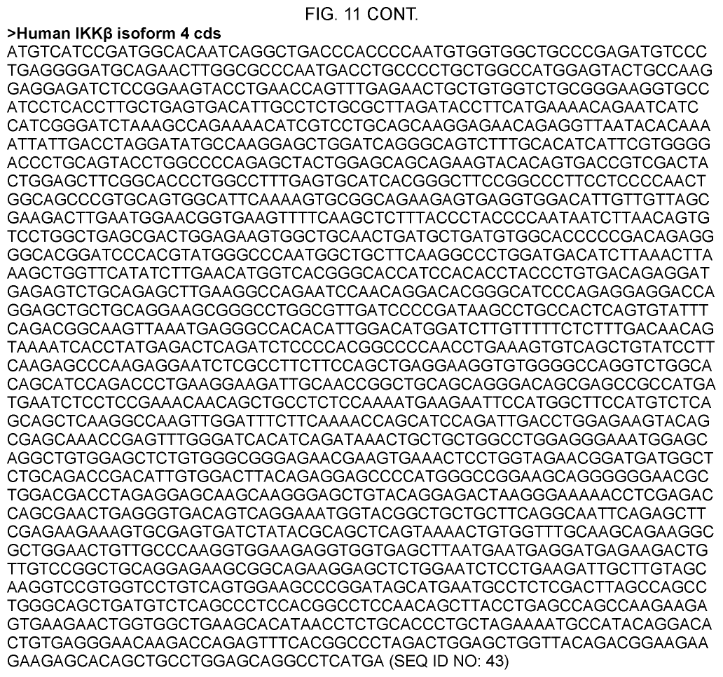

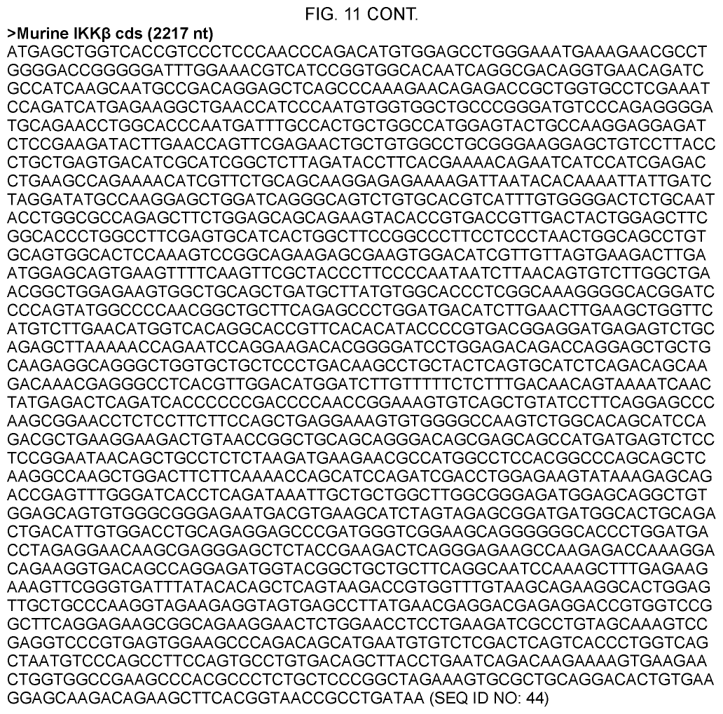

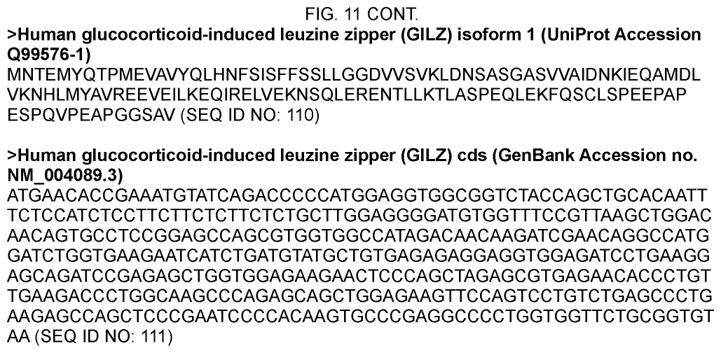

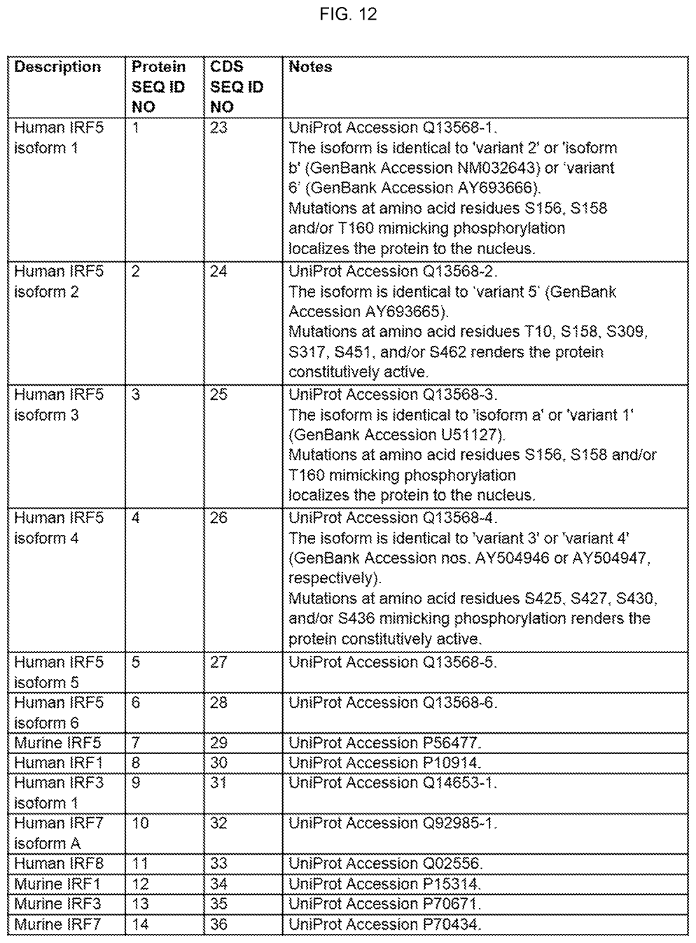

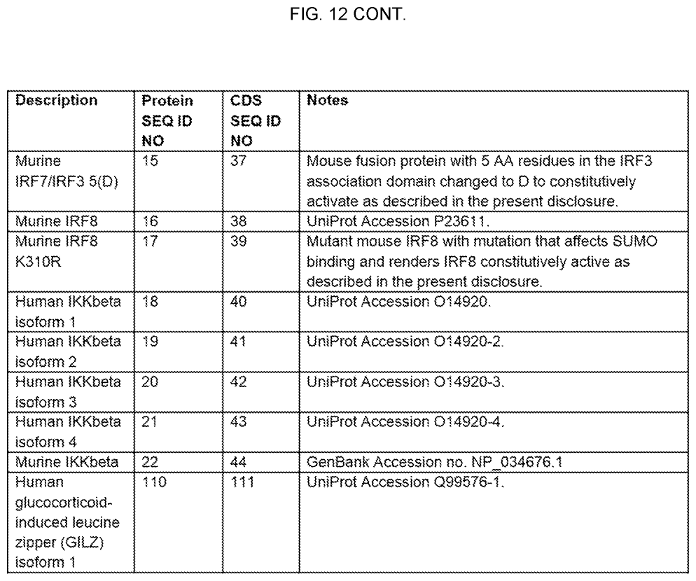

One interesting observation was that T cells contribute to the anti-tumor effects achieved with macrophage-programming nanoparticles. In fact, a multifocal dense cluster of host T cells surrounding the neoplasms was found in all IRF5/IKK β nanoparticle-treated animals, indicating that genetic programming of immune-stimulatory macrophages can restore lymphocyte migration and infiltration into solid tumors (NPs increased T cell infiltration into tumors by an average 10.6-fold (CD8) and 3.5-fold (CD4); see FIGS. 10A, 10B).

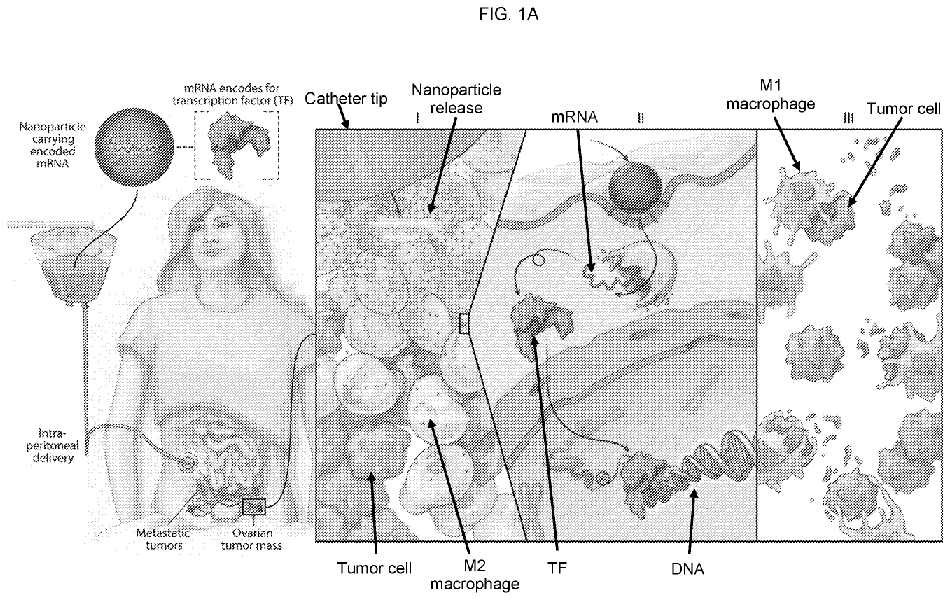

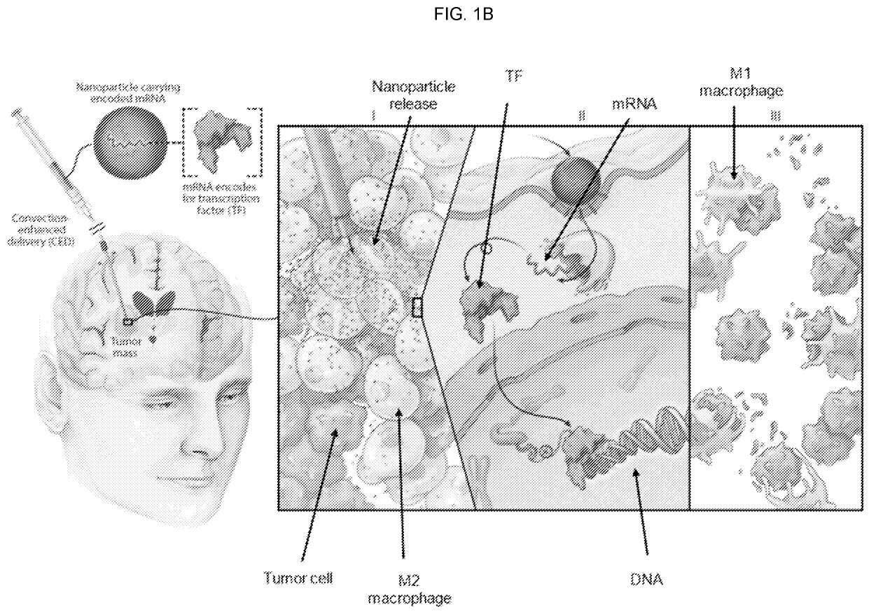

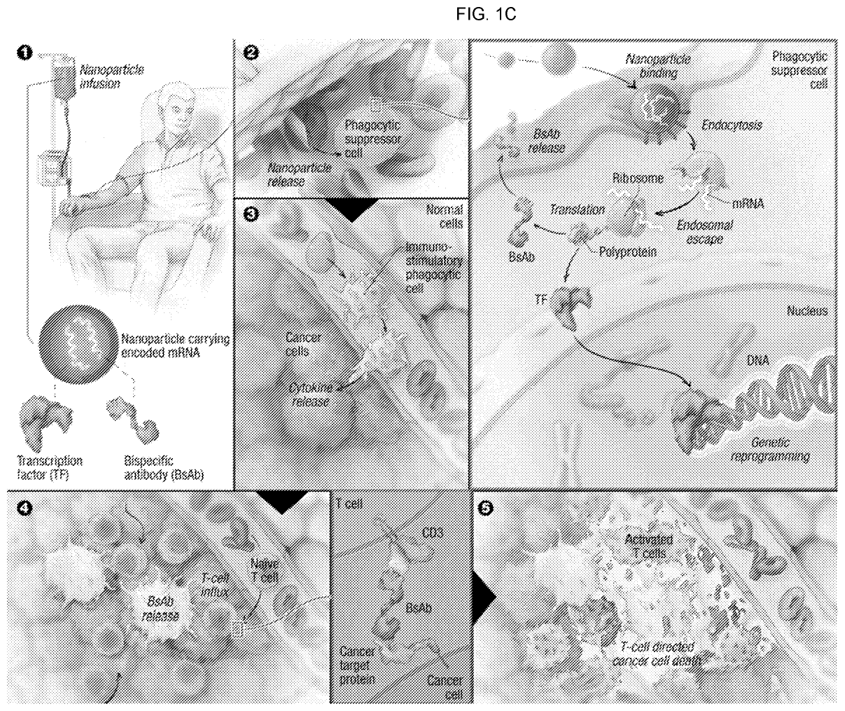

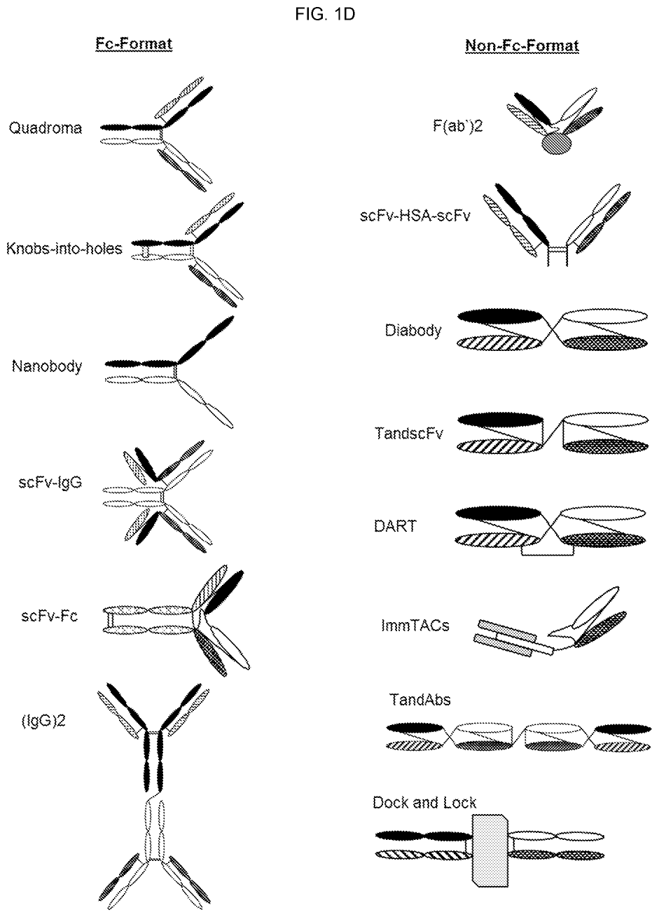

Because most of the T cells that are recruited into the tumor lack the therapeutically relevant T-cell receptor that would bind cancer antigens at a tumor site, however, the current disclosure provides for use of nanoparticles that deliver nucleotides encoding macrophage-programming transcription factors and T-cell redirecting macromolecules (such as bi-specific antibodies) to further activate innate and adaptive immune cells (illustrated in FIG. 1C).

One key advantage over existing bi-specific molecule technologies is that these molecules are directly secreted by TAMs and therefore reach the highest concentration within the tumor lesion (minimizing systemic exposure). Given the fast clearance rate of bi-specific antibodies (e.g., 2 hours in human serum), conventional bi-specific antibody therapy needs to be administered via continuous intravenous infusion and is associated with dose-limiting toxicities. This approach has shown little clinical success for the treatment of solid tumors, which are protected from T-cell attacks with myeloid-derived suppressor cells. Using the approaches described in the current disclosure, physicians can genetically modify monocytes/macrophages in vivo to (1) recruit additional immune cells to the tumor site; (2) remain activated at the tumor site providing an on-going stimulatory signal to other immune cells; and (3) secrete multi-specific immune-cell engaging molecules that bind cancer antigens at the tumor site and also bind and activate the recruited immune cells to destroy the bound cancer cell. Importantly, this therapy works from within the tumor, which is in contrast to existing combination treatments that can disrupt immune homeostasis.

Particular embodiments utilize nanoparticles to provide cells with nucleotides encoding genes encoding activation regulators such as transcription factors (e.g., Interferon Regulatory Factors (IRFs)) and/or kinases (e.g., IKKβ). These activation regulators regulate macrophage polarization. Macrophage polarization is a highly dynamic process through which the physiological activity of macrophages changes. As indicated, in most tumors, TAMs exhibit an immunosuppressed phenotype which can be an “M2” phenotype. By contrast, activated macrophages can exhibit an “M1” phenotype which results in tumor cell killing. Particular embodiments disclosed herein reverse the polarization of tumor-promoting TAMs into tumor-killing macrophages. Particular embodiments disclosed herein genetically modify monocytes to maintain an activated status upon later differentiation to macrophages so that the macrophages do not become immune-suppressed at a tumor site. These effects ameliorate the immunosuppressive milieu within the tumors by inducing inflammatory cytokines, activating other immune cells, and phagocytosing tumor cells.

“Macrophage activation” refers to the process of altering the phenotype or function of a macrophage from (i) an inactivated state to an activated state; (ii) a non-activated state to an activated state; (iii) an activated state to a more activated state; or (iv) an inactivated state to a non-activated state. An inactivated state means an immunosuppressed phenotype that facilitates tumor growth and metastasis. A non-activated state means that the macrophage neither facilitates tumor growth or metastasis nor promotes the killing of tumor cells. Activated means that the macrophage exhibits tumoricidal activity. In particular embodiments, the activated state results in an M1 phenotype as described more fully below. In particular embodiments, the inactivated state results in an M2 phenotype, also as described more fully below.

In particular embodiments, one benefit of the disclosed systems and methods is that patients can be spared from systemic toxicities because inflammation induced by treatment remains localized at the treatment site. To achieve this benefit, locally infused nanoparticles target TAMs in the tumor milieu, deliver nucleotides that selectively reprogram signaling pathways that control macrophage polarization, and are completely degradable locally by physiological pathways (Sahin et al., Nat Rev Drug Discov 13, 759-780 (2014)). Nanoparticles described herein can also be administered intravenously where they can be taken up by monocytes within the blood stream.

Achieving high expression of exogenous nucleotides in solid tumors is challenging in vivo. Before the current disclosure, nucleotide delivery systems based on viruses or conventional nanocarriers such as liposomes were limited by their restricted diffusion within tumor tissue. Jain & Stylianopoulos, Nat Rev Clin Oncol 7, 653-664 (2010). To circumvent this barrier, particular embodiments utilize nanoparticles (also referred to herein as NPs) with enhanced diffusivity so that the NPs deliver nucleotides to a large population of TAMs within a tumor. Particular embodiments utilize NPs <130 nm in size that carry a neutral surface charge.

Particular embodiments can further optionally include a targeting ligand attached to the surface of the NP. For example, macrophage mannose receptor 1 (MRC1), also known as CD206, is a type I transmembrane protein that is expressed by macrophages. CD206 also shows high expression levels in TAMs. Accordingly, in particular embodiments, di-mannose can be attached to the NP surface to enable more selective targeting to the mannose receptor (CD206) expressed on the TAM cell surface. For more information regarding CD206 binding and targeting ligands, see Zhang et al., Nature Communications, 10, 3974 (2019). Other TAM cell surface receptors that can be targeted include early growth response protein 2 (Egr2), CD163, CD23, interleukin (IL)27RA, CLEC4A, CD1a, CD1b, CD11b, CD14, CD16, CD31, CD93, CD115, CD192, CD226, IL13-Ra1, IL-4r, IL-1R type II, decoy IL-1R type II, IL-10r, macrophage scavenging receptors A and B, Ym-1, Ym-2, Low density receptor-related protein 1 (LRP1), IL-6r, CXCR1/2, CX3CR1, CXCR3, CXCR4, and PD-L1.

In particular embodiments, systems and methods disclosed herein include administering nanoparticles to a subject in need thereof. The nanoparticles are directed to monocytes in the bloodstream and/or macrophages present in tumors in the subject and are designed to be internalized by the monocytes/macrophages. Once internalized, the nanoparticles further deliver one or more nucleotides having sequences that encode IRF5 and IKKβ. The one or more nucleotides modify the monocytes/macrophages to express IRF5 and IKKβ. Without being bound by theory, the IKKβ kinase activates the IRF5 transcription factor by phosphorylation. Activated IRF5 then causes expression of type I interferon (IFN) genes, inflammatory cytokines, including tumor necrosis factor (TNF), IL-6, IL-12 and IL-23, and tumor suppressors. In M2 macrophages that have internalized one or more nucleotides encoding IRF5 and IKKβ, the expression of the aforementioned genes through IRF5 action leads to a phenotypic or functional switch of the macrophages from an M2 phenotype to an M1 phenotype, which enables the macrophages to kill or otherwise trigger the destruction of tumor cells, thereby treating cancer. In particular embodiments, the nanoparticles are internalized in the monocytes/macrophages by phagocytosis. In particular embodiments, the nanoparticles are internalized in the monocytes/macrophages by ligand-mediated endocytosis (e.g., CD-206-mediated endocytosis). In particular embodiments, delivery of the nanoparticles including the IRF5 and IKKβ genes into macrophages can include, e.g., (1) binding to the macrophages, (2) internalization of the nanoparticles by the macrophages, (3) escape from endocytic vesicles into the cytoplasm after internalization, (4) release of the one or more nucleotides, which (5) can be transported into the nucleus of the macrophages and (6) transcribed to deliver genes for expressing IRF5 and IKKβ.

As indicated previously, nanoparticles within systems disclosed herein additionally genetically modify monocytes/macrophages to produce and secrete bi-specific immune-cell activating molecules. This approach is depicted in FIG. 1C wherein a nanoparticle having nucleotides encoding transcription factors and a bi-specific antibody are encapsulated within a positively-charged core. In the approach depicted in FIG. 1C, nanoparticles are taken up by monocytes within the bloodstream. These monocytes then leave the bloodstream and arrive at a tumor site. Based on nanoparticle uptake, the cells express transcription factors that enter the nucleus and allow creation or maintenance of an activated macrophage state. The activated macrophage state attracts immune cells to the tumor site where it also secretes bi-specific antibodies. The bi-specific antibodies bind cancer antigens at the tumor site as well as activating epitopes on the recruited immune cells.

The transforming growth factor β (TGF-β) family of protein factors participates in a wide array of regulatory pathways in a wide array of different cell and tissue types, and at different stages of normal and pathological processes. Within cancers, TGF-β is a pleiotropic cytokine found at high levels in solid tumors. TGFβ induces regulatory T cells (Tregs) and inhibits CD8+ and TH1 cells in the tumor microenvironment, driving immune dysfunction. See, e.g., Ravi et al., Nature Communications 9, 741 (2018). Accordingly, particular embodiments disclosed herein reduce or neutralize TGF-β in the tumor microenvironment. For example, nanoparticles described herein can deliver nucleotides encoding a TGFβ inhibitor, such as a TGFβ antibody.

Aspects of the current disclosure are now described with additional detail and options as follows: (1) Macrophages and Macrophage Phenotypes; (2) Cellular Pathways to Affect Macrophage Polarization; (3) Targeted Antigens and Associated Binding Domains; (4) Immune Cell Activating Epitopes and Associated Binding Domains; (5) Bi-Specific Molecule Formats; (6) TGFβ. Inhibitors; (7) Nucleotides; (8) Nanoparticles; (9) Compositions for Administration; (10) Methods of Use; (11) Exemplary Embodiments; (12) Experimental Examples; and (13) Closing Paragraphs. These headings do not limit the interpretation of the disclosure and are provided for organizational purposes only.

(1) Macrophages and Macrophage Phenotypes. “Macrophage” refers to a white blood cell of the immune system differentiated from bone marrow derived monocytes. Macrophages are characterized by their phagocytic activity and their antigen presentation capacity. Macrophages are key players in both the innate and adaptive immune responses. Phenotypically macrophages express the surface marker F4/80 (Ly71) and may also express other surface markers such as CDIIb (MacI), CDIIc, CD14, CD40 or CD68.

Macrophages play an important role in both innate and adaptive immunity by activating T lymphocytes. In cancer, macrophages are one of the major populations of infiltrating leukocytes associated with solid tumors (Gordon S & Taylor P R (2005) Nature Reviews Immunology 5(12): 953-964). They can be recruited to the tumor site from surrounding tissues or by the tumor itself through the secretion of chemotactic molecules. Macrophages participate in immune responses to tumors in a polarized manner depending on their phenotype. “Phenotype” is used herein to refer to the physical attributes or biochemical characteristics of a cell as a result of the interaction of its genotype and the environment and can include functions of a cell.

Macrophages that activate Th1 T lymphocytes provide an inflammatory response and are often denoted as having an M1-polarized or “classically activated” phenotype. Macrophages in an activated state (i.e. M1 macrophages or macrophages having an M1 phenotype), also referred to as “killer macrophages,” inhibit cell proliferation, cause tissue damage, mediate resistance to pathogens, and possess strong tumoricidal activity. These macrophages can increase expression of mediators that are responsible for antigen presentation and costimulation; promoting infiltration of neutrophils to a tumor area leading to neutrophil-targeted tumor regression. An M1 phenotype can also be evidenced by increased antigen presentation as compared to a relevant control condition. In particular embodiments, an M1 phenotype can be evidenced by M1 macrophage production of reactive oxygen species (ROS) and nitric oxide (NO). NO has anti-proliferative effects integral for protection against pathogens and aberrant cells like tumor cells. In particular embodiments, an M1 phenotype can be evidenced by a pro-inflammatory state that induces Th1 immunity through the production of cytokines such as IL-12. In particular embodiments, macrophages in an activated state are classically activated macrophages that can phagocytose pathogens.

Beyond function, an M1 phenotype can also be evidenced by surface markers expressed by the macrophages; factors, proteins, or compounds produced by the macrophages upon polarization; or genes induced by the macrophages upon polarization. M1 polarization can lead to a phenotype evidenced by expression of CD80, CD86, iNOS, suppressor of cytokine signaling 3 (SOCS3), TNFα, IL-1, IL-6, IL-12, IL-23, Type I IFN, CXCL1, CXCL2, CXCL3, CXCL5, CXCL8, CXCL9, and CXCL10. In particular embodiments, an M1 phenotype includes an increase in expression of CD80. In particular embodiments, an M1 phenotype includes CD206−, MHCII+, CD11c−, and CD11b+.

On the other hand, macrophages that activate Th2 T lymphocytes provide an anti-inflammatory response and are often denoted as having an “M2” phenotype. Macrophages that are in an inactivated state (i.e. M2 macrophages or macrophages having an M2 phenotype), also referred to as “repair macrophages,” are involved in metazoan parasites containment, cell proliferation, tissue repair, tumor progression, anti-inflammation pathways, and immunosuppression. An M2 phenotype can reduce antigen presentation and decrease phagocytosis as compared to a relevant control condition. An M2 phenotype can also be evidenced by, for example, expression of one or more of arginase 1 (Arg1 (arginase activity is associated with pro-proliferative effects and tissue repair responses)), IL-10, TGF-β, PPArγ, KLF4, CD206 (MRC1), Dectin-1 (a signaling non-TLR pattern-recognition receptor), DC-SIGN (C-type lectin), scavenger receptor A, scavenger receptor B-1, CD163 (high affinity scavenger receptor for the hemoglobin-haptoglobin complex), chemokine receptors CCR2, CXCR1, and CXCR2, YM1 (chitinase 3—like 3), and Fizz1; and secretion of the chemokines CCL17, CCL22 and CCL24. In particular embodiments, macrophages in an inactivated state promote metastasis and/or resistance to chemotherapy. In particular embodiments, an M2 phenotype includes CD206+, MHCII−, CD11c+, and CD11b^{low}.

Table 1 provides particular combinations of criteria that can be used to distinguish an M1 phenotype from M2 phenotypes (including sub-phenotypes designated as M2a, M2b, M2c and M2d).

TABLE 1
Exemplary Criteria to Categorize Macrophage Phenotypes.

| | M1 | M2a | M2b | M2c | M2d |
|---|---|---|---|---|---|
| Stimulation/ | IFN-γ | IL-4 | ICs | IL-10 | IL-6 |
| Activation | LPS | IL-13 | IL-1R | TGF-β | LIF |
| | GM-CSF | Fungal and Helminth | | GCs | Adenosine |
| | | infection | | | |
| Marker | CD86 | CD163 | CD86 | CD163 | VEGF |
| Expression | CD80 | CD23 | MHC II | TLR1 | |
| | CD68 | MHC II | | TLR8 | |
| | MHC II | SR | | | |
| | IL-1R | MMR/CD206 | | | |
| | TLR2 | CD200R | | | |
| | TLR4 | TGM2 | | | |
| | iNOS | DecoyR | | | |
| | SOCS3 | IL-1R II | | | |
| | CD28 | Mouse only: | | | |
| | Gpr18 | Ym1/2 | | | |
| | Fpr2 | Fizz1 | | | |
| | CD64 | Arg-1 | | | |
| Cytokine | TNF | IL-10 | IL-1 | IL-10 | IL-10 |
| secretion | IL-1β | TGF-β | IL-6 | TGF-β | IL-12 |
| | IL-6 | IL-1ra | IL-10 TNFα | | TNFα |
| | IL-12 | | | | TGFβ |
| | IL-23 | | | | |
| Chemokine | CCL10 | CCL17 | CCL1 | CCR2 | CCL5 |
| secretion | CCL11 | CCL22 | | | CXCL10 |
| | CCL5 | CCL24 | | | CXCL16 |
| | CCL8 | | | | |
| | CCL9 | | | | |
| | CCL2 | | | | |
| | CCL3 | | | | |
| | CCL4 | | | | |

Adapted from Röszer T (2015) Mediators Inflamm 2015, 816460 and Duluc D et al. (2007) Blood 110: 4319-4330. Arg-1, arginase-1; Fizz1, resistin-like molecule-alpha (Retnl-alpha); GCs, glucocorticoids; ICs, immune complexes; IL1-ra, IL-1 receptor antagonist; LIF, leukocyte inhibitory factor; TGM2, transglutaminase 2; TGF-β, transforming growth factor-beta; TNFα, tumor necrosis factor alpha; TLR, Toll-like receptor; MMR (CD206), macrophage mannose receptor; iNOS, inducible nitric oxide synthase; SR, scavenger receptor; SOCS3, suppressor of cytokine signaling 3; VEGF, vascular endothelial growth factor; Ym1 (also known as chitinase-3-like protein-3 (Chi3I3)).

Assays to assess macrophage phenotype can take advantage of the different molecular signatures particular to the M1 or M2 phenotype. A commonly accepted marker profile for M1 macrophages is CD80+, whereas M2-macrophages can be characterized as CD163+. Thus, flow cytometry can be performed to assess for these markers. Driving macrophages towards a M1 type and away from a M2 type can also be assessed by measuring an increase of the IL-12/IL-10 ratio or the CD163−/CD163+ macrophage ratio. In particular embodiments, M1 versus M2 morphology can be assessed by light microscopy. In particular embodiments, phagocytosis assays may be used in conjunction with other assays to assess whether a macrophage is M1 type or M2 phenotype. Phagocytosis assays of different macrophage populations may be performed by incubating an entity to be phagocytosed with macrophages at a concentration that is consistent with their normalized total surface area per cell. The entity to be phagocytosed may be added to macrophage cultures. The entity to be phagocytosed may be, for example, labeled with a fluorescent label. Phagocytosis index may be determined by the median total fluorescence intensity measured per macrophage. Quantification of phagocytosis may be by, for example, flow cytometry. Tumor cell killing assays may also be utilized. In particular embodiments, an M1 phenotype includes reduced expression of signature M2 macrophage genes including SerpinB2 (inhibitor of urokinase-type plasminogen activator), CCL2 (C—C motif chemokine ligand 2), CCL11 (C—C motif chemokine ligand 11), and Retnla (resistin like alpha; Fizz1). In particular embodiments, an M1 phenotype includes increased expression of M1 differentiation genes including CCL5 (C—C motif chemokine ligand 5).

Gene expression (e.g., M1 expression of CD80, CD86 and/or other genes noted above) can be measured by assays well known to a skilled artisan. Methods to measure gene expression include NanoString nCounter® expression assays (NanoString Technologies, Inc., Seattle, WA), Northern blots, dot blots, microarrays, serial analysis of gene expression (SAGE), RNA-seq, and quantitative RT-PCR. Methods to measure gene expression products, e.g., protein level, include ELISA (enzyme linked immunosorbent assay), western blot, FACS, radioimmunological assay (RIA), sandwich assay, fluorescent in situ hybridization (FISH), immunohistological staining, immunoelectrophoresis, immunoprecipitation, and immunofluorescence using detection reagents such as an antibody or protein binding agents.

Embodiments disclosed herein can be used to genetically modify phagocytic cells, such as polymorphonuclear neutrophils, monocytes, monocyte-derived macrophages, tissue-resident macrophages, epithelial cells, fibroblasts, and dendritic cells. Phagocytic cells can be professional or non-professional. Professional phagocytes include polymorphonuclear neutrophils, monocytes, monocyte-derived macrophages, and tissue-resident macrophages. In particular embodiments, the primary function of a professional phagocytic cell is phagocytosis. Non-professional phagocytes include all other cell types that can perform phagocytosis, but it is not considered the primary function of the cell. Examples of non-professional phagocytes include epithelial cells, fibroblasts, and dendritic cells. For more information regarding professional and non-professional phagocytes, see Lim, Grinstein, and Roth, Frontiers in Cellular and Infection Microbiology, May 2017, Vol. 7, Article 191.

(2) Cellular Pathways to Affect Macrophage Polarization. Polarization of a macrophage towards an activated or inactivated phenotype results from macrophage interaction with a number of different molecules or environments. For example, M1 macrophage polarization is triggered by stimuli including Toll-like receptor (TLR) ligands (e.g. lipopolysaccharide (LPS), muramyl dipeptide, lipoteichoic acid, imiquimod, CpG), IFNγ, TNFα, and macrophage colony-stimulating factor (GM-CSF). M2 polarized macrophages can be divided into subsets, depending on the stimuli that initiates the polarization: the M2a subtype is elicited by IL-4, IL-13 or fungal and helminth infections; M2b is elicited by IL-1 receptor ligands, immune complexes and LPS; M2c is elicited by IL-10, TGF-β and glucocorticoids; and M2d is elicited by IL-6 and adenosine. M2 macrophage polarization may also be triggered by IL-21, GM-CSF, complement components, and apoptotic cells. Macrophage polarization is also modulated by local microenvironmental conditions such as hypoxia.

The aforementioned molecules and environments affect macrophage polarization by triggering different intracellular signaling pathways involving transcription factors. Transcription factors that are involved in both M1 and M2 polarization include IRFs, signal transducers and activators of transcription (STAT), SOCS3 proteins, and nuclear factor kappa-light-chain-enhancer of activated B cells (NFκB). Mitogen-activated protein kinases (MAPK) also play a role in directing macrophage function towards either the M1 or M2 phenotype.

The IRF/STAT pathways, activated by such stimuli as IFNs and TLR signaling as discussed above, polarize macrophages to the M1 activation state via STAT1. On the other hand, such stimuli as IL-4 and IL-13 skew macrophages toward the M2 activation state via STAT6 (Sica A & Bronte V (2007) J Clin Invest 117: 1155-1166). These signaling events thus result in either the promotion of an inflammatory immune response and tumoricidal activity, as in the case of M1 macrophage polarization, or in the promotion of an immunosuppressive protumor response, as in the case of M2 macrophage polarization.

Some intracellular molecules implicated in the induction of an M1 phenotype include the G-protein coupled receptor, P2Y(2)R, which plays a role in inducing NO via NOS2 (Eun S Y et al. (2014) Int Immunopharmacol 18: 270-276); SOCS3, which activates NFκB/PI-3 kinase pathways to produce NO (Arnold C E et al. (2014) Immunology 141: 96-110); and growth and differentiation factor Activin A, which promotes M1 markers and down-regulates IL-10 (Sierra-Filardi E et al. (2011) Blood 117: 5092-5101).

Other intracellular molecules involved in induction of the M1 phenotype include IRFs. IRFs are a group of transcription factors with diverse roles, including virus-mediated activation of IFN, and modulation of cell growth, differentiation, apoptosis, and immune system activity. Members of the IRF family are characterized by a conserved N-terminal DNA-binding domain containing tryptophan (W) repeats.

IRF5 is a transcription factor that possesses a helix-turn-helix DNA-binding motif and mediates virus- and IFN-induced signaling pathways. It acts as a molecular switch that controls whether macrophages will promote or inhibit inflammation. IRF5 activates type I IFN genes, inflammatory cytokines, including TNF, IL-6, IL-12 and IL-23, and tumor suppressors as well as Th1 and Th17 responses. It is encoded by the human IRF5 gene located at chromosome 7q32 (OMIM ID 607218). It is appreciated that several isoforms/transcriptional variants of IRF5 exist. In particular embodiments, isoforms of human IRF5 include isoform 1 (UniProt Accession Q13568-1, SEQ ID NO: 1), isoform 2 (UniProt Accession Q13568-2, SEQ ID NO: 2), isoform 3 (UniProt Accession Q13568-3, SEQ ID NO: 3), isoform 4 (UniProt Accession Q13568-4, SEQ ID NO: 4), isoform 5 (UniProt Accession Q13568-5, SEQ ID NO: 5) and isoform 6 (UniProt Accession Q13568-6, SEQ ID NO: 6). In particular embodiments, isoforms of human IRF5 include isoform 1 encoded by a nucleotide sequence shown in SEQ ID NO: 23, isoform 2 encoded by a nucleotide sequence shown in SEQ ID NO: 24, isoform 3 encoded by a nucleotide sequence shown in SEQ ID NO: 25, isoform 4 encoded by a nucleotide sequence shown in SEQ ID NO: 26, isoform 5 encoded by a nucleotide sequence shown in SEQ ID NO: 27 and isoform 6 encoded by a nucleotide sequence shown in SEQ ID NO: 28. In particular embodiments, murine IRF5 includes an amino acid sequence shown in SEQ ID NO: 7. In particular embodiments, murine IRF5 is encoded by a nucleotide sequence shown in SEQ ID NO: 29. M1 macrophages have been shown to upregulate IRF5.

IRF1 and IRF8 also play critical roles in the development and function of myeloid cells, including activation of macrophages by proinflammatory signals such as IFN-γ. Dror N et al. (2007) Mol Immunol. 44(4):338-346. In particular embodiments, human IRF1 includes an amino acid sequence shown in SEQ ID NO: 8. In particular embodiments, human IRF1 is encoded by a nucleotide sequence shown in SEQ ID NO: 30. In particular embodiments, murine IRF1 includes an amino acid sequence shown in SEQ ID NO: 12. In particular embodiments, murine IRF1 is encoded by a nucleotide sequence shown in SEQ ID NO: 34. In particular embodiments, human IRF8 includes an amino acid sequence shown in SEQ ID NO: 11. In particular embodiments, human IRF8 is encoded by a nucleotide sequence shown in SEQ ID NO: 33. In particular embodiments, murine IRF8 includes an amino acid sequence shown in SEQ ID NO: 16. In particular embodiments, murine IRF8 is encoded by a nucleotide sequence shown in SEQ ID NO: 38.

IRF3 is a homolog of IRF1 and IRF2. It contains several functional domains including a NES, a DBD, a C-terminal IRF association domain and several regulatory phosphorylation sites. IRF3 is found in an inactive cytoplasmic form that upon serine/threonine phosphorylation forms a complex with CREB Binding Protein, a transcriptional coactivator. This complex translocates to the nucleus and activates the transcription of IFN-α and -β, as well as other interferon-induced genes. In particular embodiments, isoforms of human IRF3 include isoform 1 (UniProt Accession Q14653-1), isoform 2 (UniProt Accession Q14653-2), isoform 3 (UniProt Accession Q14653-3), isoform 4 (UniProt Accession Q14653-4), and isoform 5 (UniProt Accession Q14653-5). In particular embodiments, human IRF3 isoform 1 includes an amino acid sequence shown in SEQ ID NO: 9. In particular embodiments, human IRF3 isoform 1 is encoded by a nucleotide sequence shown in SEQ ID NO: 31. In particular embodiments, murine IRF3 includes an amino acid sequence shown in SEQ ID NO: 13. In particular embodiments, murine IRF3 is encoded by a nucleotide sequence shown in SEQ ID NO: 35.

IRF7 has been shown to play a role in the transcriptional activation of type I IFN genes. In particular embodiments, isoforms of human IRF7 include isoform A (UniProt Accession Q92985-1), isoform B (UniProt Accession Q92985-2), isoform C (UniProt Accession Q92985-3), and isoform D (UniProt Accession Q92985-4). In particular embodiments, human IRF7 isoform A includes an amino acid sequence shown in SEQ ID NO: 10. In particular embodiments, human IRF7 isoform A is encoded by a nucleotide sequence shown in SEQ ID NO: 32. In particular embodiments, murine IRF7 includes an amino acid sequence shown in SEQ ID NO: 14. In particular embodiments, murine IRF7 is encoded by a nucleotide sequence shown in SEQ ID NO: 36.

One or more IRF mutants that contribute to IRF activation may also be used. For example: phosphomimetic mutants of human variant 3/variant 4 of IRF5 (isoform 4, SEQ ID NO: 4) that substitute amino acid residues S425, S427, S430, S436 with residues mimicking phosphorylation, such as aspartic acid residues (Chen W et al. (2008) Nat Struct Mol Biol. 15(11): 1213-1220); phosphomimetic mutants of human variant 5 of IRF5 (isoform 2, SEQ ID NO: 2) that substitute amino acid residues T10, S158, S309, S317, S451, and/or S462 with residues mimicking phosphorylation, such as aspartic acid residues (Chang Foreman H-C et al. infra); mutation of human IRF5 isoform a (variant 1, isoform 3, SEQ ID NO: 3) and isoform b (variant 2, isoform 1, SEQ ID NO: 1) residues S156, S158 and T160 to residues mimicking phosphorylation, such as aspartic acid residues, for constitutive nuclear accumulation of IRF5 (Lin R et al. (2005) J Biol Chem 280(4): 3088-3095); and IRF3 phosphomimetic mutants that substitute amino acid residue S396 of IRF3 with residues mimicking phosphorylation, such as aspartic acid (Chen W et al. infra). In particular embodiments, a fusion protein of murine IRF7/IRF3 includes Asp (D) mutations at four serine and one threonine residues in the IRF3 association domains (SEQ ID NO: 15), conferring constitutive activation and translocation of the fusion protein (Lin R et al. (1998) supra; Lin et al. (2000) Molecular and Cellular Biology 20: 6342-6353). In particular embodiments, a fusion protein of murine IRF7/IRF3 including D mutations at four serine and one threonine residues in the IRF3 association domains is encoded by a nucleotide sequence shown in SEQ ID NO: 37. In particular embodiments, a murine IRF8 mutant includes substitution of Lysine (K) at amino acid residue 310 with Arginine (R) (SEQ ID NO: 17). In particular embodiments, a murine IRF8 mutant including a substitution of K at amino acid residue 310 with R is encoded by a nucleotide sequence shown in SEQ ID NO: 39. Small ubiquitin-like modifiers (SUMO) bound to IRF8 primarily at K310 inhibit activation of IRF8 responsive genes. Sentrin-specific protease 1 (SENP1) targets SUMO 2/3. The activity of SENP1 “deSUMOylates” IRF8 (and other proteins) and causes IRF8 to go from a repressor of M1 macrophage differentiation to an activator (directly and through transactivation activities). Preventing SUMO binding to IRF8 by mutation of the K310 residue increases IRF8 specific gene transcription 2-5 fold (see Chang T-H et al. (2012) supra).

Particular embodiments of the present disclosure include engineered IRF transcription factors. In particular embodiments, engineered IRF transcription factors include IRFs that lack a functioning autoinhibitory domain and are therefore insensitive to feedback inactivation (Thompson et al. (2018) Front Immunol 9: 2622). For example, a human IRF5 with 2-3-fold increase in activity can be obtained by deleting aa 489-539 of the human IRF5 protein (Barnes et al. (2002) Mol Cell Biol 22: 5721-5740). In particular embodiments, an autoinhibitory domain of IRF4, a transcription factor that promotes an M2 phenotype, can be deleted or mutated to generate a more active IRF4 in the context of treating an autoimmune disease. In particular embodiments, an autoinhibitory domain of an IRF is found at the carboxy terminus of the IRF protein. In particular embodiments, engineered IRF transcription factors include IRFs that lack one or more functioning nuclear export signals (NES) to entrap IRFs in the nucleus and therefore enhance transcription. For example, nuclear accumulation of human IRF5 can be achieved by mutating the NES of human IRF5 by replacing two leucine residues with alanine (L157A/L159A) (Lin et al. (2000) Molecular and Cellular Biology 20: 6342-6353). In particular embodiments, engineered IRF transcription factors include fusions of one or more IRFs, fusions of fragments of one or more IRFs, and fusions of mutated IRFs.

NFκB is also a key transcription factor related to macrophage M1 activation. NFκB regulates the expression of a large number of inflammatory genes including TNFα, IL1B, cyclooxygenase 2 (COX-2), IL-6, and IL12p40. NFκB activity is modulated via the activation of the inhibitor of kappa B kinase (IKK) trimeric complex (two kinases, IKKα, IKKβ, and a regulatory protein, IKKγ). When upstream signals converge at the IKK complex, they first activate IKKβ kinase via phosphorylation, and activated IKKβ further phosphorylates the inhibitory molecule, inhibitor of kappa B (I-κB). This results in the proteosomal degradation of I-κB and the release of NFκB p65/p50 heterodimer from the NFκB/I-κB complex. The NFκB p65/p50 heterodimer is then translocated to the nucleus and binds to the promoters of inflammatory genes.

IKKβ is an activating kinase for NFκB as well as other transcription factors such as IRF5. IKKβ similarly phosphorylates several other signaling pathway components including FOXO3, NCOA3, BCL10, IRS1, NEMO/IKBKG, NFκB subunits RELA and NFκB1, as well as the IKK-related kinases TBK1 and IKBKE. In particular embodiments, isoforms of human IKKβ include isoform 1 (UniProt Accession 014920-1, SEQ ID NO: 18), isoform 2 (UniProt Accession 014920-2 SEQ ID NO: 19), isoform 3 (UniProt Accession 014920-3 SEQ ID NO: 20), and isoform 4 (UniProt Accession 014920-4 SEQ ID NO: 21). In particular embodiments, isoforms of human IKKβ include isoform 1 encoded by a nucleotide sequence shown in SEQ ID NO: 40, isoform 2 encoded by a nucleotide sequence shown in SEQ ID NO: 41, isoform 3 encoded by a nucleotide sequence shown in SEQ ID NO: 42, and isoform 4 encoded by a nucleotide sequence shown in SEQ ID NO: 43. In particular embodiments, murine IKKβ includes an amino acid sequence shown in SEQ ID NO: 22. In particular embodiments, murine IKKβ is encoded by a nucleotide sequence shown in SEQ ID NO: 44.

As indicated, hypoxia also influences macrophage polarization through hypoxia inducible factors HIF-1α and HIF-2a. HIF-1α regulates NOS2 expression and supports emergence of an M1 phenotype while HIF-2a regulates Arg1 expression and supports emergence of an M2 phenotype (Takeda N et al. (2010) Genes Dev 24: 491-501).

TABLE 2
Signaling molecules and genes involved
in macrophage polarization.

| | M1 | M2 |
|---|---|---|
| Signaling | STAT1alpha/beta | STAT6 |
| Molecules | IRF5 | KLF-4 |
| | Btk | NFκB p50 homodimers |
| | P2Y(2)R | PPARγ |
| | SOCS3 | HIF-2α |
| | Activin A | IL-21 |
| | HIF1-α | BMP-7 |
| | | FABP4 |
| | | LXRα |
| Genes | TNFα, Cox-2, CCL5, NOS2 | Arg-1, Mrc-1, Fizz1, PPARγ |

Adapted from Sica A and Mantovani A 2012 (supra) and Chávez-Gálan L et al. (2015) Front Immunol 6, 253. Arg-1, arginase-1; Fizz1, resistin-like molecule-alpha (Retnl-alpha); STAT, signal transducers and activators of transcription; IRF, interferon regulatory factor; SOCS3, suppressor of cytokine signaling 3; Btk, Bruton's tyrosine kinase; HIF-1α, hypoxia inducible factor 1; KLF-4, Kruppel-like factor 4; TNFα, tumor necrosis factor-alpha; BMP-7, bone morphogenetic protein 7; P2Y(2)R, P2Y purinoceptor 2; PPARγ, peroxisome proliferator-activated receptor γ; NFκB, nuclear factor-kappa B; FABP4, fatty acid binding protein 4; LXRα; liver X receptor alpha.

The present disclosure provides for the co-expression of IRF transcription factors with one or more molecules that can activate the IRFs to effect TAM reprogramming to an activated state for tumor killing. In particular embodiments, co-expression strategies include: co-expression of IRF5 and IKKβ; co-expression of IRF5 and TANK-binding kinase-1 (TBK-1), TNF receptor-associated factor 6 (TRAF6) adaptor, receptor interacting protein 2 (RIP2) kinase, and/or NFκB kinase-ε (IKKε) (Chang Foreman H-C et al. (2012) PLoS One 7(3): e33098); co-expression of IRF5 and protein kinase DNA-PK (Ryzhakov G et al. (2015) J of Interferon & Cytokine Res 35(2): 71-78); co-expression of IRF5 and protein kinase tyrosine kinase BCR-ABL (Massimo M et al. (2014) Carcinogenesis 35(5):1132-1143); and co-expression of IRF5 or IRF8 with one or more components of the COP9 signalosome (Korczeniewska J et al. (2013) Mol Cell Biol 33(6):1124-1138; Cohen H et al. (2000) J Biol Chem 275(50):39081-39089).

(3) Targeted Antigens and Associated Binding Domains. As used herein, antigens refer to proteins expressed by cell types of interest. Cells of interest or cell types of interest include any pre-defined cell type that is capable of recognition and destruction by the immune system. In some embodiments, cell types of interest in the present invention are cell types that have an adverse, harmful or otherwise undesirable effect (or are pre-disposed to having such an effect) on the health, viability, or well-being of a subject. Cells of interest can include, for example, (i) eukaryotic cells that are either cancerous or infected with a pathogen such as a virus, and (ii) prokaryotic cells, such as certain bacteria, fungi or yeast. Cells of interest also include autoreactive cells that may be harmful and/or cause autoimmunity. Such autoreactive cells of interest include, for example, autoreactive immune cells, autoreactive lymphocytes autoreactive T cells, autoreactive B cells. Autoreactive cells of interest may also be self-reactive cells programmed during development to control the immune response, such as regulatory T-cells. Autoreactive cells contribute to autoimmune conditions in subjects, for example, by recognizing and binding inappropriate self-antigens. In some embodiments, a cell type that may be harmful when over-represented in a localized or circulating cell population may be a cell type or interest according to the invention. For example, an inflammatory reaction may produce an over-representation of immune cells, in which case, cells of interest may include, for example, neutrophils or mast cells. Further, in some instances, cells of interest may be cells previously administered as part of a treatment, for example, genetically-modified cells (e.g., chimeric antigen receptor (CAR) expressing cells).

In particular embodiments, antigens are preferentially expressed by the cells of interest. “Preferentially expressed” means that an antigen is found at higher levels on the cells of interest as compared to other cell types. In some instances, an antigen is only expressed by the cells of interest. In other instances, the antigen is expressed on the cells of interest at least 25%, 35%, 45%, 55%, 65%, 75%, 85%, 95%, 96%, 97%, 98%, 99%, or 100% more than on other cell types.

The following provides examples of cancer cell antigens associated with different cancer types:

| Cancer Type | Associated Cancer Antigens |
|---|---|
| Ovarian Cancer | EpCam, L1-CAM, MUC16, folate receptor (FOLR), Lewis Y, |
| | ROR1, mesothelin, WT-1, PD-L1, EGFR, CD56 |
| Melanoma | Tyrosinase related protein 1 (TYRP1/gp75); GD2, PD-L1, EGFR |
| Glioblastoma | EGFR variant III (EGFRvIII), IL13Ra2 |
| Multiple Myeloma | B-cell maturation antigen (BCMA), PD-L1, EGFR |
| Prostate Cancer | PSMA, WT1, Prostate Stem Cell antigen (PSCA), SV40 T, PD- |
| | L1, EGFR |
| Breast Cancer | HER2, ERBB2, ROR1, PD-L1, EGFR, MUC16, FOLR, CEA |
| Stem Cell Cancer | CD133, PD-L1, EGFR |
| Mesothelioma | mesothelin, PD-L1, EGFR |
| Renal Cell Carcinoma | carboxy-anhydrase-IX (CAIX); PD-L1, EGFR |
| Pancreatic Cancer | mesothelin, CEA, CD24, ROR1, PD-L1, EGFR, MUC16 |
| Lung Cancer | ROR1, PD-L1, EGFR, mesothelin, MUC16, FOLR, CEA, CD56 |
| Cholangiocarcinoma | mesothelin, PD-L1, EGFR |
| Bladder Cancer | MUC16, PD-L1, EGFR, |
| Neuroblastoma | ROR1, glypican-2, CD56, disialoganglioside, PD-L1, EGFR, |
| Colorectal Cancer | CEA, PD-L1, EGFR, |
| Merkel Cell Carcinoma | CD56, PD-L1, EGFR |

Exemplary binding domains for cancer cell antigens include can be generated de novo or derived from known antibodies or binding domains specific for a selected cancer antigen.

Epithelial cell adhesion molecule (EpCam; also referred to as EGP-40, Trop-1, 17-1A, KSA, KS1/4, AUA1, GA733-2, and CD326) is overexpressed in certain cancers, including ovarian cancer. It is a 40kd surface glycoprotein having an extracellular domain with two EGF-like repeats. Antibodies targeting EpCam are commercially available (Richter et al., Am. J. Obstet. Gynecol. 2010, 203(6): 582.e1-582e7). Exemplary antibodies that bind EpCam include MT201 (adecatumumab) and Edrecolomab.

Tyrosine related protein 1 or gp75 glycoprotein (TYRP1/gp75) is a melanosomal protein that is involved in malignant melanocyte and melanoma progression (Ghanem et al., Mol. Oncol. 2011 April; 5(2): 150-155). Exemplary antibodies that bind TYRP1/gp75 include TA99 (Saenger, et al., Cancer Research, 68(23): 9884-9891, 2008), 20D7 (Patel, et al., IOS Press, 16(3-4): 127-1036, 2007), and flanvotumab (IMC-20D7S) (Khalil, et al., Clinical Cancer Research, 22(21): 5204-5210, 2016.

In particular embodiments, an antibody that binds TYRP1/gp75 is described in U.S. Pat. No. 7,951,370. In particular embodiments, the antibody that binds TYRP1/gp75 includes a CDRL1 sequence including RASQSVSSYLA (SEQ ID NO: 84), a CDRL2 sequence including DASNRAT (SEQ ID NO: 85), a CDRL3 sequence including QQRSNWLMYT (SEQ ID NO: 253), a CDRH1 sequence including GYTFTSYAMN (SEQ ID NO: 254), a CDRH2 sequence including WINTNTGNPTYAQGFTG (SEQ ID NO: 255), and a CDRH3 sequence including RYSSSWYLDY (SEQ ID NO: 256).

In particular embodiments, the antibody that binds TYRP1/gp75 includes a CDRL1 sequence including a CDRL1 sequence including RASGNIYNYLA (SEQ ID NO: 257), a CDRL2 sequence including DAKTLAD (SEQ ID NO: 258), a CDRL3 sequence including QHFWSLPFT (SEQ ID NO: 259), a CDRH1 sequence including GFNIKDYFLH (SEQ ID NO: 260), a CDRH2 sequence including WINPDNGNTVYDPKFQG (SEQ ID NO: 261), and a CDRH3 sequence including DYTYEKAALDY (SEQ ID NO: 262).

In particular embodiments, TYRP1/gp75-binding antibodies include a variable light chain including the sequence: EIVLTQSPATLSLSPGERATLSCRASQSVSSYLAWYQQKPGQAPRLLIYDASNRATGIPARFSG SGSGTDFTLTISSLEPEDFAVYYCQQRSNWLMYTFGQGTKLEIK (SEQ ID NO: 263) and a variable heavy chain including the sequence QVQLVQSGSELKKPGASVKISCKASGYTFTSYAMNWVRQAPGQGLECMGWINTNTGNPTYA QGFTGRFVFSMDTSVSTAYLQISSLKAEDTAIYYCAPRYSSSWYLDYWGQGTLVTVSS (SEQ ID NO: 264) or a variable heavy chain including the sequence QVQLVQSGSELKKPGASVKISCKASGYTFTSYAMNWVRQAPGQGLESMGWINTNTGNPTYA QGFTGRFVFSMDTSVSTAYLQISSLKAEDTAIYYCAPRYSSSWYLDYWGQGTLVTVSS (SEQ ID NO: 265).

Exemplary antibodies with binding domains that bind mesothelin include anetumab, ravtansine, Amatuximab, and HN1.

In particular embodiments, the HN1 antibody includes a CDRL1 sequence including RASEGIYHWLA (SEQ ID NO: 55), a CDRL2 sequence including KASSLAS (SEQ ID NO: 58), a CDRL3 sequence including QQYSNYPLT (SEQ ID NO: 61), a CDRH1 sequence including TYYMQ (SEQ ID NO: 64), a CDRH2 sequence including VINPSGVTSYAQKFQG (SEQ ID NO: 71), and a CDRH3 sequence including WALWGDFGMDV (SEQ ID NO: 73).

U.S. Pat. No. 8,206,710 describes mesothelin-binding antibodies including: a variable light chain including the sequence MGWSCIILFLVATATGVHSDIELTQSPAIMSASPGEKVTMTCSASSSVSYMHWYQQKSGTSPK RWIYDTSKLASGVPGRFSGSGSGNSYSLTISSVEAEDDATYYCQQWSKHPLTFGSGTKVEIKR TVAAPSVFIFPPSDEQLKSGTASVVCLLNNFYPREAKVQWKVDNALQSGNSQESVTEQDSKDS TYSLSSTLTLSKADYEKHKVYACEVTHQGLSSPVTKSFNRGEC (SEQ ID NO: 79), and a variable heavy chain including the sequence MGWSCIILFLVATATGVHSQVQLQQSGPELEKPGASVKISCKASGYSFTGYTMNWVKQSHGK SLEWIGLITPYNGASSYNQKFRGKATLTVDKSSSTAYMDLLSLTSEDSAVYFCARGGYDGRGF DYWGSGTPVTVSSASTKGPSVFPLAPSSKSTSGGTAALGCLVKDYFPEPVTVSWNSGALTSG VHTFPAVLQSSGLYSLSSVVTVPSSSLGTQTYICNVNHKPSNTKVDKKVEPKSCDKTHTCPPCP APELLGGPSVFLFPPKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAKGQPREPQVYTLPPSRD ELTKNQVSLTCLVKGFYPSDIAVEWESNGQPENNYKTTPPVLDSDGSFFLYSKLTVDKSRWQQ GNVFSCSVMHEALHNHYTQKSLSLSPGK* (SEQ ID NO: 112);

as well as an antibody having a variable light chain including the sequence MGWSCIILFLVATATGVHSEIVLTQSPATLSLSPGERATLSCSASSSVSYMHWYQQKPGQAPRL LIYDTSKLASGVPARFSGSGSGTDFTLTISSLEPEDFAVYYCQQWSKHPLTFGSGTKVEIKRTV AAPSVFIFPPSDEQLKSGTASVVCLLNNFYPREAKVQWKVDNALQSGNSQESVTEQDSKDSTY SLSSTLTLSKADYEKHKVYACEVTHQGLSSPVTKSFNRGEC (SEQ ID NO: 113), and a variable heavy chain including the sequence MGWSCIILFLVATATGVHSQVQLVQSGAEVKKPGASVKVSCKASGYSFTGYTMNWVRQAPGQ GLEWMGLITPYNGASSYNQKFRGRVTMTRDTSTSTVYMELSSLRSEDTAVYYCARGGYDGRG FDYWGSGTPVTVSSASTKGPSVFPLAPSSKSTSGGTAALGCLVKDYFPEPVTVSWNSGALTS GVHTFPAVLQSSGLYSLSSVVTVPSSSLGTQTYICNVNHKPSNTKVDKKVEPKSCDKTHTCPP CPAPELLGGPSVFLFPPKPKDTLMISRTPEVTCVVVDVSHEDPEVKFNWYVDGVEVHNAKTKP REEQYNSTYRVVSVLTVLHQDWLNGKEYKCKVSNKALPAPIEKTISKAKGQPREPQVYTLPPS RDELTKNQVSLTCLVKGFYPSDIAVEWESNGQPEN NYKTTPPVLDSDGSFFLYSKLTVDKSRW QQGNVFSCSVMHEALHNHYTQKSLSLSPGK (SEQ ID NO: 114).

Additional mesothelin-binding antibodies are described in U.S. Pat. Nos. 8,911,732, 7,081,518, 8,357,783 and 8,425,904.

MUC16 binding domains can be derived from antibodies Oregovomab, ovarex, and abagovomab. U.S. Pat. No. 7,723,485 describes a MUC16 binding antibody including a variable light chain including sequence DIQMTQSPSSLSASVGDRVTITGRASQDVNTAVAWYQQKPGKAPKLLIYSASFLYSGVPSRFS GSRSGTDFTLTISSLQPEDFATYYCQQHYTTPPTFGQGTKVEIKRTVAAPSVFIFPPSDEQLKS GTASVVCLLNNFYPREAKVQWKVDNALQSGNSQESVTEQDSKDSTYSLSSTLTLSKADYEKH KVYACEVTHQGLSSPVTKSFNRGEC (SEQ ID NO: 115), and a variable heavy chain including sequence EVQLVESGGGLVQPGGSLRLSCAASGFNIKDTYIHWVHQAPGKGLEWVARIYPTNGYTRYADS VKGRFTISADTSKNTAYLQMNSLRAEDTAVYYCSRWGGDGFYAMDYWGQGTLVTVSSASTK GPSVFPLAPSSKSTSGGTAALGCLVKDYFPEPVTVSWNSGALTSGVHTFPAVLQSSGLYSLSS VVTVPSSSLGTQTYIGNVNHKPSNTKVDKKVEPKSCDKTHTGPPCPAPELLGGPSVFLFPPKP KDTLMISRTPEVTCVVVDVSHEDPEVKFNWYVDGVEVHNAKTKPREEQYNSTYRVVSVLTVLH QDWLNGKEYKCKVSNKALPAPIEKTISKAKGQPREPQVYTLPPSREEMTKNQVSLTCLVKGFY PSDIAVEWESNGQPENNYKTTPPVLDSDGSFFLYSKLTVDKSRWQQGNVFSCSVMHEALHNH YTQKSLSLSPGK (SEQ ID NO: 116).

WO2016149368 describes a MUC16 binding antibody including a variable light chain including a CDRL1 sequence including SEDIYSG (SEQ ID NO: 117), a CDRL2 sequence including GAS, a CDRL3 sequence including GYSYSSTL (SEQ ID NO: 118), a CDRH1 sequence including TLGMGVG (SEQ ID NO: 119), a CDRH2 sequence including HIWWDDDKYYNPALKS (SEQ ID NO: 120), and a CDRH3 sequence including IGTAQATDALDY (SEQ ID NO: 121).

A folate receptor binding antibody includes farletuzumab. In particular embodiments, farletuzumab is described in U.S. Pat. No. 9,133,275. In particular embodiments, farletuzumab includes a variable light chain including a CDRL1 sequence including KASQSVSFAGTSLMH (SEQ ID NO: 122), a CDRL2 sequence including RASNLEA (SEQ ID NO: 123), and a CDRL3 sequence including QQSREYPYT (SEQ ID NO: 124), and a variable heavy chain including a CDRH1 sequence including GYFMN (SEQ ID NO: 125), a CDRH2 sequence including RIHPYDGDTFYNQKFQG (SEQ ID NO: 126), and a CDRH3 sequence including YDGSRAMDY (SEQ ID NO: 127). Additional FOLR binding antibodies are described in U.S. Ser. No. 10/101,343B2, U.S. Pat. Nos. 8,388,972, and 8,709,432.

An exemplary EGFR antibody includes cetuximab. In particular embodiments, cetuximab is described in US U.S. Pat. No. 7,598,350. In particular embodiments, cetuximab includes a variable light chain including a CDRL1 sequence including RASQSVSSYLA (SEQ ID NO: 84), a CDRL2 sequence including DASNRAT (SEQ ID NO: 85), a CDRL3 sequence including HQYGSTPLT (SEQ ID NO: 130), a CDRH1 sequence including SGDYYWS (SEQ ID NO: 131), a CDRH2 sequence including YIYYSGSTDYNPSLKS (SEQ ID NO: 132), and a CDRH3 sequence including VSIFGVGTFDY (SEQ ID NO: 133).

Additional EGFR binding domains are described in U.S. Pat. Nos. 7,247,301, 7,723,484, 7,132,511, and 5,844,093. U.S. Pat. No. 7,723,484 particularly describes an EGFR binding antibody that includes a variable light chain including sequence EIVLTQSPDFQSVTPKEKVTITCRASYSIGTNIHWYQQKPDQSPKLLIKYASESISGVPSRFSGS GSGTDFTLTINSLEAEDAATYYCQQNNNWPTTFGGGTKVEIK (SEQ ID NO: 134), and a variable heavy chain including sequence QVTLKESGPVLVKPTETLTLTCTVSGFSLSNWDVHWIRQPPGKALEWLAVIWSGGATDYNTPF NSRLTISKDTSKSQVVLTMTNMDPVDTATYYCARALDYYDYNFAYWGQGTMVTVSS (SEQ ID NO: 135).

CD19 binding domains are found within antibody FMC63, SJ25C1 and HD37. (SJ25C1: Bejcek et al. Cancer Res 2005, PMID 7538901; HD37: Pezutto et al. JI 1987, PMID 2437199). In particular embodiments, FMC63 CDRs include a CDRL1 sequence including RASQDISKYLN (SEQ ID NO: 136), a CDRL2 sequence including SRLHSGV (SEQ ID NO: 137), a CDRL3 sequence including GNTLPYTFG (SEQ ID NO: 138), a CDRH1 sequence including DYGVS (SEQ ID NO: 139), a CDRH2 sequence including VTWGSETTYYNSALKS (SEQ ID NO: 140), and a CDRH3 sequence including YAMDYWG (SEQ ID NO: 141).

A number of antibodies specific for RORI are also known to those of skill in the art and can be readily characterized for sequence, epitope binding, and affinity. See, for example, WO2008076868, WO/2008103849, WO201008069, WO2010124188, WO2011079902, WO2011054007, WO2011159847, WO2012076066, WO2012076727, WO 2012045085, and WO2012097313.

Particular examples of antibodies that bind ROR1 include R11, R12, 2A2, and Y31.

The R11 antibody includes a CDRL1 sequence including QASQSIDSNLA (SEQ ID NO: 142), a CDRL2 sequence including RASNLAS (SEQ ID NO: 143), a CDRL3 sequence including LGGVGNVSYRTS (SEQ ID NO: 144), a CDRH1 sequence including DYPIS (SEQ ID NO: 145), a CDRH2 sequence including FINSGGSTWYASWVKG (SEQ ID NO: 146), and a CDRH3 sequence including GYSTYYCDFNI (SEQ ID NO: 147).

The R12 antibody includes a CDRL1 sequence including TLSSAHKTDTID (SEQ ID NO: 148), a CDRL2 sequence including GSYTKRP (SEQ ID NO: 149), a CDRL3 sequence including GADYIGGYV (SEQ ID NO: 150), a CDRH1 sequence including AYYMS (SEQ ID NO: 151), a CDRH2 sequence including TIYPSSGKTYYATWVNG (SEQ ID NO: 152), and a CDRH3 sequence including DSYADDGALFNI (SEQ ID NO: 153).

The 2A2 antibody includes a CDRL1 sequence including KASQNVDAAVA (SEQ ID NO: 154), a CDRL2 sequence including SASNRYT (SEQ ID NO: 155), a CDRL3 sequence including QQYDIYPYT (SEQ ID NO: 156), a CDRH1 sequence including DYEMH (SEQ ID NO: 157), a CDRH2 sequence including AIDPETGGTAYNQKFKG (SEQ ID NO: 158), and a CDRH3 sequence including YYDYDSFTY (SEQ ID NO: 159).

The Y31 antibody includes a CDRL1 sequence including QASQSIGSYLA (SEQ ID NO: 160), a CDRL2 sequence including YASNLAS (SEQ ID NO: 161), a CDRL3 sequence including LGSLSNSDNV (SEQ ID NO: 162), a CDRH1 sequence including SHWMS (SEQ ID NO: 163), a CDRH2 sequence including IIAASGSTYYANWAKG (SEQ ID NO: 164), and a CDRH3 sequence including DYGDYRLVTFNI (SEQ ID NO: 165).

A Her2 binding domain can be derived from the 4D5 antibody. The 4D5 antibody includes a CDRL1 sequence including RASQDVNTAVAW (SEQ ID NO: 166), a CDRL2 sequence including YSASFLES (SEQ ID NO: 167), a CDRL3 sequence including QQHYTTPT (SEQ ID NO: 168), a CDRH1 sequence including SGFNTKDTYIHW (SEQ ID NO: 169), a CDRH2 sequence including RIYPTNGYTRYADSVKGR (SEQ ID NO: 170), and a CDRH3 sequence including WGGDGFYAMDV (SEQ ID NO: 171).

PD-L1 binding antibodies include the 3G10 antibody and those described in US 2016/0222117. In particular embodiments, binding domains derived from the 3G10 antibody include a CDRL1 sequence including RASQSVSSYL (SEQ ID NO: 172), a CDRL2 sequence including DASNRAT (SEQ ID NO: 85), a CDRL3 sequence including QQRSNWPRT (SEQ ID NO: 173), a CDRH1 sequence including DYGFS (SEQ ID NO: 174), a CDRH2 sequence including WITAYNGNTNYAQKLQG (SEQ ID NO: 175), and a CDRH3 sequence including DYFYGMDY (SEQ ID NO: 176).

PD-L1 binding domains can also include a CDRL1 sequence including RASQDVSTAVA (SEQ ID NO: 177), a CDRL2 sequence including SASFLYS (SEQ ID NO: 178), a CDRL3 sequence including QQYLYHPAT (SEQ ID NO: 179), a CDRH1 sequence including SGFTFSDSWIH (SEQ ID NO: 180), a CDRH2 sequence including WISPYGGSTYYADSVKG (SEQ ID NO: 181), and a CDRH3 sequence including RHWPGGFDY (SEQ ID NO: 182) or (ii) a CDRL1 sequence including TGTSSDVGGYNYVS (SEQ ID NO: 183), a CDRL2 sequence including DVSNRPS (SEQ ID NO: 184), a CDRL3 sequence including SSYTSSSTRV (SEQ ID NO: 185), a CDRH1 sequence including SGFTFSSYIMM (SEQ ID NO: 186), a CDRH2 sequence including SIYPSGGITFYADTVKG (SEQ ID NO: 187), and a CDRH3 sequence including IKLGTVTTVDY (SEQ ID NO: 188).

Additional antibodies with PD-L1 binding domains include Atezolizumab, Avelumab, and Durvalumab.

In particular embodiments, antigens are expressed by virally-infected cells. Exemplary viruses include adenoviruses, arenaviruses, bunyaviruses, coronavirusess, flavirviruses, hantaviruses, hepadnaviruses, herpesviruses, papilomaviruses, paramyxoviruses, parvoviruses, picornaviruses, poxviruses, orthomyxoviruses, retroviruses, reoviruses, rhabdoviruses, rotaviruses, spongiform viruses or togaviruses. In additional embodiments, viral antigens include peptides expressed by CMV, cold viruses, Epstein-Barr, flu viruses, hepatitis A, B, and C viruses, herpes simplex, HIV, influenza, Japanese encephalitis, measles, polio, rabies, respiratory syncytial, rubella, smallpox, varicella zoster or West Nile virus.

As further particular examples, coronaviral antigens include the spike (S) protein, cytomegaloviral antigens include envelope glycoprotein B and CMV pp65; Epstein-Barr antigens include EBV EBNAI, EBV P18, and EBV P23; hepatitis antigens include the S, M, and L proteins of hepatitis B virus, the pre-S antigen of hepatitis B virus, HBCAG DELTA, HBV HBE, hepatitis C viral RNA, HCV NS3 and HCV NS4; herpes simplex viral antigens include immediate early proteins and glycoprotein D; HIV antigens include gene products of the gag, pol, and env genes such as HIV gp32, HIV gp41, HIV gp120, HIV gp160, HIV P17/24, HIV P24, HIV P55 GAG, HIV P66 POL, HIV TAT, HIV GP36, the Nef protein and reverse transcriptase; influenza antigens include hemagglutinin and neuraminidase; Japanese encephalitis viral antigens include proteins E, M-E, M-E-NS1, NS1, NS1-NS2A and 80% E; measles antigens include the measles virus fusion protein; rabies antigens include rabies glycoprotein and rabies nucleoprotein; respiratory syncytial viral antigens include the RSV fusion protein and the M2 protein; rotaviral antigens include VP7sc; rubella antigens include proteins E1 and E2; and varicella zoster viral antigens include gpI and gpII. See Fundamental Virology, Second Edition, eds. Fields, B. N. and Knipe, D. M. (Raven Press, New York, 1991) for additional examples of viral antigens.

In particular embodiments, antigens are expressed by cells associated with bacterial infections. Exemplary bacteria include anthrax; gram-negative bacilli, chlamydia, diptheria, haemophilus influenza, Helicobacter pylori, malaria, Mycobacterium tuberculosis, pertussis toxin, pneumococcus, rickettsiae, staphylococcus, streptococcus and tetanus.

As particular examples of bacterial antigens, anthrax antigens include anthrax protective antigen; gram-negative bacilli antigens include lipopolysaccharides; haemophilus influenza antigens include capsular polysaccharides; diptheria antigens include diptheria toxin; Mycobacterium tuberculosis antigens include mycolic acid, heat shock protein 65 (HSP65), the 30 kDa major secreted protein and antigen 85A; pertussis toxin antigens include hemagglutinin, pertactin, FIM2, FIM3 and adenylate cyclase; pneumococcal antigens include pneumolysin and pneumococcal capsular polysaccharides; rickettsiae antigens include rompA; streptococcal antigens include M proteins; and tetanus antigens include tetanus toxin.

Superbugs. In particular embodiments, lymphocytes are modified to target multi-drug resistant “superbugs”. Examples of superbugs include Enterococcus faecium, Clostridium difficile, Acinetobacter baumannii, Pseudomonas aeruginosa, and Enterobacteriaceae (including Escherichia coli, Klebsiella pneumoniae, Enterobacter spp.).

In particular embodiments, antigens are expressed by cells associated with fungal infections. Exemplary fungi include candida, coccidiodes, cryptococcus, histoplasma, leishmania, plasmodium, protozoa, parasites, schistosomae, tinea, toxoplasma, and Trypanosoma cruzi.

As further particular examples of fungal antigens, coccidiodes antigens include spherule antigens; cryptococcal antigens include capsular polysaccharides; histoplasma antigens include heat shock protein 60 (HSP60); leishmania antigens include gp63 and lipophosphoglycan; Plasmodium falciparum antigens include merozoite surface antigens, sporozoite surface antigens, circumsporozoite antigens, gametocyte/gamete surface antigens, protozoal and other parasitic antigens including the blood-stage antigen pf 155/RESA; schistosomae antigens include glutathione-S-transferase and paramyosin; tinea fungal antigens include trichophytin; toxoplasma antigens include SAG-1 and p30; and Trypanosoma cruzi antigens include the 75-77 kDa antigen and the 56 kDa antigen.

In particular embodiments, antigens are expressed by cells associated with autoimmune or allergic conditions. Exemplary autoimmune conditions include acute necrotizing hemorrhagic encephalopathy, allergic asthma, alopecia areata, anemia, aphthous ulcer, arthritis (including rheumatoid arthritis, juvenile rheumatoid arthritis, osteoarthritis, psoriatic arthritis), asthma, autoimmune thyroiditis, celiac disease, conjunctivitis, Crohn's disease, cutaneous lupus erythematosus, dermatitis (including atopic dermatitis and eczematous dermatitis), diabetes, diabetes mellitus, erythema nodosum leprosum, keratoconjunctivitis, multiple sclerosis, myasthenia gravis, psoriasis, scleroderma, Sjogren's syndrome, including keratoconjunctivitis sicca secondary to Sjogren's syndrome, Stevens-Johnson syndrome, systemic lupus erythematosis, ulcerative colitis, vaginitis and Wegener's granulomatosis.

Examples of autoimmune antigens include glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of allergic antigens include pollen antigens such as Japanese cedar pollen antigens, ragweed pollen antigens, rye grass pollen antigens, animal derived antigens (such as dust mite antigens and feline antigens), histocompatibility antigens, and penicillin and other therapeutic drugs.

Binding domains for antigens expressed by cells of interest can also be derived from T cell receptors (TCR). There are numerous ways to identify and select particular TCR for use as binding domains. For example, the sequences of numerous TCR that bind particular antigen fragments are known and publicly available.

Useful TCR can also be identified by isolating T cells that bind a particular antigen and sequencing the TCR chains. As examples, antigen-specific T cells may be induced by in vitro cultivation of isolated human T cells in the presence of an antigen/MHC complex. TCR genes encoding TCR that bind the antigen/MHC complex can be readily cloned by, for example, the 5′ RACE procedure using primers corresponding to the sequences specific to the TCR α-chain gene and the TCR β-chain gene.

In particular embodiments, it may be necessary to pair TCR chains following sequencing (i.e., to perform paired chain analysis). Various methods can be utilized to pair isolated α and β chains. In particular embodiments post-sequencing pairing may be unnecessary or relatively simple, for example in embodiments in which the α and β chain pairing information is not lost in the procedure, such as if one were to sequence from single cells. Chain pairing may also be performed using multiwell sequencing. Assays such as PairSEQ® (Adaptive Biotechnologies Corp., Seattle, WA) have also been developed.

For particular examples of TCR that can be used within the context of the current disclosure, see, for example, WO2018/129270; WO2017/112944; WO2011/039507; U.S. Pat. No. 8,008,438; US2016/0083449; US2015/0246959; Stromnes, et al. (2015) Cancer cell 28(5): 638-652; Kobayashi, et al. (2013) Nature Medicine 19: 1542-1546); Varela-Rohena, et al. (2008) Nature Medicine. 14(12): 1390-1395); and Robbins et al. (2008) The Journal of Immunology 180(9): 6116-6131.

(4) Immune Cell Activating Epitopes and Associated Binding Domains. Immune cells that can be targeted for localized activation include, for example, T cells and natural killer (NK) cells.

T-cell activation can be mediated by two distinct signals: those that initiate antigen-dependent primary activation and provide a T-cell receptor like signal (primary cytoplasmic signaling sequences) and those that act in an antigen-independent manner to provide a secondary or co-stimulatory signal (secondary cytoplasmic signaling sequences). Combinations of bi-specific antibodies can target any combination of T cell activating epitopes that upon binding induce T-cell activation. Examples of such T cell activating epitopes are on T cell markers including CD2, CD3, CD7, CD27, CD28, CD30, CD40, CD83, 4-1BB (CD 137), OX40, lymphocyte function-associated antigen-1 (LFA-1), LIGHT, NKG2C, and B7-H3. T cell suppressive receptors that can be blocked include PD-1, LAG3, TIM-3, BTLA, CTLA-4, and CD200. Antibodies with PD-1 binding domains include Pembrolizumab and Nivolumab while a CTLA-4 blocking antibody includes Ipilimumab.

CD3 is a primary signal transduction element of T cell receptors and is expressed on all mature T cells. Binding domains for CD3 can be derived from, for example, OKT3, 20G6-F3, 4B4-D7, 4E7-C9, and 18F5-H10.

OKT3 is described in U.S. Pat. No. 5,929,212. The OKT3 antibody includes a CDRL1 sequence including SASSSVSYMN (SEQ ID NO: 189), a CDRL2 sequence including RWIYDTSKLAS (SEQ ID NO: 190), a CDRL3 sequence including QQWSSNPFT (SEQ ID NO: 191), a CDRH1 sequence including KASGYTFTRYTMH (SEQ ID NO: 192), a CDRH2 sequence including INPSRGYTNYNQKFKD (SEQ ID NO: 193), and a CDRH3 sequence including YYDDHYCLDY (SEQ ID NO: 194).

The following sequence is an scFv derived from OKT3 which retains the capacity to bind CD3: QVQLQQSGAELARPGASVKMSCKASGYTFTRYTMHWVKQRPGQGLEWIGYINPSRGY TNYNQKFKDKATLTTDKSSSTAYMQLSSLTSEDSAVYYCARYYDDHYCLDYWGQGTTLTVSS SGGGGSGGGGSGGGGSQIVLTQSPAIMSASPGEKVTMTCSASSSVSYMNWYQQKSGTSPKR WIYDTSKLASGVPAHFRGSGSGTSYSLTISGMEAEDAATYYCQQWSSNPFTFGSGTKLEI NR (SEQ ID NO: 195). It may also be used as a CD3 binding domain.

The 20G6-F3 antibody includes a CDRL1 sequence including QSLVHNNGNTY (SEQ ID NO: 196), a CDRL2 sequence including KVS, a CDRL3 sequence including GQGTQYPFT (SEQ ID NO: 197), a CDRH1 sequence including GFTFTKAW (SEQ ID NO: 198), a CDRH2 sequence including IKDKSNSYAT (SEQ ID NO: 199), and a CDRH3 sequence including RGVYYALSPFDY (SEQ ID NO: 200).

The 4B4-D7 antibody includes a CDRL1 sequence including QSLVHDNGNTY (SEQ ID NO: 201), a CDRL2 sequence including KVS, a CDRL3 sequence including GQGTQYPFT (SEQ ID NO: 197), a CDRH1 sequence including GFTFSNAW (SEQ ID NO: 202), a CDRH2 sequence including IKARSNNYAT (SEQ ID NO: 203), and a CDRH3 sequence including RGTYYASKPFDY (SEQ ID NO: 204).

The 4E7-C9 antibody includes a CDRL1 sequence including QSLEHNNGNTY (SEQ ID NO: 205), a CDRL2 sequence including KVS, a CDRL3 sequence including GQGTQYPFT (SEQ ID NO: 197), a CDRH1 sequence including GFTFSNAW (SEQ ID NO: 202), a CDRH2 sequence including IKDKSNNYAT (SEQ ID NO: 206), and a CDRH3 sequence including RYVHYGIGYAMDA (SEQ ID NO: 207).

The 18F5-H10 antibody includes a CDRL1 sequence including QSLVHTNGNTY (SEQ ID NO: 208), a CDRL2 sequence including KVS, a CDRL3 sequence including GQGTHYPFT (SEQ ID NO: 209), a CDRH1 sequence including GFTFTNAW (SEQ ID NO: 210), a CDRH2 sequence including KDKSNNYAT (SEQ ID NO: 211), and a CDRH3 sequence including RYVHYRFAYALDA (SEQ ID NO: 212).

Additional examples of anti-CD3 antibodies, binding domains, and CDRs can be found in WO2016/116626. TR66 may also be used.

CD28 is a surface glycoprotein present on 80% of peripheral T cells in humans and is present on both resting and activated T cells. CD28 binds to B7-1 (CD80) and B7-2 (CD86) and is the most potent of the known co-stimulatory molecules (June et al., Immunol. Today 15:321 (1994); Linsley et al., Ann. Rev. Immunol. 11:191 (1993)).

In particular embodiments, a CD28 binding domain can be derived from CD80, CD86, or the antibodies TGN1412, 9D7, 9.3, KOLT-2, 15E8, 248.23.2, and EX5.3D10.

In particular embodiments, a binding domain derived from TGN1412 includes a CDRL1 sequence including HASQNIYVWLN (SEQ ID NO: 213), a CDRL2 sequence including KASNLHT (SEQ ID NO: 214), a CDRL3 sequence including QQGQTYPYT (SEQ ID NO: 215), a CDRH1 sequence including SYYIH (SEQ ID NO: 216), a CDRH2 sequence including CIYPGNVNTNYNEKFKD (SEQ ID NO: 217), and a CDRH3 sequence including SHYGLDWNFDV (SEQ ID NO: 218).

In particular embodiments a CD80/CD86 binding domain is derived from one or more monoclonal antibodies described in U.S. Pat. No. 7,531,175. In particular embodiments, the CD80/CD86 binding domain includes a CDRL1 sequence including SVSSSISSSNLH (SEQ ID NO: 219), a CDRL2 sequence including GTSNLAS (SEQ ID NO: 220), a CDRL3 sequence including QQWSSYPLT (SEQ ID NO: 221), a CDRH1 sequence including DYYMH (SEQ ID NO: 222), a CDRH2 sequence including WIDPENGNTLYDPKFQG (SEQ ID NO: 223), and a CDRH3 sequence including EGLFFAY (SEQ ID NO: 224).

Activated T-cells express 4-1BB (CD137). 4-1BB, also called CD137 or TNFSF9 (UniProt ID No. Q07011) is a T-cell co-stimulatory receptor.

4-1BB binding domains can be derived from a monoclonal antibody described in U.S. Pat. No. 9,382,328B2

In particular embodiments, the 4-1BB binding domain includes a CDRL1 sequence including RASQSVS (SEQ ID NO: 225), a CDRL2 sequence including ASNRAT (SEQ ID NO: 226), a CDRL3 sequence including QRSNWPPALT (SEQ ID NO: 227), a CDRH1 sequence including YYWS (SEQ ID NO: 228), a CDRH2 sequence including INH, and a CDRH3 sequence including YGPGNYDWYFDL (SEQ ID NO: 229).

In particular embodiments, the 4-1BB binding domain includes a CDRL1 sequence including SGDNIGDQYAH (SEQ ID NO: 230), a CDRL2 sequence including QDKNRPS (SEQ ID NO: 231), a CDRL3 sequence including ATYTGFGSLAV (SEQ ID NO: 232), a CDRH1 sequence including GYSFSTYWIS (SEQ ID NO: 233), a CDRH2 sequence including KIYPGDSYTNYSPS (SEQ ID NO: 234), and a CDRH3 sequence including GYGIFDY (SEQ ID NO: 235).

Cytotoxic T-cells destroy tumor cells. These cells are also known as CD8+ T-cells because they express the CD8 glycoprotein at their surface. These cells recognize their targets by binding to antigen associated with MHC class I, which is present on the surface of nearly every cell of the body.

In particular embodiments, a CD8 binding domain can be derived from the OKT8 antibody. The OKT8 antibody includes a CDRL1 sequence including RTSRSISQYLA (SEQ ID NO: 236), a CDRL2 sequence including SGSTLQS (SEQ ID NO: 237), a CDRL3 sequence including QQHNENPLT (SEQ ID NO: 238), a CDRH1 sequence including GFNIKD (SEQ ID NO: 239), a CDRH2 sequence including RIDPANDNT (SEQ ID NO: 240), and a CDRH3 sequence including GYGYYVFDH (SEQ ID NO: 241).

In particular embodiments, immune cells can be activated by suppressing the activity of inhibitory epitopes such as PD-1, LAG3, TIM-3, BTLA, CTLA-4, VISTA and/or CD200.

PD-1, also called CD279 (UniProt ID No. Q15116) is an inhibitory cell surface receptor involved in regulating the T-cell immune response. In particular embodiments a PD-1 binding domain cam be derived from a monoclonal antibody described in U.S. Patent Publication 2011/0271358. In particular embodiments, the PD-1 binding domain includes a CDRL1 sequence including RASQSVSTSGYSYMH (SEQ ID NO: 242), a CDRL2 sequence including FGSNLES (SEQ ID NO: 243), a CDRL3 sequence including QHSWEIPYT (SEQ ID NO: 244), a CDRH1 sequence including SSWIH (SEQ ID NO: 245), a CDRH2 sequence including YIYPSTGFTEYNQKFKD (SEQ ID NO: 246), and a CDRH3 sequence including WRDSSGYHAMDY (SEQ ID NO: 247).

In particular embodiments, a PD-1 binding domain can be derived from a monoclonal antibody described in U.S. Patent Application 20090217401A1. In particular embodiments, the PD-1 binding domain includes a CDRL1 sequence including RASQSVSSYLA (SEQ ID NO: 84), a CDRL2 sequence including DASNRAT (SEQ ID NO: 85), a CDRL3 sequence including QQSSNWPRT (SEQ ID NO: 248), a CDRH1 sequence including NSGMH (SEQ ID NO: 249), a CDRH2 sequence including VLWYDGSKRYYADSVKG (SEQ ID NO: 250), and a CDRH3 sequence including NDDY (SEQ ID NO: 251).

LAG3, also called CD223 (UniProt ID No. P18627) binds to HLA class-II antigens and is involved in activation of lymphocytes. In particular embodiments a LAG3 binding domain can be derived from a monoclonal antibody described in WO/2014/008218.

TIM-3, also known as HAVcr-2 or TIMD-3 (UniProt ID No. Q9TDQ0) is a cell surface receptor that plays an inhibitory role in innate and adaptive immune responses. In particular embodiments a TIM-3 binding domain can be derived from a monoclonal antibody described in U.S. Patent Publication 2015/0218274.

BTLA, also known as CD272 (UniProt ID No. Q7Z6A9), is an inhibitory receptor that inhibits the immune response of lymphocytes. In particular embodiments a BTLA binding domain (e.g., scFv) can be derived from one or more monoclonal antibodies described in U.S. Patent Publication 2012/0288500.

CTLA-4, also known as CD152 (UniProt ID No. P16410), is an inhibitory receptor that is a major negative regulator of the T-cell response. In particular embodiments a CTLA-4 binding domain can be derived from a monoclonal antibody described in U.S. Pat. No. 6,984,720.

CD200 (also known as ox-2 membrane glycoprotein, UniProt ID No. P41217) is a protein that can deliver inhibitory signals to immune cells. In particular embodiments a CD200 binding domain can be derived from one or more monoclonal antibodies described in U.S. Patent Publication 2013/0189258.

In particular embodiments natural killer cells (also known as NK cells, K cells, and killer cells) are targeted for localized activation by bi-specifics. NK cells can induce apoptosis or cell lysis by releasing granules that disrupt cellular membranes and can secrete cytokines to recruit other immune cells.

Examples of activating proteins expressed on the surface of NK cells include NKG2D, CD8, CD16, KIR2DL4, KIR2DS1, KIR2DS2, KIR3DS1, NKG2C, NKG2E, NKG2D, and several members of the natural cytotoxicity receptor (NCR) family. Examples of NCRs that activate NK cells upon ligand binding include NKp30, NKp44, NKp46, NKp80, and DNAM-1.

Examples of commercially available antibodies that bind to an NK cell receptor and induce and/or enhance activation of NK cells include: 5C6 and 1D11, which bind and activate NKG2D (available from BioLegend® San Diego, CA); mAb 33, which binds and activates KIR2DL4 (available from BioLegend®); P44-8, which binds and activates NKp44 (available from BioLegend®); SK1, which binds and activates CD8; and 3G8 which binds and activates CD16. Additional NK cell activating antibodies are described in WO/2005/0003172 and U.S. Pat. No. 9,415,104.

In relation to CDR sequences and segments, naturally occurring antibody structural units include a tetramer. Each tetramer includes two pairs of polypeptide chains, each pair having one light chain and one heavy chain. The amino-terminal portion of each chain includes a variable region that is responsible for antigen recognition and epitope binding. The variable regions exhibit the same general structure of relatively conserved framework regions (FR) joined by three hyper variable regions, also called complementarity determining regions (CDRs). The CDRs from the two chains of each pair are aligned by the framework regions, which enables binding to a specific epitope. From N-terminal to C-terminal, both light and heavy chain variable regions include the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), or Chothia & Lesk, J. Mol. Biol., 196:901-917 (1987); Chothia et al., Nature, 342:878-883 (1989).

(5) Multi-Specific Molecule Formats. As indicated, multi-specific immune-cell engaging molecules bind both an antigen at a treatment site (e.g., a cancer antigen at a tumor site) and an immune cell activating epitope, with the goal of bringing immune cells to cells of interest to destroy them. Bi-specific antibodies bind on antigen on a cell of interest and one immune cell activating epitopes. Trispecific antibodies can bind two antigens on a cell of interest and one immune cell activating epitope or one antigen on a cell of interest and two immune cell activating epitopes (e.g., a primary activation signal (e.g., CD3) and a costimulatory activation signal (e.g., CD28 or 4-1BB). Quadspecific antibodies can bind four distinct binding partners, divided in multiple combinations between antigens on cells of interest and immune cell activating epitopes. In particular embodiments, the immune cells are T cells or natural killer (NK) cells.

Exemplary bispecific antibody formats are described in, e.g., WO2009/080251, WO2009/080252, WO2009/080253, WO2009/080254, WO2010/112193, WO2010/115589, WO2010/136172, WO2010/145792, and WO2010/145793. For a review of additional bi-specific formats that can be used, see Brinkmann & Kontermann, mAbs, 2017. 9:2, 182-212, DOI: 10.1080/19420862.2016.1268307. Yu et al., (Journal of Hematology & Oncology (2017) 10, 155) describes additional formats particularly useful in the treatment of solid tumors, such as those provided in an Fc format (quadromas, knobs-into-holes, ScFv-IgG, (IgG)2, nanobodies, and ScFv-Fc) and those provided in a non-Fc format (F(ab′)2, ScFv-HAS-scFv, TandscFv, diabodies, DARTs, ImmTACs, dock and locks, and TandAbs).

To the extent not provided above in sections (3) or (4), additional different binding domains can be derived from multiple sources such as antibodies, TCR, fibronectin, affibodies, natural ligands (e.g., CD80 and CD86 for CD28), etc. In particular embodiments, binding domains can be derived from whole antibodies or binding fragments of an antibody, e.g., Fv, Fab, Fab′, F(ab′)₂, Fc, and single chain Fv fragments (scFvs) or any biologically effective fragments of an immunoglobulin that bind specifically to a cancer antigen or immune cell activating epitope (e.g., T cell receptor). Antibodies or antigen binding fragments include all or a portion of polyclonal antibodies, monoclonal antibodies, human antibodies, humanized antibodies, synthetic antibodies, chimeric antibodies, bispecific antibodies, mini bodies, and linear antibodies.

Multi-specifics including binding domains from human origin or humanized antibodies have lowered immunogenicity in humans and have a lower number of non-immunogenic epitopes compared to non-human antibodies. Binding domains will generally be selected to have reduced antigenicity in human subjects. Binding domains can particularly include any peptide that specifically binds a selected cancer antigen or immune cell activating epitope. Sources of binding domains include antibody variable regions from various species (which can be in the form of antibodies, sFvs, scFvs, Fabs, scFv-based grababody, or soluble VH domain or domain antibodies). These antibodies can form antigen-binding regions using only a heavy chain variable region, i.e., these functional antibodies are homodimers of heavy chains only (referred to as “heavy chain antibodies”) (Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008).

Phage display libraries of partially or fully synthetic antibodies are available and can be screened for an antibody or fragment thereof that can bind a selected epitope. For example, binding domains may be identified by screening a Fab phage library for Fab fragments that specifically bind to a target of interest (see Hoet et al., Nat. Biotechnol. 23:344, 2005). Phage display libraries of human antibodies are also available. Additionally, traditional strategies for hybridoma development using a target of interest as an immunogen in convenient systems (e.g., mice, HuMAb Mouse®, TC Mouse™, KM-Mouse®, llamas, chicken, rats, hamsters, rabbits, etc.) can be used to develop binding domains. In particular embodiments, binding domains specifically bind to selected epitopes expressed by targeted cancer cells and/or T cells and do not cross react with nonspecific components or unrelated targets. Once identified, the amino acid sequence or polynucleotide sequence coding for the CDR within a binding domain can be isolated and/or determined.

An alternative source of binding domains includes sequences that encode random peptide libraries or sequences that encode an engineered diversity of amino acids in loop regions of alternative non-antibody scaffolds, such as scTCR (see, e.g., Lake et al., Int. Immunol. 11:745, 1999; Maynard et al., J. Immunol. Methods 306:51, 2005; U.S. Pat. No. 8,361,794), mAb² or Fcab™ (see, e.g., PCT Patent Application Publication Nos. WO 2007/098934; WO 2006/072620), affibodies, avimers, fynomers, cytotoxic T-lymphocyte associated protein-4 (Weidle et al., Cancer Gen. Proteo. 10:155, 2013), and the like (Nord et al., Protein Eng. 8:601, 1995; Nord et al., Nat. Biotechnol. 15:772, 1997; Nord et al., Euro. J. Biochem. 268:4269, 2001; Binz et al., Nat. Biotechnol. 23:1257, 2005; Boersma and Plückthun, Curr. Opin. Biotechnol. 22:849, 2011).

In particular embodiments, an antibody fragment is used as one or more binding domains in a multi-specific. An “antibody fragment” denotes a portion of a complete or full-length antibody that retains the ability to bind to an epitope. Examples of antibody fragments include Fv, scFv, Fab, Fab′, Fab′-SH, F(ab′)₂; diabodies; and linear antibodies.

A single chain variable fragment (scFv) is a fusion protein of the variable regions of the heavy and light chains of immunoglobulins connected with a short linker peptide. Fv fragments include the VL and VH domains of a single arm of an antibody. Although the two domains of the Fv fragment, VL and VH, are coded by separate genes, they can be joined, using, for example, recombinant methods, by a synthetic linker that enables them to be made as a single protein chain in which the VL and VH regions pair to form monovalent molecules (single chain Fv (scFv)). For additional information regarding Fv and scFv, see e.g., Bird, et al., Science 242 (1988) 423-426; Huston, et al., Proc. Natl. Acad. Sci. USA 85 (1988) 5879-5883; Plueckthun, in The Pharmacology of Monoclonal Antibodies, vol. 113, Rosenburg and Moore (eds.), Springer-Verlag, New York), (1994) 269-315; WO1993/16185; U.S. Pat. Nos. 5,571,894; and 5,587,458.

A Fab fragment is a monovalent antibody fragment including VL, VH, CL and CH1 domains. A F(ab′)2 fragment is a bivalent fragment including two Fab fragments linked by a disulfide bridge at the hinge region. For discussion of Fab and F(ab′)2 fragments having increased in vivo half-life, see U.S. Pat. No. 5,869,046. Diabodies include two epitope-binding sites that may be bivalent. See, for example, EP 0404097; WO1993/01161; and Holliger, et al., Proc. Natl. Acad. Sci. USA 90 (1993) 6444-6448. Dual affinity retargeting antibodies (DART™; based on the diabody format but featuring a C-terminal disulfide bridge for additional stabilization (Moore et al., Blood 117, 4542-51 (2011))) can also be used. Antibody fragments can also include isolated CDRs. For a review of antibody fragments, see Hudson, et al., Nat. Med. 9 (2003) 129-134.

In particular embodiments, multi-specific antibodies can also include a natural receptor or ligand for an epitope as a binding domain. For example, if a target for binding includes PD-L1, the binding domains can include PD-1 (including, e.g., a PD-1/antiCD3 fusion). One example of a receptor fusion for binding is Enbrel® (Amgen). Natural receptors or ligands can also be modified to enhance binding. For example, betalacept is a modified version of abatacept. In particular embodiments, the multi-specific can include a natural receptor or ligand that induces phagocytosis. Calreticulin (UniProt ID No. P27797) is a protein that is localized to the endoplasmic reticulum of healthy cells, but in dying cells it translocates to the cell surface and induces phagocytosis by immune cells such as macrophages. In particular embodiments, the binding domains can include calreticulin or a portion of calreticulin that is capable of inducing phagocytosis.

In particular embodiments, the multi-specific can include a single chain antibody attached to the C-terminus of a light chain (see, e.g., Oncoimmunology. 2017; 6 (3): e1267891). This format can be useful because the presence of the Fc region can help preserve the protein half-life. The presence of the Fc region can also be useful because Fc interacts with several receptors and can contribute to the immune response. Antibody-scFv fusions can also be useful because the antibody portion binds to its epitope in a dimeric fashion, which enhances avidity and the scFv portion binds its epitope in a monomeric fashion, which can be useful, for example, for binding T-cell epitopes and only allowing multimerization in the presence of a target (e.g., cancer cell). These embodiments can be “tri-specific”.

As indicated, binding domains of a multi-specifics may be joined through a linker. A linker is an amino acid sequence which can provide flexibility and room for conformational movement between the binding domains of a multi-specific. Any appropriate linker may be used. Examples of linkers can be found in Chen et al., Adv Drug Deliv Rev. 2013 Oct. 15; 65(10): 1357-1369. Linkers can be flexible, rigid, or semi-rigid, depending on the desired functional domain presentation to a target. Commonly used flexible linkers include Gly-Ser linkers such as GGSGGGSGGSG (SEQ ID NO: 252), GGSGGGSGSG (SEQ ID NO: 63) and GGSGGGSG (SEQ ID NO: 65). Additional examples include: GGGGSGGGGS (SEQ ID NO: 90); GGGSGGGS (SEQ ID NO: 128); and GGSGGS (SEQ ID NO: 129). Linkers that include one or more antibody hinge regions and/or immunoglobulin heavy chain constant regions, such as CH3 alone or a CH2CH3 sequence can also be used.

In some situations, flexible linkers may be incapable of maintaining a distance or positioning of binding domains needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

Particular examples of bi-specific antibodies are described in Yu et al., (Journal of Hematology & Oncology (2017) 10, 155) and include Catumaxomab (EpCAM/CD3; in a Triomab format and evaluated in clinical trial NCT00189345); MT110 (EpCAM/CD3; in a BiTE (Amgen) format and evaluated in clinical trial NCT00635596); Ertumaxomab (HER2/CD3; in a Triomab format and evaluated in clinical trial NCT00452140); MDX-447 (EGFR/CD64; in a 2(Fab′) format and evaluated in clinical trial NCT00005813); MM-141 (HER3/IGF-IR; in a scFv-IgG format and evaluated in clinical trial NCT01733004); AMG211 (CEA/CD3; in a BiTE format and evaluated in clinical trial NCT02760199); R06958688 (CEA/CD3; in a IgG-based format and evaluated in clinical trial NCT02324257); R06895882 (CEA/IL2; in a ScFv-IgG format and evaluated in clinical trial NCT02004106); TF2 (CEA/HSG; in a Dock and lock format and evaluated in clinical trial NCT00860860); Anti-CEAxanti-DTPA (CEA/di-DTPA-131; in a scFv-IgG format and evaluated in clinical trial NCT00467506); BAY2010112 (PSMA/CD3; in a BiTE format and evaluated in clinical trial NCT01723475); and MOR209/ES414 (PSMA/CD3; in a ScFv-Fc-scFv format and evaluated in clinical trial NCT02262910). AMG701 (BCMA-targeting) and Solitomab (EpCam/CD3 targeting) can also be used. For additional information regarding EpCam binding bi-specific molecules, see Brischwein et al., Mol. Immunol. 2006; 43: 1129-1143 and Schlereth et al., Cancer Research, 2005; 65: 2882-2889. A PD-L1/CD3 bi-specific as described in Horn et al., Oncotarget, Aug. 29, 2017 8 (35) can also be used. Additional particular examples of bi-specific antibodies are described in WO2014/167022; US2016/0208001; US 2014/0302037 and US 2014/0308285. One last example includes Blinatumomab. See Ellerman, Methods, 154 (2019) 102-117 for additional information regarding bispecific-T cell engagers.

(6) TGFβ. Inhibitors. Three highly homologous isoforms of TGFβ exist in humans: TGFβ1, TGFβ2 and TGFβ3. Numerous inhibitory TGFβ peptides and antibodies are available. In particular embodiments, monocytes/macrophages can be reprogrammed to express an inhibitory TGFβ peptide or antibody. Examples of TGFβ inhibitors include Trabedersen (AP12009; an antisense oligo evaluated in clinical trials NCT00431561, NCT00844064, and NCT00761280); Disitertide (a peptide evaluated in clinical trials NCT00574613 and NCT00781053); Lerdelimumab (a humanized antibody); Metelimumab (a humanized antibody evaluated in clinical trial NCT00043706); Fresolimumab (a humanized antibody evaluated in clinical trials NCT00464321, NCT01284322, and NCT01291784); LY2382770 (a humanized antibody evaluated in clinical trial NCT01113801); SIX-100 (an antibody evaluated in clinical trial NCT01371305); Avotermin (a recombinant protein evaluated in clinical trials NCT004322111 and NCT00656227); and IMC-TR1 (a humanized antibody evaluated in clinical trial NCT01646203).

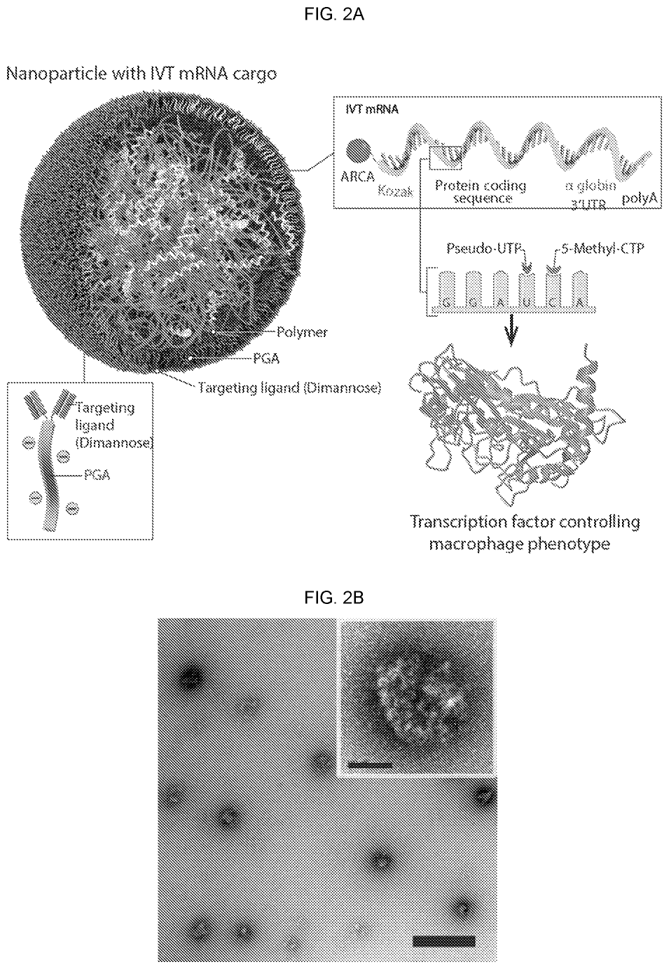

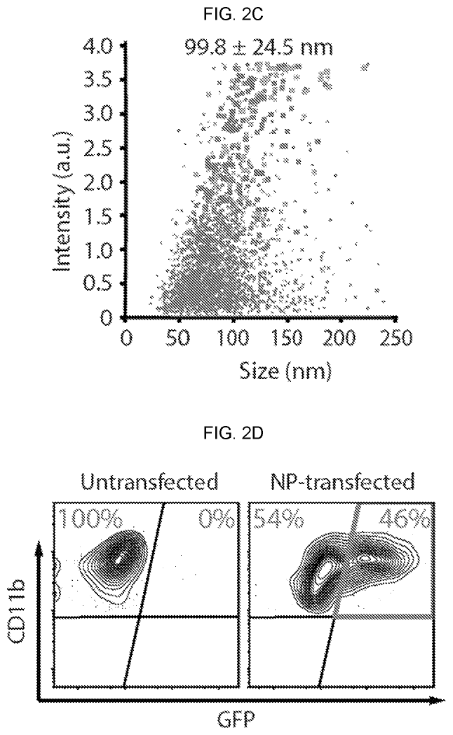

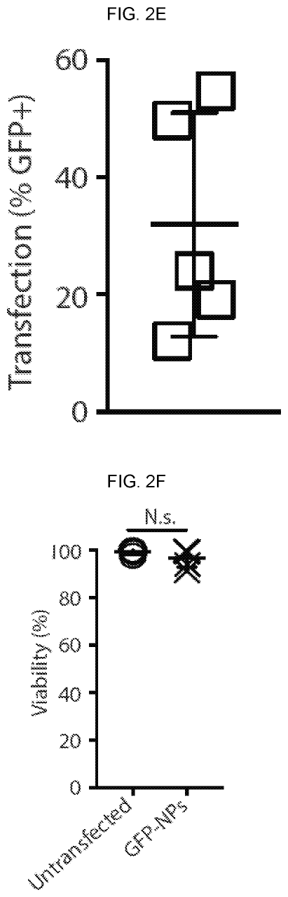

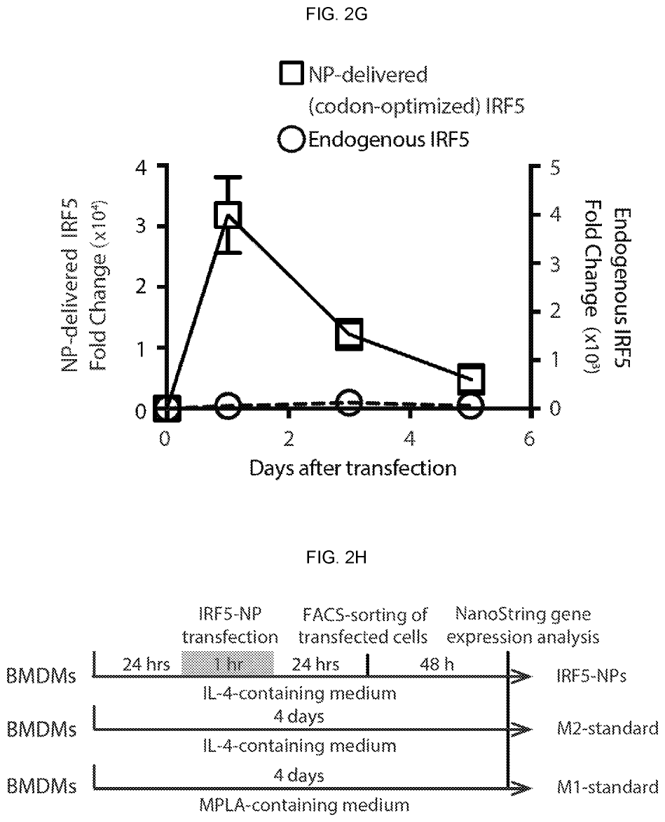

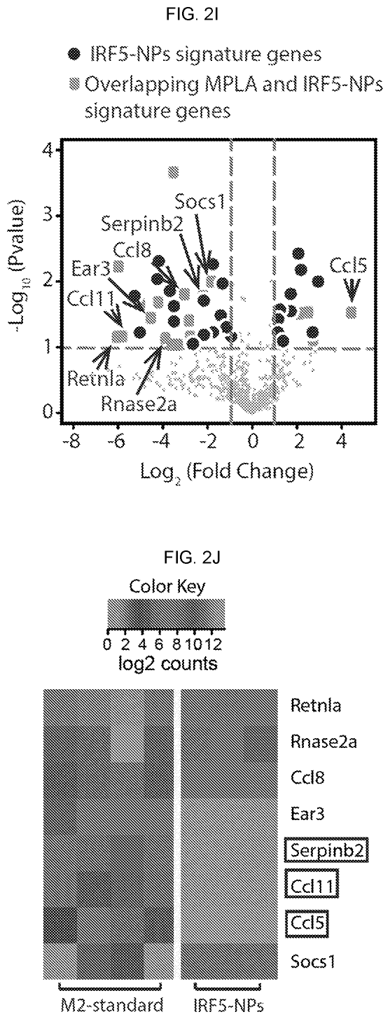

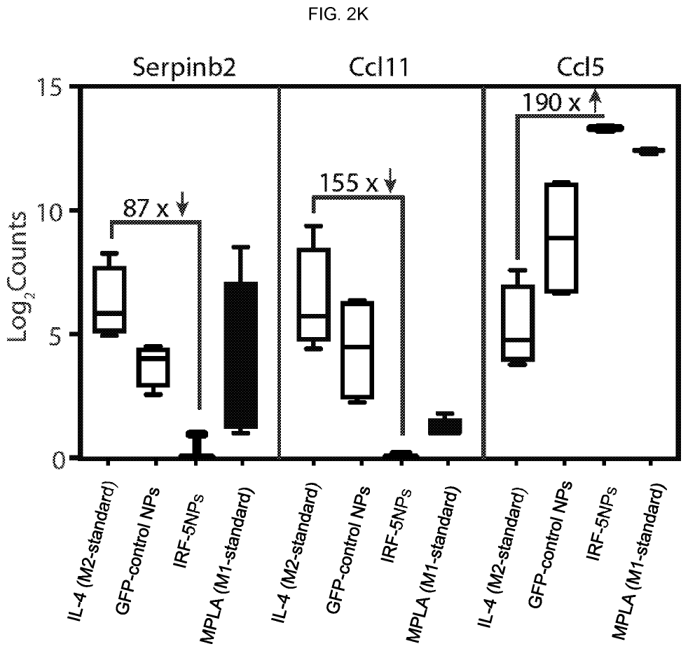

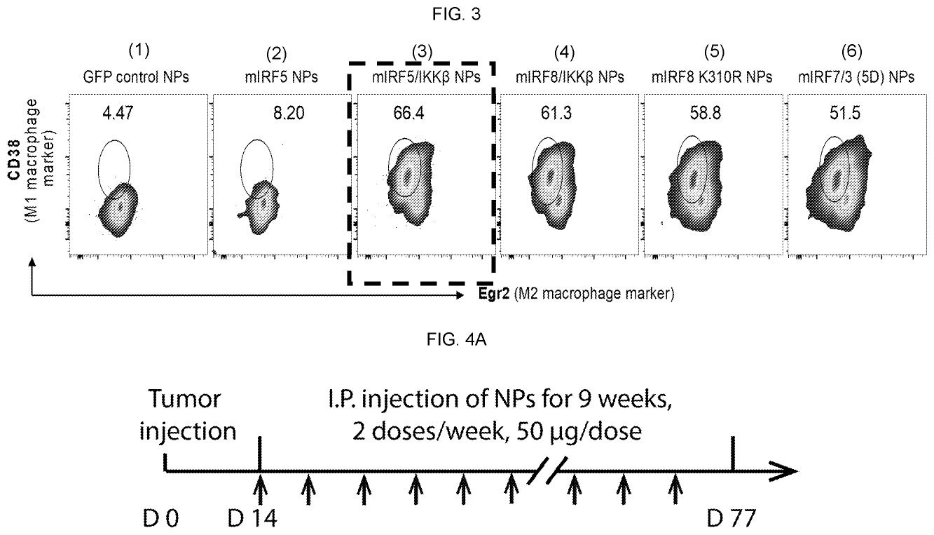

Ravi et al., (Nature Communications 9, 741 (2018)) describe bifunctional antibody—ligand traps (Y-traps) including an antibody targeting CTLA-4 or PD-L1 fused to a TGFβ receptor H ectodomain sequence that simultaneously disables autocrine/paracrine TGFβ in the target cell microenvironment (a-CTLA4-TGFβRIIecd and a-PDL1-TGFβRIIecd). FIG. 2B of Ravi et al., provides the amino add sequences of the heavy chain and light chain of a-CTLA4-TGFβRII including the ligand binding sequence of the TGFβRII extracellular domain. Cuende et al., Science Translational Medicine, Apr. 2015, 7(284) additionally describes the production of antibodies that inhibit TGFβ in vivo; for example, the anti-GARP monoclonal antibodies MHG-8 and LHG-10 block the production of active TGF-β1.

Examples of additional TGFβ inhibitors include tranilast, pirfenidone, Lefty-1 (11051 Accession Nos: NM_010094 (mouse), and NM_020997 (human)), SB-431542, SB-202190, and SB-505124 (Lindemann et al., Mol. Cancer, 2003, 2: 20; GlaxoSmithKline), NPC30345, SD093, SD908, SD208 (Scios), SM16 (Biogen Idec), LY2109761, LY364947, LY580276. LY2157299 (Lilly Research Laboratories), A-83-01 (WO 2009/146408), ALK5 inhibitor II (2-[3-[6-methylpyridin-2-yl]-1H-pyrazol-4 yl]-1,5-naphthyridine), TGβRI kinase inhibitor VIII (6-[2-tert-butyl-5-[6-methyl-pyridin-2-yl]-1H-imidazol-4-yl]-quinoxaline) and derivatives thereof.

(7) Nucleotides. Within the current disclosure, nucleotides encoding genes that regulate activation states and genes that result in expression of multi-specific molecules and optionally a TGFβ inhibitor are delivered to immune cells, such as monocytes and/or macrophages. “Gene” refers to a nucleotide sequence that encodes an encoded molecule. This definition includes various sequence polymorphisms, mutations, and/or sequence variants wherein such alterations do not affect the function of the encoded molecule. The term “gene” may include not only coding sequences but also regulatory regions such as promoters, enhancers, and termination regions. The term further can include all introns and other DNA sequences spliced from the mRNA transcript, along with variants resulting from alternative splice sites. Nucleotide sequences encoding the encoded molecule can be RNA that directs the expression of the encoded molecule. These nucleotide sequences include RNA sequences that are translated, in particular embodiments, into protein. In particular embodiments, one of ordinary skill in the art can appreciate that DNA sequences including thymine (T) bases can be equivalent to mRNA sequences having the same sequence except that T bases are replaced by uracil (U) bases. The nucleotide sequences include both the full-length nucleotide sequences as well as non-full-length sequences derived from the full-length protein. The sequences can also include degenerate codons of the native sequence or sequences that may be introduced to provide codon preference in a specific immune cell. Gene sequences to encode molecules described herein are available in publicly available databases and publications. “Encoding” refers to a property of sequences of nucleotides, such as a plasmid, a gene, cDNA, mRNA, to serve as templates for synthesis of an activation regulator, multi-specific antibody, and/or a TGFβ inhibitor.

In particular embodiments, the nucleotides include synthetic mRNA. In particular embodiments, synthetic mRNA is engineered for increased intracellular stability using 5′-capping. Multiple distinct 5′-cap structures can be used to generate the 5′-cap of a synthetic mRNA molecule. For example, the Anti-Reverse Cap Analog (ARCA) cap contains a 5′-5′-triphosphate guanine-guanine linkage where one guanine contains an N7 methyl group as well as a 3′-O-methyl group. Synthetic mRNA molecules may also be capped post-transcriptionally using enzymes responsible for generating 5′-cap structures. For example, recombinant Vaccinia Virus Capping Enzyme and recombinant 2′-O-methyltransferase enzyme can create a canonical 5′-5′-triphosphate linkage between the 5′-most nucleotide of an mRNA and a guanine nucleotide where the guanine contains an N7 methylation and the ultimate 5′-nucleotide contains a 2′-O-methyl generating the Cap1 structure. This results in a cap with higher translational-competency and cellular stability and reduced activation of cellular pro-inflammatory cytokines.

In particular embodiments, other modifications of synthetic mRNA to reduce immunogenicity, promote mRNA stability, and/or promote translation of mRNA can include 5′- and 3′-terminal untranslated regions (UTRs), a Kozak translation initiation sequence in the 5′ UTR, modified ribonucleosides, and/or a polyA tail. In particular embodiments, modified ribonucleosides can include pseudouridine (Ψ), 5-methylcytidine (5mC), N6-methyladenosine (m6A), 2-thiouridine (2sU), 5-methoxyuridine (5moU), and N-1-methylpseudouridine (m1Ψ). In particular embodiments, UTRs can include alpha- and/or beta-globin UTRs.

Particular embodiments of producing synthetic mRNA include generating a DNA template containing the coding DNA sequence of the desired protein with a 5′ T100250 overhang by PCR amplification from a corresponding DNA plasmid. The DNA template can then be used to produce the mRNA by an in vitro transcription reaction. During in vitro transcription, a 5′ cap structure (e.g., ARCA), modified ribonucleosides, and/or a 3′ poly(A) tail can be incorporated. A number of in vitro transcription systems are commercially available including from, e.g., MEGAscript T7 transcription kit (ThermoFisher Scientific, Waltham, MA), Riboprobe™ System T7 (Promega, Madison, WI), AmpliScribe™ T7 high yield transcription kit (Epicentre, Madison, WI), and HiScribe™ T7 in vitro transcription kit (New England Biolabs, Ipswich, MA). In particular embodiments, synthetic mRNA can be synthesized by companies that synthesize nucleotides (e.g., Tri Link Biotechnologies, San Diego, CA).

Synthetic mRNA or other nucleotides may be made cyclic. Such nucleotides may be cyclized, or concatemerized, to generate a translation competent molecule to assist interactions between poly-A binding proteins and 5′-end binding proteins. The mechanism of cyclization or concatemerization may occur through at least 3 different routes: 1) chemical, 2) enzymatic, or 3) ribozyme catalyzed. The newly formed 5′-/3′-linkage may be intramolecular or intermolecular.

In the first route, the 5′-end and the 3′-end of the nucleotide may contain chemically reactive groups that, when close together, form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a nucleotide molecule will undergo a nucleophilic attack on the 5′-NHS-ester moiety forming a new 5′-/3′-amide bond.

In the second route, T4 RNA ligase may be used to enzymatically link a 5′-phosphorylated nucleotide molecule to the 3′-hydroxyl group of a nucleotide forming a new phosphorodiester linkage. In an example reaction, 1 μg of a nucleotide molecule can be incubated at 37° C. for 1 hour with 1-10 units of T4 RNA ligase (New England Biolabs, Ipswich, MA) according to the manufacturer's protocol. The ligation reaction may occur in the presence of a split oligonucleotide capable of base-pairing with both the 5′- and 3′-region in juxtaposition to assist the enzymatic ligation reaction.

In the third route, either the 5′- or 3′-end of a cDNA template encodes a ligase ribozyme sequence such that during in vitro transcription, the resultant nucleotide molecule can contain an active ribozyme sequence capable of ligating the 5′-end of a nucleotide molecule to the 3′-end of a nucleotide molecule. The ligase ribozyme may be derived from the Group I Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

In particular embodiments, nucleotides include a plasmid, a cDNA, or an mRNA that can include, e.g., a sequence (e.g., a gene) for expressing an encoded molecule. Suitable plasmids include standard plasmid vectors and minicircle plasmids that can be used to transfer a gene to a monocyte/macrophage. The nucleotides (e.g., minicircle plasmids) can further include any additional sequence information to facilitate transient expression in a modified cell. For example, the nucleotides can include promoters, such as general promoters, tissue-specific promoters, cell-specific promoters, and/or promoters specific for the cytoplasm. As indicated, promoters and plasmids (e.g., minicircle plasmids) are generally well known in the art and can be prepared using conventional techniques.

For additional information regarding nucleotides that can be used within embodiments of the current disclosure, see Hardee et al., Genes (2017), 8, 65. Hardee et al., reviews methods of non-viral DNA gene therapy vectors including plasmids, minicircles, and minivectors. Plasmid vectors and minimized DNA vectors have successfully been used to deliver gene therapies for cancer. Minicircles have more recently been used to engineer T-cells to deliver bi-specific antibodies to allow the T-cells to kill B-cell lymphomas (Hardee et al., Genes (2017), 8, 65). Particular embodiments include use of double-stranded DNA (integrating and/or non-integrating), conventional plasmids, minicircles, and/or closed-ended linear ceDNA (see Li et al., PLoS One, Aug. 1, 2013 doi.org/10.1371/journal.pone. 0069879). The ceDNA is a non-viral, AAV-derived vector DNA with covalently closed ends (Li et al., PLoS One, 2013, doi.org/10.1371/journal.pone. 0069879).

In particular embodiments, a nucleotide encoding a macrophage activation regulator is used in combination with one or more additional nucleotides encoding other activation regulators (e.g., combinations of IRFs, multi-specific antibodies and/or TGFβ inhibitors). In particular embodiments, a nucleotide encoding an IRF is used in combination with one or more additional nucleotides encoding other IRFs and with a nucleotide encoding a IKKβ. In particular embodiments, a nucleotide encoding an IRF is used in combination with a nucleotide encoding a IKKβ, multi-specific antibodies and/or TGFβ inhibitors at a ratio of 0.5:1, 1:1, 2:1, 3:1, 4:1, or 5:1. In particular embodiments, a nucleotide encoding an IRF is used in combination with a nucleotide encoding a IKKβ at a ratio of 3:1.

Particular embodiments can deliver nucleotides within a gene editing system. Gene editing systems modify or affect particular sequences of a cell's endogenous genome. Gene editing systems are useful for targeted genome editing, for example gene disruption, gene editing by homologous recombination, and gene therapy to insert therapeutic genes at the appropriate chromosomal target sites with a human genome.

Particular embodiments utilize transcription activator-like effector nucleases (TALENs) as gene editing systems. TALENs refer to fusion proteins including a transcription activator-like effector (TALE) DNA binding protein and a DNA cleavage domain. TALENs are used to edit genes and genomes by inducing double strand breaks (DSBs) in the DNA, which induce repair mechanisms in cells. Generally, two TALENs must bind and flank each side of the target DNA site for the DNA cleavage domain to dimerize and induce a DSB. The DSB is repaired in the cell by non-homologous end-joining (NHEJ) or by homologous recombination (HR) with an exogenous double-stranded donor DNA fragment.

As indicated, TALENs have been engineered to bind a target sequence of, for example, an endogenous genome, and cut DNA at the location of the target sequence. The TALEs of TALENs are DNA binding proteins secreted by Xanthomonas bacteria. The DNA binding domain of TALEs include a highly conserved 33 or 34 amino acid repeat, with divergent residues at the 12th and 13th positions of each repeat. These two positions, referred to as the Repeat Variable Diresidue (RVD), show a strong correlation with specific nucleotide recognition. Accordingly, targeting specificity can be improved by changing the amino acids in the RVD and incorporating nonconventional RVD amino acids.

Examples of DNA cleavage domains that can be used in TALEN fusions are wild-type and variant FokI endonucleases. The FokI domain functions as a dimer requiring two constructs with unique DNA binding domains for sites on the target sequence. The FokI cleavage domain cleaves within a five or six base pair spacer sequence separating the two inverted half-sites.

Particular embodiments utilize MegaTALs as gene editing systems. MegaTALs have a single chain rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Particular embodiments utilize zinc finger nucleases (ZFNs) as gene editing systems. ZFNs are a class of site-specific nucleases engineered to bind and cleave DNA at specific positions. ZFNs are used to introduce DSBs at a specific site in a DNA sequence which enables the ZFNs to target unique sequences within a genome in a variety of different cells. Moreover, subsequent to double-stranded breakage, homologous recombination or non-homologous end joining takes place to repair the DSB, thus enabling genome editing.

ZFNs are synthesized by fusing a zinc finger DNA-binding domain to a DNA cleavage domain. The DNA-binding domain includes three to six zinc finger proteins which are transcription factors. The DNA cleavage domain includes the catalytic domain of, for example, FokI endonuclease.

Guide RNA can be used, for example, with gene-editing systems such as CRISPR-Cas systems. CRISPR-Cas systems include CRISPR repeats and a set of CRISPR-associated genes (Cas).

In general, any system capable of resulting in functional expression of delivered nucleotides can be used within the current disclosure. However, in particular embodiments, delivery utilizing viral vectors is excluded.

(8) Nanoparticles. In certain examples, nanoparticles used within the systems and methods disclosed herein can function to condense and protect nucleotides from enzymatic degradation. Particularly useful materials to use within nanoparticles for this purpose include positively charged lipids and/or polymers, including poly(β-amino ester) (PbAE).

Examples of positively charged polymers that can be used within nanoparticles of the current disclosure include polyamines; polyorganic amines (e.g., polyethyleneimine (PEI), polyethyleneimine celluloses); poly(amidoamines) (PAMAM); polyamino acids (e.g., polylysine (PLL), polyarginine); polysaccharides (e.g, cellulose, dextran, DEAE dextran, starch); spermine, spermidine, poly(vinylbenzyl trialkyl ammonium), poly(4-vinyl-N-alkyl-pyridiumiun), poly(acryloyl-trialkyl ammonium), and Tat proteins.

Examples of positively charged lipids include esters of phosphatidic acid with an aminoalcohol, such as an ester of dipalmitoyl phosphatidic acid or distearoyl phosphatidic acid with hydroxyethylenediamine. More particular examples of positively charged lipids include 3β-[N-(N′, N′-dimethylaminoethyl)carbamoyl) cholesterol (DC-chol); N,N′-dimethyl-N,N′-dioctacyl ammonium bromide (DDAB); N,N′-dimethyl-N,N′-dioctacyl ammonium chloride (DDAC); 1,2-dioleoyloxypropyl-3-dimethyl-hydroxyethyl ammonium chloride (DORI); 1,2-dioleoyloxy-3-[trimethylammonio]-propane (DOTAP); N-(1-(2,3-dioleyloxy)propyl)-N,N,N-trimethylammonium chloride (DOTMA); dipalmitoylphosphatidylcholine (DPPC); 1,2-dioctadecyloxy-3-[trimethylammonio]-propane (DSTAP); and the cationic lipids described in e.g. Martin et al., Current Pharmaceutical Design 2005, 11, 375-394.

Blends of lipids and polymers in any concentration and in any ratio can also be used. Blending different polymer types in different ratios using various grades can result in characteristics that borrow from each of the contributing polymers. Various terminal group chemistries can also be adopted.

Particular embodiments disclosed herein can also utilize porous nanoparticles constructed from any material capable of forming a porous network. Exemplary materials include metals, transition metals and metalloids. Exemplary metals, transition metals and metalloids include lithium, magnesium, zinc, aluminum and silica. In particular embodiments, the porous nanoparticles include silica. The exceptionally high surface area of mesoporous silica (exceeding 1,000 m2/g) enables nucleotide loading at levels exceeding conventional DNA carriers such as liposomes.

Particles can be formed in a variety of different shapes, including spheroidal, cuboidal, pyramidal, oblong, cylindrical, toroidal, and the like. The nucleotides can be included in the pores of the nanoparticles in a variety of ways. For example, the nucleotides can be encapsulated in the porous nanoparticles. In other aspects, the nucleotides can be associated (e.g., covalently and/or non-covalently) with the surface or close underlying vicinity of the surface of the porous nanoparticles. In particular embodiments, the nucleotides can be incorporated in the porous nanoparticles e.g., integrated in the material of the porous nanoparticles. For example, the nucleotides can be incorporated into a polymer matrix of polymer nanoparticles.

In particular embodiments, the nanoparticles disclosed herein include a coating. A coating can serve to shield the encapsulated nucleotides and/or reduce or prevent off-target binding. Off-target binding is reduced or prevented by reducing the surface charge of the nanoparticles to neutral or negative. As disclosed in more detail elsewhere herein, coatings can include neutral or negatively charged polymer- and/or liposome-based coatings. In particular embodiments, the coating is a dense surface coating of hydrophilic and/or neutrally charged hydrophilic polymer sufficient to prevent the encapsulated nucleotides from being exposed to the environment before release into an immune cell. In particular embodiments, the coating covers at least 80% or at least 90% of the surface of the nanoparticle. In particular embodiments, the coating includes polyglutamic acid (PGA). In particular embodiments, PGA can serve as a linker to attach a targeting ligand to a nanoparticle. In particular embodiments, PGA can serve as a linker to attach di-mannose to a nanoparticle. In particular embodiments, the coating includes hyaluronic acid.

Examples of neutrally charged polymers that can be used as coating within embodiments of the disclosure include polyethylene glycol (PEG); poly(propylene glycol); and polyalkylene oxide copolymers, (PLURONIC®, BASF Corp., Mount Olive, NJ).

Neutrally charged polymers also include zwitterionic polymers. Zwitterionic refers to the property of overall charge neutrality while having both a positive and a negative electrical charge. Zwitterionic polymers can behave like regions of cell membranes that resist cell and protein adhesion.

Zwitterionic polymers include zwitterionic constitutional units including pendant groups (i.e., groups pendant from the polymer backbone) with zwitterionic groups. Exemplary zwitterionic pendant groups include carboxybetaine groups (e.g., —Ra-N+(Rb)(Rc)-Rd-CO₂—, where Ra is a linker group that covalently couples the polymer backbone to the cationic nitrogen center of the carboxybetaine groups, Rb and Rc are nitrogen substituents, and Rd is a linker group that covalently couples the cationic nitrogen center to the carboxy group of the carboxybetaine group).

Examples of negatively charged polymers include alginic acids; carboxylic acid polysaccharides; carboxymethyl cellulose; carboxymethyl cellulose-cysteine; carrageenan (e.g., GELCARIN® 209, GELCARIN® 379, FMC Corporation, Philadelphia, PA); chondroitin sulfate; glycosaminoglycans; mucopolysaccharides; negatively charged polysaccharides (e.g., dextran sulfate); poly(acrylic acid); poly(D-aspartic acid); poly(L-aspartic acid); poly(L-aspartic acid) sodium salt; poly(D-glutamic acid); poly(L-glutamic acid); poly(L-glutamic acid) sodium salt; poly(methacrylic acid); sodium alginate (e.g., PROTANAL® LF 120M, PROTANAL® LF 200M, PROTANAL® LF 200D, FMC Biopolymer Corp., Drammen, Norway); sodium carboxymethyl cellulose (CMC); sulfated polysaccharides (heparins, agaropectins); pectin, gelatin and hyaluronic acid.

In particular embodiments, polymers disclosed herein can include “star shaped polymers,” which refer to branched polymers in which two or more polymer branches extend from a core. The core is a group of atoms having two or more functional groups from which the branches can be extended by polymerization. In particular embodiments, nanoparticles of the present disclosure include star shaped polymers. In particular embodiments, nanoparticles of the present disclosure include star shaped polymers and a coating. In particular embodiments, nanoparticles of the present disclosure include star shaped polymers and a coating including PGA. In particular embodiments, nanoparticles of the present disclosure include star shaped polymers and a coating including hyaluronic acid.

In particular embodiments, the branches are zwitterionic or negatively-charged polymeric branches. For star polymers, the branch precursors can be converted to zwitterionic or negatively-charged polymers via hydrolysis, ultraviolet irradiation, or heat. The polymers also may be obtained by any polymerization method effective for polymerization of unsaturated monomers, including atom transfer radical polymerization (ATRP), reversible addition-fragmentation chain transfer polymerization (RAFT), photo-polymerization, ring-opening polymerization (ROP), condensation, Michael addition, branch generation/propagation reaction, or other reactions.

Liposomes are microscopic vesicles including at least one concentric lipid bilayer. Vesicle-forming lipids are selected to achieve a specified degree of fluidity or rigidity of the final complex. In particular embodiments, liposomes provide a lipid composition that surrounds an aqueous core. In certain examples, the structure of a liposome can be used to encapsulate a nanoparticle within its core (i.e., a liposomal nanoparticle). In particular embodiments, nanoparticles of the present disclosure are utilized as nanoparticles within liposomal nanoparticles. Lipid nanoparticles (LNPs) are liposome-like structures that lack the continuous lipid bilayer characteristic of liposomes. Solid lipid nanoparticles (SLNs) are LNPs that are solid at room and body temperatures.

Liposomes and similar structures described in the preceding paragraph can be neutral (cholesterol) or bipolar and include phospholipids, such as phosphatidylcholine (PC), phosphatidylethanolamine (PE), phosphatidylinositol (PI), and sphingomyelin (SM) and other type of bipolar lipids including dioleoylphosphatidylethanolamine (DOPE), with a hydrocarbon chain length in the range of 14-22, and saturated or with one or more double C═C bonds. Examples of lipids capable of producing a stable liposome, alone, or in combination with other lipid components are phospholipids, such as hydrogenated soy phosphatidylcholine (HSPC), lecithin, phosphatidylethanolamine, lysolecithin, lysophosphatidylethanolamine, phosphatidylserine, phosphatidylinositol, sphingomyelin, cephalin, cardiolipin, phosphatidic acid, cerebro sides, distearoylphosphatidylethanolamine (DSPE), dioleoylphosphatidylcholine (DOPC), dipalmitoylphosphatidylcholine (DPPC), palmitoyloleoylphosphatidylcholine (POPC), palmitoyloleoylphosphatidylethanolamine (POPE) and dioleoylphosphatidylethanolamine 4-(N-maleimido-methyl)cyclohexane-1-carboxylate (DOPE-mal). Additional non-phosphorous containing lipids that can become incorporated into liposomes include stearylamine, dodecylamine, hexadecylamine, isopropyl myristate, triethanolamine-lauryl sulfate, alkyl-aryl sulfate, acetyl palmitate, glycerol ricinoleate, hexadecyl stereate, amphoteric acrylic polymers, polyethyloxylated fatty acid amides, DDAB, dioctadecyl dimethyl ammonium chloride (DODAC), 1,2-dimyristoyl-3-trimethylammonium propane (DMTAP), DOTAP, DOTMA, DC-Chol, phosphatidic acid (PA), dipalmitoylphosphatidylglycerol (DPPG), dioleoylphosphatidylglycerol, DOPG, and dicetylphosphate. In particular embodiments, lipids used to create liposomes disclosed herein include cholesterol, hydrogenated soy phosphatidylcholine (HSPC) and, the derivatized vesicle-forming lipid PEG-DSPE.

Methods of forming liposomes are described in, for example, U.S. Pat. Nos. 4,229,360; 4,224,179; 4,241,046; 4,737,323; 4,078,052; 4,235,871; 4,501,728; and 4,837,028, as well as in Szoka et al., Ann. Rev. Biophys. Bioeng. 9:467 (1980) and Hope et al., Chem. Phys. Lip. 40:89 (1986). For additional information regarding nanoparticles, see Yetisgin et al., Molecules 2020, 25, 2193.

The size of particles can vary over a wide range and can be measured in different ways. As indicated, in preferred embodiments, the particles are nanoparticles of <130 nm in size. However, NPs of the present disclosure can also have a minimum dimension of equal to or less than 500 nm, less than 150 nm, less than 140 nm, less than 120 nm, less than 110 nm, less than 100 nm, less than 90 nm, less than 80 nm, less than 70 nm, less than 60 nm, less than 50 nm, less than 40 nm, less than 30 nm, less than 20 nm, or less than 10 nm. In particular embodiments, the nanoparticles are NPs 90 to 130 nm in size.

In particular embodiments, the NPs can have a minimum dimension ranging between 5 nm and 500 nm, between 10 nm and 100 nm, between 20 nm and 90 nm, between 30 nm and 80 nm, between 40 nm and 70 nm, and between 40 nm and 60 nm. In particular embodiments, the dimension is the diameter of NPs or coated NPs. In particular embodiments, a population of nanoparticles of the present disclosure can have a mean minimum dimension of equal to or less than 500 nm, less than 100 nm, less than 90 nm, less than 80 nm, less than 70 nm, less than 60 nm, less than 50 nm, less than 40 nm, less than 30 nm, less than 20 nm, or less than 10 nm. In particular embodiments, a population of NPs in a composition of the present disclosure can have a mean diameter ranging between 5 nm and 500 nm, between 10 nm and 100 nm, between 20 nm and 90 nm, between 30 nm and 80 nm, between 40 nm and 70 nm, and between 40 nm and 60 nm, between 70 nm and 130 nm or between 75 nm and 125 nm. Dimensions of the nanoparticles can be determined using, e.g., conventional techniques, such as dynamic light scattering and/or electron microscopy. While not preferred, in particular embodiments, microparticles could also be used.

In particular embodiments, PbAE polymers are mixed with nucleotides (e.g., in vitro transcribed mRNA) in a ratio of 20:1, 30:1, 40:1, 50:1, 60:1, 70:1, 80:1, 90:1, 100:1, or more to generate PbAE-nucleotide polyplexes. In particular embodiments, PbAE polymers are mixed with nucleotides (e.g., in vitro transcribed mRNA) in a ratio of 60:1 to generate PbAE-nucleotide polyplexes. In particular embodiments, the PbAE-nucleotide polyplexes can be combined with PGA/Di-mannose to form the final NPs.

In particular embodiments, positively-charged polymer cores are PbAE formed by combining 1,4-butanediol diacrylate with 4-amino-1-butanol in a 1:1 molar ratio of diacrylate to amine monomers. The polymer can be a piperazine-capped 447 polymer. When conjugated to di-mannose, α-D-mannopyranosyl-(1→2)-α-D-mannopyranose (Di-mannose, Omicron Biochemicals Inc.) can be modified into glycosylamine before being conjugated to PGA.

Codon-optimized mRNAs can be capped with the Anti-Reverse Cap Analog 3′-O-Me-m7G(5′)ppp(5′)G (ARCA), and fully substituted with the modified ribonucleotides pseudouridine (ψ) and 5-methylcytidine (m5C).

To form nanoparticles, PbAE-447 polymers can be added to the mRNA at a ratio of 60:1 (w:w) and vortexed immediately for 15 s at a medium speed. Then the mixture can be incubated at RT for 5 min to allow the formation of PbAE-mRNA polyplexes. In the next step, 100 μg/mL PGA/Di-mannose in NaOAc buffer can be added to the polyplexes solution, vortexed for 15 s at medium speed, and incubated for 5 min at room temperature. In this process, PGA/Di-mannose coats the surfaces of PbAE-mRNA polyplexes to form the final NPs. For long-term storage, D-sucrose (60 mg/mL) can be added to the NP solutions as a cryoprotectant. The nanoparticles can be snap-frozen in dry ice, then lyophilized. Dried NPs can be stored at −20° C. or −80° C. until use. For in vivo use, lyophilized NPs can be re-suspended in water at a 1:20 (w:v) ratio.

In particular embodiments, the NP have a size of 99.8±SE/24.5, a polydispersity of 0.183, and an almost neutral surface charge (3.40±SE/2.15 mV ζ-potential). These physiochemical properties of NPs can be characterized using a Zetapals instrument (Brookhaven Instrument Corporation) at 25° C. To measure the hydrodynamic radius and polydispersity based on dynamic light scattering, NPs can be diluted fivefold into 25 mM NaOAc (pH=5.2). To measure the potential, NPs can be diluted 10-fold in 10 mM PBS (pH=7.0). To assess the stability of NPs, freshly prepared nanoparticles can be diluted in 10 mM PBS buffer (pH=7.4). The hydrodynamic radius and polydispersity of NPs were measured every 10 min for 5 h, and their sizes and particle concentrations were derived from Particle Tracking Analysis using a Nanosite 300 instrument (Malvern). Freshly made NPs (25 μL containing 0.83 μg of mRNA) were deposited on glow discharge-treated 200 mesh carbon/Formvar-coated copper grids. After 30 s, the grids were treated sequentially with 50% Karnovsky's fixative, 0.1 M cacodylate buffer, dH2O, then 1% (w/v) uranyl acetate. Samples were imaged with a JEOL JEM-1400 transmission electron microscope operating at 120 kV (JEOL USA).

In particular embodiments, nanoparticles can optionally include binding domain targeting ligands that bind cellular markers present on the surface of monocytes and/or macrophages.

M2 Binding Domains. Egr2 is expressed by M2 macrophages. Commercially available antibodies for Egr2 can be obtained from Thermo Fisher, Waltham, MA; Abcam, Cambridge, MA; Millipore Sigma, Burlington, MA; Miltenyi Biotec, Bergisch Gladbach, Germany; LifeSpan Biosciences, Inc., Seattle, WA; and Novus Biologicals, Littleton, CO. Generation of anti-Egr2 antibodies are discussed, for example, in Murakami K et al. (1993) Oncogene 8(6): 1559-1566. Anti-Egr2 antibodies include: rabbit monoclonal anti-Egr2 antibody clone EPR4004; mouse monoclonal anti-Egr2 antibody clone 1G5; mouse monoclonal anti-Egr2 antibody clone OTI1B12; rabbit polyclonal anti-Egr2 antibody recognizing AA residues 200-300 of human Egr2; rabbit polyclonal anti-Egr2 antibody recognizing AA residues 340-420 of human Egr2; and rabbit polyclonal anti-Egr2 antibody recognizing AA residues 370-420 of human Egr2. Binding domains can be derived from these antibodies and other antibodies disclosed herein.

In particular embodiments, the targeting ligand can be a nanobody including a binding domain including a CDR1 sequence including SGNIFSINAIG (SEQ ID NO: 45), a CDR2 sequence including TITLSGSTN (SEQ ID NO: 46), a CDR3 sequence including NTYSDSDVYGY (SEQ ID NO: 47). These reflect CDR sequences that bind CD206.

In particular embodiments, the targeting ligand can be a nanobody including a CDR1 sequence including PGFKLDYYAIA (SEQ ID NO: 48), a CDR2 sequence including SINSSGGST (SEQ ID NO: 49), and a CDR3 sequence including LRRYYGLNLDPGSYDY (SEQ ID NO: 50). These reflect CDR sequences that bind CD206.

In particular embodiments, the targeting ligand includes a human or humanized binding domain (e.g., nanobody) including a CDR1 sequence including GFPFNIYPMS (SEQ ID NO: 51), a CDR2 sequence including YISHGGTTT (SEQ ID NO: 52), and a CDRH3 sequence including GYARLMTDSELV (SEQ ID NO: 53). These reflect CDR sequences that bind CD206.

A number of additional antibodies specific for CD206 are known to those of skill in the art and can be readily characterized for sequence, epitope binding, and affinity. See, for example, WO 2014/140376, WO 2013/174537, and U.S. Pat. No. 7,560,534. Commercially available antibodies for CD206 can be obtained from Thermo Fisher, Waltham, MA; Proteintech, Rosemont, IL; BioLegend, San Diego, CA; R & D Systems, Minneapolis, MN; LifeSpan Biosciences, Inc., Seattle, WA; Novus Biologicals, Littleton, CO; and Bio-Rad, Hercules, CA. In particular embodiments, an anti-CD206 antibody includes a rat monoclonal anti-mouse CD206 monoclonal antibody clone C068C2 (Cat #141732, Biolegend, San Diego, CA).

In particular embodiments, the targeting ligand includes a binding domain including a CDRL1 sequence including ASQSVSHDV (SEQ ID NO: 54), a CDRL2 sequence including YTS, a CDRL3 sequence including QDYSSPRT (SEQ ID NO: 56), a CDRH1 sequence including GYSITSDY (SEQ ID NO: 57), a CDRH2 sequence including YSG, and a CDRH3 sequence including CVSGTYYFDYWG (SEQ ID NO: 59). These reflect CDR sequences of the Mac2-48 antibody that binds CD163.

In particular embodiments, the targeting ligand includes a binding domain including a CDRL1 sequence including ASQSVSSDV (SEQ ID NO: 60), a CDRL2 sequence including YAS, a CDRL3 sequence including QDYTSPRT (SEQ ID NO: 62), a CDRH1 sequence including GYSITSDY (SEQ ID NO: 57), a CDRH2 sequence including YSG, and a CDRH3 sequence including CVSGTYYFDYWG (SEQ ID NO: 59). These reflect CDR sequences of the Mac2-158 antibody that bind CD163.

A number of additional antibodies or binding domains specific for CD163 are known to those of skill in the art and can be readily characterized for sequence, epitope binding, and affinity. See, for example, WO 2011/039510, WO 2002/032941, WO 2002/076501, and US 2005/0214871. Commercially available antibodies for CD163 can be obtained from Thermo Fisher, Waltham, MA; Enzo Life Sciences, Inc., Farmingdale, NY; BioLegend, San Diego, CA; R & D Systems, Minneapolis, MN; LifeSpan Biosciences, Inc., Seattle, WA; and RDI Research Diagnostics, Flanders, NJ. In particular embodiments, anti-CD163 antibodies can include: mouse monoclonal anti-CD163 antibody clone 3D4; mouse monoclonal anti-CD163 antibody clone Ber-Mac3; mouse monoclonal anti-CD163 antibody clone EDHu-1; and mouse monoclonal anti-CD163 antibody clone GHI/61.

In particular embodiments, the targeting ligand includes a binding domain including a CDRL1 sequence including RSSKSLLYKDGKTYLN (SEQ ID NO: 66), a CDRL2 sequence including LMSTRAS (SEQ ID NO: 67), a CDRL3 sequence including QQLVEYPFT (SEQ ID NO: 68), a CDRH1 sequence including GYWMS (SEQ ID NO: 69), a CDRH2 sequence including EIRLKSDNYATHYAESVKG (SEQ ID NO: 70), and a CDRH3 sequence including FID. These reflect CDR sequences that bind CD23.

A number of antibodies or binding domains specific for CD23 are known to those of skill in the art and can be readily characterized for sequence, epitope binding, and affinity. See, for example, U.S. Pat. Nos. 7,008,623, 6,011,138 A (antibodies including 5E8, 6G5, 2C8, B3B1 and 3G12), US 2009/0252725, Rector et al. (1985) J. Immunol. 55: 481-488; Flores-Rumeo et al. (1993) Science 241: 1038-1046; Sherr et al. (1989) J. Immunol. 142: 481-489; and Pene et al., (1988) PNAS 85: 6820-6824. Commercially available antibodies for CD23 can be obtained from Thermo Fisher, Waltham, MA; Abcam, Cambridge, MA; Bioss Antibodies, Inc., Woburn, MA; Bio-Rad, Hercules, CA; LifeSpan Biosciences, Inc., Seattle, WA; and Boster Biological Technology, Pleasanton, CA. In particular embodiments, anti-CD23 antibodies can include: mouse monoclonal anti-CD23 antibody clone Tu 1; rabbit monoclonal anti-CD23 antibody clone SP23; rabbit monoclonal anti-CD23 antibody clone EPR3617; mouse monoclonal anti-CD23 antibody clone 5B5; mouse monoclonal anti-CD23 antibody clone 1B12; mouse monoclonal anti-CD23 antibody clone M-L23.4; and mouse monoclonal anti-CD23 antibody clone 3A2.

M1 Binding Domains. In particular embodiments, the targeting ligand includes a binding domain including a CDRL1 sequence including SSNIGDNY (SEQ ID NO: 72), a CDRL2 sequence including RDS, a CDRL3 sequence including QSYDSSLSGS (SEQ ID NO: 74), a CDRH1 sequence including GFTFDDYG (SEQ ID NO: 75), a CDRH2 sequence including ISWNGGKT (SEQ ID NO: 76), and a CDRH3 sequence including ARGSLFHDSSGFYFGH (SEQ ID NO: 77). These reflect CDR sequences of the Ab79 antibody that bind CD38.

In particular embodiments, the targeting ligand includes a binding domain including a CDRL1 sequence including NSNIGSNT (SEQ ID NO: 78), a CDRL2 sequence including SDS, a CDRL3 sequence including QSYDSSLSGSR (SEQ ID NO: 80), a CDRH1 sequence including GFTFNNYG (SEQ ID NO: 81), a CDRH2 sequence including ISYDGSDK (SEQ ID NO: 82), and a CDRH3 sequence including ARVYYYGFSGPSMDV (SEQ ID NO: 83). These reflect CDR sequences of the Ab19 antibody that bind CD38.

In particular embodiments, the targeting ligand includes a binding domain including a CDRL1 sequence including RASQSVSSYLA (SEQ ID NO: 84), a CDRL2 sequence including DASNRAT (SEQ ID NO: 85), a CDRL3 sequence including QQRSNWPPTF (SEQ ID NO: 86), a CDRH1 sequence including SFAMS (SEQ ID NO: 87), a CDRH2 sequence including AISGSGGGTYYADSVKG (SEQ ID NO: 88), and a CDRH3 sequence including DKILWFGEPVFDY (SEQ ID NO: 89). These reflect CDR sequences of the daratumumab antibody that bind CD38 described in U.S. Pat. No. 7,829,693.

A number of antibodies specific for CD38 are known to those of skill in the art and can be readily characterized for sequence, epitope binding, and affinity. See, for example, WO 2005/103083, WO 2006/125640, WO 2007/042309, WO 2008/047242, WO 2012/092612, WO 2006/099875, WO 2011/154453, WO 2015/130728, U.S. Pat. No. 7,829,693, and US 2016/0200828. Commercially available antibodies for CD38 can be obtained from Thermo Fisher, Waltham, MA; Abcam, Cambridge, MA; and Millipore Sigma, Burlington, MA. In particular embodiments, anti-CD23 antibodies can include: rabbit monoclonal anti-CD38 antibody clone GAD-3; mouse monoclonal anti-CD38 antibody clone HIT2; mouse monoclonal anti-CD38 antibody clone AT1; mouse monoclonal anti-CD38 antibody clone AT13/5; rat monoclonal anti-CD38 antibody clone NIMR-5; and rat monoclonal IgG2a, κ anti-CD38 antibody clone 90/CD38 (Cat #BD Biosciences, San Jose, CA).

In particular embodiments, G-protein coupled receptor 18 (Gpr18) is targeted on M1 macrophages. Commercially available antibodies for Gpr18 can be obtained from Assay Biotechnology Company Inc., Sunnyvale, CA; Thermo Fisher, Waltham, MA; Abcam, Cambridge, MA; GeneTex, Inc., Irvine, CA; and Novus Biologicals, Littleton, CO. In particular embodiments, anti-Gpr18 antibodies include: rabbit polyclonal anti-Gpr18 antibody recognizing a portion of amino acids 1-50 of human Gpr18; rabbit polyclonal anti-Gpr18 antibody recognizing a region including amino acids 160-240 of human Gpr18; rabbit polyclonal anti-Gpr18 antibody recognizing a region including amino acids 100-180 of human Gpr18; rabbit monoclonal anti-Gpr18 antibody clone EPR12359; and rabbit polyclonal anti-Gpr18 antibody recognizing a region including amino acids 140-190 of human Gpr18.

In particular embodiments, formyl peptide receptor 2 (Fpr2) is targeted on M1 macrophages. Commercially available antibodies for Fpr2 can be obtained from Atlas Antibodies, Bromma, Sweden; Biorbyt, LLC, San Francisco, CA; Cloud-Clone Corp., Katy, TX; US Biological Life Sciences, Salem, MA; and Novus Biologicals, Littleton, CO. In particular embodiments, anti-fpr2 antibodies include: mouse monoclonal anti-fpr2 antibody clone GM1D6; mouse monoclonal anti-fpr2 antibody clone 304405; recombinant anti-fpr2 antibody clone REA663; and rabbit polyclonal anti-fpr2 antibody recognizing a region including amino acids 300-350 of fpr2.

In particular embodiments, the targeting ligand includes a binding domain including a CDRL1 sequence including RASQSVSSYLA (SEQ ID NO: 84), a CDRL2 sequence including DASSRAT (SEQ ID NO: 91), a CDRL3 sequence including QLRSNWPPYT (SEQ ID NO: 92), a CDRH1 sequence including GYGMH (SEQ ID NO: 93), a CDRH2 sequence including VIWYDGSNKYYADSVKG (SEQ ID NO: 94), and a CDRH3 sequence including DTGDRFFDY (SEQ ID NO: 95). These reflect CDR sequences that bind CD64.

(9) Compositions for Administration. The nanoparticles disclosed herein can be provided as part of compositions formulated for administration to subjects. Compositions include a nanoparticle disclosed herein and a pharmaceutically acceptable carrier.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants.

Exemplary buffering agents include citrate buffers, succinate buffers, tartrate buffers, fumarate buffers, gluconate buffers, oxalate buffers, lactate buffers, acetate buffers, phosphate buffers, histidine buffers and/or trimethylamine salts.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol and 3-pentanol.

Exemplary isotonic agents include polyhydric sugar alcohols including trihydric or higher sugar alcohols, such as glycerin, erythritol, arabitol, xylitol, sorbitol or mannitol.

Exemplary stabilizers include organic sugars, polyhydric sugar alcohols, polyethylene glycol, sulfur-containing reducing agents, amino acids, low molecular weight polypeptides, proteins, immunoglobulins, hydrophilic polymers or polysaccharides.

In particular embodiments, compositions are formulated for intraperitoneal, intravenous, or intracranial injection. The compositions disclosed herein can further be formulated for intraarterial, intranodal, intralymphatic, intratumoral, intramuscular, oral, and/or subcutaneous administration and more particularly by intraarterial, intranodal, intralymphatic, intratumoral, intramuscular, and/or subcutaneous injection. The compositions disclosed herein can be formulated for administration by infusion, perfusion, or ingestion.

For injection, compositions can be formulated as aqueous solutions, such as in buffers including Hanks' solution, Ringer's solution, or physiological saline. The aqueous solutions can contain formulatory agents such as suspending, stabilizing and/or dispersing agents. Alternatively, the formulation can be in lyophilized and/or powder form for constitution with a suitable vehicle, e.g., sterile pyrogen-free water, before use.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as sparingly soluble salts.

Additionally, compositions can be formulated as sustained-release systems utilizing semipermeable matrices of solid polymers containing nanoparticles. Various sustained-release materials have been established and are well known by those of ordinary skill in the art. Sustained-release systems may, depending on their chemical nature, release nanoparticles following administration for a few weeks up to over 100 days.

For oral administration, the compositions can be formulated as tablets, pills, dragees, capsules, liquids, gels, syrups, slurries, suspensions and the like.

When formulated to treat cancer, the disclosed compositions can also include nucleotides carrying one or more anticancer genes selected from p53, RB, BRCA1, E1A, bcl-2, MDR-1, p21, p16, bax, bcl-xs, E2F, IGF-I VEGF, angiostatin, oncostatin, endostatin, GM-CSF, IL-12, IL-2, IL-4, IL-7, IFN-γ, TNFα and/or HSV-tk.

Any composition formulation disclosed herein can advantageously include any other pharmaceutically acceptable carriers which include those that do not produce significantly adverse, allergic or other untoward reactions that outweigh the benefit of administration, whether for research, prophylactic and/or therapeutic treatments. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety and purity standards as required by United States FDA Office of Biological Standards and/or other relevant foreign regulatory agencies.

In particular embodiments, the nanoparticles are provided as part of a composition that can include, for example, at least 0.1% w/v or w/w nanoparticles; at least 1% w/v or w/w nanoparticles; at least 10% w/v or w/w nanoparticles; at least 20% w/v or w/w nanoparticles; at least 30% w/v or w/w nanoparticles; at least 40% w/v or w/w nanoparticles; at least 50% w/v or w/w nanoparticles; at least 60% w/v or w/w nanoparticles; at least 70% w/v or w/w nanoparticles; at least 80% w/v or w/w nanoparticles; at least 90% w/v or w/w nanoparticles; at least 95% w/v or w/w nanoparticles; or at least 99% w/v or w/w nanoparticles.

(10) Methods of Use. Methods disclosed herein include altering the activation state of macrophages from an inactivated state to an activated state by introducing into macrophages nanoparticles including nucleotides encoding one or more IRFs and IKKβ. In particular embodiments, the altering results in reducing the percentage of macrophages in an inactivated state (e.g., M2 macrophages) in a population of macrophages treated with nanoparticles including nucleotides encoding one or more IRFs and IKKβ by 5-fold, 10-fold, 15-fold, 20-fold, or more compared to the percentage of macrophages in an inactivated state that have not been treated with the nanoparticles including nucleotides encoding one or more IRFs and IKKβ. In particular embodiments, the altering results in reducing the number of macrophages in an inactivated state (e.g., M2 macrophages) in a population of macrophages treated with the nanoparticles including nucleotides encoding one or more IRFs and IKKβ by 5-fold, 10-fold, 20-fold, 30-fold, 40-fold, 50-fold, 60-fold, 70-fold, 80-fold, 90-fold, 100-fold, or more compared to the number of macrophages in an inactivated state that have not been treated with the nanoparticles including nucleotides encoding one or more IRFs and IKKβ.

In particular embodiments, altering the activation state of macrophages from an inactivated state to an activated state by introducing into macrophages nanoparticles including nucleotides encoding one or more IRFs and IKKβ results in: restoring lymphocyte migration and infiltration into treatment sites, such as solid tumors or sites of infection or inflammation; increasing release of pro-inflammatory (anti-tumor) cytokines including IL-1β, IL-12, IFNγ, and/or TNFα by 1.5-fold, 2-fold, 2.5-fold, 3-fold, 3.5-fold, 4-fold, 5-fold, 6-fold, 7-fold, 8-fold, 9-fold, 10-fold, 15-fold, 20-fold, or more; reducing release of cytokines associated with M2 macrophage phenotype including IL-6 by 1.5-fold, 2-fold, 2.5-fold, 3-fold, 3.5-fold, 4-fold, 5-fold, 6-fold, 7-fold, 8-fold, 9-fold, 10-fold, 15-fold, 20-fold, or more.

In particular embodiments, altering the activation state of macrophages from an inactivated state to an activated state includes introducing into macrophages nanoparticles including nucleotides encoding IRF5 and IRF8. In particular embodiments, altering the activation of macrophages from an inactivated state to an activated state includes introducing into macrophages nanoparticles including nucleotides encoding mutant IRFs that are constitutively active or more active than their wild type counterpart IRFs.

Methods disclosed herein additionally result in secretion of multi-specific molecules (e.g., bi-specific molecules) from genetically modified monocytes/macrophages as well optionally TGFβ inhibitors.

Methods disclosed herein include treating subjects (humans, veterinary animals, livestock and research animals) with compositions disclosed herein. Treating subjects includes delivering a therapeutically effective amount. Therapeutically effective amounts can provide effective amounts, prophylactic treatments and/or therapeutic treatments.

An “effective amount” is the amount of a compound necessary to result in a desired physiological change in a subject. Effective amounts are often administered for research purposes. Effective amounts disclosed herein immunomodulate cells in a subject. In particular embodiments, the cells to be immunomodulated are immunosuppressed cells. In particular embodiments, the cells to be immunomodulated are macrophages. In particular embodiments, immunomodulation of macrophages includes switching immunosuppressed macrophages into activated macrophages. In particular embodiments, immunomodulation of macrophages includes switching M2 macrophages to M1 macrophages. In particular embodiments, cells to be immunomodulated include immunosuppressed cells including MDSC, Treg, DCreg, neutrophils, Th17, Breg, and/or MSC. In particular embodiments, immunomodulation of immunosuppressed cells includes phenotypic and/or functional switch of the immunosuppressed cells from being protumor to being antitumor.

A “prophylactic treatment” includes a treatment administered to a subject who does not display signs or symptoms of a disease or condition or displays only early signs or symptoms of the disease or condition such that treatment is administered for the purpose of diminishing, preventing, or decreasing the risk of developing the disease or condition further. Thus, a prophylactic treatment functions as a preventative treatment against a disease or disorder. For example, in particular embodiments, a prophylactic treatment includes administration of the compositions disclosed herein to a subject who had cancer but is in remission such that treatment is administered for the purpose of reducing or delaying the occurrence of relapse.

A “therapeutic treatment” includes a treatment administered to a subject who displays symptoms or signs of a disease or condition and is administered to the subject for the purpose of diminishing or eliminating those signs or symptoms of the disease or condition. For example, in particular embodiments, a therapeutic treatment includes administration of the compositions disclosed herein to a subject who has cancer to diminish or eliminate tumors and/or metastasis.

In particular embodiments, therapeutically effective amounts provide an anti-cancer effect in a subject. Cancer (medical term: malignant neoplasm) refers to a class of diseases in which a group of cells display uncontrolled growth (division beyond the normal limits), invasion (intrusion on and destruction of adjacent tissues), and sometimes metastasis. “Metastasis” refers to the spread of cancer cells from their original site of proliferation to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor, i.e. a secondary tumor or metastatic tumor, at the target site depends on angiogenesis. Tumor metastasis often occurs even after the removal of the primary tumor because tumor cells or components may remain and develop metastatic potential.

In particular embodiments, therapeutically effective amounts provide an anti-tumor effect in a subject. A “tumor” is a swelling or lesion formed by an abnormal growth of cells (called neoplastic cells or tumor cells). A “tumor cell” is an abnormal cell that divides by a rapid, uncontrolled cellular proliferation and continues to divide after the stimuli that initiated the new division cease. Tumors show partial or complete lack of structural organization and functional coordination with the normal tissue, and usually form a distinct mass of tissue, which may be either benign, pre-malignant or malignant.

An anti-tumor effect refers to a biological effect, which can be manifested by a decrease in the number of tumor cells, a decrease in the number of metastases, a decrease in tumor volume, an increase life expectancy, induced apoptosis of cancer cells, induced cancer cell death, induced chemo- or radiosensitivity in cancer cells, inhibited angiogenesis near cancer cells, inhibited cancer cell proliferation, inhibited tumor growth, prevented metastasis, prolonged life for a subject, reduced cancer-associated pain, reduced number of metastases, and/or reduced relapse or re-occurrence of the cancer following treatment. Accordingly, the compositions disclosed herein can be used to treat a variety of cancers, can prevent or significantly delay metastasis, and/or can prevent or significantly delay relapse. In particular embodiments, overall survival of a subject with cancer treated with a nanoparticle composition disclosed herein is improved by 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, or more as compared to a control subject with the same cancer not treated with the nanoparticle composition. In particular embodiments, the number of metastases in a subject with cancer treated with a nanoparticle composition disclosed herein is decreased by 1.5-fold, 2-fold, 3-fold, 4-fold, 5-fold, 6-fold, 7-fold, 8-fold, 9-fold, 10-fold, or more as compared to a control subject with the same cancer not treated with the nanoparticle composition.

In particular embodiments, a therapeutic treatment includes administration of the compositions disclosed herein in combination with another therapy to a subject who has cancer to diminish or eliminate tumors. In particular embodiments, the therapy to use in combination with the compositions disclosed herein include cancer vaccines, CAR immunotherapy (e.g., CAR-T immunotherapy), chemotherapy, radiotherapy, hormone therapy, signal transduction inhibitors, gene expression modulators, apoptosis inducers, angiogenesis inhibitors, and monoclonal antibodies that deliver toxic molecules. In particular embodiments, administration of a nanoparticle composition disclosed herein in combination with radiotherapy to a subject who has cancer improves overall survival by 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, or more as compared to a control subject with the same cancer not administered the nanoparticle composition in combination with radiotherapy.

Cancers that can be treated with systems and methods disclosed herein include ovarian cancer, breast cancer, brain cancer, melanomas, lung metastases, seminomas, teratomas, neuroblastomas, gliomas, rectal cancer, endometrial cancer, kidney cancer, adrenal cancer, thyroid cancer, skin cancer, cervical cancer, intestinal cancer, liver cancer, colon cancer, stomach cancer, head and neck cancer, gastrointestinal cancer, lymph node cancer, esophagus cancer, colorectal cancer, pancreatic cancer, ear, nose and throat (ENT) cancer, prostate cancer, cancer of the uterus, lung cancer, and metastases thereof.

In particular embodiments, therapeutically effective amounts provide anti-pathogen effects. Anti-pathogen effects can include anti-infection effects. Anti-infection effects can include a decrease in the occurrence of infections, a decrease in the severity of infections, a decrease in the duration of infections, a decrease in the number of infected cells, a decrease in volume of infected tissue, an increase in life expectancy, induced sensitivity of infected cells to immune clearance, reduced infection-associated pain, and/or reduction or elimination of a symptom associated with the treated infection.

In particular embodiments, therapeutically effective amounts provide anti-inflammatory effects. Anti-inflammatory effects can include reduced inflammation-associated pain, heat, redness, swelling and/or loss of function.

In particular embodiments, therapeutically effective amounts provide anti-Crohn's disease effects or anti-ulcerative colitis effects. Anti-Crohn's disease effects or anti-ulcerative colitis effects can include reduced diarrhea, reduced rectal bleeding, reduced unexplained weight loss, reduced fever, reduced abdominal pain and cramping, reduced fatigue and feelings of low energy, and/or restored appetite.

In particular embodiments, therapeutically effective amounts provide anti-arthritis effects. Anti-arthritis effects can include reduced pain, stiffness, swelling, redness in the joints and/or a restored range of motion. Types of arthritis include rheumatoid arthritis (RA), ankylosing spondylitis, and psoriatic arthritis.

In particular embodiments, therapeutically effective amounts provide anti-plaque psoriasis effects. Anti-plaque psoriasis effects can include reduced red patches, scaling spots, itching, burning, soreness, nail bed abnormalities and/or swollen or stiff joints.

For administration, therapeutically effective amounts (also referred to herein as doses) can be initially estimated based on results from in vitro assays and/or animal model studies. For example, a dose can be formulated in animal models to achieve a circulating concentration range that includes an IC₅₀ as determined in cell culture against a particular target. Such information can be used to more accurately determine useful doses in subjects of interest.

The actual dose amount administered to a particular subject can be determined by a physician, veterinarian or researcher taking into account parameters such as physical and physiological factors including target, body weight, severity of condition, type of disease, previous or concurrent therapeutic interventions, idiopathy of the subject and route of administration.

Useful doses often range from 0.1 to 5 μg/kg or from 0.5 to 1 μg/kg. In particular embodiments, a dose can include 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1000 μg/kg, 0.1 to 5 mg/kg or from 0.5 to 1 mg/kg. In particular embodiments, a dose can include 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 150 mg/kg, 200 mg/kg, 250 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly. In particular embodiments, therapeutically effective amounts can be achieved by administering repeated doses during the course of a treatment regimen.

The nanoparticle compositions described herein can be administered by injection, inhalation, infusion, perfusion, lavage or ingestion. Routes of administration can include intravenous, intradermal, intraarterial, intraparenteral, intranasal, intranodal, intralymphatic, intraperitoneal, intracranial, intralesional, intraprostatic, intravaginal, intrarectal, topical, intrathecal, intratumoral, intramuscular, intravesicular, oral, subcutaneous, and/or sublingual administration and more particularly by intravenous, intratumoral, intraperitoneal, and/or intracranial injection. Local administration includes administration of a therapeutically effective amount of a composition disclosed herein to a particular region, organ, or cavity of the body. For example, intraperitoneal injection can be used to deliver a therapeutic to treat ovarian cancer or intracranial injection can be used to deliver a therapeutic to treat a glioma. Administration of a therapeutic at a tumor site can include ligand mediated targeting of a therapeutic (e.g., nanoparticle compositions) to tumor cells and/or tumor supporting cells and not to healthy tissue using targeting ligands as described above. Administration of a therapeutic at a tumor site can include passive targeting of a therapeutic (e.g., nanoparticle compositions) to tumor cells and/or tumor supporting cells and not to healthy tissue. Particular embodiments of passive targeting can include enhanced permeability and retention (EPR) phenomenon based on size range of nanoparticles and the leaky vasculature and impaired lymphatic drainage of tumor tissues. Systemic administration, by contrast, is body-wide, and is typically achieved by intravenous injection of a composition or therapeutic into the circulation. Systemic administration of a therapeutic can be useful for less localized forms of cancer, such as cancers that have metastasized.

(11) Exemplary Embodiments.

1. A nanoparticle including:
a targeting ligand that binds to a professional phagocyte; and
a nucleic acid that encodes a protein molecule having at least a first binding domain and a second binding domain,
wherein the first binding domain is specific to a cell surface protein expressed by an immune cell, and
wherein the second binding domain is specific to a cell surface protein expressed by a cell of interest.
   2. The nanoparticle of embodiment 1, wherein the cell of interest is a cancer cell, an infected cell, an autoreactive cell, or a prokaryotic cell.
   3. The nanoparticle of embodiments 1 or 2, wherein the targeting ligand binds to a cell surface protein expressed by a monocyte, a macrophage, or both.
   4. The nanoparticle of any of embodiments 1-3, wherein the targeting ligand includes di-mannose.
   5. The nanoparticle of any of embodiments 1-4, wherein the nucleic acid includes ribonucleic acid (RNA).
   6. The nanoparticle of embodiment 5, wherein the RNA includes messenger RNA (mRNA).
   7. The nanoparticle of embodiment 6, wherein the mRNA includes synthetic RNA or in vitro transcribed RNA (IVT RNA).
   8. The nanoparticle of any of embodiments 1-7, wherein the first binding domain is specific to a cell surface protein of a lymphocyte.
   9. The nanoparticle of embodiment 8, wherein the lymphocyte is selected from the group including a T-cell, a B-cell, a natural killer (NK) cell, and a tumor-infiltrating lymphocyte (TIL) cell.
   10. The nanoparticle of any of embodiments 1-9, wherein the first binding domain is specific to a cell surface protein of a T-cell selected from the group including a CD8+ T cell, CD4+ T cell, a gamma delta T cell, and an NK T-cell.
   11. The nanoparticle of any of embodiment 1-10, wherein the first binding domain is specific to CD3.
   12. The nanoparticle of any of embodiments 1-11, wherein the protein molecule is a multi-specific T-cell engager.
   13. The nanoparticle of any of embodiments 1-12, wherein the protein molecule is an EpCAM-CD3 bi-specific T-cell engager.
   14. The nanoparticle of any of embodiments 1-13, wherein the second binding domain is specific to an antigen expressed by the cell of interest.
   15. The nanoparticle of any of embodiments 1-14, further including a second nucleic acid that encodes one or more interferon regulatory factors (IRFs).
   16. The nanoparticle of any of embodiments 1-15, further including a tumor cell proliferation inhibitor or a nucleic acid encoding a tumor cell proliferation inhibitor.
   17. The nanoparticle of any of embodiments 1-16, wherein the nucleic acid encodes an antibody, or an antigen-binding fragment of an antibody.
   18. The nanoparticle of any of embodiments 1-17, wherein the nanoparticle includes a nucleic acid encoding a CD40-CD40L inhibitor or a TGFβ inhibitor.
   19. The nanoparticle of any embodiments 1-18, wherein the nanoparticle is a liposome, a liposomal nanoparticle, a lipid nanoparticle, or a solid lipid nanoparticle.
   20. A composition including:
a first plurality of nanoparticles, wherein each of the first plurality of nanoparticles includes:
   a targeting ligand that binds to a professional phagocyte; and
a nucleic acid encoding a protein molecule having a first binding domain specific to a cell surface protein expressed by an immune cell, and a second binding domain is specific to a cell surface protein expressed by a cell of interest.
   21. The composition of embodiment 20, wherein the cell of interest is a cancer cell, an infected cell, an autoreactive cell, or a prokaryotic cell.
   22. The composition of embodiments 20 or 21, wherein the targeting ligand binds to a cell surface protein expressed by a monocyte, a macrophage, or both.
   23. The composition of any of embodiments 20-22, wherein the targeting ligand includes di-mannose.
   24. The composition of any of embodiments 20-23, wherein the nucleic acid includes RNA.
   25. The composition of embodiment 24, wherein the RNA includes mRNA.
   26. The composition of embodiment 25, wherein the mRNA includes synthetic RNA or IVT RNA.
   27. The composition of any of embodiments 20-26, wherein the first binding domain is specific to a cell surface protein of a lymphocyte.
   28. The composition of embodiment 27, wherein the lymphocyte is selected from the group including a T-cell, a B-cell, an NK cell, and a TIL cell.
   29. The composition of any of embodiments 20-28, wherein the first binding domain is specific to a cell surface protein of a T-cell selected from the group including a CD8+ T cell, CD4+ T cell, a gamma deltaT cell, and an NK T-cell.
   30. The composition of any of embodiments 20-29, wherein the first binding domain is specific to CD3.
   31. The composition of any of embodiments 20-30, wherein the protein molecule is a bi-specific T-cell engager.
   32. The composition of any of embodiments 20-31, wherein the protein molecule is an EpCAM-CD3 bi-specific T-cell engager.
   33. The composition of any of embodiments 20-32, wherein the second binding domain is specific to an antigen expressed by the cell of interest.
   34. The composition of any of embodiments 20-33, further including a pharmaceutically acceptable carrier.
   35. The composition of any of embodiments 20-34, wherein at least a subset of the first plurality of nanoparticles further includes one or more of (a) a nucleic acid encoding one or more interferon regulatory factors (IRFs), and (b) a nucleic acid encoding IKKβ.
   36. The composition of any of embodiments 20-35, wherein the first plurality of nanoparticles include a liposome, a liposomal nanoparticle, a lipid nanoparticle, or a solid lipid nanoparticle.
   37. The composition of any of embodiments 20-36, further including:

a second plurality of nanoparticles, wherein at least a subset of the second plurality of nanoparticles include one or more of (a) a nucleic acid encoding one or more interferon regulatory factors (IRFs), and (b) a nucleic acid encoding IKKβ.

38. The composition of any of embodiments 20-37, further including a tumor cell proliferation inhibitor.
39. The composition of any of embodiments 20-38, wherein at least a subset of the first or second plurality of nanoparticles further include a nucleic acid encoding a tumor cell proliferation inhibitor.
40. The composition of any of embodiments 20-39, wherein at least a subset of the first or second plurality of nanoparticles further include a nucleic acid encoding an antigen-binding fragment of an antibody of a tumor cell proliferation inhibitor.
41. The composition of any of embodiments 37-40, wherein the second plurality of nanoparticles include a liposome, a liposomal nanoparticle, a lipid nanoparticle, or a solid lipid nanoparticle.
42. The composition of any of embodiments 20-41, further including a third plurality of nanoparticles, wherein at least a subset of the third plurality of nanoparticles include a nucleic acid encoding an antigen-binding fragment of an antibody of a tumor cell proliferation inhibitor.
43. The composition of any of embodiments 38-42, wherein the tumor cell proliferation inhibitor is a CD40-CD40L inhibitor or a TGFβ inhibitor.
44. The composition of embodiment 42 or 43, including the first plurality of nanoparticles and the third plurality of nanoparticles in the absence of the second plurality of nanoparticles.
45. The composition of any of embodiments 42-44, wherein the third plurality of nanoparticles include a liposome, a liposomal nanoparticle, a lipid nanoparticle, or a solid lipid nanoparticle.
46. A composition for treating a condition in a human subject, the composition including:
a first plurality of nanoparticles, wherein each of the plurality of nanoparticles includes
   (i) a targeting ligand that binds to a monocyte, macrophage, or both; and
(ii) an mRNA encoding a protein molecule having at least a first binding domain specific to a cell surface protein expressed by a lymphocyte, and a second binding domain specific to a cell surface protein expressed by a cell of interest;
   wherein the first plurality of nanoparticles stimulates or enhances an immune response in the human subject, thereby treating the condition.
   47. The composition of embodiment 46, wherein the cell of interest is a cancer cell, an infected cell, an autoreactive cell, or a prokaryotic cell.
   48. The composition of any of embodiments 46 or 47, wherein the targeting ligand includes di-mannose.
   49. The composition of any of embodiments 46-48, wherein the mRNA includes synthetic RNA or IVT RNA.
   50. The composition of any of embodiments 46-49, wherein the first binding domain is specific to a cell surface protein of a lymphocyte.
   51. The composition of embodiment 50, wherein the lymphocyte is selected from the group including a T-cell, a B-cell, an NK cell and a TIL cell.
   52. The composition of any of embodiments 46-51, wherein the first binding domain is specific to a cell surface protein of a T-cell selected from the group including a CD8+ T cell, CD4+ T cell, a gamma delta T cell, and an NK T-cell.
   53. The composition of any of embodiments 46-52, wherein the first binding domain is specific to CD3.
   54. The composition of any of embodiments 46-53, wherein the protein molecule is a bi-specific T-cell engager.
   55. The composition of any of embodiments 46-54, wherein the protein molecule is an EpCAM-CD3 bi-specific T-cell engager.
   56. The composition of any of embodiments 46-55, wherein the second binding domain is specific to an antigen expressed by the cell of interest.
   57. The composition of any of embodiments 46-56, further including a pharmaceutically acceptable carrier.
   58. The composition of any of embodiments 46-57, wherein the at least a subset of the first plurality of nanoparticles further include one or more of (a) an mRNA encoding one or more interferon regulatory factors (IRFs), (b) an mRNA encoding IKKβ, or (c) an mRNA encoding one or more IRFs and an mRNA encoding IKKβ, and (c) an mRNA encoding a tumor cell proliferation inhibitor.
   59. The composition of any of embodiments 46-58, wherein the first plurality of nanoparticles include a liposome, a liposomal nanoparticle, a lipid nanoparticle, or a solid lipid nanoparticle.
   60. The composition of any of embodiments 46-59, further including:
a second plurality of nanoparticles, wherein each of the second plurality of nanoparticles includes
a targeting ligand that binds to a monocyte, a macrophage, or both, and
one or more of (a) an mRNA encoding one or more interferon regulatory factors (IRFs), (b) an mRNA encoding IKKβ, and (c) an mRNA encoding a tumor cell proliferation inhibitor.
   61. The composition of embodiment 60, wherein the second plurality of nanoparticles include an mRNA encoding an antigen-binding fragment of an antibody of a tumor cell proliferation inhibitor.
   62. The composition of embodiments 60 or 61, wherein the tumor cell proliferation inhibitor is a CD40-CD40L inhibitor or a TGFβ inhibitor.
   63. The composition of any of embodiments 60-62, wherein the second plurality of nanoparticles include a liposome, a liposomal nanoparticle, a lipid nanoparticle, or a solid lipid nanoparticle.
   64. A method for treating a condition in a human subject, the method including: administering to the human subject a composition including a first plurality of nanoparticles,
   wherein each of the first plurality of nanoparticles includes:
(i) a targeting ligand that binds to a monocyte, a macrophage, or both; and
(ii) an mRNA encoding a protein molecule having at least a first binding domain specific to a cell surface protein expressed by a lymphocyte, and a second binding domain specific to a cell surface protein expressed by a cell of interest;
   wherein the plurality of nanoparticles stimulates or enhances an immune response in the human subject, thereby treating the condition.
   65. The method of embodiment 64, wherein the cell of interest is a cancer cell, an infected cell, an autoreactive cell, or a prokaryotic cell.
   66. The method of embodiments 64 or 65, wherein the targeting ligand includes di-mannose.
   67. The method of any of embodiments 64-66, wherein the mRNA includes synthetic RNA or IVT RNA.

68. The method of any of embodiments 64-67, wherein the lymphocyte is selected from the group including a T-cell, a B-cell, an NK cell, and a TIL cell.

69. The method of any of embodiments 64-68, wherein the first binding domain is specific to a cell surface protein of a T-cell selected from the group including a CD8+ T cell, CD4+ T cell, a gamma delta T cell, and an NK T-cell.
70. The method of any of embodiments 64-69, wherein the first binding domain is specific to CD3.
71. The method of any of embodiments 64-70, wherein the protein molecule is a bi-specific T-cell engager.
72. The method of any of embodiments 64-71, wherein the protein molecule is an EpCAM-CD3 bi-specific T-cell engager.
73. The method of any of embodiments 64-72, wherein the second binding domain is specific to an antigen expressed by the cell of interest.
74. The method of any of embodiments 64-73, wherein the composition further including a pharmaceutically acceptable carrier.
75. The method of any of embodiments 64-74, wherein at least a subset of the first plurality of nanoparticles further include one or more of (a) a nucleic acid encoding one or more interferon regulatory factors (IRFs), (b) a nucleic acid encoding IKKβ, and (c) a nucleic acid encoding a tumor cell proliferation inhibitor.
76. The composition of any of embodiments 64-75, wherein the first plurality of nanoparticles include a liposome, a liposomal nanoparticle, a lipid nanoparticle, or a solid lipid nanoparticle.
77. The method of any of embodiments 64-76, further including:
administering to the human subject a composition including a second plurality of nanoparticles, wherein each of the second plurality of nanoparticles includes
   a targeting ligand that binds to a monocyte, a macrophage, or both, and
   one or more of (a) an mRNA encoding one or more interferon regulatory factors (IRFs), and (b) an mRNA encoding IKKβ.
      78. The method of any of embodiments 75-77, wherein at least a subset of the first or second plurality of nanoparticles further include an mRNA encoding a tumor cell proliferation inhibitor.
      79. The composition of embodiment 77 or 78, wherein the second plurality of nanoparticles include a liposome, a liposomal nanoparticle, a lipid nanoparticle, or a solid lipid nanoparticle.
      80. The method of any of embodiments 64-79, further including:
administering to the human subject a composition including a third plurality of nanoparticles, wherein each of the third plurality of nanoparticles includes
a targeting ligand that binds to a monocyte, a macrophage, or both, and
an mRNA encoding a tumor cell proliferation inhibitor.
   81. The method of any of embodiments 75-80, wherein, an mRNA encoding a tumor cell proliferation inhibitor encodes an antigen-binding fragment of an antibody of a tumor cell proliferation inhibitor.
   82. The method of any of embodiments 75-81, wherein the tumor cell proliferation inhibitor is a CD40-CD40L inhibitor or a TGFβ inhibitor.
   83. The composition of any of embodiments 80-82, wherein the third plurality of nanoparticles include a liposome, a liposomal nanoparticle, a lipid nanoparticle, or a solid lipid nanoparticle.
   84. The method of any of embodiments 77-83, wherein the step of administering a composition including the first plurality of nanoparticles and the step of administering a composition including the second plurality of nanoparticles are performed concurrently or sequentially.
   85. The method of any of embodiments 77-83, wherein the step of administering a composition including the first plurality of nanoparticles is performed after the step of administering a composition including the second plurality of nanoparticles.
   86. The method of any of embodiments 80-85, wherein the step of administering a composition including the third plurality of nanoparticles is performed concurrently or sequentially with the step of administering the first plurality of nanoparticles.
   87. The method of any of embodiments 80-85, wherein the step of administering a composition including the third plurality of nanoparticles is performed concurrently or sequentially with the step of administering the second plurality of nanoparticles.
   88. The method of any of embodiments 80-85, including the steps of administering a composition including the first plurality of nanoparticles and administering a composition including the third plurality of nanoparticles in the absence of the step of administering a composition including the second plurality of nanoparticles.
   89. A modified professional phagocyte including:

a nanoparticle loaded with a nucleic acid encoding a protein molecule having at least a first binding domain specific to a cell surface protein expressed by an immune cell and a second binding domain specific for a cell surface protein expressed by a cell of interest,

wherein the nanoparticle is adhered to the surface of the phagocyte or has been internalized by the phagocyte.
90. The modified professional phagocyte of embodiment 89, wherein the cell of interest is a cancer cell, an infected cell, an autoreactive cell, or a prokaryotic cell.
91. The modified professional phagocyte of any of embodiments 89 or 90, wherein the phagocyte is a monocyte or a macrophage.
92. The modified professional phagocyte of any of embodiments 89-91, where the phagocyte is a tumor-associated macrophage.
93. The modified professional phagocyte of any of embodiments 89-92, wherein the nucleic acid includes ribonucleic acid (RNA).
94. The modified professional phagocyte of embodiment 93, wherein the RNA includes messenger RNA (mRNA).
95. The modified professional phagocyte of embodiment 94, wherein the mRNA includes synthetic RNA or in vitro transcribed RNA (IVT RNA).
96. The modified professional phagocyte of any of embodiments 89-95, wherein the first binding domain is specific to a cell surface protein of a lymphocyte.
97. The modified professional phagocyte of embodiment 96, wherein the lymphocyte is selected from the group including a T-cell, a B-cell, an NK cell, and a TIL cell.
98. The modified professional phagocyte of any of embodiments 89-97, wherein the first binding domain is specific to a cell surface protein of a T-cell selected from the group including a CD8+ T cell, CD4+ T cell, a gamma delta T cell, and an NK T-cell.
99. The modified professional phagocyte of any of embodiments 89-98, wherein the first binding domain is specific to CD3.
100. The modified professional phagocyte of any of embodiments 89-99, wherein the protein molecule is a bi-specific T-cell engager.
101. The modified professional phagocyte of any of embodiments 89-100, wherein the protein molecule is an EpCAM-CD3 bi-specific T-cell engager.
102. The modified professional phagocyte of any of embodiments 89-101, wherein the nanoparticle is further loaded with one or more of (a) a nucleic acid encoding one or more interferon regulatory factors (IRFs), (b) a nucleic acid encoding IKKβ, and (c) a nucleic acid encoding a tumor cell proliferation inhibitor.
103. The modified professional phagocyte of any of embodiments 89-102, wherein the nanoparticle includes a liposome, a liposomal nanoparticle, a lipid nanoparticle, or a solid lipid nanoparticle.
104. The modified professional phagocyte of any of embodiments 89-103, further including:
a second nanoparticle loaded with one or more of (a) a nucleic acid encoding one or more interferon regulatory factors (IRFs), (b) a nucleic acid encoding IKKβ, and (c) a nucleic acid encoding a tumor cell proliferation inhibitor,
wherein the second nanoparticle is adhered to the surface of the phagocyte or has been internalized by the phagocyte.
   105. The modified professional phagocyte of embodiment 104, wherein the second nanoparticle includes a liposome, a liposomal nanoparticle, a lipid nanoparticle, or a solid lipid nanoparticle.
   106. The modified professional phagocyte of embodiments 104 or 105, wherein the first or second nanoparticle is loaded with a nucleic acid encoding an antibody or an antigen-binding fragment of an antibody of a tumor cell proliferation inhibitor.
   107. The modified professional phagocyte of any of embodiments 102-106, wherein the tumor cell proliferation inhibitor is a CD40-CD40L inhibitor or a TGFβ inhibitor.
   108. The modified professional phagocyte of any of embodiments 89-107, further including at least one of
a second nanoparticle loaded with one or more of (a) a nucleic acid encoding one or more interferon regulatory factors (IRFs), (b) a nucleic acid encoding IKKβ, or (c) a nucleic acid encoding a tumor cell proliferation inhibitor; and
a third nanoparticle loaded with a nucleic acid encoding a tumor cell proliferation inhibitor, wherein each of the second and third nanoparticles is adhered to the surface of the phagocyte or has been internalized by the phagocyte.
   109. The modified professional phagocyte of embodiment 108, wherein the third nanoparticle includes a liposome, a liposomal nanoparticle, a lipid nanoparticle, or a solid lipid nanoparticle.
   110. A nanoparticle including a positively-charged polymer core and a neutral or negatively-charged coating around the polymer core wherein the positively-charged polymer core encapsulates nucleotides encoding at least one binding domain that binds an immune cell activating epitope and/or at least one binding domain that binds an antigen on a cell of interest.
   111. The nanoparticle of embodiment 110, wherein the nanoparticles are <130 nm.
   112. The nanoparticle of embodiments 110 or 111, wherein the positively charged polymer includes poly(β-amino ester, poly(L-lysine), poly(ethylene imine) (PEI), poly-(amidoamine) dendrimers (PAMAMs), poly(amine-co-esters), poly(dimethylaminoethyl methacrylate) (PDMAEMA), chitosan, poly-(L-lactide-co-L-lysine), poly[α-(4-aminobutyl)-L-glycolic acid] (PAGA), or poly(4-hydroxy-L-proline ester) (PHP).
   113. The nanoparticle of any of embodiments 110-112, wherein the positively charged polymer includes poly(β-amino ester).
   114. The nanoparticle of any of embodiments 110-113, wherein the neutral or negatively-charged coating includes polyglutamic acid (PGA), poly(acrylic acid), alginic acid, or cholesteryl hemisuccinate/1,2-dioleoyl-sn-glycero-3-phosphoethanolamine.
   115. The nanoparticle of any of embodiments 110-114, wherein the neutral or negatively-charged coating includes polyglutamic acid (PGA).
   116. The nanoparticle of any of embodiments 110-115, wherein the neutral or negatively-charged coating includes a zwitterionic polymer.
   117. The nanoparticle of any of embodiments 110-116, wherein the neutral or negatively-charged coating includes a liposome.
   118. The nanoparticle of embodiment 117, wherein the liposome includes 1,2-dioleoyl-3-trimethylammonium-propane (DOTAP), 1,2-di-O-octadecenyl-3-trimethylammonium propane (DOTMA), 3ß[N-(N′,N′-dimethylaminoethane)-carbamoyl]cholesterol (DC-Chol), dioctadecyl-amidoglycylspermine (DOGS), cholesterol, 1,2-dioleoyl-sn-glycero-3-phosphoethanolamine (DOPE), or 1,2-dioleoyl-sn-glycero-3-phosphocholine (DOPC).
   119. The nanoparticle of any of embodiments 110-118, wherein the nucleotides include ribonucleic acid (RNA).
   120. The nanoparticle of embodiment 119, wherein the RNA includes synthetic RNA.
   121. The nanoparticle of embodiments 119 or 120, wherein the RNA includes in vitro transcribed mRNA.
   122. The nanoparticle of any of embodiments 110-121, wherein the nucleotides include integrating or non-integrating double-stranded DNA.
   123. The nanoparticle of any of embodiments 110-122, wherein the nucleotides are in the form of a plasmid, a minicircle plasmid, or a closed-ended linear ceDNA.
   124. The nanoparticle of any of embodiments 110-123, wherein the antigen on the cell of interest is a cancer antigen expressed by an ovarian cancer cell, a melanoma cell, a glioblastoma cell, a multiple myeloma cell, a melanoma cell, a prostate cancer cell, a breast cancer cell, a stem cell cancer cell, a mesothelioma cell, a renal cell carcinoma cell, a pancreatic cancer cell, a lung cancer cell, a cholangiocarcinoma cell, a bladder cancer cell, a neuroblastoma cell, a colorectal cancer cell, or a merkel cell carcinoma cell.
   125. The nanoparticle of any of embodiments 110-124, wherein the cancer antigen includes B-cell maturation antigen (BCMA), carboxy-anhydrase-IX (CAIX), CD19, CD24, CD56, CD133, CEA, disialoganglioside, EpCam, EGFR, EGFR variant III (EGFRvIII), ERBB2, folate receptor (FOLR), GD2, glypican-2, HER2, Lewis Y, L1-CAM, mesothelin, MUC16, PD-L1, PSMA, Prostate Stem Cell antigen (PSCA), ROR1, TYRP1/gp75, SV40 T, or WT-1.
   126. The nanoparticle of any of embodiments 110-125, wherein the binding domain that binds the cancer antigen includes the complementarity determining regions (CDRs) of antibody adecatumumab, anetumab, ravtansine, amatuximab, HN1, oregovomab, ovarex, abagovomab, edrecolomab, farletuzumab. flanvotumab, TA99, 20D7, Cetuximab, FMC63, SJ25C1, HD37, R11, R12, 2A2, Y31, 4D5, 3G10 atezolizumab, avelumab, or durvalumab or is derived from a T cell receptor (TCR).
   127. The nanoparticle of any of embodiments 110-126, wherein the binding domains that binds a cancer antigen is a protein molecule.
   128. The nanoparticle of embodiment 127, wherein nucleotides within the nanoparticle encode different protein molecules including binding domains that bind different cancer antigens.
   129. The nanoparticle of embodiment 128, wherein the different cancer antigens are expressed by the same cancer type.
   130. The nanoparticle of embodiment 129, wherein the cancer type is ovarian cancer, melanoma, or glioblastoma.
   131. The nanoparticle of any of embodiment 128-130, wherein the different cancer antigens include
at least two cancer antigens selected from EpCam, L1-CAM, MUC16, folate receptor (FOLR), Lewis Y, ROR1, mesothelin, WT-1, PD-L1, EGFR, and CD56;
at least two cancer antigens selected from Tyrosinase related protein 1 (TYRP1/gp75); GD2, PD-L1, and EGFR; or two cancer antigens selected from EGFR variant III (EGFRvIII) and IL13 Ra2.
   132. The nanoparticle of any of embodiments 110-131, wherein at least one binding domain of the protein molecule binds a viral antigen, a bacterial antigen, a superbug antigen, a fungal antigen, or an autoimmune or allergic antigen.
   133. The nanoparticle of embodiment 132, wherein
the viral antigen is expressed by adenoviruses, arenaviruses, bunyaviruses, coronavirusess, flavirviruses, hantaviruses, hepadnaviruses, herpesviruses, papilomaviruses, paramyxoviruses, parvoviruses, picornaviruses, poxviruses, orthomyxoviruses, retroviruses, reoviruses, rhabdoviruses, rotaviruses, spongiform viruses or togaviruses;
the bacterial antigen is expressed by anthrax; gram-negative bacilli, chlamydia, diptheria, haemophilus influenza, Helicobacter pylori, malaria, Mycobacterium tuberculosis, pertussis toxin, pneumococcus, rickettsiae, staphylococcus, streptococcus or tetanus;
the superbug antigen is expressed by Enterococcus faecium, Clostridium difficile, Acinetobacter baumannii, Pseudomonas aeruginosa, or Enterobacteriaceae;
the fungal antigen is expressed by candida, coccidiodes, cryptococcus, histoplasma, leishmania, plasmodium, protozoa, parasites, schistosomae, tinea, toxoplasma, or Trypanosoma cruzi; or
the autoimmune or allergic antigen is expressed by a subject having acute necrotizing hemorrhagic encephalopathy, allergic asthma, alopecia areata, anemia, aphthous ulcer, arthritis, asthma, autoimmune thyroiditis, conjunctivitis, Crohn's disease, cutaneous lupus erythematosus, dermatitis, diabetes, diabetes mellitus, erythema nodosum leprosum, keratoconjunctivitis, multiple sclerosis, myasthenia gravis, psoriasis, scleroderma, Sjogren's syndrome, including keratoconjunctivitis sicca secondary to Sjogren's syndrome, Stevens-Johnson syndrome, systemic lupus erythematosis, ulcerative colitis, vaginitis and/or Wegener's granulomatosis
and/or wherein the binding domain for the antigen is derived from a TCR.
   134. The nanoparticle of any of embodiments 110-133, wherein the at least one binding domain of the protein molecule binds an immune cell activating epitope expressed by a T cell or a natural killer (NK) cell.
   135. The nanoparticle of any of embodiments 110-134, wherein the immune cell activating epitope is expressed by a T cell.
   136. The nanoparticle of embodiment 135, wherein the immune cell activating epitope expressed by the T cell includes CD2, CD3, CD7, CD8, CD27, CD28, CD30, CD40, CD83, 4-1BB, OX40, lymphocyte function-associated antigen-1 (LFA-1), LIGHT, NKG2C, or B7-H3.
   137. The nanoparticle of embodiment 135, wherein the immune cell activating epitope expressed by the T cell includes CD3, CD28, or 4-1BB.
   138. The nanoparticle of any of embodiments 110-137, wherein the binding domains that bind an immune cell activating epitope include a protein molecule.
   139. The nanoparticle of embodiment 138, wherein nucleotides within the nanoparticle encode different protein molecules including binding domains that bind different immune cell activating epitopes.
   140. The nanoparticle of embodiment 139, wherein the different immune cell activating epitopes include CD3 and CD28 or CD3 and 4-1BB.
   141. The nanoparticle of any of embodiments 110-140, wherein at least one binding domain includes the CDRs of antibody OKT3, 20G6-F3, 4B4-D7, 4E7-C9, 18F5-H10, TGN1412, 9D7, 9.3, KOLT-2, 15E8, 248.23.2, EX5.3D10, OKT8 or the SK1.
   142. The nanoparticle of any of embodiments 110-134, wherein the immune cell activating epitope is expressed by an NK cell.
   143. The nanoparticle of embodiment 142, wherein the immune cell activating epitope expressed by the NK cell includes NKG2D, CD8, CD16, KIR2DL4, KIR2DS1, KIR2DS2, KIR3DS1, NKG2C, NKG2E, NKG2D, NKp30, NKp44, NKp46, NKp80, or DNAM-1.
   144. The nanoparticle of any of embodiments 110-143, wherein at least one binding domain includes the CDRs of antibody 5C6, 1D11, mAb 33, P44-8, SK1, or 3G8.
   145. The nanoparticle of any of embodiments 110-144, wherein the binding domains are linked through a protein linker.
   146. The nanoparticle of embodiment 145, wherein the protein linker includes a Gly-Ser linker.
   147. The nanoparticle of embodiment 145 or 146, wherein the protein linker includes a proline-rich linker.
   148. The nanoparticle of any of embodiments 110-147, wherein the protein molecule includes a single chain variable fragment (scFv).
   149. The nanoparticle of any of embodiments 110-148, wherein the protein molecule includes
at least one binding domain binds CEA and at least one binding domain binds CD3, CD28, or 4-1BB;
at least one binding domain binds EGFR and at least one binding domain binds CD3, CD28, or 4-1BB;
at least one binding domain binds EpCam and at least one binding domain binds CD3, CD28, or 4-1BB;
at least one binding domain binds HER2 and at least one binding domain binds CD3, CD28, or 4-1BB;
at least one binding domain binds PD-L1 and at least one binding domain binds CD3, CD28, or 4-1BB;
at least one binding domain binds PSMA and at least one binding domain binds CD3, CD28, or 4-1BB; or
at least one binding domain binds [TYRP1/gp75] and at least one binding domain binds CD3, CD28, or 4-1BB.
   150. The nanoparticle of embodiment 149, wherein the protein molecule includes catumaxomab, MT110, ertumaxomab, MDX-447, MM-141, AMG211, R06958688, R06895882, TF2, BAY2010112, AMG701, solitomab, or blinatumomab.
   151. A nanoparticle of any of embodiments 110-150, wherein the positively-charged polymer core further encapsulates nucleotides encoding one or more interferon regulatory factors (IRFs).
   152. The nanoparticle of embodiment 151, wherein the one or more IRFs lack a functional autoinhibitory domain.
   153. The nanoparticle of embodiments 151 or 152, wherein the one or more IRFs lack a functional nuclear export signal.
   154. The nanoparticle of any of embodiments 151-153, wherein the one or more IRFs are selected from IRF1, IRF3, IRF5, IRF7, IRF8, and/or a fusion of IRF7 and IRF3.
   155. The nanoparticle of any of embodiments 151-154, wherein the one or more IRFs are selected from a sequence having >90%, >95%, or greater than 98% identity to a sequence as set forth in SEQ ID NOs: 1-17.
   156. The nanoparticle of any of embodiments 151-155, wherein the one or more IRFs include IRF5 selected from a sequence as set forth in SEQ ID NOs: 1-7.
   157. The nanoparticle of any of embodiments 154-156, wherein the IRF5 includes a sequence as set forth in SEQ ID NO: 1 or SEQ ID NO: 3 with one or more mutations selected from S156D, S158D and T160D.
   158. The nanoparticle of any of embodiments 154-157, wherein the IRF5 includes a sequence as set forth in SEQ ID NO: 2 with one or more mutations selected from T10D, S158D, 5309D, S317D, S451D, and S462D.
   159. The nanoparticle of any of embodiments 154-158, wherein the IRF5 includes a sequence as set forth in SEQ ID NO: 4 with one or more mutations selected from S425D, S427D, 5430D, and S436D.
   160. The nanoparticle of any of embodiments 151-159, wherein the one or more IRFs include IRF1 including a sequence as set forth in SEQ ID NOs: 8 or 12.
   161. The nanoparticle of any of embodiments 151-160, wherein the one or more IRFs include IRF8 including a sequence as set forth in SEQ ID NOs: 11, 16, or 17.
   162. The nanoparticle of any of embodiments 154-161, wherein the IRF8 includes a sequence as set forth in SEQ ID NO: 11 with a K310R mutation.
   163. The nanoparticle of any of embodiments 151-162, wherein the one or more IRFs include an IRF7/IRF3 fusion protein including an N-terminal IRF7 DNA binding domain, a constitutively active domain, and a C-terminal IRF3 nuclear export signal.
   164. The nanoparticle of embodiment 163, wherein the IRF7/IRF3 fusion protein includes a sequence as set forth in SEQ ID NO: 15.
   165. The nanoparticle of any of embodiments 151-164, wherein the one or more IRFs include IRF4.
   166. The nanoparticle of any of embodiments 110-165, wherein at least a subset of the nanoparticles include nucleotides encoding IKKβ.
   167. The nanoparticle of embodiment 166, wherein the IKKβ is selected from a sequence having >90%, >95%, or >98% identity to a sequence as set forth in a sequence selected from SEQ ID NOs: 18-22.
   168. The nanoparticle of embodiments 166 or 167, wherein the IKKβ includes a sequence as set forth in a sequence selected from SEQ ID NOs: 18-22.
   169. The nanoparticle of any of embodiments 166-168, wherein the nucleotides include a sequence as set forth in a sequence selected from SEQ ID NOs: 23-44.
   170. The nanoparticle of any of embodiments 166-169, wherein the nucleotides encoding one or more IRFs and the nucleotides encoding IKKβ are encapsulated within the same nanoparticle.
   171. The nanoparticle of any of embodiments 151-170, wherein the nucleotides encoding one or more IRFs and the nucleotides encoding IKKβ are encapsulated within the same nanoparticle core.
   172. The nanoparticle of any of embodiments 151-170, wherein the nucleotides encoding one or more IRFs and the nucleotides encoding IKKβ are encapsulated in different nanoparticles.
   173. The nanoparticle of any of embodiments 151-172, wherein the nucleotides encoding at least one or more binding domains are encapsulated within the same nanoparticle as the nucleotides encoding one or more IRFs and/or IKKβ.
   174. The nanoparticle of any of embodiments 151-172, wherein the nucleotides encoding at least one or more binding domains are encapsulated within different nanoparticles than those encapsulating nucleotides encoding one or more IRFs and/or IKKβ.
   175. The nanoparticle of any of embodiments 110-174, further including a transforming growth factor beta (TGFβ) inhibitor.
   176. The nanoparticle of embodiment 175, wherein the TGFβ inhibitor includes nucleotides encoding the TGFβ inhibitor.
   177. The nanoparticle of embodiments 175 or 176, wherein the TGFβ inhibitor includes the CDRs of an antibody that suppresses the activity of TGFβ.
   178. The nanoparticle of any of embodiments 175-177, wherein the TGFβ inhibitor includes an antibody that suppresses the activity of TGFβ.
   179. The nanoparticle of embodiment 177 or 178, wherein the antibody includes trabedersen, disitertide, metelimumab, fresolimumab, LY2382770, SIX-100, avotermin, and/or IMC-TR1.
   180. The nanoparticle of any of embodiments 110-179, wherein the nanoparticles further include nucleotides encoding glucocorticoid-induced leucine zipper (GILZ).
   181. The nanoparticle of any of embodiments 110-180, wherein the nanoparticles further include nucleotides including an anticancer gene selected from p53, RB, BRCA1, E1A, bcl-2, MDR-1, p21, p16, bax, bcl-xs, E2F, IGF-I VEGF, angiostatin, oncostatin, endostatin, GM-CSF, IL-12, IL-2, IL-4, IL-7, IFN-γ, TNFα and/or HSV-tk.
   182. A system including:
nanoparticles
   wherein at least a subset of the nanoparticles include nucleotides encoding one or more interferon regulatory factors (IRFs) and
   wherein at least a subset of the nanoparticles include nucleotides encoding a protein molecule having at least two binding domains
   wherein one binding domain binds an antigen expressed by a cell of interest at a treatment site and
   wherein one binding domain binds an immune cell activating epitope.
      183. The system of embodiment 182, wherein the cell of interest is a cancer cell, an infected cell, an autoreactive cell, or a prokaryotic cell.
      184. The system of embodiments 182 or 183, wherein the cell of interest is a cancer cell and the treatment site is a tumor site.
      185. The system of any of embodiments 182-184, wherein the nanoparticles are <130 nm.
      186. The system of any of embodiments 182-185, wherein the nanoparticles include a positively-charged core and a neutrally or negatively-charged coating on the outer surface of the core.
      187. The system of embodiment 186, wherein the positively-charged core includes a positively-charged lipid and/or a positively-charged polymer.
      188. The system of embodiment 186 or 187, wherein the positively charged polymer includes poly(β-amino ester, poly(L-lysine), poly(ethylene imine) (PEI), poly-(amidoamine) dendrimers (PAMAMs), poly(amine-co-esters), poly(dimethylaminoethyl methacrylate) (PDMAEMA), chitosan, poly-(L-lactide-co-L-lysine), poly[α-(4-aminobutyl)-L-glycolic acid] (PAGA), or poly(4-hydroxy-L-proline ester) (PHP).
      189. The system of any of embodiments 186-188, wherein the positively charged polymer includes poly(β-amino ester).
      190. The system of any of embodiment 186-189, wherein the neutral or negatively-charged coating includes polyglutamic acid (PGA), poly(acrylic acid), alginic acid, or cholesteryl hemisuccinate/1,2-dioleoyl-sn-glycero-3-phosphoethanolamine.
      191. The system of any of embodiments 186-190, wherein the neutral or negatively-charged coating includes polyglutamic acid (PGA).
      192. The system of any of embodiments 186-191, wherein the neutral or negatively-charged coating includes a zwitterionic polymer.
      193. The system of any of embodiments 186-192, wherein the neutral or negatively-charged coating includes a liposome.
      194. The system of embodiment 193, wherein the liposome includes 1,2-dioleoyl-3-trimethylammonium-propane (DOTAP), 1,2-di-O-octadecenyl-3-trimethylammonium propane (DOTMA), 3ß[N-(N′,N′-dimethylaminoethane)-carbamoyl]cholesterol (DC-Chol), dioctadecyl-amidoglycylspermine (DOGS), cholesterol, 1,2-dioleoyl-sn-glycero-3-phosphoethanolamine (DOPE), or 1,2-dioleoyl-sn-glycero-3-phosphocholine (DOPC).
      195. The system of any of embodiments 182-194, wherein the nucleotides include ribonucleic acid (RNA).
      196. The system of embodiment 195, wherein the RNA includes synthetic RNA.
      197. The system of embodiments 195 or 196, wherein the RNA includes in vitro transcribed mRNA.
      198. The system of any of embodiments 182-197, wherein the nucleotides include integrating or non-integrating double-stranded DNA.
      199. The system of any of embodiments 182-198, wherein the nucleotides are in the form of a plasmid, a minicircle plasmid, or a closed-ended linear ceDNA.
      200. The system of any of embodiments 182-199, wherein the nucleotides are encapsulated within the positively-charged core.
      201. The system of any of embodiments 182-200, wherein the one or more IRFs lack a functional autoinhibitory domain.
      202. The system of any of embodiments 182-201, wherein the one or more IRFs lack a functional nuclear export signal.
      203. The system of any of embodiments 182-202, wherein the one or more IRFs are selected from IRF1, IRF3, IRF5, IRF7, IRF8, and/or a fusion of IRF7 and IRF3.
      204. The system of any of embodiments 182-203, wherein the one or more IRFs are selected from a sequence having >90%, >95%, or greater than 98% identity to a sequence as set forth in SEQ ID NOs: 1-17.
      205. The system of any of embodiments 182-204, wherein the one or more IRFs include IRF5 selected from a sequence as set forth in SEQ ID NOs: 1-7.
      206. The system of embodiment 205, wherein the IRF5 includes a sequence as set forth in SEQ ID NO: 1 or SEQ ID NO: 3 with one or more mutations selected from S156D, S158D and T160D.
      207. The system of embodiment 205 or 206, wherein the IRF5 includes a sequence as set forth in SEQ ID NO: 2 with one or more mutations selected from T10D, S158D, 5309D, S317D, S451D, and S462D.
      208. The system of any of embodiments 205-207, wherein the IRF5 includes a sequence as set forth in SEQ ID NO: 4 with one or more mutations selected from S425D, S427D, 5430D, and S436D.
      209. The system of any of embodiments 182-208, wherein the one or more IRFs include IRF1 including a sequence as set forth in SEQ ID NOs: 8 or 12.
      210. The system of any of embodiments 182-209, wherein the one or more IRFs include IRF8 including a sequence as set forth in SEQ ID NOs: 11, 16, or 17.
      211. The system of any of embodiments 182-210, wherein the IRF8 includes a sequence as set forth in SEQ ID NO: 11 with a K310R mutation.
      212. The system of any of embodiments 182-211, wherein the one or more IRFs include an IRF7/IRF3 fusion protein including an N-terminal IRF7 DNA binding domain, a constitutively active domain, and a C-terminal IRF3 nuclear export signal.
      213. The system of embodiment 212, wherein the IRF7/IRF3 fusion protein includes a sequence as set forth in SEQ ID NO: 15.
      214. The system of any of embodiments 182-213, wherein the one or more IRFs include IRF4.
      215. The system of any of embodiments 182-214, wherein at least a subset of the nanoparticles include nucleotides encoding IKKβ.
      216. The system of embodiment 215, wherein the IKKβ is selected from a sequence having >90%, >95%, or >98% identity to a sequence as set forth in a sequence selected from SEQ ID NOs: 18-22.
      217. The system of embodiments 215 or 216, wherein the IKKβ includes a sequence as set forth in a sequence selected from SEQ ID NOs: 18-22.
      218. The system of any of embodiments 182-217, wherein the nucleotides include a sequence as set forth in a sequence selected from SEQ ID NOs: 23-44.
      219. The system of any of embodiments 182-218, wherein the nucleotides encoding one or more IRFs and the nucleotides encoding IKKβ are encapsulated within the same nanoparticle.
      220. The system of embodiment 219, wherein the nucleotides encoding one or more IRFs and the nucleotides encoding IKKβ are encapsulated within the same nanoparticle core.
      221. The system of any of embodiments 182-218, wherein the nucleotides encoding one or more IRFs and the nucleotides encoding IKKβ are encapsulated in different nanoparticles.
      222. The system of any of embodiments 182-221, wherein at least one binding domain of the protein molecule binds a cancer antigen expressed by an ovarian cancer cell, a melanoma cell, a glioblastoma cell, a multiple myeloma cell, a melanoma cell, a prostate cancer cell, a breast cancer cell, a stem cell cancer cell, a mesothelioma cell, a renal cell carcinoma cell, a pancreatic cancer cell, a lung cancer cell, a cholangiocarcinoma cell, a bladder cancer cell, a neuroblastoma cell, a colorectal cancer cell, or a merkel cell carcinoma cell.
      223. The system of embodiment 222, wherein the cancer antigen includes B-cell maturation antigen (BCMA), carboxy-anhydrase-IX (CAIX), CD19, CD24, CD56, CD133, CEA, disialoganglioside, EpCam, EGFR, EGFR variant Ill (EGFRvIII), ERBB2, folate receptor (FOLR), GD2, glypican-2, HER2, Lewis Y, L1-CAM, mesothelin, MUC16, PD-L1, PSMA, Prostate Stem Cell antigen (PSCA), ROR1, TYRP1/gp75, SV40 T, or WT-1.
      224. The system of any of embodiments 182-223, wherein at least one binding domain of the protein molecule includes the complementarity determining regions (CDRs) of antibody adecatumumab, anetumab, ravtansine, amatuximab, HN1, oregovomab, ovarex, abagovomab, edrecolomab, farletuzumab. flanvotumab, TA99, 20D7, Cetuximab, FMC63, SJ25C1, HD37, R11, R12, 2A2, Y31, 4D5, 3G10 atezolizumab, avelumab, or durvalumab or a TCR.
      225. The system of any of embodiments 182-224, wherein different protein molecules within the system include binding domains that bind different cancer antigens.
      226. The system of embodiment 225, wherein the different cancer antigens are expressed by the same cancer type.
      227. The system of embodiment 226, wherein the cancer type is ovarian cancer, melanoma, or glioblastoma.
      228. The system of any of embodiments 225-227, wherein the different cancer antigens include
at least two cancer antigens selected from EpCam, L1-CAM, MUC16, folate receptor (FOLR), Lewis Y, ROR1, mesothelin, WT-1, PD-L1, EGFR, and CD56;
at least two cancer antigens selected from Tyrosinase related protein 1 (TYRP1/gp75); GD2, PD-L1, and EGFR; or
two cancer antigens selected from EGFR variant III (EGFRvIII) and IL13Ra2.
   229. The system of any of embodiments 182-228, wherein at least one binding domain of the protein molecule binds a viral antigen, a bacterial antigen, a superbug antigen, a fungal antigen, or an autoimmune or allergic antigen.
   230. The system of embodiment 229, wherein:
the viral antigen is expressed by adenoviruses, arenaviruses, bunyaviruses, coronavirusess, flavirviruses, hantaviruses, hepadnaviruses, herpesviruses, papilomaviruses, paramyxoviruses, parvoviruses, picornaviruses, poxviruses, orthomyxoviruses, retroviruses, reoviruses, rhabdoviruses, rotaviruses, spongiform viruses or togaviruses;
the bacterial antigen is expressed by anthrax; gram-negative bacilli, chlamydia, diptheria, haemophilus influenza, Helicobacter pylori, malaria, Mycobacterium tuberculosis, pertussis toxin, pneumococcus, rickettsiae, staphylococcus, streptococcus or tetanus;
the superbug antigen is expressed by Enterococcus faecium, Clostridium difficile, Acinetobacter baumannii, Pseudomonas aeruginosa, or Enterobacteriaceae;
the fungal antigen is expressed by candida, coccidiodes, cryptococcus, histoplasma, leishmania, plasmodium, protozoa, parasites, schistosomae, tinea, toxoplasma, or Trypanosoma cruzi; or
the autoimmune or allergic antigen is expressed by a subject having acute necrotizing hemorrhagic encephalopathy, allergic asthma, alopecia areata, anemia, aphthous ulcer, arthritis, asthma, autoimmune thyroiditis, conjunctivitis, Crohn's disease, cutaneous lupus erythematosus, dermatitis, diabetes, diabetes mellitus, erythema nodosum leprosum, keratoconjunctivitis, multiple sclerosis, myasthenia gravis, psoriasis, scleroderma, Sjogren's syndrome, including keratoconjunctivitis sicca secondary to Sjogren's syndrome, Stevens-Johnson syndrome, systemic lupus erythematosis, ulcerative colitis, vaginitis and/or Wegener's granulomatosis.
   231. The system of any of embodiments 182-230, wherein at least one binding domain of the protein molecule binds an immune cell activating epitope expressed by a T cell or a natural killer cell.
   232. The system of embodiment 231, wherein the immune cell activating epitope is expressed by a T cell.
   233. The system of embodiment 232, wherein the immune cell activating epitope expressed by the T cell includes CD2, CD3, CD7, CD8, CD27, CD28, CD30, CD40, CD83, 4-1BB, OX40, lymphocyte function-associated antigen-1 (LFA-1), LIGHT, NKG2C, or B7-H3.
   234. The system of embodiment 233, wherein the immune cell activating epitope expressed by the T cell includes CD3, CD28, or 4-1BB.
   235. The system of any of embodiments 182-234, wherein different protein molecules within the system include binding domains that bind different immune cell activating epitopes.
   236. The system of embodiment 235, wherein the different immune cell activating epitopes include CD3 and CD28 or CD3 and 4-1BB.
   237. The system of any of embodiments 182-236, wherein at least one binding domain includes the CDRs of antibody OKT3, 20G6-F3, 4B4-D7, 4E7-C9, 18F5-H10, TGN1412, 9D7, 9.3, KOLT-2, 15E8, 248.23.2, EX5.3D10, OKT8 or the SK1.
   238. The system of embodiment 231, wherein the immune cell activating epitope is expressed by a NK cell.
   239. The system of embodiment 238, wherein the immune cell activating epitope expressed by the NK cell includes NKG2D, CD8, CD16, KIR2DL4, KIR2DS1, KIR2DS2, KIR3DS1, NKG2C, NKG2E, NKG2D, NKp30, NKp44, NKp46, NKp80, or DNAM-1.
   240. The system of any of embodiments 182-239, wherein at least one binding domain includes the CDRs of antibody 5C6, 1D11, mAb 33, P44-8, SK1, or 3G8.
   241. The system of any of embodiments 182-240, wherein the binding domains of the protein molecule are linked through a protein linker.
   242. The system of embodiment 241, wherein the protein linker includes a Gly-Ser linker.
   243. The system of embodiment 241 or 242, wherein the protein linker includes a proline-rich linker.
   244. The system of any of embodiments 182-243, wherein the protein molecule includes a single chain variable fragment (scFv).
   245. The system of any of embodiments 182-244, wherein the protein molecule includes
at least one binding domain binds CEA and at least one binding domain binds CD3, CD28, or 4-1BB;
at least one binding domain binds EGFR and at least one binding domain binds CD3, CD28, or 4-1BB;
at least one binding domain binds EpCam and at least one binding domain binds CD3, CD28, or 4-1BB;
at least one binding domain binds HER2 and at least one binding domain binds CD3, CD28, or 4-1BB;
at least one binding domain binds PD-L1 and at least one binding domain binds CD3, CD28, or 4-1BB;
at least one binding domain binds PSMA and at least one binding domain binds CD3, CD28, or 4-1BB; or
at least one binding domain binds [TYRP1/gp75] and at least one binding domain binds CD3, CD28, or 4-1BB.
   246. The system of any of embodiments 182-245, wherein the protein molecule includes catumaxomab, MT110, ertumaxomab, MDX-447, MM-141, AMG211, R06958688, R06895882, TF2, BAY2010112, AMG701, solitomab, or blinatumomab.
   247. The system of any of embodiments 182-246, wherein the nucleotides encoding at least two binding domains are encapsulated within the same nanoparticle as the nucleotides encoding one or more IRFs and/or IKKβ.
   248. The system of embodiment 247, wherein the nucleotides encoding at least two binding domains are encapsulated within the same nanoparticle core as the nucleotides encoding one or more IRFs and/or IKKβ.
   249. The system of any of embodiments 182-246, wherein the nucleotides encoding at least two binding domains are encapsulated within different nanoparticles than those encapsulating nucleotides encoding one or more IRFs and/or IKKβ.
   250. The system of any of embodiments 182-249, further including a transforming growth factor beta (TGFβ) inhibitor.
   251. The system of embodiment 250, wherein the TGFβ inhibitor includes nucleotides encoding the TGFβ inhibitor.
   252. The system of embodiments 250 or 251, wherein the TGFβ inhibitor includes the CDRs of an antibody that suppresses the activity of TGFβ.
   253. The system of any of embodiments 250-252, wherein the TGFβ inhibitor includes an antibody that suppresses the activity of TGFβ.
   254. The system of embodiments 252 or 253, wherein the antibody includes trabedersen, disitertide, metelimumab, fresolimumab, LY2382770, SIX-100, avotermin, and/or IMC-TR1.
   255. The system of any of embodiments 182-254, wherein the nanoparticles further include nucleotides encoding glucocorticoid-induced leucine zipper (GILZ).
   256. The system of any of embodiments 182-255, wherein the nanoparticles further include nucleotides including an anticancer gene selected from p53, RB, BRCA1, E1A, bcl-2, MDR-1, p21, p16, bax, bcl-xs, E2F, IGF-I VEGF, angiostatin, oncostatin, endostatin, GM-CSF, IL-12, IL-2, IL-4, IL-7, IFN-γ, TNFα and/or HSV-tk.
   257. The system of any of embodiments 182-256, further including a pharmaceutically acceptable carrier.
   258. A monocyte or macrophage genetically modified to express the nucleotides of a system of any of embodiments 182-257.
   259. A method of modulating the macrophage activation state at a treatment site within a subject, recruiting immune cells to the treatment site, and activating the recruited immune cells including:
administering the system of any of embodiments 182-257 to the subject, thereby modulating the macrophage activation state at the treatment site within the subject, recruiting immune cells to the treatment site, and activating the recruited immune cells.
   260. The method of embodiment 259, wherein the treatment site is a tumor site.
   261. The method of embodiments 259 or 260, wherein the administering includes intravenous administering and the nanoparticles are taken up by monocytes within the blood stream.
   262. The method of embodiment 261, wherein the monocytes migrate to the tumor site and differentiate into macrophages.
   263. The method of embodiment 262, wherein the differentiated macrophages are resistant to tumor suppression.
   264. The method of any of embodiments 259-263, wherein the administering includes locally administering at the tumor site and the nanoparticles are taken up by tumor-associated macrophages (TAM).
   265. The method of embodiment 264, wherein the local administering includes intraperitoneally administering or intracranially administering.
   266. The method of embodiments 264 or 265, wherein the TAM undergo a phenotype transformation from a suppressed to an activated state.
   267. The method of any of embodiments 264-266, wherein the tumor site includes an ovarian cancer tumor site, a glioblastoma tumor site, or a melanoma cancer tumor site.
   268. The method of any of embodiments 259-267, wherein the recruited and activated immune cells are T cells or NK cells.
   269. The method of any of embodiments 259-268, including administering nanoparticles including nucleotides encoding one or more IRFs before administering nanoparticles including nucleotides encoding at least two binding domains.
   270. The method of any of embodiments 259-269, including administering nanoparticles including nucleic acids encoding one or more IRFs at least 24 hours before administering nanoparticles including nucleotides encoding at least two binding domains.

(12) Experimental Examples. Example 1. Materials and Methods. PbAE synthesis. The methods used to synthesize the polymer were described previously (Mangraviti A et al. (2015) ACS Nano 9: 1236-1249). 1,4-butanediol diacrylate was combined with 4-amino-1-butanol in a 1:1 molar ratio of diacrylate to amine monomers. Acrylate-terminated poly(4-amino-1-butanol-co-1,4-butanediol diacrylate) was formed by heating the mixture to 90° C. with stirring for 24 hours. 2.3 g of this polymer was dissolved in 2 mL tetrahydrofuran (THF). To form the piperazine-capped 447 polymer, 786 mg of 1-(3-aminopropyl)-4-methylpiperazine in 13 mL THF was added to the polymer/THF solution and stirred at room temperature (RT) for 2 hours. The capped polymer was precipitated with 5 volumes of diethyl ether, washed with 2 volumes of fresh ether, and dried under vacuum for 1 day. Neat polymer was dissolved in dimethyl sulfoxide (DMSO) to a concentration of 100 mg/mL and stored at −20° C.

PGA conjugation to Di-mannose. α-D-mannopyranosyl-(1→2)-α-D-mannopyranose (Di-mannose, Omicron Biochemicals Inc.) was modified into glycosylamine before being conjugated to polyglutamic acid (PGA). First, the Di-mannose (157 mg) was dissolved in 10.5 mL of saturated aqueous ammonium carbonate, then stirred at RT for 24 hours. On the second day, more solid ammonium carbonate was added until the Di-mannose precipitated from the reaction solution. The mixture was stirred until completion, as measured by TLC, followed by lyophilization to remove the excess ammonium carbonate. Complete removal of volatile salt was accomplished by re-dissolving the solid in methanol. These procedures created an amine on the anomeric carbon for future conjugation with PGA.

To conjugate aminated Di-mannose to PGA, the substrate was dissolved in water to 30 mg m/L, then sonicated for 10 minutes. Ethyl-N′-(3-dimethylaminopropyl) carbodiimide·HCl in water (4 mg/mL, 30 equiv.) was added with mixing at RT for 4 min. N-hydroxysulfosuccinimide in water (30 mg/mL, 35 equiv.) was incubated with the PGA/EDC solution for 1 minute. Aminated Dimannose in phosphate-buffered saline (PBS) was combined with the resulting activated PGA in a 44:1 molar ratio and mixed at RT for 6 h. Excess reagents were removed by dialysis against water for 24 hours.

mRNA synthesis. Codon-optimized mRNA for eGFP, IRF5, and IKK (TriLink Biotechnologies) were capped with the Anti-Reverse Cap Analog 3′-O-Me-m7G(5′)ppp(5′)G (ARCA), and fully substituted with the modified ribonucleotides pseudouridine (4)) and 5-methylcytidine (m5C).

Nanoparticle preparation. IRF5 and IKKβ mRNAs were combined at a 3:1 (w:w) ratio and diluted to 100 μg/mL in 25 mM sodium acetate (NaOAc) buffer (pH=5.2). Poly(β-amino esters)-447 (PbAE-447) polymer in DMSO (prepared as described above) was diluted from 100 μg/μL to 6 μg/μL, also in NaOAc buffer. To form the nanoparticles, PbAE-447 polymers were added to the mRNA at a ratio of 60:1 (w:w) and vortexed immediately for 15 seconds at a medium speed, then the mixture was incubated at RT for 5 min to allow the formation of PbAE-mRNA polyplexes. In the next step, 100 μg/mL PGA/Di-mannose in NaOAc buffer was added to the polyplexes solution, vortexed for 15 seconds at medium speed, and incubated for 5 min at room temperature. In this process, PGA/Di-mannose coated the surfaces of PbAE-mRNA polyplexes to form the final NPs. For long-term storage, D-sucrose (60 mg/mL) was added to the NP solutions as a cryoprotectant. The nanoparticles were snap-frozen in dry ice, then lyophilized. The dried NPs were stored at −20° C. or −80° C. until use. For in vivo experiments, lyophilized NPs were re-suspended in water at a 1:20 (w:v) ratio.

Characterization of nanoparticle size distribution and ζ-potential. The physiochemical properties of NPs (including hydrodynamic radius, polydispersity, ζ-potential, and stability) were characterized using a Zetapals instrument (Brookhaven Instrument Corporation) at 25° C. To measure the hydrodynamic radius and polydispersity based on dynamic light scattering, NPs were diluted 5-fold into 25 mM NaOAc (pH=5.2). To measure the ζ-potential, NPs were diluted 10-fold in 10 mM PBS (pH=7.0). To assess the stability of NPs, freshly prepared nanoparticles were diluted in 10 mM PBS buffer (pH=7.4). The hydrodynamic radius and polydispersity of NPs were measured every 10 minutes for 5 hours, and their sizes and particle concentrations were derived from Particle Tracking Analysis using a Nanosite 300 instrument (Malvern). To characterize the NPs using transmission electron microscopy, previously described protocols were followed (Smith T T et al. (2017) Nat Nanotechnol 12: 813-820). Freshly made NPs (25 μL containing 0.83 μg of mRNA) were deposited on glow discharge-treated 200 mesh carbon/Formvar-coated copper grids. After 30 seconds, the grids were treated sequentially with 50% Karnovsky's fixative, 0.1 M cacodylate buffer, dH2O, then 1% (w/v) uranyl acetate. Samples were imaged with a JEOL JEM-1400 transmission electron microscope operating at 120 kV (JEOL USA).

Bone marrow derived macrophages (BMDMs) and other cell lines. To prepare BMDMs, bone marrow progenitor cells were harvested from mouse femurs following established protocols (Zhang X et al. (2008) Curr Protoc Immunol Chapter 14: Unit 14 11). These cells were cultured in complete medium [DMEM supplemented with 4.5 g/L D-glucose, L-glutamine, 10% heat-inactivated fetal bovine serum (FBS), 100 U/mL penicillin and 100 μg/mL, Glutamax 50 mL/500 mL, supplemented with 20 ng/mL M-CSF (Peprotech, cat #315-02)] at a seeding density of 0.5-1.0 e6/ml. Cells were allowed to differentiate into BMDMs ex vivo for 7 days under 5% CO2 at 37° C. Next, they were conditioned with macrophage-conditioned medium [macrophage complete medium supplemented with 20 ng/mL MPLA (Sigma, cat #L6895) or 20 ng/mL IL4 (eBioscience, cat #34-8041)]. BMDMs were used between 7-21 days ex vivo. The murine ovarian cancer cell line ID8, a gift from Dr. Katherine Roby (University of Kansas Medical Center, Kansas City, KS), was cultured in DMEM supplemented with 10% FBS, 100 U/mL penicillin, 5 μg/mL insulin, 5 μg/mL transferrin, and 5 ng/mL sodium selenite (all Sigma-Aldrich). To generate the more aggressive vascular endothelial growth factor (VEGF)-expressing ID8 strain, ID8 tumor cells were transfected with the pUNO1 plasmid (Invivogen) encoding murine VEGF along with the blasticidin-resistance gene. To obtain stable transfectants, tumor cells were cultured in complete medium containing 10 μg/mL blasticidin (Invivogen) for 3 weeks. The B16F10 melanoma cell line (American Type Culture Collection) was cultured in complete RPMI 1640 medium with 10% FBS, 100 U/mL penicillin, 2 mM/L-glutamine, 1.5 g/L sodium bicarbonate, 4.5 g/L glucose, 10 mM HEPES, 1.0 mM sodium pyruvate, and 0.05 mM 2-mercaptoethanol. For in vivo bioluminescent imaging, both ID8-VEGF and B16F10 cell lines were retrovirally transduced with firefly luciferase. The DF-1 cell line carrying RACS-PDGFβ or RCAS-cre retrovirus was cultured in complete medium supplemented with 10% FBS and 100 U/mL penicillin under 5% CO2 at 39° C.

mRNA transfection of BMDMs. One day prior to transfection, BMDMs were reseeded on 24-well plates in macrophage complete medium at a concentration of 250,000/well. Before transfection, the complete medium was replaced with 300 μL unsupplemented DMEM. To transfect these cells, NPs containing 2 μg mRNA were added into the base medium and co-cultured with the BMDMs at 37° C. After 1 hour, medium containing NPs was removed, and the cells were cultured an additional 24 hours before evaluation of transfection efficiency and cell viability.

Transfection of BMDMs for macrophage signature gene analysis. BMDMs were reseeded on 24-well plates in conditioned medium 24 hours prior to transfection, allowing transformation of the cells into their phenotypes. M2-like macrophages were then exposed to either IRF5/IKKβ. NPs carrying 25% eGFP mRNA as a reporter, or eGFP NPs (control) containing 2 μg mRNA, following the transfection protocol described above. After 24 hours, the top 10% percent of highly transfected BMDMs (as measured by eGFP expression) were sorted at 24 hours after transfection and were re-challenged in low-dose (10 ng/mL) IL4 medium for another 48 hours before RNA isolation. RNAs extracted from these cells were compared to those from standard M1- or M2-like macrophages so that signature genes associated with IRF5-N P treatment could be identified.

RNA isolation and preparation. To harvest RNAs, BMDMs were lysed in Trizol reagent (Ambion), and total RNAs were extracted and purified using RNeasy® Plus Universal Mini-Kits (QIAGEN) following the manufacturer's instructions. Sample RNA was quantified using a NanoDrop Microvolume Spectrophotometer (Thermo Fisher) and then subjected to quality control performed by the FHCRC Genomics Shared Resource with an Agilent 4200 TapeStation analyzer (Agilent).

Macrophage signature gene analysis by NanoString Technology. Gene expression values from stimulated BMDM cultures were measured using the nCounter® Myeloid Innate Immunity Panel (NanoString Technologies, Seattle, WA), which analyzes 770 genes occurring in 19 different pathways and processes them across 7 different myeloid cell types. The samples were tested using an nCounter Analysis System (NanoString Technologies, Seattle, WA). Raw data were processed and checked for quality using the R/Bioconductor NanoStringQCPro software package (Nickles D, Sandmann T, Ziman R and Bourgon R (2018) NanoStringQCPro: Quality metrics and data processing methods for NanoString mRNA gene expression data. R package version 1.10.0.). Expression values were normalized to the geometric mean of housekeeping genes and log 2-transformed using nSolver 4.0 software (NanoString Technologies, Seattle, WA). False Discovery Rates for ratio data were calculated from the p-values returned by the t-tests using the Benjamini-Yekutieli method.

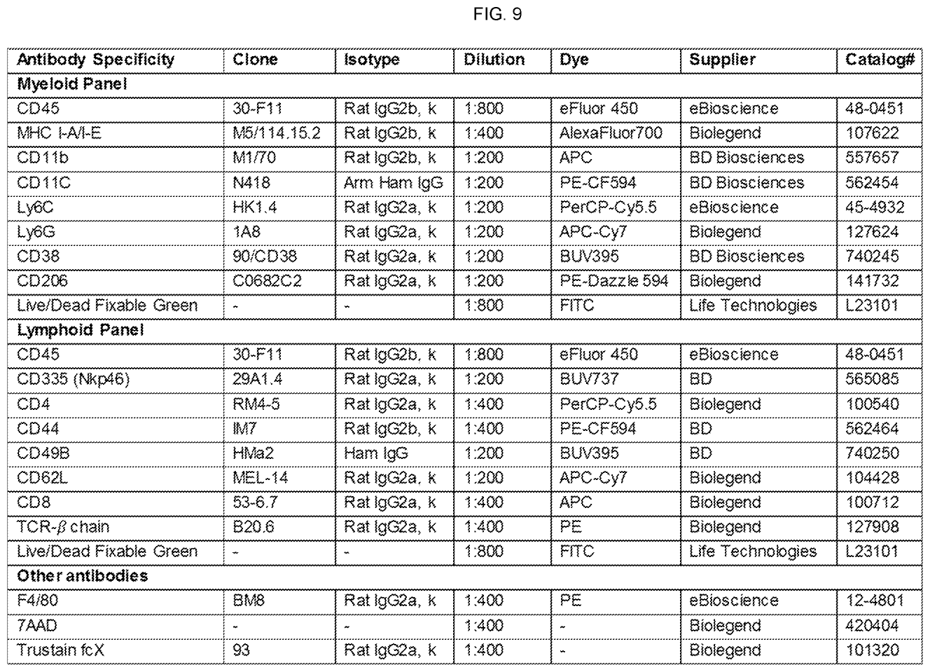

Flow Cytometry and cell sorting. Cells obtained from spleen, blood, peritoneal lavage, and bronchoalveolar lavage were analyzed by flow cytometry with myeloid and lymphoid immunophenotyping panels using the anti-mouse antibody probes listed in FIG. 9. Data were collected using a BD LSRFortessa analyzer running FACSDIVA software (Beckton Dickinson). CD11b+ and F4/80+ peritoneal macrophages were sorted using BD FACS ARIA II. All collected data were analyzed using FlowJo 10.0 software.

Cytokine analysis. Cytokine levels were evaluated using a Luminex 200 system (Luminex) at the FHCRC Immune Monitoring Shared Resource center. For ex vivo studies, cell culture supernatant was collected for the measurement of IL-6, IL-12p70, INFy, and TNFα concentrations. For in vivo studies, plasma concentration of GM-CSF, INFy, IL-12p70, IL-2, IL-6, and TN Fa were measured.

qRT-PCR analysis. Gene expression levels were determined by qRT-PCR. To measure selected macrophage signature genes (SerpinB2, Retnla, Ccl5, CcI11, codon-optimized IRF5, endogenous IRF5, and housekeeping GAPD genes), total RNA was isolated with RNeasy mini-columns (Qiagen) according to the manufacturer's instructions. cDNA was synthesized using a qScript cDNA Synthesis Kit (Quanta). For each sample, qRT-PCR was performed in triplicate via PerfeCTa qPCR SuperMix Low ROX (Quanta) using gene-specific probes from the Roche's Universal Probe Library (UPL) and PCR primers optimized by ProbeFinder (Roche): SerpinB2, UPL-049, F-ACTGGGGCAGTTATGACAGG (SEQ ID NO: 96), R-GATGATCGGCCACAAACTG (SEQ ID NO: 97); Retnla, UPL-078, F-TTGTTCCCTTCTCATCTGCAT (SEQ ID NO: 98), R-CCTTGACCTTATTCTCCACGA (SEQ ID NO: 99); Ccl5, UPL-105, F-CCTACTCCCACTCGGTCCT (SEQ ID NO: 100), R-CTGATTTCTTGGGTTTGCTGT (SEQ ID NO: 101); CcI11, UPL-018, F-AGAGCTCCACAGCGCTTC (SEQ ID NO: 102), R-CAGCACCTGGGAGGTGAA (SEQ ID NO: 103); codon-optimized IRF5, UPL-022, F-TCTTAAAGACCACATGGTAGAACAGT (SEQ ID NO: 104), R-AGCTGCTGTTGGGATTGC (SEQ ID NO: 105); endogenous IRF5, UPL-011, F-GCTGTGCCCTTAACAAAAGC (SEQ ID NO: 106), R-GGCTGAGGTGGCATGTCT (SEQ ID NO: 107). Signature gene mRNA levels were normalized based on amplification of GAPD, UPL-060, F-AGCCACATCGCTCAGACAC (SEQ ID NO: 108) and R-GCCCAATACGACCAAATCC (SEQ ID NO: 109). All qRT-PCR reactions were performed using Quant Studio5 RT-PCR machines running QuantStudio6 software (Applied Biosystems). In cases when the amplification plot did not cross the threshold and no Ct value was obtained (“undetermined”), a Ct value equal to the highest cycle number of in the assay (40 cycles) was used for comparisons of relative expression.

Mice and in vivo tumor models. Except for the brain tumor model-related experiments, the mice used in these experiments were obtained from Jackson Laboratory; the others were bred and housed in the FHCRC animal facility. All of the mice were used in the context of a protocol approved by the center's Institutional Animal Care and Use Committee. To model ovarian tumors, 5×106 vascular epithelial growth factor (VEGFP)-expressing ID8 cells were injected intraperitoneally (i.p.) into 4- to 6-week-old female albino B6 (C57BL/6J-Tyr<c-2J>) mice and allowed to establish for 2 weeks. For survival studies, the animals were treated i.p. with IRF5 NPs/eGFP NPs carrying 50 μg mRNA (two doses per week for 9 weeks, or until health conditions reached euthanizing requirements). For mechanism studies, the treatments for either 1, 2, or 3 weeks, were used followed by euthanization at 48 hours following the last dose. Peritoneal lavage was performed to collect the peritoneal cells. To compare the efficacy of IRF5/IKKβ NPs with status quo macrophage targeting therapies, one group of mice received treatment with IRF5/IKKβ NPs carrying 50 μg mRNA for 3 weeks with 2 doses per week; the second received oral gavage of 15 mg/kg PI3Ky inhibitor IPI-594 (MedKoo Biosciences Inc) formulated in vehicle (5% 1-methyl-2-pyrrolidinone in polyethylene glycol 400) daily for 3 weeks; the third group received i.p. injection of 30 mg/kg CSF1R inhibitor Pexidartinib (PLX3397, MedKoo Biosciences Inc) formulated in the same vehicle daily for 3 weeks.

To model metastatic lung cancer, 2.5×104 16F10 cells transduced with F-luc and suspended in 200 μL RPMI medium were injected into 4- to 6-week-old female albino B6 (C57BLJ6J-Tyr<c-2J>) mice (Jackson Laboratories) and allowed to establish for 1 week. For survival studies, mice were treated retro-orbitally with (or without) IRF5/IKKβ or eGFP NPs carrying 30 μg mRNA suspended in PBS. Mice were treated with 3 doses/wk for 3 weeks or until health conditions reached euthanizing requirements. For mechanism studies, the mice received the same treatments for 2 weeks. Bronchoalveolar lavage was performed to collect alveolar cells for analysis.

Mice bearing glioma were generated following published protocols (Uhrbom L et al. (2004) Nat Med 10: 1257-1260). Avian DF-1 cells producing RCAS-PDGF8 and RCAS-cre retroviruses were injected intracranially into both brain hemispheres (coordinates: 1 mm caudal from bregma, 2 mm lateral, depth of 2 mm from the dural surface) of Nestin-tv-a/Ink4a-arf−/−; Pten−/− mice (C57BLJ6) between 4-6 weeks of age. Tumors were allowed to establish for 2 weeks. At day 15, mice received 10Gy radiation to one hemisphere, while the unirradiated hemisphere was shielded with lead. The next day, mice received retro-orbital injections of IRF5/IKKβ NPs carrying 30 μg mRNA (3 doses/wk for 3 weeks) or were assigned to the PBS control group.

In vivo bioluminescence imaging. D-Luciferin (Xenogen) in PBS (15 mg/mL) was used as a substrate for firefly luciferase imaging. Bioluminescence images were collected with a Xenogen IVIS Spectrum Imaging System (Xenogen). Mice were anesthetized with 2% isoflurane (Forane, Baxter Healthcare) before and during imaging. For ID8-VEGF ovarian tumors, each mouse was injected i.p. with 300 μg of D-Luciferin, and images were collected 10 minutes later. For B16F10 lung metastatic tumors, mice were injected i.p. with 3 mg of D-Luciferin, and images were collected 15 minutes afterwards. For brain tumor models, the mice received retro-orbital injection of 75 mg/kg body weight D-Luciferin, and images were collected 4 minutes later. Acquisition times ranged from 10 s to 5 min.

Biodistribution analysis. To determine the biodistribution of IRF5 NPs in the ID8-VEGF ovarian tumor model, mice in 7-8 groups received an i.p. or retro-orbital dose of NPs carrying 50 μg mRNA. Twenty-four hours after injection, whole blood was collected, and mice were euthanized with CO2 to retrieve organs (liver, spleen, lung, kidney, heart, intestine, pancreases, and diaphragm). All tissues were stabilized with RNAlater, then frozen on dry ice. The codon-optimized IRF5 mRNA levels in each organ were measured using RT-qPCR.

Toxicity analysis. To measure potential in vivo toxicities of repeatedly infusing macrophage-targeting NPs, we injected mice (5/group) intravenously with 6 sequential doses of IRF5/IKKβ or eGFP NPs carrying 50 μg mRNA over the course of 3 weeks. Controls received no treatment. Twenty-four hours after the final infusion, mice were anesthetized and blood was collected by retro-orbital bleed to determine the complete blood counts. Blood was also collected for serum chemistry and cytokine profile analyses (performed by Phoenix Central Laboratories, Mukilteo, WA). Animals were then euthanized with CO2 to retrieve organs, which were washed with deionized water before fixation in 4% paraformaldehyde. The tissues were processed routinely, and sections were stained with hematoxylin and eosin. The specimens were interpreted by Dr. Smitha Pillai MVSc, PhD, DACVP, a board-certified staff pathologist, in a blinded fashion.

Cytokine assays. Cytokine levels were evaluated using a Luminex 200 system (Luminex) at the FHCRC Immune Monitoring Shared Resources. For ex vivo studies, cell culture supernatant was collected for the measurement of IL-6, IL12p70, INFγ, and TNFα concentrations. For in vivo studies, we measured plasma concentrations of GM-CSF, INFγ, IL-12p70, IL-2, IL-6, and TNFα.

Statistical analysis. The statistical significance of observed differences were analyzed using the unpaired, two-tailed one-way ANOVA test. The P values for each measurement are listed in the figure or figure legends. Survival data was characterized using the Log-rank test. All statistical analyses were performed either using GraphPad Prism software version 6.0 or R software.

Results. Designing NPs to choreograph IVT mRNA transfection of TAMs. A targeted mRNA delivery system was developed that can introduce robust gene expression in the targeted cells by taking advantage of electrostatic interactions between cationic poly(β-amino ester) (PbAE) polymers and anionic mRNA (FIG. 2A). To improve the stability and translation of the mRNA encapsulated in the resulting nanocarriers, synthetic versions of the message were used that incorporate the modified ribonucleotides pseudouridine (ψ) (Kariko K et al. (2008) Mol Ther 16: 1833-1840) and 5-methylcytidine (m5C), and that are capped with ARCA (Anti-Reverse Cap Analog) (Quabius E S et al. (2015) N Biotechnol 32: 229-235). The mRNA is released from the mRNA-PbAE complex intracellularly by hydrolytic cleavage of ester bonds in the PbAE backbone. Efficient in vivo T cell transfection was previously demonstrated using this system (Smith T T et al. (2017) Nat Nanotechnol). To target the nanoparticles to TAMs as well as further stabilize the mRNA-PbAE complexes they contain, Di-mannose moieties were engineered onto their surfaces using polyglutamic acid (PGA) as a linker (FIG. 2A). The NPs were manufactured utilizing a simple two-step, charge driven self-assembly process. First, the synthetic mRNA was complexed with a positively charged PBAE polymer, which condenses the mRNA into nano-sized complexes. This step was followed by the addition of PGA functionalized with Di-mannose, which shields the positive charge of the PBAE-mRNA nanoparticles and confers macrophage-targeting. The resulting mRNA nanocarriers had a size of 99.8±24.5 nm, a polydispersity of 0.183, and a neutral surface charge (3.40±2.15 mV ζ-potential, FIG. 2B-2C). The transfection efficiency was first tested in murine bone marrow-derived macrophages (BMDMs) using NPs formulated with green fluorescent protein-encoding mRNA (GFP-NPs). Briefly, 50,000 BMDMs were exposed to NPs containing 1 μg mRNA for 1 hour, followed by flow cytometry measurements of GFP expression the next day. Following a single NP application, we routinely transfected 31.9% (±8.5%) of these primary macrophages without reducing their viability (FIG. 2E-2F). Surface modification of nanoparticles with Di-mannose was relevant, as transfection rates with untargeted (but PGA-coated) nanocarriers dropped to an average of 25% (±2.1%) in this inherently phagocytic cell type. The NPs selectively targeted the CD11b+, F4/80+ macrophage population, with 46% of macrophages transfected and expressing high levels of eGFP (FIG. 2D). This high transfection efficiency demonstrates the potency of the disclosed systems and methods in targeted delivery of mRNA to TAMs. Based on the results of an in vitro screen for transcription factor candidates that induce macrophage polarization, two mRNAs were selected for inclusion in the NP: the first encodes IRF5, a key member of the IRF family that favors the polarization of macrophages toward the M1 phenotype, and the second encodes IKKβ, a kinase that phosphorylates and activates IRF5.

Programming immunosuppressive macrophages into proinflammatory phenotypes. To induce macrophage polarization, two mRNAs were selected for inclusion into the NPs: the first encodes IRF5, a key member of the interferon regulatory factor family that favors the polarization of macrophages toward the M1 phenotype (Krausgruber T et al. (2011) Nat Immunol 12: 231-238); the second encodes IKKβ, a kinase that phosphorylates and activates IRF5 (Ren J et al. (2014) Proc Natl Acad Sci USA 111: 17438-17443). A ratio of 3 IRF5 mRNAs to 1 IKKβ. mRNA was used. Using real-time quantitative PCR specific for the NP-delivered (and codon-optimized) IRF5 mRNA, it was found that mRNA expression in macrophages was maximal at day 1, resulting in a 1,500-fold increase in IRF5 relative to endogenous factor levels (FIG. 2A). As expected, gene expression was transient but IRF5 levels remained strongly upregulated through day 3 (581-fold increased) and day 5 (87-fold increased) before returning to baseline.

To determine if IRF5/IKKβ-encoding NPs can reprogram M2 macrophages into the therapeutically desirable anti-cancer M1 phenotype, NanoString gene expression analysis was used. BMDMs were first cultured in the presence of interleukin-4 (IL-4) to induce a suppressive M2 phenotype (FIG. 2H). Following transfection with either control GFP-mRNA nanoparticles or IRF5/IKKβ. mRNA-containing NPs, gene expression profiles were analyzed and compared with inflammatory macrophages, which were generated separately by exposing BMDMs to the TLR4 agonist Monophosphoryl Lipid A. Despite being cultured in suppressive IL-4-containing medium, macrophages transfected with IRF5/IKKβ. mRNA NPs display gene expression profiles similar to inflammatory macrophages (FIG. 2I). Signature M2 macrophage genes, such as Serpinb2 and Ccl2 (Jablonski K et al. (2015) Plos One 10: e0145342; Varga T et al. (2016) J Immunol 196: 4771-4782), were strongly downregulated while key M1 differentiation genes, such as Ccl5 (Sica A et al. (2012) J Clin Invest 122: 787-795), were upregulated (FIG. 2J, 2K). These data establish that NP-mediated expression of IRF5 and its kinase skews suppressive macrophages toward a proinflammatory phenotype.

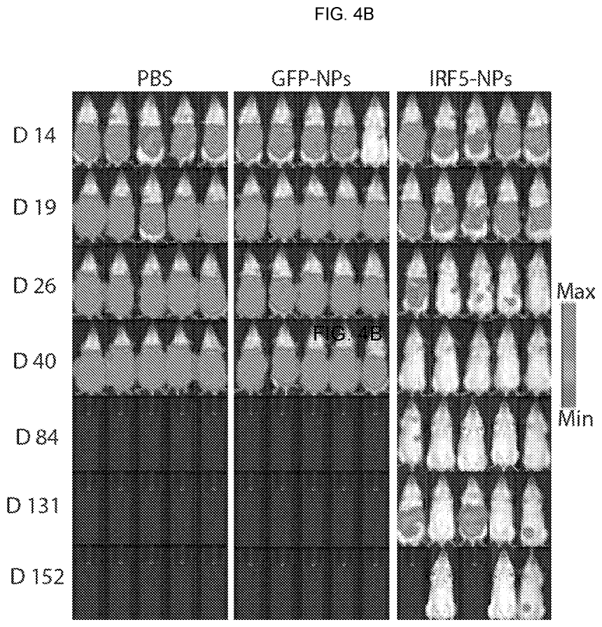

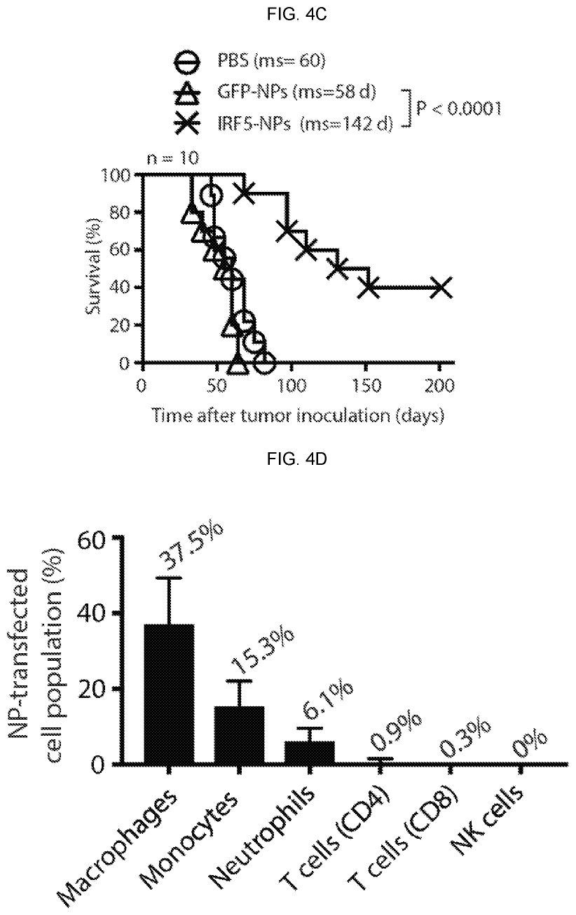

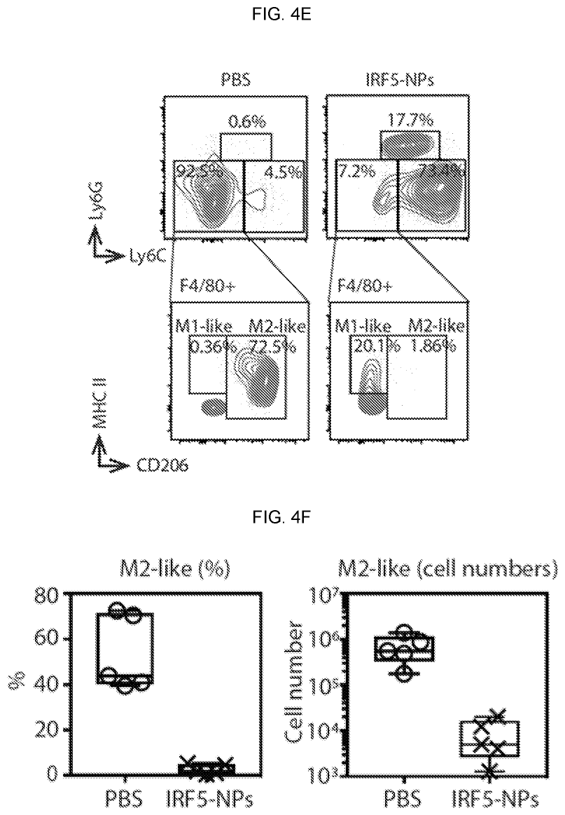

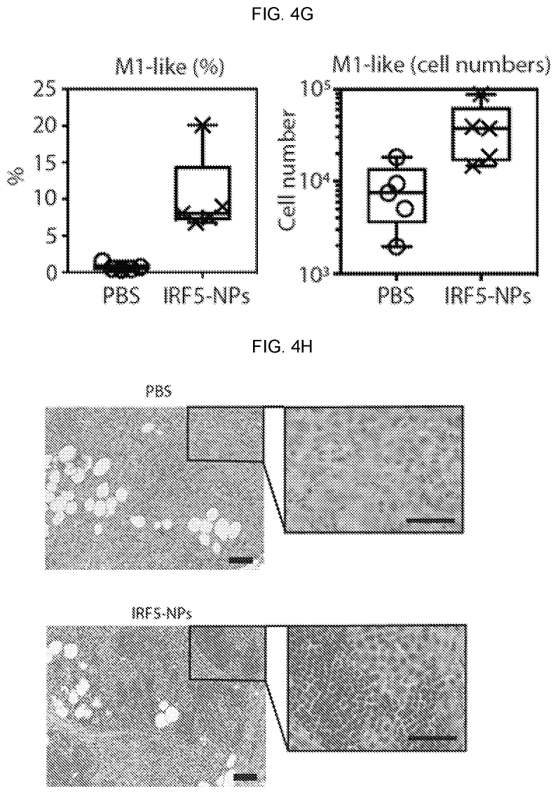

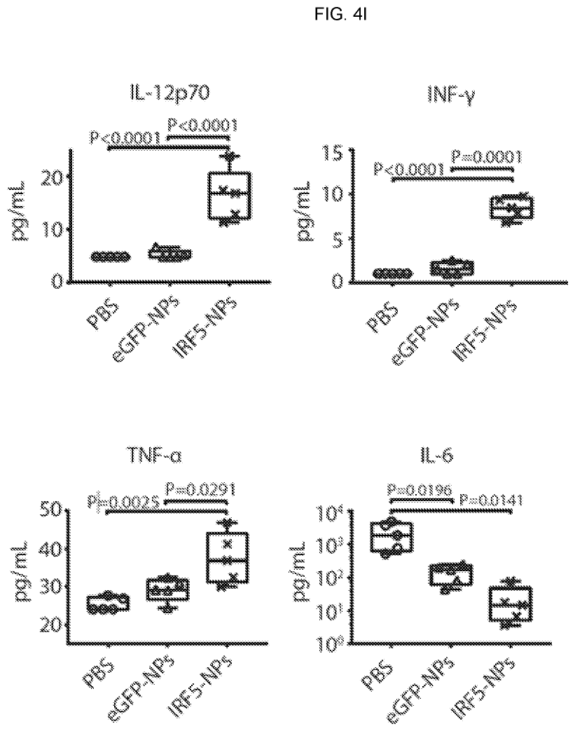

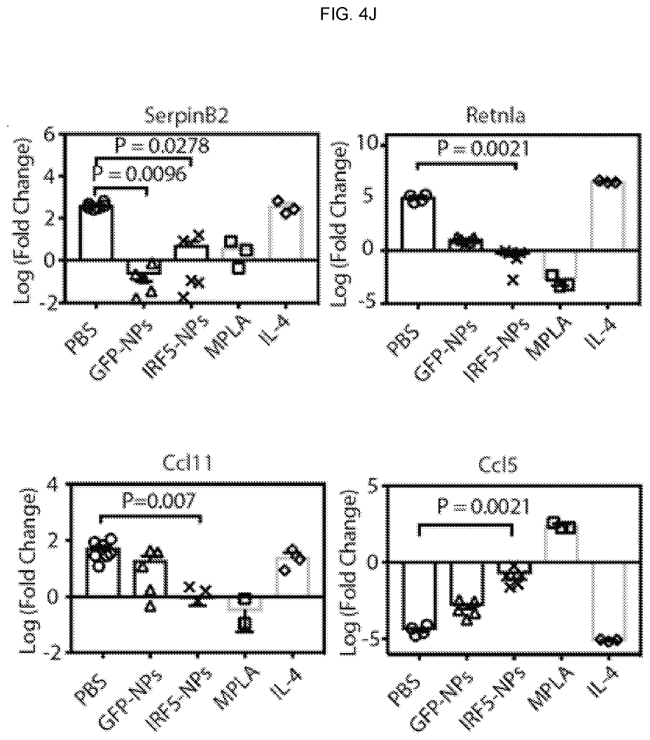

Example 2. Therapeutic effects of NP-delivered pro-M1 genes for disseminated ovarian cancer. To evaluate this treatment approach in a clinically-relevant in vivo test system, a model that recapitulates late-stage, unresectable ovarian tumors in C57BL/6 mice was used; these animals are injected with 1D8 ovarian cancer cells which were tagged with luciferase to enable serial bioluminescent imaging of tumor growth (Liao J B et al. (2015) J Immunother Cancer 3: 16; Stephan S B et al. (2015) Nat Biotechnol 33: 97-101). The tumors were allowed to establish for two weeks. By this stage, the mice have developed nodules throughout the peritoneal wall and in the intestinal mesentery. The animals were divided into 3 groups that received PBS (control), GFPNPs (sham), or IRF5/IKKβ. NP treatment at an i.p. dose of 100 μg mRNA/mouse/week for 9 weeks (FIG. 4A). It was observed that in the IRF5/IKKβ. NP treated group, the disease regressed and was eventually cleared in 40% of animals (overall 142 d median survival versus 60 d in controls; FIG. 4B-4C). To understand the underlying mechanisms of IRF5/IKKβ. NP-mediated anti-tumor effects, how exclusively mannose receptor-targeting confined NP interaction to phagocytes was first examined. Flow cytometry of peritoneal lavage fluid collected 24 h after the first dose of NPs targeted with Di-mannose revealed preferential gene transfer into macrophages and monocytes (average 37.1% and 15.3%, respectively, FIG. 4D), while transfection into off-target cells was low or undetectable. A detailed phenotypic and functional analysis of macrophage/monocyte populations in the peritoneum of mice with established ovarian cancer following treatment with IRF5/IKKβ nanoparticles or PBS over a 3-week period (two weekly injections) was conducted next. Flow cytometric analysis revealed that IRF5/IKKβ. NPs reduced the population of immune-suppressive macrophages (Ly6C−, F4/80+, CD206+) to an average 2.6%±2.1% versus 43%±15.6% in controls (FIG. 4E-4F). Conversely, the fraction of M1-like macrophages increased from 0.5%±0.2% to 10.2%±4.1% (FIG. 4E, 4G). IRF5 gene therapy also affected the population of other immune cells. In particular, inflammatory monocytes (CD11b+, Ly6C+, Ly6G−) were more abundant (73.4%±3.6% compared to 4.5%±1.9% in untreated mice). One interesting finding in all IRF5 NP-treated animals were multifocal dense clusters of lymphocytes present within or surrounding the neoplasms (FIG. 4H), indicating that genetic programming of immune stimulatory macrophages may restore lymphocyte migration and infiltration into solid tumors.

Peritoneal macrophages were isolated by fluorescence-activated cell sorting to analyze their cytokine secretion, and detected a robust increase in the release of pro-inflammatory (anti-tumor) cytokines IL-12 (3.4-fold higher), IFN-g (8.4-fold higher), and TNF-α (1.5-fold higher), whereas the levels of IL-6, a regulatory cytokine associated with differentiation toward alternatively activated (M2-like) macrophages, were reduced by 97-fold; FIG. 4I). Genome expression profiling confirmed differentiation toward an M1-like macrophage phenotype in IRF5/IKKβ nanoparticle-treated mice. Gene expression levels of macrophages cultured ex vivo in MPLA or IL-4 were included to provide reference values for classic M1-like or M2-like macrophages, respectively (FIG. 4J).

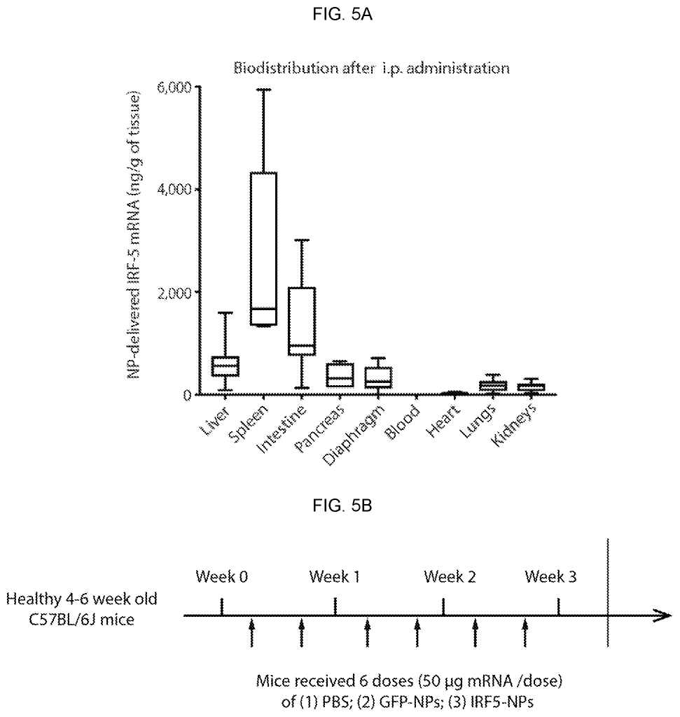

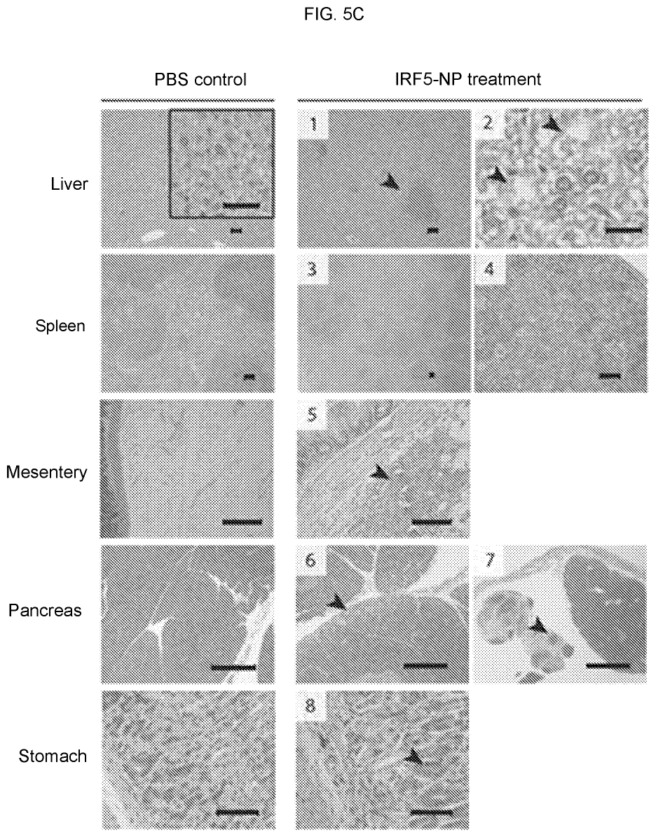

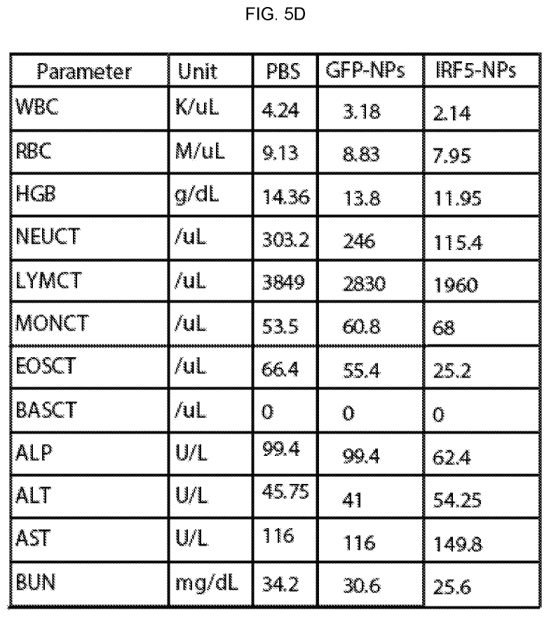

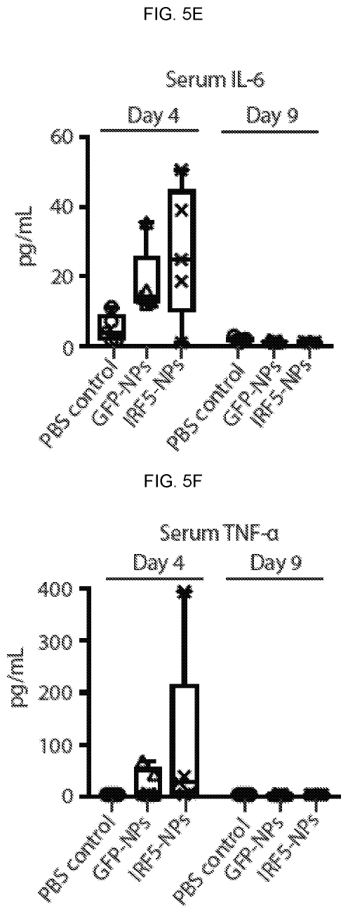

Biodistribution and safety. The distribution of nanoparticles in various organs 24 h after intraperitoneal injection using RT-qPCR assays designed to detect only nanoparticle-delivered (codon optimized) IRF5 was next quantified. The highest concentrations of IVT mRNA were found in organs located in the peritoneum, including liver, spleen, intestine, pancreas, and diaphragm (FIG. 5A). Small amounts of particle-delivered mRNA in organs that lie outside of the peritoneum (heart, lungs, kidneys) were detected, suggesting that a fraction of i.p. injected nanocarriers entered the systemic circulation. Guided by the distribution data, we next assessed whether these nanoreagents are biocompatible and safe for repeated dosing. Mice were injected with a total of 8 doses of IRF5/IKKβ. NPs (two 50 μg mRNA doses/week for 4 weeks, FIG. 5B). They were euthanized 24 h after the final dose, body weight was recorded, blood was collected by retroorbital bleed for serum chemistry, and a complete gross necropsy was performed. There was no difference in body weights between groups. The following tissues were evaluated by a board-certified staff pathologist: liver, spleen, mesentery, pancreas, stomach, kidney, heart, and lungs. Histopathological evaluation revealed in all cases multifocal dense clusters of lymphocytes within or surrounding tumor lesions, but no evidence of inflammation or frank necrosis was observed in tissues where neoplastic cells were not present (FIG. 5C). Also, serum chemistry of IRF5/IKKβ NP-treated mice was comparable to that of PBS controls, indicating that systemic toxicities did not occur (FIG. 5D). Because small amounts of IRF5-mRNA were detected systemically in biodistribution studies, parallel experiments were designed to quantitate inflammatory cytokines in the peripheral blood. Following a single i.p. injection of IRF5/IKKβ NPs, moderate and transient increase was measured in serum levels of interleukin-6 (IL-6) to an average of 26.8 μg/mL (FIG. 5E), and tumor necrosis factor-α (TNF-α) to an average 94.7 μg/mL (FIG. 5F). Based on previous reports, these levels are 500-fold lower than those associated with pathological findings and thus can be considered safe Tarrant J. M. (2010) Toxicol Sci 117: 4-16; Copeland S et al. (2005) Clin Diagn Lab Immunol 12: 60-67).

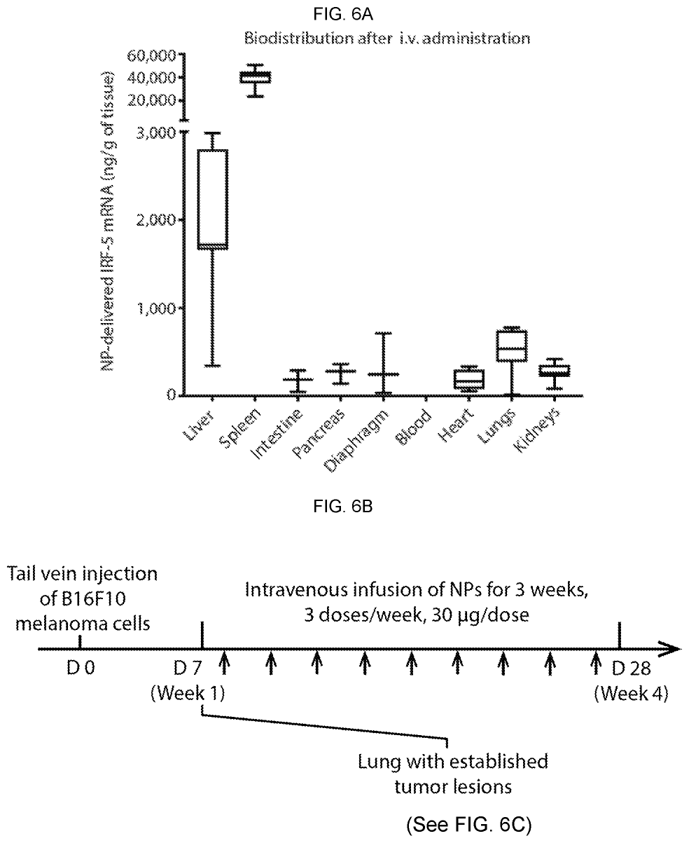

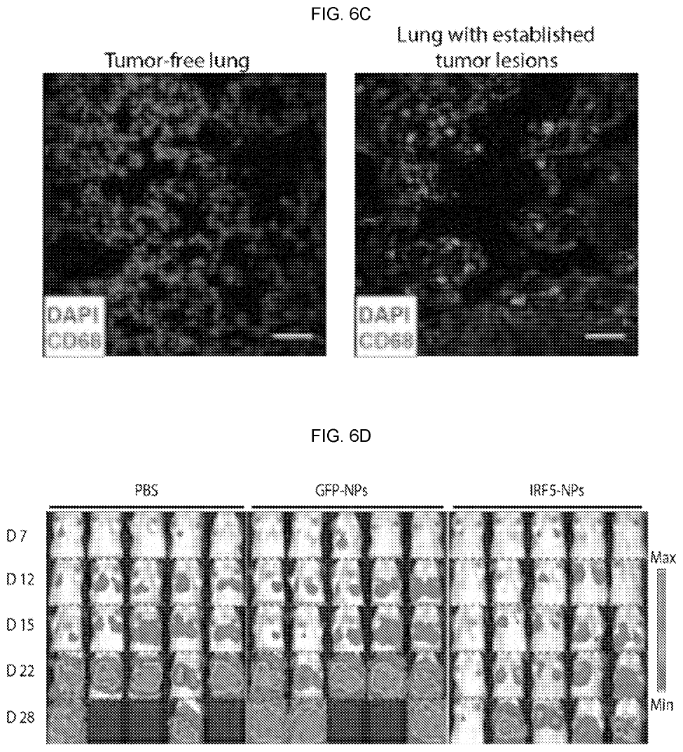

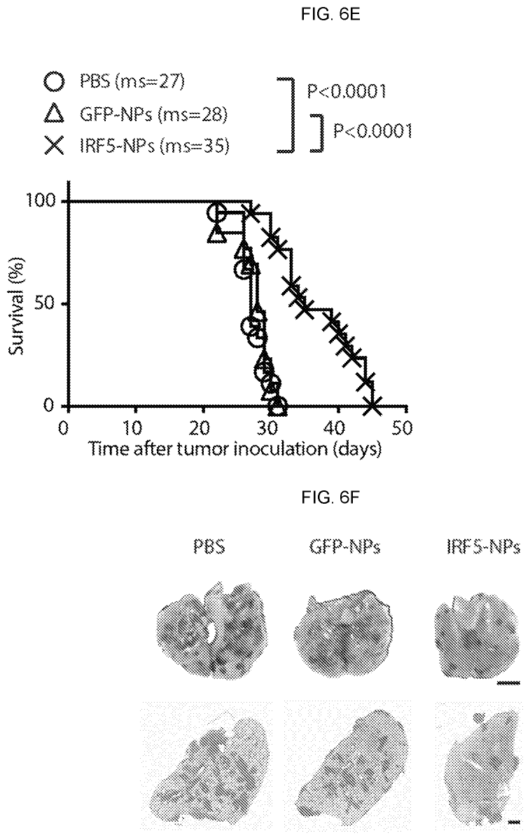

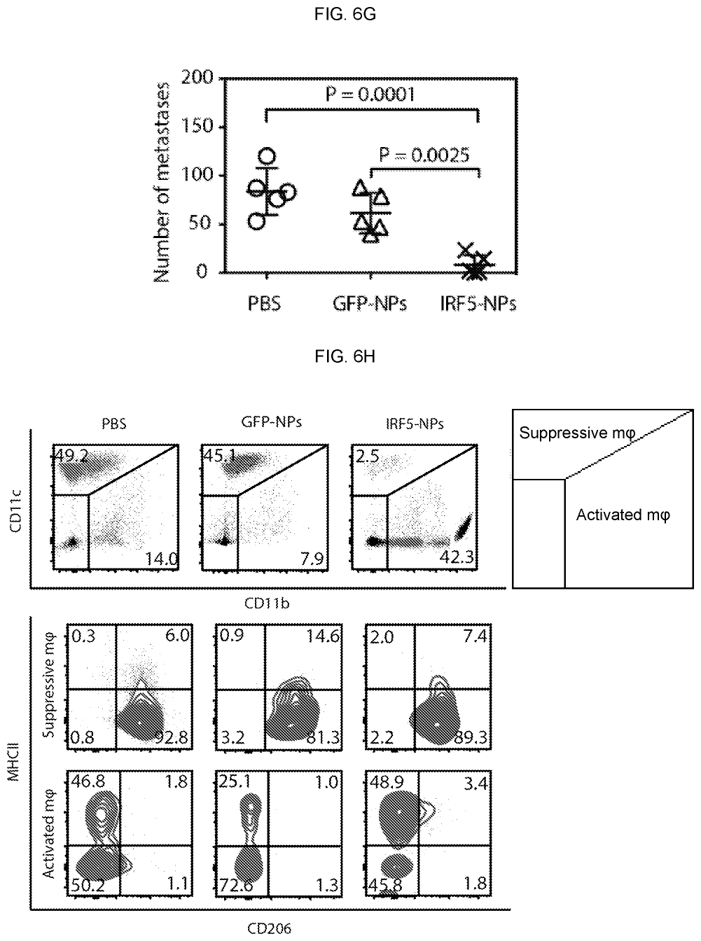

Controlling systemic tumor metastases with intravenous infusions of IRF5/IKKβ nanoparticles. Based on the therapeutic responses achieved with IRF5/IKKβ NPs administered directly into the peritoneal cavity to treat tumor lesions spread throughout the peritoneum, the next question asked was whether intravenously infused mRNA nanocarriers could program macrophages systemically to control disseminated disease. RT-qPCR biodistribution studies revealed that i.v.-infused nanocarriers preferentially deliver their mRNA cargo to organs with high levels of resident macrophages/phagocytes, mostly the spleen, liver, and lungs (FIG. 6A). To measure anti-tumor responses in a clinically relevant in vivo test system, nanoparticles containing IRF5/IKKβ mRNA were administered into mice with disseminated pulmonary melanoma metastases (FIG. 6B). Recent work describes the foundational role of monocytes and macrophages in establishing metastases caused by this disease (Butler K L et al. (2017) Sci Rep 7: 45593; Nielsen S R et al. (2017) Mediators Inflamm 2017: 9624760), and it was confirmed by confocal microscopy that tumor engraftment was coordinate with phagocyte accumulation in the lungs (FIG. 6C). Tumor burdens were determined by bioluminescent imaging, and mice with detectable cancers were sorted into groups that had matching levels. Groups were then randomly assigned to treatment conditions, receiving no therapy (PBS), or intravenous injections of GFP- or IRF5/IKKβ-encapsulating nanoparticles. Only IRF/IKKβ nanoparticle therapy substantially reduced tumor burdens in the lungs; in fact, they improved overall survival by a mean 1.3-fold (FIG. 6D-6E). In parallel experiments, mice were sacrificed 22 days after tumor inoculation to validate bioluminescence tumor signals with counts of pulmonary metastases and to assess macrophage polarization by flow cytometry. The total number of metastases in the lungs of IRF5/IKK NP-treated animals was 8.7-fold reduced (average 40±16 metastases) compared to PBS controls (average 419±139 metastases; FIG. 6F-6G). Flow cytometry of bronchoalveolar lavage fluid cells revealed a strong shift from immune-suppressive (CD206+, MHCII−, CD11c+, CD11blow) macrophages toward activated (CD206−, MHCII+, CD11c−, CD11b+) phagocytes (FIG. 6H-6I).

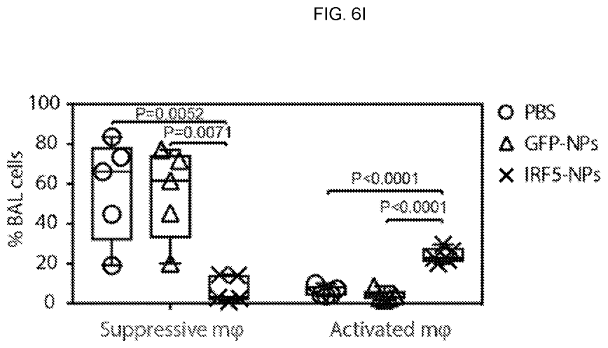

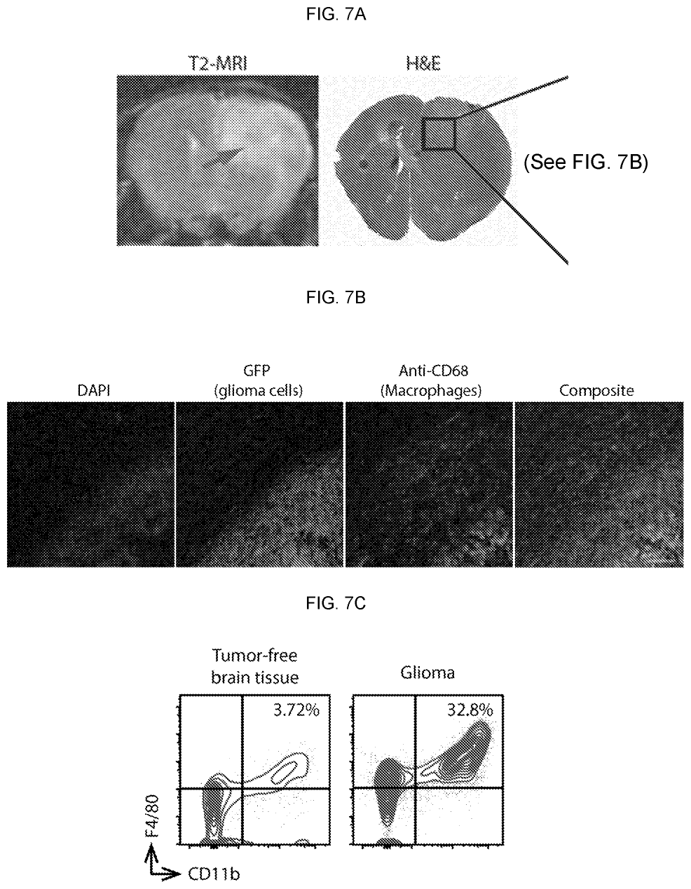

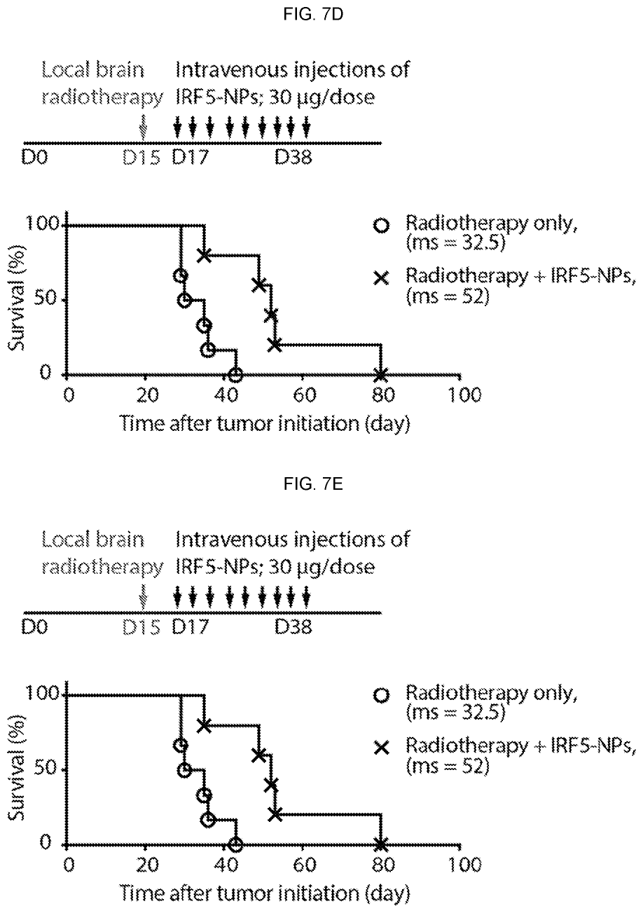

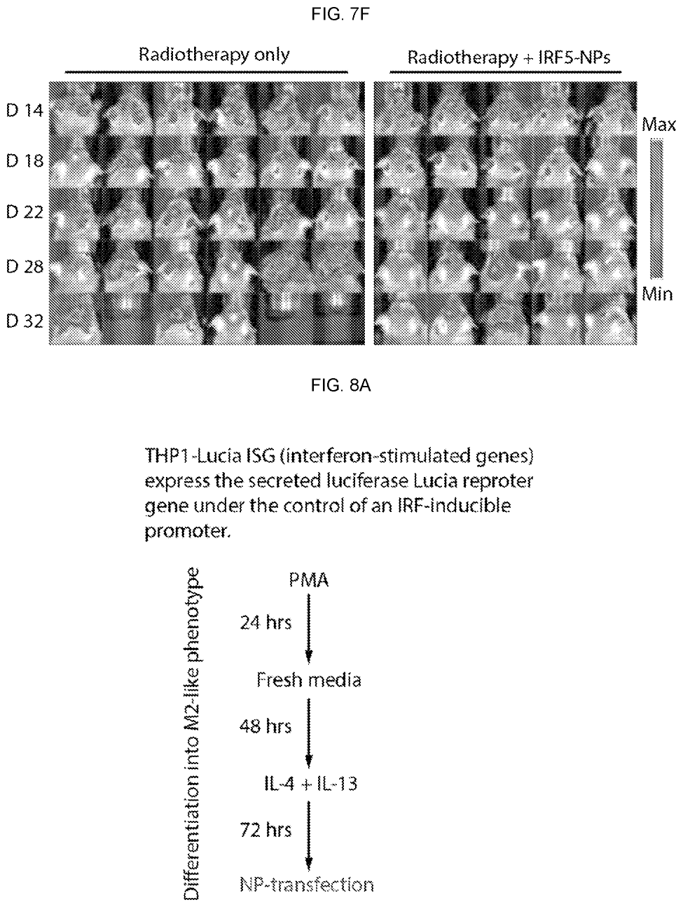

Programming tumor-suppressing phagocytes to treat glioma. For a third in vivo test system glioma was examined, which is a difficult to manage cancer type where M2-like macrophages represent the majority of non-neoplastic cells and promote tumor growth and invasion (Hambardzumyan D et al. (2016) Nat Neurosci 19: 20-27). Currently, the standard of care for this disease is radiotherapy, which unfortunately offers only a temporary stabilization or reduction of symptoms and extends median survival by 3 months (Mann J et al. (2017) Front Neurol 8: 748). To recapitulate the genetic events and subsequent molecular evolution of the disease, the RCAS-PDGF-B/Nestin-Tv-a; Ink4a/Arf−/−; Pten−/− transgenic mouse model of PDGFβ-driven glioma (PDG mice (Hambardzumyan D et al. (2009) Transl Oncol 2: 89-95; Quail D F et al. (2016) Science 352: aad3018)) was used. Brain tissue was stereotactically injected with a mixture of DF-1 cells transfected with either RCAS-PDGFβ or RCAS-cre retrovirus (1:1 mixture, 2 μL). Overexpression of the PDGFβ oncogene and the absence of the tumor suppressor genes Ink4a-arf and Pten in glioma progenitors led to the formation of 4-5 mm diameter tumors (FIG. 7A) with a nearly complete penetrance within 21 days (as established previously (Hambardzumyan D et al. (2009) Transl Oncol 2: 89-95)). Using immunofluorescence, the presence of tumor-infiltrating (CD68+) macrophages (FIG. 7B, indicated in third panel from the left) were confirmed in established gliomas (shown in second panel from the left). Flow cytometry revealed that the F4/80+, CD11b+ macrophage population accounted for 32.8% of total cells in the tumor, which is 9-fold higher than seen in age-matched healthy control mice (3.7%) (FIG. 7C). The PDG mice in the experiments express the firefly luciferase gene linked to a key cancer gene promoter. Bioluminescence from this reporter has been demonstrated to be positively correlated with tumor grade (Uhrbom L et al. (2004) Nat Med 10: 1257-1260), so it was used to monitor tumor development every four days after the onset of treatment. IRF/IKKβ. NPs as a monotherapy was first tested: PDG mice received intravenous infusions of 9 doses of NPs loaded with IRF5/IKKβ mRNA, or PBS in the control group (3 doses/week for 3 weeks). IRF/IKKβ. NP treatments only modestly suppressed tumor progression (producing on average only a 5-day survival advantage compared to untreated controls; FIG. 7D). However, combining radiotherapy as the standard-of-care with IRF5/IKKβ. NP injections substantially reduced tumor growth and more than doubled the survival of treated mice compared to the PBS control group (52 d versus 25 days, respectively; FIG. 7E-7F).

In conclusion, in vivo results from three preclinical solid tumor models demonstrate that nanoparticles, administered either locally or systemically, can deliver genes encoding master regulators of macrophage polarization to re-program immunosuppressive macrophages into tumor-clearing phenotypes.

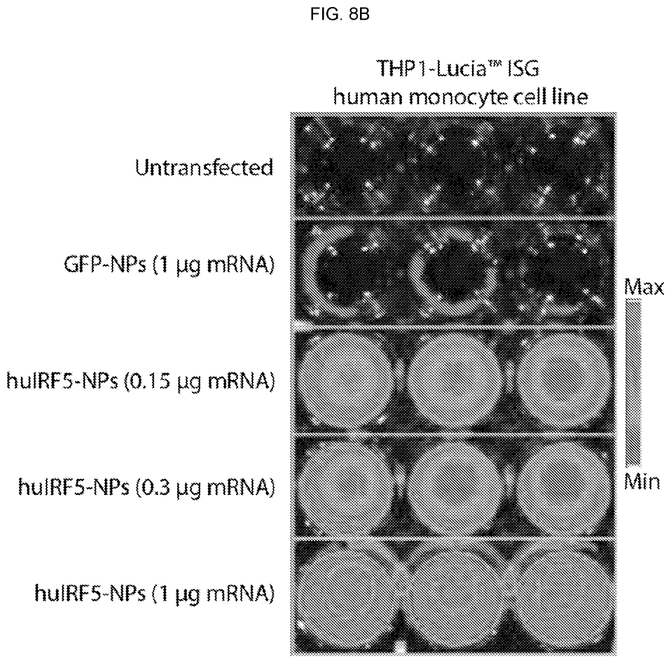

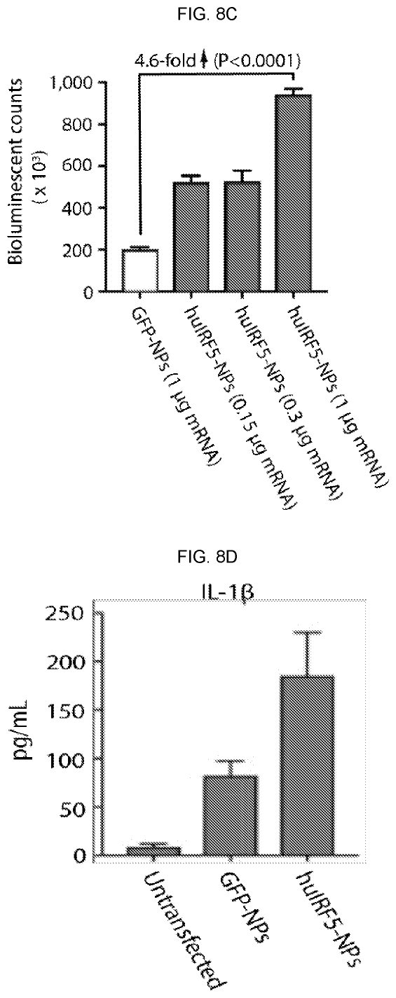

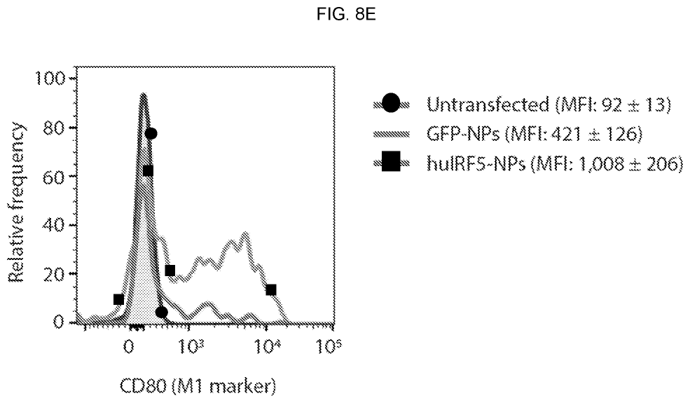

Translation from murine to human macrophages. To confirm that the data acquired in mice has relevance to treat human disease, NPs delivering IVT mRNA encoding human IRF5 and IKKβ. (huIRF5 NPs) were fabricated. The human monocytic cell line THP-1 was used as a well-established M1 and M2 macrophage polarization model to test these nanocarriers (Li C et al. (2016) Sci Rep 6: 21044; Surdziel E et al. (2017) Plos One 12: e0183679). M2-type macrophages were generated by treating THP-1 cells with PMA and polarizing them with IL-4 and IL-13 (FIG. 8A). To confirm that huIRF5 NPs are functional and activate the IRF pathway, THP1-Lucia™ ISG cells were transfected with nanoparticles loaded with huIRF5/IKKβ or GFP control mRNAs. THP1-Lucia™ ISG cells secrete the fluorescent Lucia reporter under the control of an IRF-inducible promoter. This composite promoter includes five IFN-stimulated response elements (ISRE) fused to an ISG54 minimal promoter, which is unresponsive to activators of the NF-κB or AP-1 pathways. As a result, THP1-Lucia™ ISG cells allow the monitoring of the IRF pathway by determining the activity of the Lucia luciferase. It was found that huIRF5 NPs strongly upregulated luciferase expression in M2-polarized THP-1 cells, indicating that the mRNA constructs are functional in human cells (FIG. 8B-8C). To determine whether IRF5 pathway activation can reprogram M2-polarized THP-1 cells toward an M1-like phenotype, secretion of the pro-inflammatory cytokine IL-1β following NP transfection was measured. Production of IL-1β was significantly increased in THP-1 cells transfected with huIRF5 NPs versus untransfected controls (mean 21-fold; P<0.0001, FIG. 8D), which correlated with a robust upregulation (10.9-fold increased MFI, P<0.0001) of the M1 macrophage cell surface marker CD80 (FIG. 8E).

Example 3. Nanoparticles delivering IRF5/IKKb and EpCAM-CD3 bi-specific antibody mRNA will be administered in a preclinical mouse model of disseminated stage 4 ovarian cancer and a 4T1 breast cancer lung metastasis model. Underlying mechanisms (changes in the composition of the tumor microenvironment) and bi-specific antibody serum levels (versus concentrations of bi-specific antibody directly at the tumor site) will be assessed. A side-by-side comparison of in situ-generated versus intravenously administered bi-specific antibody proteins will also be performed.

(13) Closing Paragraphs. The nucleotide sequences described herein are shown using standard letter abbreviations for nucleotide bases, as defined in 37 C.F.R. § 1.822. Only one strand of each nucleotide sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gin), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleotide, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleotide, or gene sequences disclosed herein.

“% sequence identity” refers to a relationship between two or more sequences, as determined by comparing the sequences. In the art, “identity” also means the degree of sequence relatedness between protein, nucleotide, or gene sequences as determined by the match between strings of such sequences. “Identity” (often referred to as “similarity”) can be readily calculated by known methods, including those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleotide molecules that hybridizes under stringent hybridization conditions to a sequence disclosed herein and provide the same function as the reference sequence. Exemplary stringent hybridization conditions include an overnight incubation at 42° C. in a solution including 50% formamide, 5XSSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5XSSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding domain (of, for example, a CAR binding domain or a nanoparticle selected cell targeting ligand) to its cognate binding molecule with an affinity or Ka (i.e., an equilibrium association constant of a particular binding interaction with units of 1/M) equal to or greater than 10⁵ M⁻¹, while not significantly associating with any other molecules or components in a relevant environment sample. “Specifically binds” is also referred to as “binds” herein. Binding domains may be classified as “high affinity” or “low affinity”. In particular embodiments, “high affinity” binding domains refer to those binding domains with a Ka of at least 107 M−1, at least 108 M−1, at least 109 M−1, at least 1010 M−1, at least 1011 M−1, at least 1012 M−1, or at least 1013 M−1. In particular embodiments, “low affinity” binding domains refer to those binding domains with a Ka of up to 107 M−1, up to 106 M−1, up to 105 M−1. Alternatively, affinity may be defined as an equilibrium dissociation constant (Kd) of a particular binding interaction with units of M (e.g., 10-5 M to 10-13 M). In certain embodiments, a binding domain may have “enhanced affinity,” which refers to a selected or engineered binding domains with stronger binding to a cognate binding molecule than a wild type (or parent) binding domain. For example, enhanced affinity may be due to a Ka (equilibrium association constant) for the cognate binding molecule that is higher than the reference binding domain or due to a Kd (dissociation constant) for the cognate binding molecule that is less than that of the reference binding domain, or due to an off-rate (Koff) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to obtain a claimed effect according to a relevant experimental method described in the current disclosure.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A nanoparticle comprising:
a targeting ligand that binds to a professional phagocyte; and
a nucleic acid that encodes a protein molecule having at least a first binding domain and a second binding domain,
wherein the first binding domain is specific to a cell surface protein expressed by an immune cell, and
wherein the second binding domain is specific to a cell surface protein expressed by a cancer cell.

2. The nanoparticle of claim 1, wherein the targeting ligand binds to a cell surface protein expressed by a monocyte, a macrophage, or both.

3. The nanoparticle of claim 1, wherein the targeting ligand comprises di-mannose.

4. The nanoparticle of claim 1, wherein the nucleic acid comprises ribonucleic acid (RNA).

5. The nanoparticle of claim 4, wherein the RNA comprises messenger RNA (mRNA).

6. The nanoparticle of claim 5, wherein the mRNA comprises synthetic RNA or in vitro transcribed RNA (IVT RNA).

7. The nanoparticle of claim 1, wherein the first binding domain is specific to a cell surface protein of a lymphocyte.

8. The nanoparticle of claim 7, wherein the lymphocyte is selected from the group consisting of a T-cell, a B-cell, a natural killer (NK) cell, and a tumor-infiltrating lymphocyte (TIL) cell.

9. The nanoparticle of claim 1, wherein the first binding domain is specific to a cell surface protein of a T-cell selected from the group consisting of a CD8+ T cell, CD4+ T cell, a gamma delta T cell, and an NK T-cell.

10. The nanoparticle of claim 9, wherein the first binding domain is specific to CD3.

11. The nanoparticle of claim 1, wherein the protein molecule is a bi-specific T-cell engager.

12. The nanoparticle of claim 11, wherein the protein molecule is an EpCAM-CD3 bi-specific T-cell engager.

13. The nanoparticle of claim 1, wherein the second binding domain is specific to an antigen expressed by the cancer cell.

14. The nanoparticle of claim 1, further comprising a second nucleic acid that encodes one or more interferon regulatory factors (IRFs).

15. The nanoparticle of claim 1, further comprising a tumor cell proliferation inhibitor or a nucleic acid encoding a tumor cell proliferation inhibitor.

16. The nanoparticle of claim 15, wherein the nucleic acid encodes an antibody, or an antigen-binding fragment of an antibody.

17. The nanoparticle of claim 15, wherein the nanoparticle comprises a nucleic acid encoding a CD40-CD40L inhibitor or a TGFβ inhibitor.

18. The nanoparticle of claim 1, wherein the nanoparticle is a liposome, a liposomal nanoparticle, a lipid nanoparticle, or a solid lipid nanoparticle.

19. A composition comprising:
a first plurality of nanoparticles, wherein each of the first plurality of nanoparticles comprises:
a targeting ligand that binds to a professional phagocyte; and
a nucleic acid encoding a protein molecule having a first binding domain specific to a cell surface protein expressed by an immune cell, and a second binding domain is specific to a cell surface protein expressed by a cancer cell.

20. The composition of claim 19, wherein the targeting ligand binds to a cell surface protein expressed by a monocyte, a macrophage, or both.

21. The composition of claim 19, wherein the targeting ligand comprises di-mannose.

22. The composition of claim 19, wherein the nucleic acid comprises RNA.

23. The composition of claim 22, wherein the RNA comprises mRNA.

24. The composition of claim 23, wherein the mRNA comprises synthetic RNA or IVT RNA.

25. The composition of claim 19, wherein the first binding domain is specific to a cell surface protein of a lymphocyte.

26. The composition of claim 25, wherein the lymphocyte is selected from the group consisting of a T-cell, a B-cell, an NK cell, and a TIL cell.

27. The composition of claim 19, wherein the first binding domain is specific to a cell surface protein of a T-cell selected from the group consisting of a CD8+ T cell, CD4+ T cell, a gamma deltaT cell, and an NK T-cell.

28. The composition of claim 27, wherein the first binding domain is specific to CD3.

29. The composition of claim 19, wherein the protein molecule is a bi-specific T-cell engager.

30. The composition of claim 29, wherein the protein molecule is an EpCAM-CD3 bi-specific T-cell engager.

31. The composition of claim 19, wherein the second binding domain is specific to an antigen expressed by the cancer cell.

32. The composition of claim 19, further comprising a pharmaceutically acceptable carrier.

33. The composition of any of claim Nos. 19-32, wherein at least a subset of the first plurality of nanoparticles further comprises one or more of (a) a nucleic acid encoding one or more interferon regulatory factors (IRFs), and (b) a nucleic acid encoding IKKβ.

34. The composition of any of claim Nos. 19-32, further comprising:
a second plurality of nanoparticles, wherein at least a subset of the second plurality of nanoparticles comprise one or more of (a) a nucleic acid encoding one or more interferon regulatory factors (IRFs), and (b) a nucleic acid encoding IKKβ.

35. The composition of any of claim Nos. 19-34, further comprising a tumor cell proliferation inhibitor.

36. The composition of any of claim Nos. 19-35, wherein at least a subset of the first or second plurality of nanoparticles further comprise a nucleic acid encoding a tumor cell proliferation inhibitor.

37. The composition of any of claim 34, wherein at least a subset of the first or second plurality of nanoparticles further comprise a nucleic acid encoding an antigen-binding fragment of an antibody of a tumor cell proliferation inhibitor.

38. The composition of any of claim Nos. 19 or 34-36, further comprising a third plurality of nanoparticles, wherein at least a subset of the third plurality of nanoparticles comprise a nucleic acid encoding an antigen-binding fragment of an antibody of a tumor cell proliferation inhibitor.

39. The composition of any of claim Nos. 35-38, wherein the tumor cell proliferation inhibitor is a CD40-CD40L inhibitor or a TGFβ inhibitor.

40. The composition of claim 38, comprising the first plurality of nanoparticles and the third plurality of nanoparticles in the absence of the second plurality of nanoparticles.

41. The composition of claim 38, wherein the first, second, and/or third plurality of nanoparticles comprise a liposome, a liposomal nanoparticle, a lipid nanoparticle, or a solid lipid nanoparticle.

42. A composition for treating cancer in a human subject, the composition comprising:
a first plurality of nanoparticles, wherein each of the plurality of nanoparticles comprises
(i) a targeting ligand that binds to a monocyte, macrophage, or both; and
(ii) an mRNA encoding a protein molecule having at least a first binding domain specific to a cell surface protein expressed by a lymphocyte, and a second binding domain specific to a cell surface protein expressed by a cancer cell;
wherein the first plurality of nanoparticles stimulates or enhances an immune response in the human subject, thereby treating cancer.

43. The composition of claim 42, wherein the targeting ligand comprises di-mannose.

44. The composition of claim 42, wherein the mRNA comprises synthetic RNA or IVT RNA.

45. The composition of claim 42, wherein the first binding domain is specific to a cell surface protein of a lymphocyte.

46. The composition of claim 42, wherein the lymphocyte is selected from the group consisting of a T-cell, a B-cell, an NK cell and a TIL cell.

47. The composition of claim 42, wherein the first binding domain is specific to a cell surface protein of a T-cell selected from the group consisting of a CD8+ T cell, CD4+ T cell, a gamma delta T cell, and an NK T-cell.

48. The composition of claim 47, wherein the first binding domain is specific to CD3.

49. The composition of claim 42, wherein the protein molecule is a bi-specific T-cell engager.

50. The composition of claim 49, wherein the protein molecule is an EpCAM-CD3 bi-specific T-cell engager.

51. The composition of claim 42, wherein the second binding domain is specific to an antigen expressed by the cancer cell.

52. The composition of claim 42, further comprising a pharmaceutically acceptable carrier.

53. The composition of any of claim Nos. 42-52, wherein the at least a subset of the first plurality of nanoparticles further comprise one or more of (a) an mRNA encoding one or more interferon regulatory factors (IRFs), (b) an mRNA encoding IKKβ, or (c) an mRNA encoding one or more IRFs and an mRNA encoding IKKβ, and (c) an mRNA encoding a tumor cell proliferation inhibitor.

54. The composition of any of claim Nos. 42-53, further comprising:
a second plurality of nanoparticles, wherein each of the second plurality of nanoparticles comprises
a targeting ligand that binds to a monocyte, a macrophage, or both, and
one or more of (a) an mRNA encoding one or more interferon regulatory factors (IRFs), (b) an mRNA encoding IKKβ, and (c) an mRNA encoding a tumor cell proliferation inhibitor.

55. The composition of claim 54, wherein the second plurality of nanoparticles comprise an mRNA encoding an antigen-binding fragment of an antibody of a tumor cell proliferation inhibitor.

56. The composition of any of claim Nos. 53-55, wherein the tumor cell proliferation inhibitor is a CD40-CD40L inhibitor or a TGFβ inhibitor.

57. The composition of claim 54, wherein the first and/or second plurality of nanoparticles comprise a liposome, a liposomal nanoparticle, a lipid nanoparticle, or a solid lipid nanoparticle.

58. A method for treating cancer in a human subject, the method comprising:
administering to the human subject a composition comprising a first plurality of nanoparticles, wherein each of the first plurality of nanoparticles comprises:
(i) a targeting ligand that binds to a monocyte, a macrophage, or both; and
(ii) an mRNA encoding a protein molecule having at least a first binding domain specific to a cell surface protein expressed by a lymphocyte, and a second binding domain specific to a cell surface protein expressed by a cancer cell;
wherein the plurality of nanoparticles stimulates or enhances an immune response in the human subject, thereby treating cancer.

59. The method of claim 58, wherein the targeting ligand comprises di-mannose.

60. The method of claim 58, wherein the mRNA comprises synthetic RNA or IVT RNA.

61. The method of claim 58, wherein the lymphocyte is selected from the group consisting of a T-cell, a B-cell, an NK cell, and a TIL cell.

62. The method of claim 58, wherein the first binding domain is specific to a cell surface protein of a T-cell selected from the group consisting of a CD8+ T cell, CD4+ T cell, a gamma delta T cell, and an NK T-cell.

63. The method of claim 62, wherein the first binding domain is specific to CD3.

64. The method of claim 58, wherein the protein molecule is a bi-specific T-cell engager.

65. The method of claim 64, wherein the protein molecule is an EpCAM-CD3 bi-specific T-cell engager.

66. The method of claim 58, wherein the second binding domain is specific to an antigen expressed by the cancer cell.

67. The method of claim 58, wherein the composition further comprising a pharmaceutically acceptable carrier.

68. The method of any of claim Nos. 58-67, wherein at least a subset of the first plurality of nanoparticles further comprise one or more of (a) a nucleic acid encoding one or more interferon regulatory factors (IRFs), (b) a nucleic acid encoding IKKβ, and (c) a nucleic acid encoding a tumor cell proliferation inhibitor.

69. The method of any of claim Nos. 58-68, further comprising:
administering to the human subject a composition comprising a second plurality of nanoparticles, wherein each of the second plurality of nanoparticles comprises
a targeting ligand that binds to a monocyte, a macrophage, or both, and
one or more of (a) an mRNA encoding one or more interferon regulatory factors (IRFs), and (b) an mRNA encoding IKKβ.

70. The method of claim 68 or 69, wherein at least a subset of the first or second plurality of nanoparticles further comprise an mRNA encoding a tumor cell proliferation inhibitor.

71. The method of any of claim Nos. 58-70, further comprising:
administering to the human subject a composition comprising a third plurality of nanoparticles, wherein each of the third plurality of nanoparticles comprises
a targeting ligand that binds to a monocyte, a macrophage, or both, and
an mRNA encoding a tumor cell proliferation inhibitor.

72. The method of claim 70 or 71, wherein, an mRNA encoding a tumor cell proliferation inhibitor encodes an antigen-binding fragment of an antibody of a tumor cell proliferation inhibitor.

73. The method of claim 72, wherein the tumor cell proliferation inhibitor is a CD40-CD40L inhibitor or a TGFβ inhibitor.

74. The composition of claim 71, wherein the first, second, and/or third plurality of nanoparticles comprise a liposome, a liposomal nanoparticle, a lipid nanoparticle, or a solid lipid nanoparticle.

75. The method of claim Nos. 58 or 69, wherein the step of administering a composition comprising the first plurality of nanoparticles and the step of administering a composition comprising the second plurality of nanoparticles are performed concurrently or sequentially.

76. The method of any of claim Nos. 58 or 69, wherein the step of administering a composition comprising the first plurality of nanoparticles is performed after the step of administering a composition comprising the second plurality of nanoparticles.

77. The method of claim 71, wherein the step of administering a composition comprising the third plurality of nanoparticles is performed concurrently or sequentially with the step of administering the first plurality of nanoparticles.

78. The method of claim 71, wherein the step of administering a composition comprising the third plurality of nanoparticles is performed concurrently or sequentially with the step of administering the second plurality of nanoparticles.

79. The method of claim 69, comprising the steps of administering a composition comprising the first plurality of nanoparticles and administering a composition comprising the third plurality of nanoparticles in the absence of the step of administering a composition comprising the second plurality of nanoparticles.

80. A modified professional phagocyte comprising:
a nanoparticle loaded with a nucleic acid encoding a protein molecule having at least a first binding domain specific to a cell surface protein expressed by an immune cell and a second binding domain specific for a cell surface protein expressed by cancer cell,
wherein the nanoparticle is adhered to the surface of the phagocyte or has been internalized by the phagocyte.

81. The modified professional phagocyte of claim 80, wherein the phagocyte is a monocyte or a macrophage.

82. The modified professional phagocyte of claim 80, where the phagocyte is a tumor-associated macrophage.

83. The modified professional phagocyte of claim 80, wherein the nucleic acid comprises ribonucleic acid (RNA).

84. The modified professional phagocyte of claim 83, wherein the RNA comprises messenger RNA (mRNA).

85. The modified professional phagocyte of claim 84, wherein the mRNA comprises synthetic RNA or in vitro transcribed RNA (IVT RNA).

86. The modified professional phagocyte of claim 80, wherein the first binding domain is specific to a cell surface protein of a lymphocyte.

87. The modified professional phagocyte of claim 86, wherein the lymphocyte is selected from the group consisting of a T-cell, a B-cell, an NK cell, and a TIL cell.

88. The modified professional phagocyte of claim 80, wherein the first binding domain is specific to a cell surface protein of a T-cell selected from the group consisting of a CD8+ T cell, CD4+ T cell, a gamma delta T cell, and an NK T-cell.

89. The modified professional phagocyte of claim 80, wherein the first binding domain is specific to CD3.

90. The modified professional phagocyte of claim 80, wherein the protein molecule is a bi-specific T-cell engager.

91. The modified professional phagocyte of any of claim Nos. 80-90, wherein the protein molecule is an EpCAM-CD3 bi-specific T-cell engager.

92. The modified professional phagocyte of any of claim Nos. 80-91, wherein the nanoparticle is further loaded with one or more of (a) a nucleic acid encoding one or more interferon regulatory factors (IRFs), (b) a nucleic acid encoding IKKβ, and (c) a nucleic acid encoding a tumor cell proliferation inhibitor.

93. The modified professional phagocyte of any of claim Nos. 80-92, further comprising:
a second nanoparticle loaded with one or more of (a) a nucleic acid encoding one or more interferon regulatory factors (IRFs), (b) a nucleic acid encoding IKKβ, and (c) a nucleic acid encoding a tumor cell proliferation inhibitor,
wherein the second nanoparticle is adhered to the surface of the phagocyte or has been internalized by the phagocyte.

94. The modified professional phagocyte of claim No. 92 or 93, wherein the first or second nanoparticle is loaded with a nucleic acid encoding an antibody or an antigen-binding fragment of an antibody of a tumor cell proliferation inhibitor.

95. The modified professional phagocyte of claim 94, wherein the tumor cell proliferation inhibitor is a CD40-CD40L inhibitor or a TGFβ inhibitor.

96. The modified professional phagocyte of claim 80, further comprising at least one of a second nanoparticle loaded with one or more of (a) a nucleic acid encoding one or more interferon regulatory factors (IRFs), (b) a nucleic acid encoding IKKβ, or (c) a nucleic acid encoding a tumor cell proliferation inhibitor; and
a third nanoparticle loaded with a nucleic acid encoding a tumor cell proliferation inhibitor, wherein each of the second and third nanoparticles is adhered to the surface of the phagocyte or has been internalized by the phagocyte.

97. The modified professional phagocyte of claim 96, wherein the first, second, and/or third nanoparticle comprises a liposome, a liposomal nanoparticle, a lipid nanoparticle, or a solid lipid nanoparticle.

98. A nanoparticle comprising a positively-charged polymer core and a neutral or negatively-charged coating around the polymer core wherein the positively-charged polymer core encapsulates nucleotides encoding at least one binding domain that binds an immune cell activating epitope and/or at least one binding domain that binds a cancer antigen.

99. The nanoparticle of claim 98, wherein the nanoparticles are <130 nm.

100. The nanoparticle of claim 98, wherein the positively charged polymer comprises poly(β-amino ester, poly(L-lysine), poly(ethylene imine) (PEI), poly-(amidoamine) dendrimers (PAMAMs), poly(amine-co-esters), poly(dimethylaminoethyl methacrylate) (PDMAEMA), chitosan, poly-(L-lactide-co-L-lysine), poly[α-(4-aminobutyl)-L-glycolic acid] (PAGA), or poly(4-hydroxy-L-proline ester) (PHP).

101. The nanoparticle of claim 100, wherein the positively charged polymer comprises poly(β-amino ester).

102. The nanoparticle of claim 98, wherein the neutral or negatively-charged coating comprises polyglutamic acid (PGA), poly(acrylic acid), alginic acid, or cholesteryl hemisuccinate/1,2-dioleoyl-sn-glycero-3-phosphoethanolamine.

103. The nanoparticle of claim 102, wherein the neutral or negatively-charged coating comprises polyglutamic acid (PGA).

104. The nanoparticle of claim 98, wherein the neutral or negatively-charged coating comprises a zwitterionic polymer.

105. The nanoparticle of claim 98, wherein the neutral or negatively-charged coating comprises a liposome.

106. The nanoparticle of claim 105, wherein the liposome comprises 1,2-dioleoyl-3-trimethylammonium-propane (DOTAP), 1,2-di-O-octadecenyl-3-trimethylammonium propane (DOTMA), 3ß[N-(N′,N′-dimethylaminoethane)-carbamoyl]cholesterol (DC-Chol), dioctadecyl-amidoglycylspermine (DOGS), cholesterol, 1,2-dioleoyl-sn-glycero-3-phosphoethanolamine (DOPE), or 1,2-dioleoyl-sn-glycero-3-phosphocholine (DOPC).

107. The nanoparticle of claim 98, wherein the nucleotides comprise ribonucleic acid (RNA).

108. The nanoparticle of claim 107, wherein the RNA comprises synthetic RNA.

109. The nanoparticle of claim 107, wherein the RNA comprises in vitro transcribed mRNA.

110. The nanoparticle of claim 98, wherein the nucleotides comprise integrating or non-integrating double-stranded DNA.

111. The nanoparticle of claim 98, wherein the nucleotides are in the form of a plasmid, a minicircle plasmid, or a closed-ended linear ceDNA.

112. The nanoparticle of claim 98, wherein the cancer antigen is expressed by an ovarian cancer cell, a melanoma cell, a glioblastoma cell, a multiple myeloma cell, a melanoma cell, a prostate cancer cell, a breast cancer cell, a stem cell cancer cell, a mesothelioma cell, a renal cell carcinoma cell, a pancreatic cancer cell, a lung cancer cell, a cholangiocarcinoma cell, a bladder cancer cell, a neuroblastoma cell, a colorectal cancer cell, or a merkel cell carcinoma cell.

113. The nanoparticle of claim 98, wherein the cancer antigen comprises B-cell maturation antigen (BCMA), carboxy-anhydrase-IX (CAIX), CD19, CD24, CD56, CD133, CEA, disialoganglioside, EpCam, EGFR, EGFR variant III (EGFRvIII), ERBB2, folate receptor (FOLR), GD2, glypican-2, HER2, Lewis Y, L1-CAM, mesothelin, MUC16, PD-L1, PSMA, Prostate Stem Cell antigen (PSCA), ROR1, TYRP1/gp75, SV40 T, or WT-1.

114. The nanoparticle of claim 98, wherein the binding domain that binds the cancer antigen comprises the complementarity determining regions (CDRs) of antibody adecatumumab, anetumab, ravtansine, amatuximab, HN1, oregovomab, ovarex, abagovomab, edrecolomab, farletuzumab. flanvotumab, TA99, 20D7, Cetuximab, FMC63, SJ25C1, HD37, R11, R12, 2A2, Y31, 4D5, 3G10 atezolizumab, avelumab, or durvalumab.

115. The nanoparticle of claim 98, wherein the binding domains that binds a cancer antigen is a protein molecule.

116. The nanoparticle of claim 115, wherein the different protein molecules within the nanoparticle comprise binding domains that bind different cancer antigens.

117. The nanoparticle of claim 116, wherein the different cancer antigens are expressed by the same cancer type.

118. The nanoparticle of claim 117, wherein the cancer type is ovarian cancer, melanoma, or glioblastoma.

119. The nanoparticle of claim 116, wherein the different cancer antigens comprise
at least two cancer antigens selected from EpCam, L1-CAM, MUC16, folate receptor (FOLR), Lewis Y, ROR1, mesothelin, WT-1, PD-L1, EGFR, and CD56;
at least two cancer antigens selected from Tyrosinase related protein 1 (TYRP1/gp75); GD2, PD-L1, and EGFR; or two cancer antigens selected from EGFR variant III (EGFRvIII) and IL13Ra2.

120. The nanoparticle of claim 98, wherein the at least one binding domain of the protein molecule binds an immune cell activating epitope expressed by a T cell or a natural killer (NK) cell.

121. The nanoparticle of claim 120, wherein the immune cell activating epitope is expressed by a T cell.

122. The nanoparticle of claim 121, wherein the immune cell activating epitope expressed by the T cell comprises CD2, CD3, CD7, CD8, CD27, CD28, CD30, CD40, CD83, 4-1BB, OX40, lymphocyte function-associated antigen-1 (LFA-1), LIGHT, NKG2C, or B7-H3.

123. The nanoparticle of claim 122, wherein the immune cell activating epitope expressed by the T cell comprises CD3, CD28, or 4-1BB.

124. The nanoparticle of claim 98, wherein the binding domains that bind an immune cell activating epitope comprise a protein molecule.

125. The nanoparticle of claim 124, wherein the different protein molecules within the nanoparticle comprise binding domains that bind different immune cell activating epitopes.

126. The nanoparticle of claim 125, wherein the different immune cell activating epitopes comprise CD3 and CD28 or CD3 and 4-1BB.

127. The nanoparticle of claim 126, wherein at least one binding domain comprises the CDRs of antibody OKT3, 20G6-F3, 4B4-D7, 4E7-C9, 18F5-H10, TGN1412, 9D7, 9.3, KOLT-2, 15E8, 248.23.2, EX5.3D10, OKT8 or the SK1.

128. The nanoparticle of claim 120, wherein the immune cell activating epitope is expressed by a NK cell.

129. The nanoparticle of claim 128, wherein the immune cell activating epitope expressed by the NK cell comprises NKG2D, CD8, CD16, KIR2DL4, KIR2DS1, KIR2DS2, KIR3DS1, NKG2C, NKG2E, NKG2D, NKp30, NKp44, NKp46, NKp80, or DNAM-1.

130. The nanoparticle of claim 129, wherein at least one binding domain comprises the CDRs of antibody 5C6, 1D11, mAb 33, P44-8, SK1, or 3G8.

131. The nanoparticle of claim 98, wherein the binding domains are linked through a protein linker.

132. The nanoparticle of claim 131, wherein the protein linker comprises a Gly-Ser linker.

133. The nanoparticle of claim 131, wherein the protein linker comprises a proline-rich linker.

134. The nanoparticle of claim 124, wherein the protein molecule comprises a single chain variable fragment (scFv).

135. The nanoparticle of claim 124, wherein the protein molecule comprises
at least one binding domain binds CEA and at least one binding domain binds CD3, CD28, or 4-1BB;
at least one binding domain binds EGFR and at least one binding domain binds CD3, CD28, or 4-1BB;
at least one binding domain binds EpCam and at least one binding domain binds CD3, CD28, or 4-1BB;
at least one binding domain binds HER2 and at least one binding domain binds CD3, CD28, or 4-1BB;
at least one binding domain binds PD-L1 and at least one binding domain binds CD3, CD28, or 4-1BB;
at least one binding domain binds PSMA and at least one binding domain binds CD3, CD28, or 4-1BB; or
at least one binding domain binds [TYRP1/gp75] and at least one binding domain binds CD3, CD28, or 4-1BB.

136. The nanoparticle of claim 135, wherein the protein molecule comprises catumaxomab, MT110, ertumaxomab, MDX-447, MM-141, AMG211, R06958688, R06895882, TF2, BAY2010112, AMG701, solitomab, or blinatumomab.

137. A nanoparticle of claim 98, wherein the positively-charged polymer core further encapsulates nucleotides encoding one or more interferon regulatory factors (IRFs).

138. The nanoparticle of claim 137, wherein the one or more IRFs lack a functional autoinhibitory domain.

139. The nanoparticle of claim 137, wherein the one or more IRFs lack a functional nuclear export signal.

140. The nanoparticle of claim 137, wherein the one or more IRFs are selected from IRF1, IRF3, IRF5, IRF7, IRF8, and/or a fusion of IRF7 and IRF3.

141. The nanoparticle of claim 137, wherein the one or more IRFs are selected from a sequence having >90%, >95%, or greater than 98% identity to a sequence as set forth in SEQ ID NOs: 1-17.

142. The nanoparticle of claim 137, wherein the one or more IRFs comprise IRF5 selected from a sequence as set forth in SEQ ID NOs: 1-7.

143. The nanoparticle of claim 142, wherein the IRF5 comprises a sequence as set forth in SEQ ID NO: 1 or SEQ ID NO: 3 with one or more mutations selected from S156D, S158D and T160D.

144. The nanoparticle of claim 142, wherein the IRF5 comprises a sequence as set forth in SEQ ID NO: 2 with one or more mutations selected from T10D, S158D, 5309D, S317D, S451D, and S462D.

145. The nanoparticle of claim 142, wherein the IRF5 comprises a sequence as set forth in SEQ ID NO: 4 with one or more mutations selected from S425D, S427D, S430D, and S436D.

146. The nanoparticle of claim 137, wherein the one or more IRFs comprise IRF1 comprising a sequence as set forth in SEQ ID NOs: 8 or 12.

147. The nanoparticle of claim 137, wherein the one or more IRFs comprise IRF8 comprising a sequence as set forth in SEQ ID NOs: 11, 16, or 17.

148. The nanoparticle of claim 147, wherein the IRF8 comprises a sequence as set forth in SEQ ID NO: 11 with a K310R mutation.

149. The nanoparticle of claim 137, wherein the one or more IRFs comprise an IRF7/IRF3 fusion protein comprising an N-terminal IRF7 DNA binding domain, a constitutively active domain, and a C-terminal IRF3 nuclear export signal.

150. The nanoparticle of claim 149, wherein the IRF7/IRF3 fusion protein comprises a sequence as set forth in SEQ ID NO: 15.

151. The nanoparticle of claim 137, wherein the one or more IRFs comprise IRF4.

152. The nanoparticle of claim 137, wherein at least a subset of the nanoparticles comprise nucleotides encoding IKKβ.

153. The nanoparticle of claim 152, wherein the IKKβ is selected from a sequence having >90%, >95%, or >98% identity to a sequence as set forth in a sequence selected from SEQ ID NOs: 18-22.

154. The nanoparticle of claim 152, wherein the IKKβ comprises a sequence as set forth in a sequence selected from SEQ ID NOs: 18-22.

155. The nanoparticle of claim 152, wherein the nucleotides comprise a sequence as set forth in a sequence selected from SEQ ID NOs: 23-44.

156. The nanoparticle of claim 152, wherein the nucleotides encoding one or more IRFs and the nucleotides encoding IKKβ are encapsulated within the same nanoparticle.

157. The nanoparticle of claim 137, wherein the nucleotides encoding one or more IRFs and the nucleotides encoding IKKβ are encapsulated within the same nanoparticle core.

158. The nanoparticle of claim 137, wherein the nucleotides encoding one or more IRFs and the nucleotides encoding IKKβ are encapsulated in different nanoparticles.

159. The nanoparticle of claim 137, wherein the nucleotides encoding at least one or more binding domains are encapsulated within the same nanoparticle as the nucleotides encoding one or more IRFs and/or IKKβ.

160. The nanoparticle of claim 137, wherein the nucleotides encoding at least one or more binding domains are encapsulated within different nanoparticles than those encapsulating nucleotides encoding one or more IRFs and/or IKKβ.

161. The nanoparticle of claim 98, further comprising a transforming growth factor beta (TGFβ) inhibitor.

162. The nanoparticle of claim 161, wherein the TGFβ inhibitor comprises nucleotides encoding the TGFβ inhibitor.

163. The nanoparticle of claim 161, wherein the TGFβ inhibitor comprises the CDRs of an antibody that suppresses the activity of TGFβ.

164. The nanoparticle of claim 161, wherein the TGFβ inhibitor comprises an antibody that suppresses the activity of TGFβ.

165. The nanoparticle of claim 163 or 164, wherein the antibody comprises trabedersen, disitertide, metelimumab, fresolimumab, LY2382770, SIX-100, avotermin, and/or IMC-TR1.

166. The nanoparticle of claim 98, wherein the nanoparticles further comprise nucleotides encoding glucocorticoid-induced leucine zipper (GILZ).

167. The nanoparticle of claim 98, wherein the nanoparticles further comprise nucleotides comprising an anticancer gene selected from p53, RB, BRCA1, E1A, bcl-2, MDR-1, p21, p16, bax, bcl-xs, E2F, IGF-I VEGF, angiostatin, oncostatin, endostatin, GM-CSF, IL-12, IL-2, IL-4, IL-7, IFN-γ, TNFα and/or HSV-tk.

168. A system comprising:
nanoparticles
wherein at least a subset of the nanoparticles comprise nucleotides encoding one or more interferon regulatory factors (IRFs) and
wherein at least a subset of the nanoparticles comprise nucleotides encoding a protein molecule having at least two binding domains
wherein one binding domain binds an antigen expressed by a cancer cell at a tumor site and
wherein one binding domain binds an immune cell activating epitope.

169. The system of claim 168, wherein the nanoparticles are <130 nm.

170. The system of claim 168, wherein the nanoparticles comprise a positively-charged core and a neutrally or negatively-charged coating on the outer surface of the core.

171. The system of claim 170, wherein the positively-charged core comprises a positively-charged lipid and/or a positively-charged polymer.

172. The system of claim 171, wherein the positively charged polymer comprises poly(β-amino ester, poly(L-lysine), poly(ethylene imine) (PEI), poly-(amidoamine) dendrimers (PAMAMs), poly(amine-co-esters), poly(dimethylaminoethyl methacrylate) (PDMAEMA), chitosan, poly-(L-lactide-co-L-lysine), poly[α-(4-aminobutyl)-L-glycolic acid] (PAGA), or poly(4-hydroxy-L-proline ester) (PHP).

173. The system of claim 172, wherein the positively charged polymer comprises poly(β-amino ester).

174. The system of claim 170, wherein the neutral or negatively-charged coating comprises polyglutamic acid (PGA), poly(acrylic acid), alginic acid, or cholesteryl hemisuccinate/1,2-dioleoyl-sn-glycero-3-phosphoethanolamine.

175. The system of claim 174, wherein the neutral or negatively-charged coating comprises polyglutamic acid (PGA).

176. The system of claim 170, wherein the neutral or negatively-charged coating comprises a zwitterionic polymer.

177. The system of claim 170, wherein the neutral or negatively-charged coating comprises a liposome.

178. The system of claim 177, wherein the liposome comprises 1,2-dioleoyl-3-trimethylammonium-propane (DOTAP), 1,2-di-O-octadecenyl-3-trimethylammonium propane (DOTMA), 3ß[N-(N′,N′-dimethylaminoethane)-carbamoyl]cholesterol (DC-Chol), dioctadecyl-amidoglycylspermine (DOGS), cholesterol, 1,2-dioleoyl-sn-glycero-3-phosphoethanolamine (DOPE), or 1,2-dioleoyl-sn-glycero-3-phosphocholine (DOPC).

179. The system of claim 168, wherein the nucleotides comprise ribonucleic acid (RNA).

180. The system of claim 179, wherein the RNA comprises synthetic RNA.

181. The system of claim 179, wherein the RNA comprises in vitro transcribed mRNA.

182. The system of claim 168, wherein the nucleotides comprise integrating or non-integrating double-stranded DNA.

183. The system of claim 168, wherein the nucleotides are in the form of a plasmid, a minicircle plasmid, or a closed-ended linear ceDNA.

184. The system of claim 168, wherein the nucleotides are encapsulated within the positively-charged core.

185. The system of claim 168, wherein the one or more IRFs lack a functional autoinhibitory domain.

186. The system of claim 168, wherein the one or more IRFs lack a functional nuclear export signal.

187. The system of claim 168, wherein the one or more IRFs are selected from IRF1, IRF3, IRF5, IRF7, IRF8, and/or a fusion of IRF7 and IRF3.

188. The system of claim 168, wherein the one or more IRFs are selected from a sequence having >90%, >95%, or greater than 98% identity to a sequence as set forth in SEQ ID NOs: 1-17.

189. The system of claim 168, wherein the one or more IRFs comprise IRF5 selected from a sequence as set forth in SEQ ID NOs: 1-7.

190. The system of claim 189, wherein the IRF5 comprises a sequence as set forth in SEQ ID NO: 1 or SEQ ID NO: 3 with one or more mutations selected from S156D, S158D and T160D.

191. The system of claim 189, wherein the IRF5 comprises a sequence as set forth in SEQ ID NO: 2 with one or more mutations selected from T10D, S158D, 5309D, S317D, S451D, and 5462 D.

192. The system of claim 189, wherein the IRF5 comprises a sequence as set forth in SEQ ID NO: 4 with one or more mutations selected from S425D, S427D, S430D, and S436D.

193. The system of claim 168, wherein the one or more IRFs comprise IRF1 comprising a sequence as set forth in SEQ ID NOs: 8 or 12.

194. The system of claim 168, wherein the one or more IRFs comprise IRF8 comprising a sequence as set forth in SEQ ID NOs: 11, 16, or 17.

195. The system of claim 194, wherein the IRF8 comprises a sequence as set forth in SEQ ID NO: 11 with a K310R mutation.

196. The system of claim 168, wherein the one or more IRFs comprise an IRF7/IRF3 fusion protein comprising an N-terminal IRF7 DNA binding domain, a constitutively active domain, and a C-terminal IRF3 nuclear export signal.

197. The system of claim 196, wherein the IRF7/IRF3 fusion protein comprises a sequence as set forth in SEQ ID NO: 15.

198. The system of claim 168, wherein the one or more IRFs comprise IRF4.

199. The system of claim 168, wherein at least a subset of the nanoparticles comprise nucleotides encoding IKKβ.

200. The system of claim 199, wherein the IKKβ is selected from a sequence having >90%, >95%, or >98% identity to a sequence as set forth in a sequence selected from SEQ ID NOs: 18-22.

201. The system of claim 199, wherein the IKKβ comprises a sequence as set forth in a sequence selected from SEQ ID NOs: 18-22.

202. The system of claim 168, wherein the nucleotides comprise a sequence as set forth in a sequence selected from SEQ ID NOs: 23-44.

203. The system of claim 168, wherein the nucleotides encoding one or more IRFs and the nucleotides encoding IKKβ are encapsulated within the same nanoparticle.

204. The system of claim 199, wherein the nucleotides encoding one or more IRFs and the nucleotides encoding IKKβ are encapsulated within the same nanoparticle core.

205. The system of claim 168, wherein the nucleotides encoding one or more IRFs and the nucleotides encoding IKKβ are encapsulated in different nanoparticles.

206. The system of claim 168, wherein at least one binding domain of the protein molecule binds a cancer antigen expressed by an ovarian cancer cell, a melanoma cell, a glioblastoma cell, a multiple myeloma cell, a melanoma cell, a prostate cancer cell, a breast cancer cell, a stem cell cancer cell, a mesothelioma cell, a renal cell carcinoma cell, a pancreatic cancer cell, a lung cancer cell, a cholangiocarcinoma cell, a bladder cancer cell, a neuroblastoma cell, a colorectal cancer cell, or a merkel cell carcinoma cell.

207. The system of claim 206, wherein the cancer antigen comprises B-cell maturation antigen (BCMA), carboxy-anhydrase-IX (CAIX), CD19, CD24, CD56, CD133, CEA, disialoganglioside, EpCam, EGFR, EGFR variant III (EGFRvIII), ERBB2, folate receptor (FOLR), GD2, glypican-2, HER2, Lewis Y, L1-CAM, mesothelin, MUC16, PD-L1, PSMA, Prostate Stem Cell antigen (PSCA), ROR1, TYRP1/gp75, SV40 T, or WT-1.

208. The system of claim 168, wherein at least one binding domain of the protein molecule comprises the complementarity determining regions (CDRs) of antibody adecatumumab, anetumab, ravtansine, amatuximab, HN1, oregovomab, ovarex, abagovomab, edrecolomab, farletuzumab. flanvotumab, TA99, 20D7, Cetuximab, FMC63, SJ25C1, HD37, R11, R12, 2A2, Y31, 4D5, 3G10 atezolizumab, avelumab, or durvalumab.

209. The system of claim 168, wherein different protein molecules within the system comprise binding domains that bind different cancer antigens.

210. The system of claim 209, wherein the different cancer antigens are expressed by the same cancer type.

211. The system of claim 210, wherein the cancer type is ovarian cancer, melanoma, or glioblastoma.

212. The system of claim 209, wherein the different cancer antigens comprise
at least two cancer antigens selected from EpCam, L1-CAM, MUC16, folate receptor (FOLR), Lewis Y, ROR1, mesothelin, WT-1, PD-L1, EGFR, and CD56;
at least two cancer antigens selected from Tyrosinase related protein 1 (TYRP1/gp75); GD2, PD-L1, and EGFR; or
two cancer antigens selected from EGFR variant III (EGFRvIII) and IL13Ra2.

213. The system of claim 168, wherein at least one binding domain of the protein molecule binds an immune cell activating epitope expressed by a T cell or a natural killer cell.

214. The system of claim 213, wherein the immune cell activating epitope is expressed by a T cell.

215. The system of claim 214, wherein the immune cell activating epitope expressed by the T cell comprises CD2, CD3, CD7, CD8, CD27, CD28, CD30, CD40, CD83, 4-1BB, OX40, lymphocyte function-associated antigen-1 (LFA-1), LIGHT, NKG2C, or B7-H3.

216. The system of claim 215, wherein the immune cell activating epitope expressed by the T cell comprises CD3, CD28, or 4-1BB.

217. The system of claim 168, wherein different protein molecules within the system comprise binding domains that bind different immune cell activating epitopes.

218. The system of claim 217, wherein the different immune cell activating epitopes comprise CD3 and CD28 or CD3 and 4-1BB.

219. The system of claim 218, wherein at least one binding domain comprises the CDRs of antibody OKT3, 20G6-F3, 4B4-D7, 4E7-C9, 18F5-H10, TGN1412, 9D7, 9.3, KOLT-2, 15E8, 248.23.2, EX5.3D10, OKT8 or the SK1.

220. The system of claim 213, wherein the immune cell activating epitope is expressed by a NK cell.

221. The system of claim 220, wherein the immune cell activating epitope expressed by the NK cell comprises NKG2D, CD8, CD16, KIR2DL4, KIR2DS1, KIR2DS2, KIR3DS1, NKG2C, NKG2E, NKG2D, NKp30, NKp44, NKp46, NKp80, or DNAM-1.

222. The system of claim 221, wherein at least one binding domain comprises the CDRs of antibody 5C6, 1D11, mAb 33, P44-8, SK1, or 3G8.

223. The system of claim 168, wherein the binding domains of the protein molecule are linked through a protein linker.

224. The system of claim 223, wherein the protein linker comprises a Gly-Ser linker.

225. The system of claim 223, wherein the protein linker comprises a proline-rich linker.

226. The system of claim 168, wherein the protein molecule comprises a single chain variable fragment (scFv).

227. The system of claim 168, wherein the protein molecule comprises
at least one binding domain binds CEA and at least one binding domain binds CD3, CD28, or 4-1BB;
at least one binding domain binds EGFR and at least one binding domain binds CD3, CD28, or 4-1BB;
at least one binding domain binds EpCam and at least one binding domain binds CD3, CD28, or 4-1BB;
at least one binding domain binds HER2 and at least one binding domain binds CD3, CD28, or 4-1BB;
at least one binding domain binds PD-L1 and at least one binding domain binds CD3, CD28, or 4-1BB;
at least one binding domain binds PSMA and at least one binding domain binds CD3, CD28, or 4-1BB; or
at least one binding domain binds [TYRP1/gp75] and at least one binding domain binds CD3, CD28, or 4-1BB.

228. The system of claim 227, wherein the protein molecule comprises catumaxomab, MT110, ertumaxomab, MDX-447, MM-141, AMG211, R06958688, R06895882, TF2, BAY2010112, AMG701, solitomab, or blinatumomab.

229. The system of claim 168, wherein the nucleotides encoding at least two binding domains are encapsulated within the same nanoparticle as the nucleotides encoding one or more IRFs and/or IKKβ.

230. The system of claim 168, wherein the nucleotides encoding at least two binding domains are encapsulated within the same nanoparticle core as the nucleotides encoding one or more IRFs and/or IKKβ.

231. The system of claim 168, wherein the nucleotides encoding at least two binding domains are encapsulated within different nanoparticles than those encapsulating nucleotides encoding one or more IRFs and/or IKKβ.

232. The system of claim 168, further comprising a transforming growth factor beta (TGFβ) inhibitor.

233. The system of claim 232, wherein the TGFβ inhibitor comprises nucleotides encoding the TGFβ inhibitor.

234. The system of claim 232, wherein the TGFβ inhibitor comprises the CDRs of an antibody that suppresses the activity of TGFβ.

235. The system of claim 232, wherein the TGFβ inhibitor comprises an antibody that suppresses the activity of TGFβ.

236. The system of claim 234 or 235, wherein the antibody comprises trabedersen, disitertide, metelimumab, fresolimumab, LY2382770, SIX-100, avotermin, and/or IMC-TR1.

237. The system of claim 168, wherein the nanoparticles further comprise nucleotides encoding glucocorticoid-induced leucine zipper (GILZ).

238. The system of claim 168, wherein the nanoparticles further comprise nucleotides comprising an anticancer gene selected from p53, RB, BRCA1, E1A, bcl-2, MDR-1, p21, p16, bax, bcl-xs, E2F, IGF-I VEGF, angiostatin, oncostatin, endostatin, GM-CSF, IL-12, IL-2, IL-4, IL-7, IFN-γ, TNFα and/or HSV-tk.

239. The system of claim 168, further comprising a pharmaceutically acceptable carrier.

240. A monocyte or macrophage genetically modified to express the nucleotides of a system of claim 168.

241. A method of modulating the macrophage activation state at a tumor site within a subject, recruiting immune cells to the tumor site, and activating the recruited immune cells comprising:
Administering the system of claim 168 to the subject, thereby modulating the macrophage activation state at the tumor site within the subject, recruiting immune cells to the tumor site, and activating the recruited immune cells.

242. The method of claim 241, wherein the administering comprises intravenous administering and the nanoparticles are taken up by monocytes within the blood stream.

243. The method of claim 242, wherein the monocytes migrate to the tumor site and differentiate into macrophages.

244. The method of claim 243, wherein the differentiated macrophages are resistant to tumor suppression.

245. The method of claim 241, wherein the administering comprises locally administering at the tumor site and the nanoparticles are taken up by tumor-associated macrophages (TAM).

246. The method of claim 245, wherein the local administering comprises intraperitoneally administering or intracranially administering.

247. The method of claim 245, wherein the TAM undergo a phenotype transformation from a suppressed to an activated state.

248. The method of claim 245, wherein the tumor site comprises an ovarian cancer tumor site, a glioblastoma tumor site, or a melanoma cancer tumor site.

249. The method of claim 241, wherein the recruited and activated immune cells are T cells or NK cells.

250. The method of claim 241, comprising administering nanoparticles comprising nucleotides encoding one or more IRFs before administering nanoparticles comprising nucleotides encoding at least two binding domains.

251. The method of claim 241, comprising administering nanoparticles comprising nucleic acids encoding one or more IRFs at least 24 hours before administering nanoparticles comprising nucleotides encoding at least two binding domains.


SEQUENCE LISTING (265 sequences)

SEQ ID NO: 1 - protein, 498 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTEDVKWPPTLQPPTLRPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 2 - protein, 514 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTDAVQSGPHMTPYSLLKEDVKWPPTLQPPTLRPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 3 - protein, 504 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTDAVQSGPHMTPYSLLKEDVKWPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 4 - protein, 488 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTEDVKWPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 5 - protein, 412 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTVTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 6 - protein, 147 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYETPSPLRITLLVQERRRKKRKSCRGCCQA

SEQ ID NO: 7 - protein, 497 aa, Mus musculus
MNHSAPGIPPPPRRVRLKPWLVAQVNSCQYPGLQWVNGEKKLFYIPWRHATRHGPSQDGDNTIFKAWAKETGKYTEGVDEADPAKWKANLRCALNKSRDFQLFYDGPRDMPPQPYKIYEVCSNGPAPTESQPTDDYVLGEEEEEEEEELQRMLPGLSITEPALPGPPNAPYSLPKEDTKWPPALQPPVGLGPPVPDPNLLAPPSGNPAGFRQLLPEVLEPGPLASSQPPTEPLLPDLLISPHMLPLTDLEIKFQYRGRAPRTLTISNPQGCRLFYSQLEATQEQVELFGPVTLEQVRFPSPEDIPSDKQRFYTNQLLDVLDRGLILQLQGQDLYAIRLCQCKVFWSGPCALAHGSCPNPIQREVKTKLFSLEQFLNELILFQKGQTNTPPPFEIFFCFGEEWPDVKPREKKLITVQVVPVAARLLLEMFSGELSWSADSIRLQISNPDLKDHMVEQFKELHHLWQSQQQLQPMVQAPPVAGLDASQGPWPMHPVGMQ

SEQ ID NO: 8 - protein, 325 aa, Homo sapiens
MPITRMRMRPWLEMQINSNQIPGLIWINKEEMIFQIPWKHAAKHGWDINKDACLFRSWAIHTGRYKAGEKEPDPKTWKANFRCAMNSLPDIEEVKDQSRNKGSSAVRVYRMLPPLTKNQRKERKSKSSRDAKSKAKRKSCGDSSPDTFSDGLSSSTLPDDHSSYTVPGYMQDLEVEQALTPALSPCAVSSTLPDWHIPVEVVPDSTSDLYNFQVSPMPSTSEATTDEDEEGKLPEDIMKLLEQSEWQPTNVDGKGYLLNEPGVQPTSVYGDFSCKEEPEIDSPGGDIGLSLQRVFTDLKNMDATWLDSLLTPVRLPSIQAIPCAP

SEQ ID NO: 9 - protein, 427 aa, Homo sapiens
MGTPKPRILPWLVSQLDLGQLEGVAWVNKSRTRFRIPWKHGLRQDAQQEDFGIFQAWAEATGAYVPGRDKPDLPTWKRNFRSALNRKEGLRLAEDRSKDPHDPHKIYEFVNSGVGDFSQPDTSPDTNGGGSTSDTQEDILDELLGNMVLAPLPDPGPPSLAVAPEPCPQPLRSPSLDNPTPFPNLGPSENPLKRLLVPGEEWEFEVTAFYRGRQVFQQTISCPEGLRLVGSEVGDRTLPGWPVTLPDPGMSLTDRGVMSYVRHVLSCLGGGLALWRAGQWLWAQRLGHCHTYWAVSEELLPNSGHGPDGEVPKDKEGGVFDLGPFIVDLITFTEGSGRSPRYALWFCVGESWPQDQPWTKRLVMVKVVPTCLRALVEMARVGGASSLENTVDLHISNSHPLSLTSDQYKAYLQDLVEGMDFQGPGES

SEQ ID NO: 10 - protein, 503 aa, Homo sapiens
MALAPERAAPRVLFGEWLLGEISSGCYEGLQWLDEARTCFRVPWKHFARKDLSEADARIFKAWAVARGRWPPSSRGGGPPPEAETAERAGWKTNFRCALRSTRRFVMLRDNSGDPADPHKVYALSRELCWREGPGTDQTEAEAPAAVPPPQGGPPGPFLAHTHAGLQAPGPLPAPAGDKGDLLLQAVQQSCLADHLLTASWGADPVPTKAPGEGQEGLPLTGACAGGPGLPAGELYGWAVETTPSPGPQPAALTTGEAAAPESPHQAEPYLSPSPSACTAVQEPSPGALDVTIMYKGRTVLQKVVGHPSCTFLYGPPDPAVRATDPQQVAFPSPAELPDQKQLRYTEELLRHVAPGLHLELRGPQLWARRMGKCKVYWEVGGPPGSASPSTPACLLPRNCDTPIFDFRVFFQELVEFRARQRRGSPRYTIYLGFGQDLSAGRPKEKSLVLVKLEPWLCRVHLEGTQREGVSSLDSSSLSLCLSSANSLYDDIECFLMELEQPA

SEQ ID NO: 11 - protein, 426 aa, Homo sapiens
MCDRNGGRRLRQWLIEQIDSSMYPGLIWENEEKSMFRIPWKHAGKQDYNQEVDASIFKAWAVFKGKFKEGDKAEPATWKTRLRCALNKSPDFEEVTDRSQLDISEPYKVYRIVPEEEQKCKLGVATAGCVNEVTEMECGRSEIDELIKEPSVDDYMGMIKRSPSPPEACRSQLLPDWWAQQPSTGVPLVTGYTTYDAHHSAFSQMVISFYYGGKLVGQATTTCPEGCRLSLSQPGLPGTKLYGPEGLELVRFPPADAIPSERQRQVTRKLFGHLERGVLLHSSRQGVFVKRLCQGRVFCSGNAVVCKGRPNKLERDEVVQVFDTSQFFRELQQFYNSQGRLPDGRVVLCFGEEFPDMAPLRSKLILVQIEQLYVRQLAEEAGKSCGAGSVMQAPEEPPPDQVFRMFPDICASHQRSFFRENQQITV

SEQ ID NO: 12 - protein, 329 aa, Mus musculus
MPITRMRMRPWLEMQINSNQIPGLIWINKEEMIFQIPWKHAAKHGWDINKDACLFRSWAIHTGRYKAGEKEPDPKTWKANFRCAMNSLPDIEEVKDQSRNKGSSAVRVYRMLPPLTRNQRKERKSKSSRDTKSKTKRKLCGDVSPDTFSDGLSSSTLPDDHSSYTTQGYLGQDLDMERDITPALSPCVVSSSLSEWHMQMDIIPDSTTDLYNLQVSPMPSTSEAATDEDEEGKIAEDLMKLFEQSEWQPTHIDGKGYLLNEPGTQLSSVYGDFSCKEEPEIDSPRGDIGIGIQHVFTEMKNMDSIMWMDSLLGNSVRLPPSIQAIPCAP

SEQ ID NO: 13 - protein, 419 aa, Mus musculus
METPKPRILPWLVSQLDLGQLEGVAWLDESRTRFRIPWKHGLRQDAQMADFGIFQAWAEASGAYTPGKDKPDVSTWKRNFRSALNRKEVLRLAADNSKDPYDPHKVYEFVTPGARDFVHLGASPDTNGKSSLPHSQENLPKLFDGLILGPLKDEGSSDLAIVSDPSQQLPSPNVNNFLNPAPQENPLKQLLAEEQWEFEVTAFYRGRQVFQQTLFCPGGLRLVGSTADMTLPWQPVTLPDPEGFLTDKLVKEYVGQVLKGLGNGLALWQAGQCLWAQRLGHSHAFWALGEELLPDSGRGPDGEVHKDKDGAVFDLRPFVADLIAFMEGSGHSPRYTLWFCMGEMWPQDQPWVKRLVMVKVVPTCLKELLEMAREGGASSLKTVDLHISNSQPISLTSDQYKAYLQDLVEDMDFQATGNI

SEQ ID NO: 14 - protein, 457 aa, Mus musculus
MAEVRGVQRVLFGDWLLGEVSSGQYEGLQWLNEARTVFRVPWKHFGRRDLDEEDAQIFKAWAVARGRWPPSGVNLPPPEAEAAERRERRGWKTNFRCALHSTGRFILRQDNSGDPVDPHKVYELSRELGSTVGPATENREEVSLSNALPTQGVSPGSFLARENAGLQTPSPLLSSDAGDLLLQVLQYSHILESESGADPVPPQAPGQEQDRVYEEPYAAWQVEAVPSPRPQQPALTERSLGFLDVTIMYKGRTVLQAVVGHPRCVFLYSPMAPAVRTSEPQPVIFPSPAELPDQKQLHYTETLLQHVSPGLQLELRGPSLWALRMGKCKVYWEVGSPMGTTGPSTPPQLLERNRHTPIFDFSTFFRELEEFRARRRQGSPHYTIYLGFGQDLSAGRPKEKTLILVKLEPWVCKAYLEGVQREGVSSLDSSSLGLCLSSTNSLYEDIEHFLMDLGQWP

SEQ ID NO: 15 - protein, 525 aa, Mus musculus
MAEVRGVQRVLFGDWLLGEVSSGQYEGLQWLNEARTVFRVPWKHFGRRDLDEEDAQIFKAWAVARGRWPPSGVNLPPPEAEAAERRERRGWKTNFRCALHSTGRFILRQDNSGDPVDPHKVYELSRELGSTVGPATENREEVSLSNALPTQGVSPGSFLARENAGLQTPSPLLSSDAGDLLLQVLQYSHILESESGADPVPPQAPGQEQDRVYEEPYAAWQVEAVPSPRPQQPALTERSLGFLDVTKLFDGLILGPLKDEGSSDLAIVSDPSQQLPSPNVNNFLNPAPQENPLKQLLAEEQWEFEVTAFYRGRQVFQQTLFCPGGLRLVGSTADMTLPWQPVTLPDPEGFLTDKLVKEYVGQVLKGLGNGLALWQAGQCLWAQRLGHSHAFWALGEELLPDSGRGPDGEVHKDKDGAVFDLRPFVADLIAFMEGSGHSPRYTLWFCMGEMWPQDQPWVKRLVMVKVVPTCLKELLEMAREGGASSLKTVDLHIDNDQPIDLDDDQYKAYLQDLVEDMDFQATGNI

SEQ ID NO: 16 - protein, 424 aa, Mus musculus
MCDRNGGRRLRQWLIEQIDSSMYPGLIWENDEKTMFRIPWKHAGKQDYNQEVDASIFKAWAVFKGKFKEGDKAEPATWKTRLRCALNKSPDFEEVTDRSQLDISEPYKVYRIVPEEEQKCKLGVAPAGCMSEVPEMECGRSEIEELIKEPSVDEYMGMTKRSPSPPEACRSQILPDWWVQQPSAGLPLVTGYAAYDTHHSAFSQMVISFYYGGKLVGQATTTCLEGCRLSLSQPGLPKLYGPDGLEPVCFPTADTIPSERQRQVTRKLFGHLERGVLLHSNRKGVFVKRLCQGRVFCSGNAVVCKGRPNKLERDEVVQVFDTNQFIRELQQFYATQSRLPDSRVVLCFGEEFPDTVPLRSKLILVQVEQLYARQLVEEAGKSCGAGSLMPALEEPQPDQAFRMFPDICTSHQRPFFRENQQITV

SEQ ID NO: 17 - protein, 424 aa, Mus musculus
MCDRNGGRRLRQWLIEQIDSSMYPGLIWENDEKTMFRIPWKHAGKQDYNQEVDASIFKAWAVFKGKFKEGDKAEPATWKTRLRCALNKSPDFEEVTDRSQLDISEPYKVYRIVPEEEQKCKLGVAPAGCMSEVPEMECGRSEIEELIKEPSVDEYMGMTKRSPSPPEACRSQILPDWWVQQPSAGLPLVTGYAAYDTHHSAFSQMVISFYYGGKLVGQATTTCLEGCRLSLSQPGLPKLYGPDGLEPVCFPTADTIPSERQRQVTRKLFGHLERGVLLHSNRKGVFVKRLCQGRVFCSGNAVVCKGRPNRLERDEVVQVFDTNQFIRELQQFYATQSRLPDSRVVLCFGEEFPDTVPLRSKLILVQVEQLYARQLVEEAGKSCGAGSLMPALEEPQPDQAFRMFPDICTSHQRPFFRENQQITV

SEQ ID NO: 18 - protein, 756 aa, Homo sapiens
MSWSPSLTTQTCGAWEMKERLGTGGFGNVIRWHNQETGEQIAIKQCRQELSPRNRERWCLEIQIMRRLTHPNVVAARDVPEGMQNLAPNDLPLLAMEYCQGGDLRKYLNQFENCCGLREGAILTLLSDIASALRYLHENRIIHRDLKPENIVLQQGEQRLIHKIIDLGYAKELDQGSLCTSFVGTLQYLAPELLEQQKYTVTVDYWSFGTLAFECITGFRPFLPNWQPVQWHSKVRQKSEVDIVVSEDLNGTVKFSSSLPYPNNLNSVLAERLEKWLQLMLMWHPRQRGTDPTYGPNGCFKALDDILNLKLVHILNMVTGTIHTYPVTEDESLQSLKARIQQDTGIPEEDQELLQEAGLALIPDKPATQCISDGKLNEGHTLDMDLVFLFDNSKITYETQISPRPQPESVSCILQEPKRNLAFFQLRKVWGQVWHSIQTLKEDCNRLQQGQRAAMMNLLRNNSCLSKMKNSMASMSQQLKAKLDFFKTSIQIDLEKYSEQTEFGITSDKLLLAWREMEQAVELCGRENEVKLLVERMMALQTDIVDLQRSPMGRKQGGTLDDLEEQARELYRRLREKPRDQRTEGDSQEMVRLLLQAIQSFEKKVRVIYTQLSKTVVCKQKALELLPKVEEVVSLMNEDEKTVVRLQEKRQKELWNLLKIACSKVRGPVSGSPDSMNASRLSQPGQLMSQPSTASNSLPEPAKKSEELVAEAHNLCTLLENAIQDTVREQDQSFTALDWSWLQTEEEEHSCLEQAS

SEQ ID NO: 19 - protein, 754 aa, Homo sapiens
MFSGGCHSPGFGRPSPAFPAPGSPPPAPRPCRQETGEQIAIKQCRQELSPRNRERWCLEIQIMRRLTHPNVVAARDVPEGMQNLAPNDLPLLAMEYCQGGDLRKYLNQFENCCGLREGAILTLLSDIASALRYLHENRIIHRDLKPENIVLQQGEQRLIHKIIDLGYAKELDQGSLCTSFVGTLQYLAPELLEQQKYTVTVDYWSFGTLAFECITGFRPFLPNWQPVQWHSKVRQKSEVDIVVSEDLNGTVKFSSSLPYPNNLNSVLAERLEKWLQLMLMWHPRQRGTDPTYGPNGCFKALDDILNLKLVHILNMVTGTIHTYPVTEDESLQSLKARIQQDTGIPEEDQELLQEAGLALIPDKPATQCISDGKLNEGHTLDMDLVFLFDNSKITYETQISPRPQPESVSCILQEPKRNLAFFQLRKVWGQVWHSIQTLKEDCNRLQQGQRAAMMNLLRNNSCLSKMKNSMASMSQQLKAKLDFFKTSIQIDLEKYSEQTEFGITSDKLLLAWREMEQAVELCGRENEVKLLVERMMALQTDIVDLQRSPMGRKQGGTLDDLEEQARELYRRLREKPRDQRTEGDSQEMVRLLLQAIQSFEKKVRVIYTQLSKTVVCKQKALELLPKVEEVVSLMNEDEKTVVRLQEKRQKELWNLLKIACSKVRGPVSGSPDSMNASRLSQPGQLMSQPSTASNSLPEPAKKSEELVAEAHNLCTLLENAIQDTVREQDQSFTALDWSWLQTEEEEHSCLEQAS

SEQ ID NO: 20 - protein, 256 aa, Homo sapiens
MSWSPSLTTQTCGAWEMKERLGTGGFGNVIRWHNQETGEQIAIKQCRQELSPRNRERWCLEIQIMRRLTHPNVVAARDVPEGMQNLAPNDLPLLAMEYCQGGDLRKYLNQFENCCGLREGAILTLLSDIASALRYLHENRIIHRDLKPENIVLQQGEQRLIHKIIDLGYAKELDQGSLCTSFVGTLQYLAPELLEQQKYTVTVDYWSFGTLAFECITGFRPFLPNWQPVQCVRMWPGTVAHSCNPSTLGGRGRWIS

SEQ ID NO: 21 - protein, 697 aa, Homo sapiens
MSSDGTIRLTHPNVVAARDVPEGMQNLAPNDLPLLAMEYCQGGDLRKYLNQFENCCGLREGAILTLLSDIASALRYLHENRIIHRDLKPENIVLQQGEQRLIHKIIDLGYAKELDQGSLCTSFVGTLQYLAPELLEQQKYTVTVDYWSFGTLAFECITGFRPFLPNWQPVQWHSKVRQKSEVDIVVSEDLNGTVKFSSSLPYPNNLNSVLAERLEKWLQLMLMWHPRQRGTDPTYGPNGCFKALDDILNLKLVHILNMVTGTIHTYPVTEDESLQSLKARIQQDTGIPEEDQELLQEAGLALIPDKPATQCISDGKLNEGHTLDMDLVFLFDNSKITYETQISPRPQPESVSCILQEPKRNLAFFQLRKVWGQVWHSIQTLKEDCNRLQQGQRAAMMNLLRNNSCLSKMKNSMASMSQQLKAKLDFFKTSIQIDLEKYSEQTEFGITSDKLLLAWREMEQAVELCGRENEVKLLVERMMALQTDIVDLQRSPMGRKQGGTLDDLEEQARELYRRLREKPRDQRTEGDSQEMVRLLLQAIQSFEKKVRVIYTQLSKTVVCKQKALELLPKVEEVVSLMNEDEKTVVRLQEKRQKELWNLLKIACSKVRGPVSGSPDSMNASRLSQPGQLMSQPSTASNSLPEPAKKSEELVAEAHNLCTLLENAIQDTVREQDQSFTALDWSWLQTEEEEHSCLEQAS

SEQ ID NO: 22 - protein, 738 aa, Mus musculus
MSWSPSLPTQTCGAWEMKERLGTGGFGNVIRWHNQATGEQIAIKQCRQELSPKNRDRWCLEIQIMRRLNHPNVVAARDVPEGMQNLAPNDLPLLAMEYCQGGDLRRYLNQFENCCGLREGAVLTLLSDIASALRYLHENRIIHRDLKPENIVLQQGEKRLIHKIIDLGYAKELDQGSLCTSFVGTLQYLAPELLEQQKYTVTVDYWSFGTLAFECITGFRPFLPNWQPVQWHSKVRQKSEVDIVVSEDLNGAVKFSSSLPFPNNLNSVLAERLEKWLQLMLMWHPRQRGTDPQYGPNGCFRALDDILNLKLVHVLNMVTGTVHTYPVTEDESLQSLKTRIQEDTGILETDQELLQEAGLVLLPDKPATQCISDSKTNEGLTLDMDLVFLFDNSKINYETQITPRPQPESVSCILQEPKRNLSFFQLRKVWGQVWHSIQTLKEDCNRLQQGQRAAMMSLLRNNSCLSKMKNAMASTAQQLKAKLDFFKTSIQIDLEKYKEQTEFGITSDKLLLAWREMEQAVEQCGRENDVKHLVERMMALQTDIVDLQRSPMGRKQGGTLDDLEEQARELYRRLREKPRDQRTEGDSQEMVRLLLQAIQSFEKKVRVIYTQLSKTVVCKQKALELLPKVEEVVSLMNEDERTVVRLQEKRQKELWNLLKIACSKVRGPVSGSPDSMNVSRLSHPGQLMSQPSSACDSLPESDKKSEELVAEAHALCSRLESALQDTVKEQDRSFTVTA

SEQ ID NO: 23 - DNA, 1497 bp, Homo sapiens
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagaggatgtcaagtggccgcccactctgcagccgcccactctgcggccgcctactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctctgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataa

SEQ ID NO: 24 - DNA, 1545 bp, Homo sapiens
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagatgcagtgcagtctggcccccacatgacaccctattctttactcaaagaggatgtcaagtggccgcccactctgcagccgcccactctgcggccgcctactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctctgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataa

SEQ ID NO: 25 - DNA, 1514 bp, Homo sapiens
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagatgcagtgcagtctggcccccacatgacaccctattctttactcaaagaggatgtcaagtggccgcccactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctctgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataa

SEQ ID NO: 26 - DNA, 1467 bp, Homo sapiens
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagaggatgtcaagtggccgcccactctgcagccgcccactctgcagccgcccgtggtgctgggtccccctgctccagaccccagccccctggctcctccccctggcaaccctgctggcttcagggagcttctctctgaggtcctggagcctgggcccctgcctgccagcctgccccctgcaggcgaacagctcctgccagacctgctgatcagcccccacatgctgcctctgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataa

SEQ ID NO: 27 - DNA, 1239 bp, Homo sapiens
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgaggtctgctccaatggccctgctcccacagactcccagccccctgaggattactcttttggtgcaggagaggaggaggaagaagaggaagagctgcagaggatgttgccaagcctgagcctcacagtgaccgacctggagatcaagtttcagtaccgggggcggccaccccgggccctcaccatcagcaacccccatggctgccggctcttctacagccagctggaggccacccaggagcaggtggaactcttcggccccataagcctggagcaagtgcgcttccccagccctgaggacatccccagtgacaagcagcgcttctacacgaaccagctgctggatgtcctggaccgcgggctcatcctccagctacagggccaggacctttatgccatccgcctgtgtcagtgcaaggtgttctggagcgggccttgtgcctcagcccatgactcatgccccaaccccatccagcgggaggtcaagaccaagcttttcagcctggagcattttctcaatgagctcatcctgttccaaaagggccagaccaacaccccaccacccttcgagatcttcttctgctttggggaagaatggcctgaccgcaaaccccgagagaagaagctcattactgtacaggtggtgcctgtagcagctcgactgctgctggagatgttctcaggggagctatcttggtcagctgatagtatccggctacagatctcaaacccagacctcaaagaccgcatggtggagcaattcaaggagctccatcacatctggcagtcccagcagcggttgcagcctgtggcccaggcccctcctggagcaggccttggtgttggccaggggccctggcctatgcacccagctggcatgcaataa

SEQ ID NO: 28 - DNA, 444 bp, Homo sapiens
atgaaccagtccatcccagtggctcccaccccaccccgccgcgtgcggctgaagccctggctggtggcccaggtgaacagctgccagtacccagggcttcaatgggtcaacggggaaaagaaattattctgcatcccctggaggcatgccacaaggcatggtcccagccaggacggagataacaccatcttcaaggcctgggccaaggagacagggaaatacaccgaaggcgtggatgaagccgatccggccaagtggaaggccaacctgcgctgtgcccttaacaagagccgggacttccgcctcatctacgacgggccccgggacatgccacctcagccctacaagatctacgagactcccagccccctgaggattactcttttggtgcaggagaggaggaggaagaagaggaagagctgcagaggatgttgccaagcctga

SEQ ID NO: 29 - DNA, 1494 bp, Mus musculus
atgaatcatagtgcacccgggatccctcctcctccaagacgagtacgcctcaagccctggttggtagctcaagtcaactcatgccaataccctgggcttcagtgggtgaacggtgagaagaaattgttttatatcccatggcgacacgcaacaagacatggcccatcacaggatggagataacaccatatttaaggcatgggcaaaggaaacaggaaagtacactgagggcgttgatgaggccgatcctgcaaaatggaaagcaaatttgcgatgcgctctcaataaatcacgagatttccaactcttttacgacggcccaagggacatgccaccacaaccttataaaatctacgaggtatgttccaacggtccagccccaactgaatcccagcctactgacgactatgtccttggagaagaggaggaagaagaagaggaggaacttcagcggatgttgcctgggttgtccataactgagcctgccttgccaggaccccctaatgcaccatactcccttcccaaagaagatacaaaatggccccccgcattgcaaccccccgttggtttgggaccacctgtgcccgacccaaatctcttggccccaccaagcggtaacccagccggatttcgacaacttctgcccgaagtccttgagccaggtcccttggcctcttctcagccccctacagaacctctgctccccgatctcttgatatctccccacatgcttcccttgactgatttggagataaaatttcagtatcgcggccgagctcccagaacactgactatatcaaatccccaaggttgccgcctgttttacagtcagttggaggcaactcaggagcaagtagagctctttgggccagttactctggagcaggtgagattccctagtccagaggacataccaagcgataagcaaagattttacacaaatcaacttctggatgtacttgatcgaggtttgatccttcagttgcagggccaagatttgtatgccattcgactctgtcaatgcaaggtattttggagcggcccatgtgcccttgctcatggcagctgccctaatcccatccaaagagaagtaaagactaaacttttcagcctggaacaatttctcaacgaactcattctgtttcaaaaaggtcagaccaacacaccccctcctttcgagattttcttctgcttcggcgaagagtggcctgatgtgaagccccgcgaaaaaaagcttatcaccgttcaagtggtacccgtcgcagccaggctccttcttgaaatgtttagcggtgaactctcatggtccgctgacagtatccggctccaaatatcaaaccctgatcttaaagaccacatggtagaacagtttaaagaactccaccacctgtggcaatcccaacagcagctccagccaatggttcaagctcctccagtcgctgggctggacgcctcacaaggaccctggcccatgcaccccgtcgggatgcagtaa

SEQ ID NO: 30 - DNA, 978 bp, Homo sapiens
atgcccatcactcggatgcgcatgagaccctggctagagatgcagattaattccaaccaaatcccggggctcatctggattaataaagaggagatgatcttccagatcccatggaagcatgctgccaagcatggctgggacatcaacaaggatgcctgtttgttccggagctgggccattcacacaggccgatacaaagcaggggaaaaggagccagatcccaagacgtggaaggccaactttcgctgtgccatgaactccctgccagatatcgaggaggtgaaagaccagagcaggaacaagggcagctcagctgtgcgagtgtaccggatgcttccacctctcaccaagaaccagagaaaagaaagaaagtcgaagtccagccgagatgctaagagcaaggccaagaggaagtcatgtggggattccagccctgataccttctctgatggactcagcagctccactctgcctgatgaccacagcagctacacagttccaggctacatgcaggacttggaggtggagcaggccctgactccagcactgtcgccatgtgctgtcagcagcactctccccgactggcacatcccagtggaagttgtgccggacagcaccagtgatctgtacaacttccaggtgtcacccatgccctccacctctgaagctacaacagatgaggatgaggaagggaaattacctgaggacatcatgaagctcttggagcagtcggagtggcagccaacaaacgtggatgggaaggggtacctactcaatgaacctggagtccagcccacctctgtctatggagactttagctgtaaggaggagccagaaattgacagcccagggggggatattgggctgagtctacagcgtgtcttcacagatctgaagaacatggatgccacctggctggacagcctgctgaccccagtccggttgccctccatccaggccattccctgtgcaccgtag

SEQ ID NO: 31 - DNA, 1284 bp, Homo sapiens
atgggaaccccaaagccacggatcctgccctggctggtgtcgcagctggacctggggcaactggagggcgtggcctgggtgaacaagagccgcacgcgcttccgcatcccttggaagcacggcctacggcaggatgcacagcaggaggatttcggaatcttccaggcctgggccgaggccactggtgcatatgttcccgggagggataagccagacctgccaacctggaagaggaatttccgctctgccctcaaccgcaaagaagggttgcgtttagcagaggaccggagcaaggaccctcacgacccacataaaatctacgagtttgtgaactcaggagttggggacttttcccagccagacacctctccggacaccaatggtggaggcagtacttctgatacccaggaagacattctggatgagttactgggtaacatggtgttggccccactcccagatccgggacccccaagcctggctgtagcccctgagccctgccctcagcccctgcggagccccagcttggacaatcccactcccttcccaaacctggggccctctgagaacccactgaagcggctgttggtgccgggggaagagtgggagttcgaggtgacagccttctaccggggccgccaagtcttccagcagaccatctcctgcccggagggcctgcggctggtggggtccgaagtgggagacaggacgctgcctggatggccagtcacactgccagaccctggcatgtccctgacagacaggggagtgatgagctacgtgaggcatgtgctgagctgcctgggtgggggactggctctctggcgggccgggcagtggctctgggcccagcggctggggcactgccacacatactgggcagtgagcgaggagctgctccccaacagcgggcatgggcctgatggcgaggtccccaaggacaaggaaggaggcgtgtttgacctggggcccttcattgtagatctgattaccttcacggaaggaagcggacgctcaccacgctatgccctctggttctgtgtgggggagtcatggccccaggaccagccgtggaccaagaggctcgtgatggtcaaggttgtgcccacgtgcctcagggccttggtagaaatggcccgggtagggggtgcctcctccctggagaatactgtggacctgcacatttccaacagccacccactctccctcacctccgaccagtacaaggcctacctgcaggacttggtggagggcatggatttccagggccctggggagagctga

SEQ ID NO: 32 - DNA, 1512 bp, Homo sapiens
atggccttggctcctgagagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgggataactcgggggacccggccgacccgcacaaggtgtacgcgctcagccgggagctgtgctggcgagaaggcccaggcacggaccagactgaggcagaggcccccgcagctgtcccaccaccacagggtgggcccccagggccattcctggcacacacacatgctggactccaagccccaggccccctccctgccccagctggtgacaagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcccagggctccctgctggggagctgtacgggtgggcagtagagacgacccccagccccgggccccagcccgcggcactaacgacaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctag

SEQ ID NO: 33 - DNA, 1281 bp, Homo sapiens
atgtgtgaccggaatggtggtcggcggcttcgacagtggctgatcgagcagattgacagtagcatgtatccaggactgatttgggagaatgaggagaagagcatgttccggatcccttggaaacacgctggcaagcaagattataatcaggaagtggatgcctccatttttaaggcctgggcagtttttaaagggaagtttaaagaaggggacaaagctgaaccagccacttggaagacgaggttacgctgtgctttgaataagagcccagattttgaggaagtgacggaccggtcccaactggacatttccgagccatacaaagtttaccgaattgttcctgaggaagagcaaaaatgcaaactaggcgtggcaactgctggctgcgtgaatgaagttacagagatggagtgcggtcgctctgaaatcgacgagctgatcaaggagccttctgtggacgattacatggggatgatcaaaaggagcccttccccgccggaggcctgtcggagtcagctccttccagactggtgggcgcagcagcccagcacaggcgtgccgctggtgacggggtacaccacctacgacgcgcaccattcagcattctcccagatggtgatcagcttctactatgggggcaagctggtgggccaggccaccaccacctgccccgagggctgccgcctgtccctgagccagcctgggctgcccggcaccaagctgtatgggcccgagggcctggagctggtgcgcttcccgccggccgacgccatccccagcgagcgacagaggcaggtgacgcggaagctgttcgggcacctggagcgcggggtgctgctgcacagcagccggcagggcgtgttcgtcaagcggctgtgccagggccgcgtgttctgcagcggcaacgccgtggtgtgcaaaggcaggcccaacaagctggagcgtgatgaggtggtccaggtcttcgacaccagccagttcttccgagagctgcagcagttctataacagccagggccggcttcctgacggcagggtggtgctgtgctttggggaagagtttccggatatggcccccttgcgctccaaactcattctcgtgcagattgagcagctgtatgtccggcaactggcagaagaggctgggaagagctgtggagccggctctgtgatgcaggcccccgaggagccgccgccagaccaggtcttccggatgtttccagatatttgtgcctcacaccagagatcatttttcagagaaaaccaacagatcaccgtctaa

SEQ ID NO: 34 - DNA, 990 bp, Mus musculus
atgccaatcactcgaatgcggatgagaccctggctagagatgcagattaattccaaccaaatcccagggctgatctggatcaataaagaagagatgatcttccagattccatggaagcacgctgctaagcacggctgggacatcaacaaggatgcctgtctgttccggagctgggccattcacacaggccgatacaaagcaggagaaaaagagccagatcccaagacatggaaggcaaacttccgttgtgccatgaactccctgccagacatcgaggaagtgaaggatcagagtaggaacaagggcagctctgctgtgcgggtgtaccggatgctgccacccctcaccaggaaccagaggaaagagagaaagtccaagtccagccgagacactaagagcaaaaccaagaggaagctgtgtggagatgttagcccggacactttctctgatggactcagcagctctaccctacctgatgaccacagcagttacaccactcagggctacctgggtcaggacttggatatggaaagggacataactccagcactgtcaccgtgtgtcgtcagcagcagtctctctgagtggcatatgcagatggacattataccagatagcaccactgatctgtataacctacaggtgtcacccatgccttccacctccgaagccgcaacagacgaggatgaggaagggaagatagccgaagaccttatgaagctctttgaacagtctgagtggcagccgacacacatcgatggcaagggatacttgctcaatgagccagggacccagctctcttctgtctatggagacttcagctgcaaagaggaaccagagattgacagccctcgaggggacattgggataggcatacaacatgtcttcacggagatgaagaatatggactccatcatgtggatggacagcctgctgggcaactctgtgaggctgccgccctctattcaggccattccttgtgcaccatag

SEQ ID NO: 35 - DNA, 1260 bp, Mus musculus
atggaaaccccgaaaccgcggattttgccctggctggtgtcacagctggacctggggcagctggaaggcgtggcctggctggacgagagccgaacgaggttcaggatcccgtggaagcatggcctacggcaggacgcacagatggctgactttggcatcttccaggcctgggcagaagccagtggtgcctacaccccggggaaggataagccggacgtgtcaacctggaagaggaatttccggtcagccctgaaccggaaagaagtgttgcggttagctgctgacaatagcaaggacccttatgaccctcataaagtgtatgagtttgtgactccaggggcgcgggacttcgtacatctgggtgcctctcctgacaccaatggcaaaagcagcctgcctcactcccaggaaaacctaccgaagttatttgatggcctgatcttggggcccctcaaagatgaggggtcctcagatctggctattgtttctgatccttctcaacaactgccaagccccaatgtgaacaacttcctaaaccctgcaccccaagaaaatccactgaagcagctgctagctgaggaacaatgggagttcgaggtgaccgccttctaccgaggccgccaggtcttccagcagacactcttttgcccggggggcctgcggctggtgggcagcacagctgacatgacactgccctggcagccagtcaccctgcccgatcctgaggggtttctgacggacaagcttgtgaaggagtacgtggggcaggtgctcaaagggctgggcaatgggctggcactgtggcaggctgggcagtgcctctgggcccagcgcctaggccactcccacgccttctgggctctgggggaggagctgcttccagacagtgggcgagggcctgatggagaggtccacaaggacaaggacggagccgtgttcgacctcaggcccttcgtggcagatctgattgccttcatggaaggaagtggacactccccacgctacactctgtggttctgcatgggggaaatgtggccccaggaccagccatgggtcaagaggcttgtgatggtcaaggttgttcctacatgtcttaaggagctgttagagatggcccgggaagggggagcctcttcactgaaaaccgtggacttgcacatctccaacagccagcctatctcccttacctctgaccagtacaaggcctacctccaggacttggtggaggacatggacttccaggccactggaaatatctga

SEQ ID NO: 36 - DNA, 1374 bp, Mus musculus
atggctgaagtgaggggggtccagcgagtgctgtttggagactggctattgggggaggtcagcagcggccagtacgaggggctgcagtggctgaacgaggctcgcacagtcttccgcgtaccctggaagcatttcggtcgtagggatctggatgaagaagatgcacagatcttcaaggcctgggctgtggcccgagggaggtggccacctagtggagttaacctgccacccccagaggctgaggctgctgagcgaagagagcgaagaggctggaagaccaacttccgctgtgcactccacagcacagggcgttttatcttgcgccaagacaattcaggggatccagttgatccgcataaggtgtacgaacttagccgggagcttggatctactgtgggcccagccacggaaaatagggaagaagtgagcctcagcaatgctctgcccacacagggtgtgtccccaggatcatttctggcaagagaaaatgctgggctccaaaccccaagccctctgctttctagtgatgccggggacctcttgcttcaggttctgcagtacagccacatactggaatccgagtctggggcagaccccgtcccaccacaggctcctggccaggagcaagaccgtgtttacgaggaaccctatgcagcatggcaggtggaagctgtccccagtcccaggcctcaacagccagctctcaccgagcgcagccttgggttcctggatgtgaccatcatgtacaagggccgcacagtgctacaggcagtggtggggcaccccagatgcgtgttcctgtacagccccatggccccagcagtaagaacttcagagccccagccggtgatctttcccagtcctgctgagctcccagatcagaagcagctgcactacacagagacgcttctccagcatgtgtctcccggccttcagctggagcttcgaggaccgtcactgtgggccctgcgtatgggcaagtgcaaggtgtactgggaggtaggcagccctatgggcactaccggcccctccaccccaccccagctgctggagcgcaaccgccacacccccatcttcgacttcagcactttcttccgagaactggaggagtttcgggctcggaggcggcaagggtcaccacactacaccatctacctgggttttgggcaagacttgtcagcagggaggcccaaggagaagaccctgatcctggtgaagctggagccatgggtatgcaaggcatacctggagggcgtgcagcgtgagggtgtgtcctccctggacagcagcagtctcggcttgtgcttgtctagcaccaacagtctctacgaagacatcgaacacttcctcatggacctgggtcagtggccttga

SEQ ID NO: 37 - DNA, 1578 bp, Mus musculus
atggccgaagttcgaggagtacagcgcgtgctgtttggggactggttgcttggtgaagtctcttctggtcagtatgaaggcctgcaatggcttaatgaggcacgcacagtttttcgagtgccatggaaacacttcggtaggcgcgatctcgacgaagaggatgcccagattttcaaggcatgggcagtcgcacggggcaggtggcccccttcaggcgtaaatttgccccccccagaggctgaagctgctgaacgcagagaacgccggggatggaagactaactttcgatgtgcccttcacagtacaggcaggttcatcttgcggcaggataatagtggcgaccctgtagacccacacaaggtttatgagctgagccgggagcttggctcaacagtcggtcctgcaaccgagaacagagaagaggtgtccttgtctaacgccctcccaactcagggtgtgtctcccggtagcttcctggcacgcgaaaacgctggactccaaaccccctccccactgttgtccagtgatgccggtgatcttctccttcaggtgctccaatactcccatatactggagagcgagtcaggggctgatcccgtgccccctcaagctcctggacaggaacaagatcgcgtctacgaggagccatatgctgcctggcaggtcgaggctgtgccatcacctcggcctcaacagcccgctctcaccgagcgctcacttgggtttttggatgtcactaaacttttcgacggcctgatacttggcccattgaaggacgagggatcatccgatcttgccatagtaagtgacccatcacagcagttgccctcaccaaacgtcaacaacttcctcaatccagctccccaggagaaccccctcaaacagcttctcgcagaagagcaatgggagtttgaggtgactgctttctatagaggtaggcaggtgttccaacaaactctgttttgccccggaggtctgcgccttgtaggtagcaccgcagacatgacacttccctggcaacctgtgacacttcccgatcctgagggatttctcacagataaactcgttaaggaatatgtggggcaagtactcaaaggtctgggcaatgggttggccctttggcaagctggtcaatgtctctgggctcaacgactcgggcactcacatgctttttgggctcttggcgaggagctgctccccgacagcgggcgcggacctgacggggaggttcataaggacaaagacggcgccgtatttgatcttagacccttcgtggcagatctgatcgctttcatggaaggatcaggtcatagccccaggtacacactttggttttgcatgggtgaaatgtggcctcaggaccaaccttgggtcaagcgcttggtcatggttaaggtggttcccacttgcctcaaagagttgttggagatggctagggaaggtggggcttcctcactgaaaaccgtagatctccacattgataatgatcagcctatagatttggacgacgaccaatacaaagcttatctccaggacctggttgaagatatggactttcaggctacaggtaacatctaa

SEQ ID NO: 38 - DNA, 1275 bp, Mus musculus
atgtgtgacaggaacggcggtagaagactgagacagtggctgatcgagcaaattgacagctcaatgtaccctgggttgatatgggaaaacgatgaaaagacaatgttcagaataccctggaagcacgctggaaagcaggattacaaccaggaagtggacgccagtatttttaaggcttgggctgtcttcaaagggaagtttaaagagggcgacaaagcagagccagcaacctggaaaacccgcttgaggtgtgcactcaataagtcacccgacttcgaggaagtcactgaccgcagtcaattggacatatcagaaccatacaaagtctacaggatagtccccgaagaagagcagaaatgcaaactcggtgtagcacctgctggctgtatgagtgaagtgcctgaaatggaatgcggcagatcagaaatcgaagaactcataaaagaaccaagtgtagatgagtatatgggaatgaccaaaagatccccatcccccccagaagcctgtcggagccaaatcttgcctgactggtgggtacagcaaccctccgccggacttccccttgtgacaggctatgccgcttacgatactcatcacagcgcttttagccagatggttatttccttctattatggaggaaaactggtcggccaagccacaaccacctgcctcgaggggtgtcgcttgagtttgagtcaacccggtcttcccaaactctatggccccgatgggcttgaacctgtctgctttcccactgctgatactattccctcagagagacaacgacaagtcacccgaaaattgtttggccacctcgagaggggagtactcttgcactctaacaggaagggtgtctttgtgaaacgcctctgtcaaggtagggtattctgttctggaaatgcagttgtttgcaaaggcaggcctaacaaactggaacgggatgaagtcgtacaagtgttcgataccaatcagtttattcgggagttgcagcagttttacgctacacaaagtcgcctccctgacagtcgggttgtgttgtgcttcggggaggagtttcccgacactgtacccctccgaagcaaactcatactggtacaggtagaacaactttatgccaggcaactggtggaagaggccggtaagtcctgtggcgcaggatccctgatgccagccctggaagagccccagcctgaccaagcatttaggatgtttcccgacatttgtacctcacaccagaggccttttttccgcgaaaaccagcagataaccgtgtaa

SEQ ID NO: 39 - DNA, 1275 bp, Mus musculus
atgtgtgacaggaacggcggtagaagactgagacagtggctgatcgagcaaattgacagctcaatgtaccctgggttgatatgggaaaacgatgaaaagacaatgttcagaataccctggaagcacgctggaaagcaggattacaaccaggaagtggacgccagtatttttaaggcttgggctgtcttcaaagggaagtttaaagagggcgacaaagcagagccagcaacctggaaaacccgcttgaggtgtgcactcaataagtcacccgacttcgaggaagtcactgaccgcagtcaattggacatatcagaaccatacaaagtctacaggatagtccccgaagaagagcagaaatgcaaactcggtgtagcacctgctggctgtatgagtgaagtgcctgaaatggaatgcggcagatcagaaatcgaagaactcataaaagaaccaagtgtagatgagtatatgggaatgaccaaaagatccccatcccccccagaagcctgtcggagccaaatcttgcctgactggtgggtacagcaaccctccgccggacttccccttgtgacaggctatgccgcttacgatactcatcacagcgcttttagccagatggttatttccttctattatggaggaaaactggtcggccaagccacaaccacctgcctcgaggggtgtcgcttgagtttgagtcaacccggtcttcccaaactctatggccccgatgggcttgaacctgtctgctttcccactgctgatactattccctcagagagacaacgacaagtcacccgaaaattgtttggccacctcgagaggggagtactcttgcactctaacaggaagggtgtctttgtgaaacgcctctgtcaaggtagggtattctgttctggaaatgcagttgtttgcaaaggcaggcctaacagactggaacgggatgaagtcgtacaagtgttcgataccaatcagtttattcgggagttgcagcagttttacgctacacaaagtcgcctccctgacagtcgggttgtgttgtgcttcggggaggagtttcccgacactgtacccctccgaagcaaactcatactggtacaggtagaacaactttatgccaggcaactggtggaagaggccggtaagtcctgtggcgcaggatccctgatgccagccctggaagagccccagcctgaccaagcatttaggatgtttcccgacatttgtacctcacaccagaggccttttttccgcgaaaaccagcagataaccgtgtaa

SEQ ID NO: 40 - DNA, 2271 bp, Homo sapiens
atgagctggtcaccttccctgacaacgcagacatgtggggcctgggaaatgaaagagcgccttgggacagggggatttggaaatgtcatccgatggcacaatcaggaaacaggtgagcagattgccatcaagcagtgccggcaggagctcagcccccggaaccgagagcggtggtgcctggagatccagatcatgagaaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatga

SEQ ID NO: 41 - DNA, 2265 bp, Homo sapiens
atgttttcaggggggtgtcatagccccgggtttggccgccccagccccgccttccccgccccggggagcccgccccctgccccgcgtccctgccgacaggaaacaggtgagcagattgccatcaagcagtgccggcaggagctcagcccccggaaccgagagcggtggtgcctggagatccagatcatgagaaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatga

SEQ ID NO: 42 - DNA, 771 bp, Homo sapiens
atgagctggtcaccttccctgacaacgcagacatgtggggcctgggaaatgaaagagcgccttgggacagggggatttggaaatgtcatccgatggcacaatcaggaaacaggtgagcagattgccatcaagcagtgccggcaggagctcagcccccggaaccgagagcggtggtgcctggagatccagatcatgagaaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtgcgtaagaatgtggccgggtacagtggctcactcctgtaatcccagcactttgggaggccgaggcaggtggatcagttga

SEQ ID NO: 43 - DNA, 2094 bp, Homo sapiens
atgtcatccgatggcacaatcaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatga

SEQ ID NO: 44 - DNA, 2220 bp, Mus musculus
atgagctggtcaccgtccctcccaacccagacatgtggagcctgggaaatgaaagaacgcctggggaccgggggatttggaaacgtcatccggtggcacaatcaggcgacaggtgaacagatcgccatcaagcaatgccgacaggagctcagcccaaagaacagagaccgctggtgcctcgaaatccagatcatgagaaggctgaaccatcccaatgtggtggctgcccgggatgtcccagaggggatgcagaacctggcacccaatgatttgccactgctggccatggagtactgccaaggaggagatctccgaagatacttgaaccagttcgagaactgctgtggcctgcgggaaggagctgtccttaccctgctgagtgacatcgcatcggctcttagataccttcacgaaaacagaatcatccatcgagacctgaagccagaaaacatcgttctgcagcaaggagagaaaagattaatacacaaaattattgatctaggatatgccaaggagctggatcagggcagtctgtgcacgtcatttgtggggactctgcaatacctggcgccagagcttctggagcagcagaagtacaccgtgaccgttgactactggagcttcggcaccctggccttcgagtgcatcactggcttccggcccttcctccctaactggcagcctgtgcagtggcactccaaagtccggcagaagagcgaagtggacatcgttgttagtgaagacttgaatggagcagtgaagttttcaagttcgctacccttccccaataatcttaacagtgtcttggctgaacggctggagaagtggctgcagctgatgcttatgtggcaccctcggcaaaggggcacggatccccagtatggccccaacggctgcttcagagccctggatgacatcttgaacttgaagctggttcatgtcttgaacatggtcacaggcaccgttcacacataccccgtgacggaggatgagagtctgcagagcttaaaaaccagaatccaggaagacacggggatcctggagacagaccaggagctgctgcaagaggcagggctggtgctgctccctgacaagcctgctactcagtgcatctcagacagcaagacaaacgagggcctcacgttggacatggatcttgtttttctctttgacaacagtaaaatcaactatgagactcagatcaccccccgaccccaaccggaaagtgtcagctgtatccttcaggagcccaagcggaacctctccttcttccagctgaggaaagtgtggggccaagtctggcacagcatccagacgctgaaggaagactgtaaccggctgcagcagggacagcgagcagccatgatgagtctcctccggaataacagctgcctctctaagatgaagaacgccatggcctccacggcccagcagctcaaggccaagctggacttcttcaaaaccagcatccagatcgacctggagaagtataaagagcagaccgagtttgggatcacctcagataaattgctgctggcttggcgggagatggagcaggctgtggagcagtgtgggcgggagaatgacgtgaagcatctagtagagcggatgatggcactgcagactgacattgtggacctgcagaggagcccgatgggtcggaagcaggggggcaccctggatgacctagaggaacaagcgagggagctctaccgaagactcagggagaagccaagagaccaaaggacagaaggtgacagccaggagatggtacggctgctgcttcaggcaatccaaagctttgagaagaaagttcgggtgatttatacacagctcagtaagaccgtggtttgtaagcagaaggcactggagttgctgcccaaggtagaagaggtagtgagccttatgaacgaggacgagaggaccgtggtccggcttcaggagaagcggcagaaggaactctggaacctcctgaagatcgcctgtagcaaagtccgaggtcccgtgagtggaagcccagacagcatgaatgtgtctcgactcagtcaccctggtcagctaatgtcccagccttccagtgcctgtgacagcttacctgaatcagacaagaaaagtgaagaactggtggccgaagcccacgccctctgctcccggctagaaagtgcgctgcaggacactgtgaaggagcaagacagaagcttcacggtaaccgcctgataa

SEQ ID NO: 45 - protein, 11 aa, Artificial Sequence
  CDR1 of nanobody that binds CD206
SGNIFSINAIG

SEQ ID NO: 46 - protein, 9 aa, Artificial Sequence
  CDR2 of nanobody that binds CD206
TITLSGSTN

SEQ ID NO: 47 - protein, 11 aa, Artificial Sequence
  CDR3 of nanobody that binds CD206
NTYSDSDVYGY

SEQ ID NO: 48 - protein, 11 aa, Artificial Sequence
  CDR1 of nanobody that binds CD206
PGFKLDYYAIA

SEQ ID NO: 49 - protein, 9 aa, Artificial Sequence
  CDR2 of nanobody that binds CD206
SINSSGGST

SEQ ID NO: 50 - protein, 16 aa, Artificial Sequence
  CDR3 of nanobody that binds CD206
LRRYYGLNLDPGSYDY

SEQ ID NO: 51 - protein, 10 aa, Artificial Sequence
  CDR1 of nanobody that binds CD206
GFPFNIYPMS

SEQ ID NO: 52 - protein, 9 aa, Artificial Sequence
  CDR2 of nanobody that binds CD206
YISHGGTTT

SEQ ID NO: 53 - protein, 12 aa, Artificial Sequence
  CDR3 of nanobody that binds CD206
GYARLMTDSELV

SEQ ID NO: 54 - protein, 9 aa, Artificial Sequence
  CDRL1 of binding domain that binds CD163
ASQSVSHDV

SEQ ID NO: 55 - protein, 11 aa, Artificial Sequence
  CDRL1 of binding domain that binds mesothelin
RASEGIYHWLA

SEQ ID NO: 56 - protein, 8 aa, Artificial Sequence
  CDRL3 of binding domain that binds CD163
QDYSSPRT

SEQ ID NO: 57 - protein, 8 aa, Artificial Sequence
  CDRH1 of binding domain that binds CD163
GYSITSDY

SEQ ID NO: 58 - protein, 7 aa, Artificial Sequence
  CDRL2 of binding domain that binds mesothelin
KASSLAS

SEQ ID NO: 59 - protein, 12 aa, Artificial Sequence
  CDRH3 of binding domain that binds CD163
CVSGTYYFDYWG

SEQ ID NO: 60 - protein, 9 aa, Artificial Sequence
  CDRL1 of binding domain that binds CD163
ASQSVSSDV

SEQ ID NO: 61 - protein, 9 aa, Artificial Sequence
  CDRL3 of binding domain that binds mesothelin
QQYSNYPLT

SEQ ID NO: 62 - protein, 8 aa, Artificial Sequence
  CDRL3 of binding domain that binds CD163
QDYTSPRT

SEQ ID NO: 63 - protein, 10 aa, Artificial Sequence
  Gly-Ser linker
GGSGGGSGSG

SEQ ID NO: 64 - protein, 5 aa, Artificial Sequence
  CDRH1 of binding domain that binds mesothelin
TYYMQ

SEQ ID NO: 65 - protein, 8 aa, Artificial Sequence
  Gly-Ser linker
GGSGGGSG

SEQ ID NO: 66 - protein, 16 aa, Artificial Sequence
  CDRL1 of binding domain that binds CD23
RSSKSLLYKDGKTYLN

SEQ ID NO: 67 - protein, 7 aa, Artificial Sequence
  CDRL2 of binding domain that binds CD23
LMSTRAS

SEQ ID NO: 68 - protein, 9 aa, Artificial Sequence
  CDRL3 of binding domain that binds CD23
QQLVEYPFT

SEQ ID NO: 69 - protein, 5 aa, Artificial Sequence
  CDRH1 of binding domain that binds CD23
GYWMS

SEQ ID NO: 70 - protein, 19 aa, Artificial Sequence
  CDRH2 of binding domain that binds CD23
EIRLKSDNYATHYAESVKG

SEQ ID NO: 71 - protein, 16 aa, Artificial Sequence
  CDRH2 of binding domain that binds mesothelin
VINPSGVTSYAQKFQG

SEQ ID NO: 72 - protein, 8 aa, Artificial Sequence
  CDRL1 of binding domain that binds CD38
SSNIGDNY

SEQ ID NO: 73 - protein, 11 aa, Artificial Sequence
  CDRH3 of binding domain that binds mesothelin
WALWGDFGMDV

SEQ ID NO: 74 - protein, 10 aa, Artificial Sequence
  CDRL3 of binding domain that binds CD38
QSYDSSLSGS

SEQ ID NO: 75 - protein, 8 aa, Artificial Sequence
  CDRH1 of binding domain that binds CD38
GFTFDDYG

SEQ ID NO: 76 - protein, 8 aa, Artificial Sequence
  CDRH2 of binding domain that binds CD38
ISWNGGKT

SEQ ID NO: 77 - protein, 16 aa, Artificial Sequence
  CDRH3 of binding domain that binds CD38
ARGSLFHDSSGFYFGH

SEQ ID NO: 78 - protein, 8 aa, Artificial Sequence
  CDRL1 of binding domain that binds CD38
NSNIGSNT

SEQ ID NO: 79 - protein, 232 aa, Artificial Sequence
  VL of binding domain that binds mesothelin
MGWSCIILFLVATATGVHSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 11 aa, Artificial Sequence
  CDRL3 of binding domain that binds CD38
QSYDSSLSGSR

SEQ ID NO: 81 - protein, 8 aa, Artificial Sequence
  CDRH1 of binding domain that binds CD38
GFTFNNYG

SEQ ID NO: 82 - protein, 8 aa, Artificial Sequence
  CDRH2 of binding domain that binds CD38
ISYDGSDK

SEQ ID NO: 83 - protein, 15 aa, Artificial Sequence
  CDRH3 of binding domain that binds CD38
ARVYYYGFSGPSMDV

SEQ ID NO: 84 - protein, 11 aa, Artificial Sequence
  CDRL1 of binding domain that binds CD38, CD64, EGFR, PD-1, and TYRP1/gp75
RASQSVSSYLA

SEQ ID NO: 85 - protein, 7 aa, Artificial Sequence
  CDRL2 of binding domain that binds CD38, EGFR, PD-L1, PD-1, and TYRP1/gp75
DASNRAT

SEQ ID NO: 86 - protein, 10 aa, Artificial Sequence
  CDRL3 of binding domain that binds CD38
QQRSNWPPTF

SEQ ID NO: 87 - protein, 5 aa, Artificial Sequence
  CDRH1 of binding domain that binds CD38
SFAMS

SEQ ID NO: 88 - protein, 17 aa, Artificial Sequence
  CDRH2 of binding domain that binds CD38
AISGSGGGTYYADSVKG

SEQ ID NO: 89 - protein, 13 aa, Artificial Sequence
  CDRH3 of binding domain that binds CD38
DKILWFGEPVFDY

SEQ ID NO: 90 - protein, 10 aa, Artificial Sequence
  Gly-Ser linker
GGGGSGGGGS

SEQ ID NO: 91 - protein, 7 aa, Artificial Sequence
  CDRL2 of binding domain that binds CD64
DASSRAT

SEQ ID NO: 92 - protein, 10 aa, Artificial Sequence
  CDRL3 of binding domain that binds CD64
QLRSNWPPYT

SEQ ID NO: 93 - protein, 5 aa, Artificial Sequence
  CDRH1 of binding domain that binds CD64
GYGMH

SEQ ID NO: 94 - protein, 17 aa, Artificial Sequence
  CDRH2 of binding domain that binds CD64
VIWYDGSNKYYADSVKG

SEQ ID NO: 95 - protein, 9 aa, Artificial Sequence
  CDRH3 of binding domain that binds CD64
DTGDRFFDY

SEQ ID NO: 96 - DNA, 20 bp, Artificial Sequence
  SerpinB2 forward primer
actggggcagttatgacagg

SEQ ID NO: 97 - DNA, 19 bp, Artificial Sequence
  SerpinB2 reverse primer
gatgatcggccacaaactg

SEQ ID NO: 98 - DNA, 21 bp, Artificial Sequence
  Retnla forward primer
ttgttcccttctcatctgcat

SEQ ID NO: 99 - DNA, 21 bp, Artificial Sequence
  Retnla reverse primer
ccttgaccttattctccacga

SEQ ID NO: 100 - DNA, 19 bp, Artificial Sequence
  Ccl5 forward primer
cctactcccactcggtcct

SEQ ID NO: 101 - DNA, 21 bp, Artificial Sequence
  Ccl5 reverse primer
ctgatttcttgggtttgctgt

SEQ ID NO: 102 - DNA, 18 bp, Artificial Sequence
  Ccl11 forward primer
agagctccacagcgcttc

SEQ ID NO: 103 - DNA, 18 bp, Artificial Sequence
  Ccl11 reverse primer
cagcacctgggaggtgaa

SEQ ID NO: 104 - DNA, 26 bp, Artificial Sequence
  codon-optimized IRF5 forward primer
tcttaaagaccacatggtagaacagt

SEQ ID NO: 105 - DNA, 18 bp, Artificial Sequence
  codon-optimized IRF5 reverse primer
agctgctgttgggattgc

SEQ ID NO: 106 - DNA, 20 bp, Artificial Sequence
  endogenous IRF5 forward primer
gctgtgcccttaacaaaagc

SEQ ID NO: 107 - DNA, 18 bp, Artificial Sequence
  endogenous IRF5 reverse primer
ggctgaggtggcatgtct

SEQ ID NO: 108 - DNA, 19 bp, Artificial Sequence
  GAPD forward primer
agccacatcgctcagacac

SEQ ID NO: 109 - DNA, 19 bp, Artificial Sequence
  GAPD reverse primer
gcccaatacgaccaaatcc

SEQ ID NO: 110 - protein, 134 aa, Homo sapiens
MNTEMYQTPMEVAVYQLHNFSISFFSSLLGGDVVSVKLDNSASGASVVAIDNKIEQAMDLVKNHLMYAVREEVEILKEQIRELVEKNSQLERENTLLKTLASPEQLEKFQSCLSPEEPAPESPQVPEAPGGSAV

SEQ ID NO: 111 - DNA, 405 bp, Homo sapiens
atgaacaccgaaatgtatcagacccccatggaggtggcggtctaccagctgcacaatttctccatctccttcttctcttctctgcttggaggggatgtggtttccgttaagctggacaacagtgcctccggagccagcgtggtggccatagacaacaagatcgaacaggccatggatctggtgaagaatcatctgatgtatgctgtgagagaggaggtggagatcctgaaggagcagatccgagagctggtggagaagaactcccagctagagcgtgagaacaccctgttgaagaccctggcaagcccagagcagctggagaagttccagtcctgtctgagccctgaagagccagctcccgaatccccacaagtgcccgaggcccctggtggttctgcggtgtaa

SEQ ID NO: 112 - protein, 468 aa, Artificial Sequence
  VH of binding domain that binds mesothelin
MGWSCIILFLVATATGVHSQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 232 aa, Artificial Sequence
  VL of binding domain that binds mesothelin
MGWSCIILFLVATATGVHSEIVLTQSPATLSLSPGERATLSCSASSSVSYMHWYQQKPGQAPRLLIYDTSKLASGVPARFSGSGSGTDFTLTISSLEPEDFAVYYCQQWSKHPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 468 aa, Artificial Sequence
  VH of binding domain that binds mesothelin
MGWSCIILFLVATATGVHSQVQLVQSGAEVKKPGASVKVSCKASGYSFTGYTMNWVRQAPGQGLEWMGLITPYNGASSYNQKFRGRVTMTRDTSTSTVYMELSSLRSEDTAVYYCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 214 aa, Artificial Sequence
  VL of binding domain that binds MUC16
DIQMTQSPSSLSASVGDRVTITGRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 450 aa, Artificial Sequence
  VH of binding domain that binds MUC16
EVQLVESGGGLVQPGGSLRLSCAASGFNIKDTYIHWVHQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYIGNVNHKPSNTKVDKKVEPKSCDKTHTGPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 7 aa, Artificial Sequence
  CDRL1 of binding domain that binds MUC16
SEDIYSG

SEQ ID NO: 118 - protein, 8 aa, Artificial Sequence
  CDRL3 of binding domain that binds MUC16
GYSYSSTL

SEQ ID NO: 119 - protein, 7 aa, Artificial Sequence
  CDRH1 of binding domain that binds MUC16
TLGMGVG

SEQ ID NO: 120 - protein, 16 aa, Artificial Sequence
  CDRH2 of binding domain that binds MUC16
HIWWDDDKYYNPALKS

SEQ ID NO: 121 - protein, 12 aa, Artificial Sequence
  CDRH3 of binding domain that binds MUC16
IGTAQATDALDY

SEQ ID NO: 122 - protein, 15 aa, Artificial Sequence
  CDRL1 of binding domain that binds folate receptor
KASQSVSFAGTSLMH

SEQ ID NO: 123 - protein, 7 aa, Artificial Sequence
  CDRL2 of binding domain that binds folate receptor
RASNLEA

SEQ ID NO: 124 - protein, 9 aa, Artificial Sequence
  CDRL3 of binding domain that binds folate receptor
QQSREYPYT

SEQ ID NO: 125 - protein, 5 aa, Artificial Sequence
  CDRH1 of binding domain that binds folate receptor
GYFMN

SEQ ID NO: 126 - protein, 17 aa, Artificial Sequence
  CDRH2 of binding domain that binds folate receptor
RIHPYDGDTFYNQKFQG

SEQ ID NO: 127 - protein, 9 aa, Artificial Sequence
  CDRH3 of binding domain that binds folate receptor
YDGSRAMDY

SEQ ID NO: 128 - protein, 8 aa, Artificial Sequence
  Gly-Ser linker
GGGSGGGS

SEQ ID NO: 129 - protein, 6 aa, Artificial Sequence
  Gly-Ser linker
GGSGGS

SEQ ID NO: 130 - protein, 9 aa, Artificial Sequence
  CDRL3 of binding domain that binds EGFR
HQYGSTPLT

SEQ ID NO: 131 - protein, 7 aa, Artificial Sequence
  CDRH1 of binding domain that binds EGFR
SGDYYWS

SEQ ID NO: 132 - protein, 16 aa, Artificial Sequence
  CDRH2 of binding domain that binds EGFR
YIYYSGSTDYNPSLKS

SEQ ID NO: 133 - protein, 11 aa, Artificial Sequence
  CDRH3 of binding domain that binds EGFR
VSIFGVGTFDY

SEQ ID NO: 134 - protein, 107 aa, Artificial Sequence
  VL of binding domain that binds EGFR
EIVLTQSPDFQSVTPKEKVTITCRASYSIGTNIHWYQQKPDQSPKLLIKYASESISGVPSRFSGSGSGTDFTLTINSLEAEDAATYYCQQNNNWPTTFGGGTKVEIK

SEQ ID NO: 135 - protein, 119 aa, Artificial Sequence
  VH of binding domain that binds EGFR
QVTLKESGPVLVKPTETLTLTCTVSGFSLSNWDVHWIRQPPGKALEWLAVIWSGGATDYNTPFNSRLTISKDTSKSQVVLTMTNMDPVDTATYYCARALDYYDYNFAYWGQGTMVTVSS

SEQ ID NO: 136 - protein, 11 aa, Artificial Sequence
  CDRL1 of binding domain that binds CD19
RASQDISKYLN

SEQ ID NO: 137 - protein, 7 aa, Artificial Sequence
  CDRL2 of binding domain that binds CD19
SRLHSGV

SEQ ID NO: 138 - protein, 9 aa, Artificial Sequence
  CDRL3 of binding domain that binds CD19
GNTLPYTFG

SEQ ID NO: 139 - protein, 5 aa, Artificial Sequence
  CDRH1 of binding domain that binds CD19
DYGVS

SEQ ID NO: 140 - protein, 16 aa, Artificial Sequence
  CDRH2 of binding domain that binds CD19
VTWGSETTYYNSALKS

SEQ ID NO: 141 - protein, 7 aa, Artificial Sequence
  CDRH3 of binding domain that binds CD19
YAMDYWG

SEQ ID NO: 142 - protein, 11 aa, Artificial Sequence
  CDRL1 of binding domain that binds ROR1
QASQSIDSNLA

SEQ ID NO: 143 - protein, 7 aa, Artificial Sequence
  CDRL2 of binding domain that binds ROR1
RASNLAS

SEQ ID NO: 144 - protein, 12 aa, Artificial Sequence
  CDRL3 of binding domain that binds ROR1
LGGVGNVSYRTS

SEQ ID NO: 145 - protein, 5 aa, Artificial Sequence
  CDRH1 of binding domain that binds ROR1
DYPIS

SEQ ID NO: 146 - protein, 16 aa, Artificial Sequence
  CDRH2  of binding domain that binds ROR1
FINSGGSTWYASWVKG

SEQ ID NO: 147 - protein, 11 aa, Artificial Sequence
  CDRH3 of binding domain that binds ROR1
GYSTYYCDFNI

SEQ ID NO: 148 - protein, 12 aa, Artificial Sequence
  CDRL1 of binding domain that binds ROR1
TLSSAHKTDTID

SEQ ID NO: 149 - protein, 7 aa, Artificial Sequence
  CDRL2 of binding domain that binds ROR1
GSYTKRP

SEQ ID NO: 150 - protein, 9 aa, Artificial Sequence
  CDRL3 of binding domain that binds ROR1
GADYIGGYV

SEQ ID NO: 151 - protein, 5 aa, Artificial Sequence
  CDRH1 of binding domain that binds ROR1
AYYMS

SEQ ID NO: 152 - protein, 17 aa, Artificial Sequence
  CDRH2 of binding domain that binds ROR1
TIYPSSGKTYYATWVNG

SEQ ID NO: 153 - protein, 12 aa, Artificial Sequence
  CDRH3 of binding domain that binds ROR1
DSYADDGALFNI

SEQ ID NO: 154 - protein, 11 aa, Artificial Sequence
  CDRL1 of binding domain that binds ROR1
KASQNVDAAVA

SEQ ID NO: 155 - protein, 7 aa, Artificial Sequence
  CDRL2 of binding domain that binds ROR1
SASNRYT

SEQ ID NO: 156 - protein, 9 aa, Artificial Sequence
  CDRL3 of binding domain that binds ROR1
QQYDIYPYT

SEQ ID NO: 157 - protein, 5 aa, Artificial Sequence
  CDRH1 of binding domain that binds ROR1
DYEMH

SEQ ID NO: 158 - protein, 17 aa, Artificial Sequence
  CDRH2 of binding domain that binds ROR1
AIDPETGGTAYNQKFKG

SEQ ID NO: 159 - protein, 9 aa, Artificial Sequence
  CDRH3 of binding domain that binds ROR1
YYDYDSFTY

SEQ ID NO: 160 - protein, 11 aa, Artificial Sequence
  CDRL1 of binding domain that binds ROR1
QASQSIGSYLA

SEQ ID NO: 161 - protein, 7 aa, Artificial Sequence
  CDRL2 of binding domain that binds ROR1
YASNLAS

SEQ ID NO: 162 - protein, 10 aa, Artificial Sequence
  CDRL3 of binding domain that binds ROR1
LGSLSNSDNV

SEQ ID NO: 163 - protein, 5 aa, Artificial Sequence
  CDRH1 of binding domain that binds ROR1
SHWMS

SEQ ID NO: 164 - protein, 16 aa, Artificial Sequence
  CDRH2 of binding domain that binds ROR1
IIAASGSTYYANWAKG

SEQ ID NO: 165 - protein, 12 aa, Artificial Sequence
  CDRH3 of binding domain that binds ROR1
DYGDYRLVTFNI

SEQ ID NO: 166 - protein, 12 aa, Artificial Sequence
  CDRL1 of binding domain that binds Her2
RASQDVNTAVAW

SEQ ID NO: 167 - protein, 8 aa, Artificial Sequence
  CDRL2 of binding domain that binds Her2
YSASFLES

SEQ ID NO: 168 - protein, 8 aa, Artificial Sequence
  CDRL3 of binding domain that binds Her2
QQHYTTPT

SEQ ID NO: 169 - protein, 12 aa, Artificial Sequence
  CDRH1 of binding domain that binds Her2
SGFNTKDTYIHW

SEQ ID NO: 170 - protein, 18 aa, Artificial Sequence
  CDRH2 of binding domain that binds Her2
RIYPTNGYTRYADSVKGR

SEQ ID NO: 171 - protein, 11 aa, Artificial Sequence
  CDRH3 of binding domain that binds Her2
WGGDGFYAMDV

SEQ ID NO: 172 - protein, 10 aa, Artificial Sequence
  CDRL1 of binding domain that binds PD-L1
RASQSVSSYL

SEQ ID NO: 173 - protein, 9 aa, Artificial Sequence
  CDRL3 of binding domain that binds PD-L1
QQRSNWPRT

SEQ ID NO: 174 - protein, 5 aa, Artificial Sequence
  CDRH1 of binding domain that binds PD-L1
DYGFS

SEQ ID NO: 175 - protein, 17 aa, Artificial Sequence
  CDRH2 of binding domain that binds PD-L1
WITAYNGNTNYAQKLQG

SEQ ID NO: 176 - protein, 8 aa, Artificial Sequence
  CDRH3 of binding domain that binds PD-L1
DYFYGMDY

SEQ ID NO: 177 - protein, 11 aa, Artificial Sequence
  CDRL1 of binding domain that binds PD-L1
RASQDVSTAVA

SEQ ID NO: 178 - protein, 7 aa, Artificial Sequence
  CDRL2 of binding domain that binds PD-L1
SASFLYS

SEQ ID NO: 179 - protein, 9 aa, Artificial Sequence
  CDRL3 of binding domain that binds PD-L1
QQYLYHPAT

SEQ ID NO: 180 - protein, 11 aa, Artificial Sequence
  CDRH1 of binding domain that binds PD-L1
SGFTFSDSWIH

SEQ ID NO: 181 - protein, 17 aa, Artificial Sequence
  CDRH2 of binding domain that binds PD-L1
WISPYGGSTYYADSVKG

SEQ ID NO: 182 - protein, 9 aa, Artificial Sequence
  CDRH3 of binding domain that binds PD-L1
RHWPGGFDY

SEQ ID NO: 183 - protein, 14 aa, Artificial Sequence
  CDRL1 of binding domain that binds PD-L1
TGTSSDVGGYNYVS

SEQ ID NO: 184 - protein, 7 aa, Artificial Sequence
  CDRL2 of binding domain that binds PD-L1
DVSNRPS

SEQ ID NO: 185 - protein, 10 aa, Artificial Sequence
  CDRL3 of binding domain that binds PD-L1
SSYTSSSTRV

SEQ ID NO: 186 - protein, 11 aa, Artificial Sequence
  CDRH1 of binding domain that binds PD-L1
SGFTFSSYIMM

SEQ ID NO: 187 - protein, 17 aa, Artificial Sequence
  CDRH2 of binding domain that binds PD-L1
SIYPSGGITFYADTVKG

SEQ ID NO: 188 - protein, 11 aa, Artificial Sequence
  CDRH3 of binding domain that binds PD-L1
IKLGTVTTVDY

SEQ ID NO: 189 - protein, 10 aa, Artificial Sequence
  CDRL1 of binding domain that binds CD3
SASSSVSYMN

SEQ ID NO: 190 - protein, 11 aa, Artificial Sequence
  CDRL2 of binding domain that binds CD3
RWIYDTSKLAS

SEQ ID NO: 191 - protein, 9 aa, Artificial Sequence
  CDRL3 of binding domain that binds CD3
QQWSSNPFT

SEQ ID NO: 192 - protein, 13 aa, Artificial Sequence
  CDRH1 of binding domain that binds CD3
KASGYTFTRYTMH

SEQ ID NO: 193 - protein, 16 aa, Artificial Sequence
  CDRH2 of binding domain that binds CD3
INPSRGYTNYNQKFKD

SEQ ID NO: 194 - protein, 10 aa, Artificial Sequence
  CDRH3 of binding domain that binds CD3
YYDDHYCLDY

SEQ ID NO: 195 - protein, 242 aa, Artificial Sequence
  scFv that binds CD3
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 196 - protein, 11 aa, Artificial Sequence
  CDRL1 of binding domain that binds CD3
QSLVHNNGNTY

SEQ ID NO: 197 - protein, 9 aa, Artificial Sequence
  CDRL3 of binding domain that binds CD3
GQGTQYPFT

SEQ ID NO: 198 - protein, 8 aa, Artificial Sequence
  CDRH1 of binding domain that binds CD3
GFTFTKAW

SEQ ID NO: 199 - protein, 10 aa, Artificial Sequence
  CDRH2 of binding domain that binds CD3
IKDKSNSYAT

SEQ ID NO: 200 - protein, 12 aa, Artificial Sequence
  CDRH3 of binding domain that binds CD3
RGVYYALSPFDY

SEQ ID NO: 201 - protein, 11 aa, Artificial Sequence
  CDRL1 of binding domain that binds CD3
QSLVHDNGNTY

SEQ ID NO: 202 - protein, 8 aa, Artificial Sequence
  CDRH1 of binding domain that binds CD3
GFTFSNAW

SEQ ID NO: 203 - protein, 10 aa, Artificial Sequence
  CDRH2 of binding domain that binds CD3
IKARSNNYAT

SEQ ID NO: 204 - protein, 12 aa, Artificial Sequence
  CDRH3 of binding domain that binds CD3
RGTYYASKPFDY

SEQ ID NO: 205 - protein, 11 aa, Artificial Sequence
  CDRL1 of binding domain that binds CD3
QSLEHNNGNTY

SEQ ID NO: 206 - protein, 10 aa, Artificial Sequence
  CDRH2 of binding domain that binds CD3
IKDKSNNYAT

SEQ ID NO: 207 - protein, 13 aa, Artificial Sequence
  CDRH3 of binding domain that binds CD3
RYVHYGIGYAMDA

SEQ ID NO: 208 - protein, 11 aa, Artificial Sequence
  CDRL1 of binding domain that binds CD3
QSLVHTNGNTY

SEQ ID NO: 209 - protein, 9 aa, Artificial Sequence
  CDRL3 of binding domain that binds CD3
GQGTHYPFT

SEQ ID NO: 210 - protein, 8 aa, Artificial Sequence
  CDRH1 of binding domain that binds CD3
GFTFTNAW

SEQ ID NO: 211 - protein, 9 aa, Artificial Sequence
  CDRH2 of binding domain that binds CD3
KDKSNNYAT

SEQ ID NO: 212 - protein, 13 aa, Artificial Sequence
  CDRH3 of binding domain that binds CD3
RYVHYRFAYALDA

SEQ ID NO: 213 - protein, 11 aa, Artificial Sequence
  CDRL1 of binding domain that binds CD28
HASQNIYVWLN

SEQ ID NO: 214 - protein, 7 aa, Artificial Sequence
  CDRL2 of binding domain that binds CD28
KASNLHT

SEQ ID NO: 215 - protein, 9 aa, Artificial Sequence
  CDRL3 of binding domain that binds CD28
QQGQTYPYT

SEQ ID NO: 216 - protein, 5 aa, Artificial Sequence
  CDRH1 of binding domain that binds CD28
SYYIH

SEQ ID NO: 217 - protein, 17 aa, Artificial Sequence
  CDRH2 of binding domain that binds CD28
CIYPGNVNTNYNEKFKD

SEQ ID NO: 218 - protein, 11 aa, Artificial Sequence
  CDRH3 of binding domain that binds CD28
SHYGLDWNFDV

SEQ ID NO: 219 - protein, 12 aa, Artificial Sequence
  CDRL1 of binding domain that binds CD80/CD86
SVSSSISSSNLH

SEQ ID NO: 220 - protein, 7 aa, Artificial Sequence
  CDRL2 of binding domain that binds CD80/CD86
GTSNLAS

SEQ ID NO: 221 - protein, 9 aa, Artificial Sequence
  CDRL3 of binding domain that binds CD80/CD86
QQWSSYPLT

SEQ ID NO: 222 - protein, 5 aa, Artificial Sequence
  CDRH1 of binding domain that binds CD80/CD86
DYYMH

SEQ ID NO: 223 - protein, 17 aa, Artificial Sequence
  CDRH2 of binding domain that binds CD80/CD86
WIDPENGNTLYDPKFQG

SEQ ID NO: 224 - protein, 7 aa, Artificial Sequence
  CDRH3 of binding domain that binds CD80/CD86
EGLFFAY

SEQ ID NO: 225 - protein, 7 aa, Artificial Sequence
  CDRL1 of binding domain that binds 4-1BB
RASQSVS

SEQ ID NO: 226 - protein, 6 aa, Artificial Sequence
  CDRL2 of binding domain that binds 4-1BB
ASNRAT

SEQ ID NO: 227 - protein, 10 aa, Artificial Sequence
  CDRL3 of binding domain that binds 4-1BB
QRSNWPPALT

SEQ ID NO: 228 - protein, 4 aa, Artificial Sequence
  CDRH1 of binding domain that binds 4-1BB
YYWS

SEQ ID NO: 229 - protein, 12 aa, Artificial Sequence
  CDRH3 of binding domain that binds 4-1BB
YGPGNYDWYFDL

SEQ ID NO: 230 - protein, 11 aa, Artificial Sequence
  CDRL1 of binding domain that binds 4-1BB
SGDNIGDQYAH

SEQ ID NO: 231 - protein, 7 aa, Artificial Sequence
  CDRL2 of binding domain that binds 4-1BB
QDKNRPS

SEQ ID NO: 232 - protein, 11 aa, Artificial Sequence
  CDRL3 of binding domain that binds 4-1BB
ATYTGFGSLAV

SEQ ID NO: 233 - protein, 10 aa, Artificial Sequence
  CDRH1 of binding domain that binds 4-1BB
GYSFSTYWIS

SEQ ID NO: 234 - protein, 14 aa, Artificial Sequence
  CDRH2 of binding domain that binds 4-1BB
KIYPGDSYTNYSPS

SEQ ID NO: 235 - protein, 7 aa, Artificial Sequence
  CDRH3 of binding domain that binds 4-1BB
GYGIFDY

SEQ ID NO: 236 - protein, 11 aa, Artificial Sequence
  CDRL1 of binding domain that binds CD8
RTSRSISQYLA

SEQ ID NO: 237 - protein, 7 aa, Artificial Sequence
  CDRL2 of binding domain that binds CD8
SGSTLQS

SEQ ID NO: 238 - protein, 9 aa, Artificial Sequence
  CDRL3 of binding domain that binds CD8
QQHNENPLT

SEQ ID NO: 239 - protein, 6 aa, Artificial Sequence
  CDRH1 of binding domain that binds CD8
GFNIKD

SEQ ID NO: 240 - protein, 9 aa, Artificial Sequence
  CDRH2 of binding domain that binds CD8
RIDPANDNT

SEQ ID NO: 241 - protein, 9 aa, Artificial Sequence
  CDRH3 of binding domain that binds CD8
GYGYYVFDH

SEQ ID NO: 242 - protein, 15 aa, Artificial Sequence
  CDRL1 of binding domain that binds PD-1
RASQSVSTSGYSYMH

SEQ ID NO: 243 - protein, 7 aa, Artificial Sequence
  CDRL2 of binding domain that binds PD-1
FGSNLES

SEQ ID NO: 244 - protein, 9 aa, Artificial Sequence
  CDRL3 of binding domain that binds PD-1
QHSWEIPYT

SEQ ID NO: 245 - protein, 5 aa, Artificial Sequence
  CDRH1 of binding domain that binds PD-1
SSWIH

SEQ ID NO: 246 - protein, 17 aa, Artificial Sequence
  CDRH2 of binding domain that binds PD-1
YIYPSTGFTEYNQKFKD

SEQ ID NO: 247 - protein, 12 aa, Artificial Sequence
  CDRH3 of binding domain that binds PD-1
WRDSSGYHAMDY

SEQ ID NO: 248 - protein, 9 aa, Artificial Sequence
  CDRL3 of binding domain that binds PD-1
QQSSNWPRT

SEQ ID NO: 249 - protein, 5 aa, Artificial Sequence
  CDRH1 of binding domain that binds PD-1
NSGMH

SEQ ID NO: 250 - protein, 17 aa, Artificial Sequence
  CDRH2 of binding domain that binds PD-1
VLWYDGSKRYYADSVKG

SEQ ID NO: 251 - protein, 4 aa, Artificial Sequence
  CDRH3 of binding domain that binds PD-1
NDDY

SEQ ID NO: 252 - protein, 11 aa, Artificial Sequence
  Gly-Ser linker
GGSGGGSGGSG

SEQ ID NO: 253 - protein, 10 aa, Artificial Sequence
  CDRL3 of binding domain that binds TYRP1/gp75
QQRSNWLMYT

SEQ ID NO: 254 - protein, 10 aa, Artificial Sequence
  CDRH1 of binding domain that binds TYRP1/gp75
GYTFTSYAMN

SEQ ID NO: 255 - protein, 17 aa, Artificial Sequence
  CDRH2 of binding domain that binds TYRP1/gp75
WINTNTGNPTYAQGFTG

SEQ ID NO: 256 - protein, 10 aa, Artificial Sequence
  CDRH3 of binding domain that binds TYRP1/gp75
RYSSSWYLDY

SEQ ID NO: 257 - protein, 11 aa, Artificial Sequence
  CDRL1 of binding domain that binds TYRP1/gp75
RASGNIYNYLA

SEQ ID NO: 258 - protein, 7 aa, Artificial Sequence
  CDRL2 of binding domain that binds TYRP1/gp75
DAKTLAD

SEQ ID NO: 259 - protein, 9 aa, Artificial Sequence
  CDRL3 of binding domain that binds TYRP1/gp75
QHFWSLPFT

SEQ ID NO: 260 - protein, 10 aa, Artificial Sequence
  CDRH1 of binding domain that binds TYRP1/gp75
GFNIKDYFLH

SEQ ID NO: 261 - protein, 17 aa, Artificial Sequence
  CDRH2 of binding domain that binds TYRP1/gp75
WINPDNGNTVYDPKFQG

SEQ ID NO: 262 - protein, 11 aa, Artificial Sequence
  CDRH3 of binding domain that binds TYRP1/gp75
DYTYEKAALDY

SEQ ID NO: 263 - protein, 108 aa, Artificial Sequence
  VL of binding domain that binds TYRP1/gp75
EIVLTQSPATLSLSPGERATLSCRASQSVSSYLAWYQQKPGQAPRLLIYDASNRATGIPARFSGSGSGTDFTLTISSLEPEDFAVYYCQQRSNWLMYTFGQGTKLEIK

SEQ ID NO: 264 - protein, 119 aa, Artificial Sequence
  VH of binding domain that binds TYRP1/gp75
QVQLVQSGSELKKPGASVKISCKASGYTFTSYAMNWVRQAPGQGLECMGWINTNTGNPTYAQGFTGRFVFSMDTSVSTAYLQISSLKAEDTAIYYCAPRYSSSWYLDYWGQGTLVTVSS

SEQ ID NO: 265 - protein, 119 aa, Artificial Sequence
  VH of binding domain that binds TYRP1/gp75
QVQLVQSGSELKKPGASVKISCKASGYTFTSYAMNWVRQAPGQGLESMGWINTNTGNPTYAQGFTGRFVFSMDTSVSTAYLQISSLKAEDTAIYYCAPRYSSSWYLDYWGQGTLVTVSS